(19) United States
(12) Patent Application Publication
(10) Pub. No.: US 2015/0197764 A1
(43) Pub. Date: Jul. 16, 2015

(54) TRANSGENIC PLANTS WITH ENHANCED AGRONOMIC TRAITS

(71) Applicant: MONSANTO TECHNOLOGY LLC, St. Louis, MO (US)

(72) Inventors: Eric D. Aasen, Woodland, CA (US); Mark Scott Abad, Webster Groves, MO (US); Edwards Allen, O'Fallon, MO (US); Thandoni Rao Ambika, Karnataka (IN); Veena S. Anil, Bangalore (IN); Alice Clara Augustine, Karnataka (IN); Stephanie L. Back, St. Louis, MO (US); Pranesh Badami, Bangalore (IN); Amarjit Basra, Chesterfield, MO (US); Kraig L. Brustad, Saunderstown, RI (US); Molian Deng, Grover, MO (US); Todd Dezwaan, Apex, NC (US); Charles Dietrich, Chesterfield, MO (US); Stephen M. Duff, St. Louis, MO (US); Karen K. Gabbert, St. Louis, MO (US); Steve He, St. Louis, MO (US); Bill L. Hendrix, West Sacramento, CA (US); Hongwu Jia, Creve Coeur, MO (US); Vidya Kammaradi Ramanath Bhat, Karnataka (IN); Balasulojini Karunanandaa, Creve Coeur, MO (US); Kevin R. Kosola, Wildwood, MO (US); Kempagangaiah Krishnamurthy, Karnataka (IN); Ganesh Kumar, Chesterfield, MO (US); Saritha V. Kuriakose, Kerela (IN); Shantala Lakkanna, Bangalore (IN); Mark E. Leibman, St. Louis, MO (US); Linda Lutfiyya, St. Louis, MO (US); Savitha Madappa, Karnataka (IN); Sivalinganna Manjunath, Chesterfield, MO (US); Robert J. Meister, St. Peters, MO (US); Bellur Narasimha Prasad, Bangalore (IN); Sasha Preuss, Webster Groves, MO (US); Qungang Qi, Chesterfield, MO (US); Dhanalakshmi Ramachandra, Bangalore (IN); Aniruddha Raychaudhuri, Wildwood, MO (US); S. Sangeetha, Karnataka (IN); Scott Saracco, St. Louis, MO (US); Chitresh Sharma, Himachal Pradesh (IN); Vijay K. Sharma, Wildwood, MO (US); Rajani Monnanda Somaiah, St. Louis, MO (US); Padmini Sudarshana, Bangalore (IN); Rebecca L. Thompson-Mize, St. Charles, MO (US); Srikanth Venkatachalayya, Karnataka (IN); Tyamagondlu V. Venkatesh, St. Louis, MO (US); Huai Wang, Chesterfield, MO (US); Jingrui Wu, Johnston, IA (US); Xiaoyun Wu, Chesterfield, MO (US); Xiaofeng Sean Yang, Chesterfield, MO (US); Qin Zeng, Maryland Heights, MO (US); Jianmin Zhao, St. Louis, MO (US)

(73) Assignee: MONSANTO TECHNOLOGY LLC, St. Louis, MO (US)

(21) Appl. No.: 14/632,018

(22) Filed: Feb. 26, 2015

Related U.S. Application Data
(63) Continuation of application No. 12/998,243, filed on Sep. 16, 2011.
(60) Provisional application No. 61/226,953, filed on Jul. 20, 2009.
(60) Provisional application No. 61/182,785, filed on Jun. 1, 2009.
(60) Provisional application No. 61/164,664, filed on Mar. 30, 2009.
(60) Provisional application No. 61/155,950, filed on Feb. 27, 2009.
(60) Provisional application No. 61/148,438, filed on Jan. 30, 2009.
(60) Provisional application No. 61/139,164, filed on Dec. 19, 2008.
(60) Provisional application No. 61/101,722, filed on Oct. 1, 2008.

(51) Int. Cl. C12N 15/82

(52) U.S. Cl. CPC C12N 15/8251; C12N 15/8273; C12N 15/8261; C12N 15/8247; C12N 15/8274; C12N 15/8275

(57) ABSTRACT

This invention provides transgenic plant cells with recombinant DNA for expression of proteins that are useful for imparting enhanced agronomic trait(s) to transgenic crop plants. This invention also provides transgenic plants and progeny seed comprising the transgenic plant cells where the plants are selected for having an enhanced trait selected from the group of traits consisting of enhanced water use efficiency, enhanced cold tolerance, increased yield, enhanced nitrogen use efficiency, enhanced seed protein and enhanced seed oil. Also disclosed are methods for manufacturing transgenic seed and plants with enhanced trait.

### CROSS REFERENCE TO RELATED APPLICATIONS

This application claims benefit under 35 USC §119(e) of U.S. provisional application Ser. Nos. 61/101,722, filed Oct. 1, 2008, 61/139,164, filed Dec. 19, 2008, 61/148,438, filed Jan. 30, 2009, 61/155,950, filed Feb. 27, 2009, 61/164,664, filed Mar. 30, 2009, 61/182,785, filed Jun. 1, 2009 and 61/226953, filed Jul. 20, 2009, each of which is incorporated herein by reference in its entirety.

### INCORPORATION OF SEQUENCE LISTING

The sequence listing file named “56202_B_seq_listing.txt”, which is 138,581,829 bytes (measured in MS-WINDOWS) which was electronically filed on a CD-ROM which was created on Sep. 28, 2009 is incorporated herein by reference.

### FIELD OF THE INVENTION

Disclosed herein are recombinant DNA useful for providing enhanced traits to transgenic plants, seeds, pollen, plant cells and plant nuclei of such transgenic plants, methods of making and using such recombinant DNA, plants, seeds, pollen, plant cells and plant nuclei. Also disclosed are methods of producing hybrid corn seed comprising such recombinant DNA.

All genetic resources disclosed herein were directly obtained from sources that are currently common to the United States; the ancestral sources of each specific genetic material is unknown.

### SUMMARY OF THE INVENTION

An aspect of this invention provides recombinant DNA constructs comprising polynucleotides characterized by an encoded protein having amino acids representing a protein family domain module as described in Table 10. Another aspect of this invention provides recombinant DNA constructs comprising polynucleotides characterized by an encoded protein with an amino acid sequence that is at least 90% identical to a corresponding consensus sequence defined in table 8. Yet another aspect of this invention provides recombinant DNA constructs comprising polynucleotides characterized by reference to SEQ ID NO:1-307 and the cognate proteins with amino acid sequences having reference to SEQ ID NO:308-614. The recombinant DNA constructs are useful for providing enhanced traits when stably integrated into the chromosomes and expressed in the nuclei of transgenic plants cells. In some aspects of the invention the recombinant DNA constructs, when expressed in a plant cell, provide for expression of cognate proteins. In those aspects of the invention, the recombinant DNA constructs for expressing cognate proteins are characterized by cognate amino acid sequences having a sequence selected from SEQ ID NOs: 308, 310, 312-315, 317-323, 325-343, 345, 347-349, 352-354, 356, 358-359, 366-372, 374-383, 389-392, 394, 396, 401-403, 405-412, 414, 417-424, 427-453, 455-473, 475, 488-501, 503-517, 519-531, 533-540, 542-543, and 546-614; having at least 90% identity over at least 90% of the length of a sequence selected from the group consisting of SEQ ID NOs: 308, 310, 312-315, 317-323, 325-343, 345, 347-349, 352-354, 356, 358-359, 366-372, 374-383, 389-392, 394, 396, 401-403, 405-412, 414, 417-424, 427-453, 455-473, 475, 488-501, 503-517, 519-531, 533-540, 542-543, and 546-614 or that are homologous to a sequence selected from the group consisting of SEQ ID NOs: 308, 310, 312-315, 317-323, 325-343, 345, 347-349, 352-354, 356, 358-359, 366-372, 374-383, 389-392, 394, 396, 401-403, 405-412, 414, 417-424, 427-453, 455-473, 475, 488-501, 503-517, 519-531, 533-540, 542-543, and 546-614.

In other aspects of the invention the recombinant DNA constructs provide for suppression of a native protein. In those other aspects of the invention the recombinant DNA constructs are characterized as being constructed with sense-oriented and anti-sense-oriented polynucleotides, e.g. polynucleotides derived from genes having SEQ ID NOs: 2, 4, 9, 17, 37, 39, 43-44, 48, 50, 53-58, 66, 77-81, 86, 88, 90-93, 97, 106, 108-109, 118-119, 147, 167, 169-180, 195, 211, 225, 234, or 237-238 or homologous genes. When the recombinant DNA construct is expressed in corn plants, the endogenous protein is a corn protein with an amino acid sequence of SEQ ID NO:316, 344, 346, 350-351, 355, 357, 360-365, 384-388, 393, 397-400, 404, 413, 415-416, 425-426, 474, 476-487, 502, 532, 541, or 544-545 or the corn homolog of SEQ ID NOs:309, 311, 324, 373, 395, 454, or 518; when the recombinant DNA construct is expressed in soybean plants, the endogenous protein is a soybean protein with an amino acid sequence of SEQ ID NO: 309, 324, 373, 395, 518 or is a soybean homolog of SEQ ID NOs: 311, 316, 344, 346, 350-351, 355, 357, 360-365, 384-388, 393, 397-400, 404, 413, 415-416, 425-426, 454, 474, 476-487, 502, 532, 541, or 544-545; and when the recombinant DNA construct is expressed in a plant other than a corn or a soybean plant, the endogenous protein is the other plant's endogenous protein that has an amino acid sequence homologous to SEQ ID NO: 309, 311, 316, 324, 344, 346, 350-351, 355, 357, 360-365, 373, 384-388, 393, 395, 397-400, 404, 413, 415-416, 425-426, 454, 474, 476-487, 502, 518, 532, 541, or 544-545.

In practical aspects of this invention the recombinant DNA constructs of the invention are stably integrated into the chromosome of a plant cell nucleus.

This invention also provides transgenic plant cells comprising the stably integrated recombinant DNA constructs of the invention, transgenic plants and seeds comprising a plurality of such transgenic plant cells and transgenic pollen of such plants. Such transgenic plants are selected from a population of transgenic plants regenerated from plant cells transformed with recombinant DNA constructs by screening transgenic plants for an enhanced trait as compared to control plants. The enhanced trait is one or more of enhanced water use efficiency, enhanced cold tolerance, increased yield, enhanced nitrogen use efficiency, enhanced seed protein and enhanced seed oil.

In another aspect of the invention the plant cells, plants, seeds, and pollen further comprise DNA expressing a protein that provides tolerance from exposure to an herbicide applied at levels that are lethal to a wild type plant cell.

This invention also provides methods for manufacturing non-natural, transgenic seed that can be used to produce a crop of transgenic plants with an enhanced trait resulting from expression of a stably-integrated recombinant DNA construct. More specifically, the method comprises (a) screening a population of plants for an enhanced trait and a recombinant DNA construct, where individual plants in the population can exhibit the trait at a level less than, essentially the same as or greater than the level that the trait is exhibited in control plants, (b) selecting from the population one or more plants that exhibit the trait at a level greater than the level that said trait is exhibited in control plants, (c) collecting seed from a selected plant, (d) verifying that the recombinant DNA is stably integrated in said selected plants, (e) analyzing tissue of a selected plant to determine the production or suppression of a protein having the function of a protein encoded by nucleotides in a sequence of one of SEQ ID NOs:1-307. In one aspect of the invention, the plants in the population further comprise DNA expressing a protein that provides tolerance to exposure to a herbicide applied at levels that are lethal to wild type plant cells and the selecting is affected by treating the population with the herbicide, e.g. a glyphosate, dicamba, or glufosinate compound. In another aspect of the invention the plants are selected by identifying plants with the enhanced trait. The methods are especially useful for manufacturing corn, soybean, cotton, canola, alfalfa, wheat, rice, sugarcane or sugar beet seed.

Another aspect of the invention provides a method of producing hybrid corn seed comprising acquiring hybrid corn seed from a herbicide tolerant corn plant which also has stably-integrated, recombinant DNA construct comprising a promoter that is (a) functional in plant cells and (b) is operably linked to DNA that encodes or suppresses a protein having the function of a protein encoded by nucleotides in a sequence of one of SEQ ID NOs:1-307. The methods further comprise producing corn plants from said hybrid corn seed, wherein a fraction of the plants produced from said hybrid corn seed is homozygous for said recombinant DNA, a fraction of the plants produced from said hybrid corn seed is hemizygous for said recombinant DNA, and a fraction of the plants produced from said hybrid corn seed has none of said recombinant DNA; selecting corn plants which are homozygous and hemizygous for said recombinant DNA by treating with an herbicide; collecting seed from herbicide-treated-surviving corn plants and planting said seed to produce further progeny corn plants; repeating the selecting and collecting steps at least once to produce an inbred corn line; and crossing the inbred corn line with a second corn line to produce hybrid seed.

Another aspect of the invention provides a method of selecting a plant comprising plant cells of the invention by using an immunoreactive antibody to detect the presence or absence of protein expressed or suppressed by recombinant DNA in seed or plant tissue. Yet another aspect of the invention provides anti-counterfeit milled seed having, as an indication of origin, plant cells of this invention.

Still other aspects of this invention relate to transgenic plants with enhanced water use efficiency or enhanced nitrogen use efficiency. For instance, this invention provides methods of growing a corn, cotton, soybean, or canola crop without irrigation water comprising planting seed having plant cells of the invention which are selected for enhanced water use efficiency. Alternatively methods comprise applying reduced irrigation water, e.g. providing up to 300 millimeters of ground water during the production of a corn crop. This invention also provides methods of growing a corn, cotton, soybean or canola crop without added nitrogen fertilizer comprising planting seed having plant cells of the invention which are selected for enhanced nitrogen use efficiency.

### DETAILED DESCRIPTION OF THE INVENTION

In the attached sequence listing:

SEQ ID NO:1-307 are nucleotide sequences of the coding strand of DNA for “genes” used in the recombinant DNA imparting an enhanced trait in plant cells, i.e. each represents a coding sequence for a protein;

SEQ ID NO: 308-614 are amino acid sequences of the cognate protein of the “genes” with nucleotide coding sequences 1-307;

SEQ ID NO: 615-36442 are amino acid sequences of homologous proteins;

SEQ ID NO: 36443 is a nucleotide sequence of a base plasmid vector useful for corn transformation;

SEQ ID NO: 36444 is a nucleotide sequence of a base plasmid vector useful for soybean and canola transformation;

SEQ ID NO: 36445 is a nucleotide sequence of a base plasmid vector useful for cotton transformation;

SEQ ID NO: 36446 is a nucleotide sequence of a base plasmid vector useful for co-transformation to produce gene stacks in corn;

SEQ ID NO: 36447-36478 are consensus amino acid sequences.

Table 8 lists the protein SEQ ID NOs and their corresponding consensus SEQ ID NOs.

As used herein a “plant cell” means a plant cell that is transformed with stably-integrated, non-natural, recombinant DNA, e.g. by Agrobacterium-mediated transformation or by bombardment using microparticles coated with recombinant DNA or other means. A plant cell of this invention can be an originally-transformed plant cell that exists as a microorganism or as a progeny plant cell that is regenerated into differentiated tissue, e.g. into a transgenic plant with stably-integrated, non-natural recombinant DNA, or seed or pollen derived from a progeny transgenic plant.

As used herein a “transgenic plant” means a plant whose genome has been altered by the stable integration of recombinant DNA. A transgenic plant includes a plant regenerated from an originally-transformed plant cell and progeny transgenic plants from later generations or crosses of a transformed plant.

As used herein “recombinant DNA” means DNA which has been a genetically engineered and constructed outside of a cell including DNA containing naturally occurring DNA or cDNA or synthetic DNA.

As used herein “consensus sequence” means an artificial sequence of amino acids in a conserved region of an alignment of amino acid sequences of homologous proteins, e.g. as determined by a CLUSTALW alignment of amino acid sequence of homolog proteins.

As used herein a “homolog” means a protein in a group of proteins that perform the same biological function, e.g. proteins that belong to the same Pfam protein family and that provide a common enhanced trait in transgenic plants of this invention. Homologs are expressed by homologous genes. With reference to homologous genes, homologs include orthologs, i.e. genes expressed in different species that evolved from a common ancestral genes by speciation and encode proteins retain the same function, but do not include paralogs, i.e. genes that are related by duplication but have evolved to encode proteins with different functions. Homologous genes include naturally occurring alleles and artificially-created variants. Degeneracy of the genetic code provides the possibility to substitute at least one base of the protein encoding sequence of a gene with a different base without causing the amino acid sequence of the polypeptide produced from the gene to be changed. When optimally aligned, homolog proteins have at least 60% identity, 65% identity, 70% identity, 75% identity, 80%, identity, 85% identity, 90% identity, 95, 96, 97, 98, or 99% identity over the full length of a protein identified as being associated with imparting an enhanced trait when expressed in plant cells. In one aspect of the invention homolog proteins have an amino acid sequence that has at least 90% identity to a consensus amino acid sequence of proteins and homologs disclosed herein.

Homologs are identified by comparison of amino acid sequence, e.g. manually or by use of a computer-based tool using known homology-based search algorithms such as the suite of BLAST programs available from NCBI. A local sequence alignment program, e.g. BLAST, can be used to search a database of sequences to find similar sequences, and the summary Expectation value (E-value) used to measure the sequence base similarity. Because a protein hit with the best E-value for a particular organism may not necessarily be an ortholog, i.e. have the same function, or be the only ortholog, a reciprocal query is used to filter hit sequences with significant E-values for ortholog identification. The reciprocal query entails search of the significant hits against a database of amino acid sequences from the base organism that are similar to the sequence of the query protein. A hit can be identified as an ortholog, when the reciprocal query's best hit is the query protein itself or a protein encoded by a duplicated gene after speciation. A further aspect of the homologs encoded by DNA useful in the transgenic plants of the invention are those proteins that differ from a disclosed protein as the result of deletion or insertion of one or more amino acids in a native sequence.

Percent identity describes the extent to which the sequences of DNA or protein segments are invariant in an alignment of sequences, for example nucleotide sequences or amino acid sequences. An alignment of sequences is created by manually aligning two sequences, e.g. a stated sequence, as provided herein, as a reference, and another sequence, to produce the highest number of matching elements, e.g. individual nucleotides or amino acids, while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” (“% identity”) as used herein is the identity fraction times 100.

“Pfam” is a large collection of multiple sequence alignments and hidden Markov models covering many common protein families, e.g. Pfam version 19.0 (December 2005) contains alignments and models for 8183 protein families and is based on the Swissprot 47.0 and SP-TrEMBL 30.0 protein sequence databases. See S. R. Eddy, “Profile Hidden Markov Models”, Bioinformatics 14:755-763, 1998. The Pfam database is currently maintained and updated by the Pfam Consortium. The alignments represent some evolutionary conserved structure that has implications for the protein's function. Profile hidden Markov models (profile HMMs) built from the protein family alignments are useful for automatically recognizing that a new protein belongs to an existing protein family even if the homology by alignment appears to be low.

Protein domains are identified by querying the amino acid sequence of a protein against Hidden Markov Models which characterize protein family domains (“Pfam domains”) using HMMER software, which is available from the Pfam Consortium. The HMMER software is also disclosed in patent application publication US 2008/0148432 A1 incorporated herein by reference. A protein domain meeting the gathering cutoff for the alignment of a particular Pfam domain is considered to contain the Pfam domain.

A “Pfam domain module” is a representation of Pfam domains in a protein, in order from N terminus to C terminus. In a Pfam domain module individual Pfam domains are separated by double colons “::”. The order and copy number of the Pfam domains from N to C terminus are attributes of a Pfam domain module. Although the copy number of repetitive domains is important, varying copy number often enables a similar function. Thus, a Pfam domain module with multiple copies of a domain should define an equivalent Pfam domain module with variance in the number of multiple copies. A Pfam domain module is not specific for distance between adjacent domains, but contemplates natural distances and variations in distance that provide equivalent function. The Pfam database contains both narrowly- and broadly-defined domains, leading to identification of overlapping domains on some proteins. A Pfam domain module is characterized by non-overlapping domains. Where there is overlap, the domain having a function that is more closely associated with the function of the protein (based on the E value of the Pfam match) is selected.

Once one DNA is identified as encoding a protein which imparts an enhanced trait when expressed in transgenic plants, other DNA encoding proteins with the same Pfam domain module are identified by querying the amino acid sequence of protein encoded by candidate DNA against the Hidden Markov Models which characterizes the Pfam domains using HMMER software. Candidate proteins meeting the same Pfam domain module are in the protein family and have cognate DNA that is useful in constructing recombinant DNA for the use in the plant cells of this invention. Hidden Markov Model databases for use with HMMER software in identifying DNA expressing protein with a common Pfam domain module for recombinant DNA in the plant cells of this invention are available from the Pfam Consortium (ftp.sanger.ac.uk/pub/databases/Pfam/) and are incorporated herein by reference.

The HMMER software and Pfam databases (version 23.0) were used to identify known domains in the proteins corresponding to amino acid sequence of SEQ ID NOs: 308-310, 312-313, 315, 317-332, 334-343, 345, 347-364, 366-372, 374, 382, 387-403, 406-412, 414-425, 427-448, 450-460, 462-465, 467-477, 479-482, 484-487, 493-511, 513-539, 542, 545-558, 560-578, 580-582, 584-598, 600-602, 604-608, 612-614. All DNA encoding proteins that have scores higher than the gathering cutoff disclosed in Table 11 by Pfam analysis disclosed herein can be used in recombinant DNA of the plant cells of this invention, e.g. for selecting transgenic plants having enhanced agronomic traits. The relevant Pfams modules for use in this invention, as more specifically disclosed below, are PHD::SET, CBFB_NFYA, 2-Hacid_dh_C, 60KD_IMP, A_thal_{—}3526, AA_kinase, AA_kinase::NAD_binding_{—}3::Homoserine_dh, AA_permease, Aa_trans, ABC_membrane::ABC_tran, ABCtran, Acetate_kinase, Acid_phosphat_B, Acyl-ACP_TE, AlaDh_PNT_N::AlaDh_PNT_C, Amino_oxidase, Aminotran_{—}1_{—}2 Aminotran_{—}5, Ammonium_transp, Ank::Ank::Ank::Ank::Ank::Ank::Ank::Ank, AP2, Arginase, Arginosuc_synth, AsnA, Asp, Asp_decarbox, ATP-sulfurylase, Auxin inducible, BCCT, BPD_transp_{—}2, BTB, bZIP_{—}1, bZIP_{—}1::MethyltransfD12, bZIP_{—}2, bZIP_{—}2::bZIP_{—}1, C4dic_mal_tran, CBFB_NFYA, CCT, Clp_N::Clp_N::AAA::AAA_{—}2::C1pB_D2-small, cNMP_binding::Crp, CSD, DEAD::Helicase_C, DEAD_{—}2::DUF1227, Dehydrin Dimerisation::Methyltransf_{—}2, DSPc, DUF1292, DUF506, DUF640, DUF647, DUF828::PH_{—}2, eIF-5a, eRF1_{—}1::eRF1_{—}2::eRF1_{—}3, FAD_binding_{—}3::FHA, FAD_binding_{—}4::ALO, FAD_binding_{—}4::Lact-deh-memb, F-box::Tub, Fe-ADH, Form_Nir_trans, FTCD_N, Gal_Lectin, GATase_{—}2::Asn_synthase, GATase_{—}2::Glu_syn_centrah:Glu_synthase::GXGXG, GDPD, GII3, Gln-synt_N::Gln-synt_C, Globin::FAD_binding_{—}6::NAD_binding_{—}1, Glt_symporter, Glutaredoxin, Glutaredoxin::Glutaredoxin::Glutaredoxin, Glyco_transf_{—}20::Trehalose_PPase, GTP1_OBG::MMR_HSR1::DUF1967, HLH, HMG_box Homeobox::HALZ, Homeobox::START, HR_lesion, HSP20, HSP70, LIM, LIM::LIM, LisH::WD40::WD40::WD40::WD40::WD40::WD40::WD40 LRR_{—}1::LRR_{—}1::LRR_{—}1::LRR_{—}1::Pkinase_Tyr, RR_{—}1::LRR_{—}1::LRR_{—}1::LRR_{—}1::Pkinase, LRRNT_{—}2::LRR_{—}1::Pkinase_Tyr, LysM, MBD, MethyltransfD12. MIT::AAA::Vps4_C, Mito_carr::Mito_carr::Mito_carr, MMR_HSR1::KH_{—}2, Monooxygenase_B, Myb_DNA-binding::Myb_DNA-binding, NAD_binding_{—}1, NAD_binding_{—}2, NAM, Ndr, NIR_SIR_ferr::NIR_SIR::NIR_SIR_ferr, Nitroreductase, NUDIX, OKR_DC_{—}1::OKR_DC_{—}1_C, OPT, p450, PAS_{—}2::GAF::Phytochrome::PAS::PAS::HisKA::HATPase_c, PBP, Peptidase_C2::Calpain_III, Peptidase_S10, peroxidase, Pkinase, Pkinase::NAF, PLAC8, PMEI, POX::Homeobox, PP2C, PTA_PTB, PTR2 RCC1::RCC1::RCC1, Response_reg::Myb_DNA-binding, Ribosomal_L21p, RolB_RolC::Amino_oxidase, RRM_{—}1, RRM_{—}1::RRM_{—}1, RRM_{—}1::zf-CCHC RWP-RK::PB1, SBP, SBP_bac_{—}3, Sina, SIS::CBS, SOUL, SPRY, SRF-TF, SRF-TF::K-box, SSF, Ss11::C1_{—}4, Sulfate_transp::STAS, Thg1::Thg1 Thiolase_N::Thiolase_C, Thioredoxin::Glutaredoxin::Glutaredoxin::Glutaredoxin, TPP_enzyme_N::TPP_enzyme_M::TPP_enzyme_C Transaldolase, tRNA_synt_{—}1c_R1::tRNA_synt_{—}1c_R2::tRNA-synt_{—}1c::tRNA-synt_{—}1c_C, ubiquitin, U-box, Usp, WD40::WD40, WRKY, WRKY::WRKY zf-B_box, zf-B_box::MethyltransfD12, zf-B_box::zf-B_box::CCT, zf-C3HC4, zf-CCCH, zf-CCIIC::Plus-3, and zf-Dof for which databases are included in the appended computer listing.

As used herein “promoter” means regulatory DNA for initializing transcription. A “plant promoter” is a promoter capable of initiating transcription in plant cells whether or not its origin is a plant cell, e.g. is it well known that Agrobacterium promoters are functional in plant cells. Thus, plant promoters include promoter DNA obtained from plants, plant viruses and bacteria such as Agrobacterium and Bradyrhizobium bacteria. Examples of promoters under developmental control include promoters that preferentially initiate transcription in certain tissues, such as leaves, roots, or seeds. Such promoters are referred to as “tissue preferred”. Promoters that initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions.

As used herein “operably linked” means the association of two or more DNA fragments in a recombinant DNA construct so that the function of one, e.g. protein-encoding DNA, is controlled by the other, e.g. a promoter.

As used herein “expressed” means produced, e.g. a protein is expressed in a plant cell when its cognate DNA is transcribed to mRNA that is translated to the protein.

As used herein “suppressed” means decreased, e.g. a protein is suppressed in a plant cell when there is a decrease in the amount and/or activity of the protein in the plant cell. The presence or activity of the protein can be decreased by any amount up to and including a total loss of protein expression and/or activity.

As used herein a “control plant” means a plant that does not contain the recombinant DNA that imparts an enhanced trait. A control plant is used to identify and select a transgenic plant that has an enhanced trait. A suitable control plant can be a non-transgenic plant of the parental line used to generate a transgenic plant, i.e. devoid of recombinant DNA. A suitable control plant may in some cases be a progeny of a hemizygous transgenic plant line that does not contain the recombinant DNA, known as a negative segregant.

As used herein an “enhanced trait” means a characteristic of a transgenic plant that includes, but is not limited to, an enhance agronomic trait characterized by enhanced plant morphology, physiology, growth and development, yield, nutritional enhancement, disease or pest resistance, or environmental or chemical tolerance. In more specific aspects of this invention enhanced trait is selected from group of enhanced traits consisting of enhanced water use efficiency, enhanced cold tolerance, increased yield, enhanced nitrogen use efficiency, enhanced seed protein and enhanced seed oil. In an important aspect of the invention the enhanced trait is enhanced yield including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), car number, seed number per car, seed size, composition of seed (starch, oil, protein) and characteristics of seed fill.

Increased yield of a transgenic plant of the present invention can be measured in a number of ways, including test weight, seed number per plant, seed weight, seed number per unit area (i.e. seeds, or weight of seeds, per acre), bushels per acre, tons per acre, or kilo per hectare. For example, corn yield may be measured as production of shelled corn kernels per unit of production area, for example in bushels per acre or metric tons per hectare, often reported on a moisture adjusted basis, for example at 15.5 percent moisture. Increased yield may result from improved utilization of key biochemical compounds, such as nitrogen, phosphorous and carbohydrate, or from improved responses to environmental stresses, such as cold, heat, drought, salt, and attack by pests or pathogens. Recombinant DNA used in this invention can also be used to provide plants having improved growth and development, and ultimately increased yield, as the result of modified expression of plant growth regulators or modification of cell cycle or photosynthesis pathways. Also of interest is the generation of transgenic plants that demonstrate enhanced yield with respect to a seed component that may or may not correspond to an increase in overall plant yield. Such properties include enhancements in seed oil, seed molecules such as protein and starch, oil components as may be manifest by an alterations in the ratios of seed components.

Recombinant DNA constructs are assembled using methods well known to persons of ordinary skill in the art and typically comprise a promoter operably linked to DNA, the expression of which provides the enhanced agronomic trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876 which discloses a rice actin promoter, U.S. Pat. No. 7,151,204 which discloses a maize chloroplast aldolase promoter and a maize aldolase (FDA) promoter, and US Patent Application Publication 2003/0131377 A1 which discloses a maize nicotianamine synthase promoter. These and numerous other promoters that function in plant cells are known to those skilled in the art and available for use in recombinant polynucleotides of the present invention to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene. See also US Patent Application Publication 2002/0192813A1 which discloses 5′, 3′ and intron elements useful in the design of effective plant expression vectors.

In other aspects of the invention, sufficient expression in plant seed tissues is desired to affect improvements in seed composition. Exemplary promoters for use for seed composition modification include promoters from seed genes such as napin as disclosed in U.S. Pat. No. 5,420,034, maize L3 oleosin as disclosed in U.S. Pat. No. 6,433,252), zein Z27 as disclosed by Russell et al. (1997) Transgenic Res. 6(2):157-166), globulin 1 as disclosed by Belanger et al (1991) Genetics 129:863-872), glutelin 1 as disclosed by Russell (1997) supra), and peroxiredoxin antioxidant (Per1) as disclosed by Stacy et al. (1996) Plant Mol Biol. 31(6):1205-1216.

Recombinant DNA constructs useful in this invention will also generally include a 3′ element that typically contains a polyadenylation signal and site. Well-known 3′ elements include those from Agrobacterium tumefaciens genes such as nos 3′, tml 3′, tmr 3′, tms 3′, ocs 3′, tr7 3′, for example disclosed in U.S. Pat. No. 6,090,627; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in US Patent Application Publication 2002/0192813 A1; and the pea (Pisum sativum) ribulose biphosphate carboxylase gene (rbs 3′), and 3′ elements from the genes within the host plant.

Constructs and vectors may also include a transit peptide for targeting of a gene to a plant organelle, particularly to a chloroplast, leucoplast or other plastid organelle. For descriptions of the use of chloroplast transit peptides see U.S. Pat. No. 5,188,642 and U.S. Pat. No. 5,728,925. For description of the transit peptide region of an Arahidopsis EPSPS gene useful in the present invention, see Klee, H. J. et al (MGG (1987) 210:437-442).

Recombinant DNA constructs for gene suppression can be designed for any of a number the well-known methods for suppressing transcription of a gene, the accumulation of the mRNA corresponding to that gene or preventing translation of the transcript into protein. Posttranscriptional gene suppression can be practically effected by transcription of RNA that forms double-stranded RNA (dsRNA) having homology to mRNA produced from a gene targeted for suppression.

Gene suppression can also be achieved by insertion mutations created by transposable elements may also prevent gene function. For example, in many dicot plants, transformation with the T-DNA of Agrobacterium may be readily achieved and large numbers of transformants can be rapidly obtained. Also, some species have lines with active transposable elements that can efficiently be used for the generation of large numbers of insertion mutations, while some other species lack such options. Mutant plants produced by Agrobacterium or transposon mutagenesis and having altered expression of a polypeptide of interest can be identified using the polynucleotides of the present invention. For example, a large population of mutated plants may be screened with polynucleotides encoding the polypeptide of interest to detect mutated plants having an insertion in the gene encoding the polypeptide of interest.

Transgenic plants may comprise a stack of one or more polynucleotides disclosed herein resulting in the production or suppression of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene, and co-transformation of genes into a single plant cell. Co-transformation of genes can be carried out using single transformation vectors comprising multiple genes or genes carried separately on multiple vectors.

Transgenic plants comprising or derived from plant cells of this invention transformed with recombinant DNA can be further enhanced with stacked traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in US Patent Application Publication 2003/0083480 A1 also for imparting glyphosate tolerance; dicamba monooxygenase disclosed in US Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in US Patent Application Publication 2003/010609 A1 for imparting N-amino methyl phosphonic acid tolerance; polynucleotide molecules disclosed in U.S. Pat. No. 6,107,549 for impartinig pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and US Patent Application Publication 2002/0112260. Molecules and methods for imparting insect/nematode/virus resistance are disclosed in U.S. Pat. Nos. 5,250,515; 5,880,275; 6,506,599; 5,986,175 and US Patent Application Publication 2003/0150017 A1.

### Plant Cell Transformation Methods

Numerous methods for transforming chromosomes in a plant cell nucleus with recombinant DNA are known in the art and are used in methods of preparing a transgenic plant cell nucleus cell, and plant. Two effective methods for such transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice) and Agrobacterium-mediated transformation is described in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 5,846,797 (cotton); U.S. Pat. No. 6,384,301 (soybean), U.S. Pat. No. 7,026,528 (wheat) and 6,329,571 (rice), US Patent Application Publication 2004/0087030 A1 (cotton), and US Patent Application Publication 2001/0042257 A1 (sugar beet), all of which are incorporated herein by reference for enabling the production of transgenic plants. Transformation of plant material is practiced in tissue culture on a nutrient media, i.e. a mixture of nutrients that will allow cells to grow in vitro. Recipient cell targets include, but are not limited to, meristem cells, hypocotyls, calli, immature embryos and gametic cells such as microspores, pollen, sperm and egg cells. Callus may be initiated from tissue sources including, but not limited to, immature embryos, hypocotyls, seedling apical meristems, microspores and the like. Cells containing a transgenic nucleus are grown into transgenic plants.

In addition to direct transformation of a plant material with a recombinant DNA, a transgenic plant cell nucleus can be prepared by crossing a first plant having cells with a transgenic nucleus with recombinant DNA with a second plant lacking the transgenic nucleus. For example, recombinant DNA can be introduced into a nucleus from a first plant line that is amenable to transformation to transgenic nucleus in cells that are grown into a transgenic plant which can be crossed with a second plant line to introgress the recombinant DNA into the second plant line. A transgenic plant with recombinant DNA providing an enhanced trait, e.g. enhanced yield, can be crossed with transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line

In the practice of transformation DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or a herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue, and plant species. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of enhanced agronomic trait.

### Transgenic Plants and Seeds

Transgenic plants derived from transgenic plant cells having a transgenic nucleus of this invention are grown to generate transgenic plants having an enhanced trait as compared to a control plant and produce transgenic seed and haploid pollen of this invention. Such plants with enhanced traits are identified by selection of transformed plants or progeny seed for the enhanced trait. For efficiency a selection method is designed to evaluate multiple transgenic plants (events) comprising the recombinant DNA, for example multiple plants from 2 to 20 or more transgenic events. Transgenic plants grown from transgenic seed provided herein demonstrate improved agronomic traits that contribute to increased yield or other trait that provides increased plant value, including, for example, improved seed quality. Of particular interest are plants having enhanced water use efficiency, enhanced cold tolerance, increased yield, enhanced nitrogen use efficiency, enhanced seed protein and enhanced seed oil.

Table 1 provides a list of protein encoding DNA (“genes”) that are useful as recombinant DNA for production of transgenic plants with enhanced agronomic trait, the elements of Table 1 are described by reference to:
“PEP SEQ ID NO” identifies an amino acid sequence from SEQ ID NO: 308 to 614.
“NUC SEQ ID NO” identifies a DNA sequence from SEQ ID NO:1 to 307.
“Gene ID” refers to an arbitrary identifier.
“Gene Name” denotes a common name for the protein encoded by the recombinant DNA preceded by the abbreviated genus and species as fully defined in the sequence listing. The + or − preceding the gene name indicates whether the protein is expressed (+) or suppressed (−) in plants to provide an enhanced trait.

TABLE 1

| NUC | PEP | | |
|---|---|---|---|
| SEQ | SEQ | | |
| ID | ID | | |
| NO | NO | Gene ID | Gene Name |
| 1 | 308 | 1174936:1 | +Os.AlaAT |
| 2 | 309 | Mnom000960 | −Gm.G1543 like 1 |
| 3 | 310 | Mnom001103 | +Gm.Hyh |
| 4 | 311 | 1194741:1 | −Gh.IDA-like 1:3:1 |
| 5 | 312 | Mnom001209 | +Zm.SCPL21 |
| 6 | 313 | Mnom001263 | +Zm.Tga1 |
| 7 | 314 | Mnom001273 | +Zm.A1ZM023275_at_Adenosine |
| | | | 5′-phosphosulfate reductase 8 |
| 8 | 315 | Mnom001274 | +Zm.A1ZM056190_at_B0806H07.1 |
| | | | protein |
| 9 | 316 | Mnom001282 | −Zm.A1ZM009253_at_Hypothetical |
| | | | protein OJ1254_E07.2-1 |
| 10 | 317 | 1174117:1 | +Ss.CGPG7432_hypothetical |
| | | | protein S\|\|1109 |
| 11 | 318 | 1174129:1 | +Xn.CGPG6757_Aspartate |
| | | | Aminotransferase |
| 12 | 319 | 1169877:1 | +Cg.D-lactate |
| | | | dehydrogenase |
| | | | (EC:1.1.1.28) |
| 13 | 320 | Mnom001350 | +Ns.glycerol |
| | | | dehydrogenase (NADP+) |
| | | | (EC:1.1.1.72) |
| 14 | 321 | PHE0015383 | +Bs.AldA (L-ALANINE |
| | | | DEHYDROGENASE) |
| | | | (EC:1.4.1.1) |
| 15 | 322 | Mnom001361 | +Mt.Thiolase |
| 16 | 323 | Mnom001363 | +Gh.Thiolase |
| 17 | 324 | Mnom001367 | −Gm.Soul |
| 18 | 325 | Mnom001370 | +Gm.X. bovienii spartate |
| | | | kinase_T359I |
| 19 | 326 | Mnom001372 | +Gm.X.nematophila AK_T359I |
| 20 | 327 | Mnom001373 | +Ec. metL AK-HSDS |
| 21 | 328 | 1186474:1 | +Gm.PIL-B |
| 22 | 329 | PHE0024821 | +Gm.PIL-B |
| 23 | 330 | 1184139:1 | +At.PIF4 |
| 24 | 331 | PHE0003348 | +At.PIF4 |
| 25 | 332 | 1181794:1 | +Gm.PICOT |
| 26 | 333 | 1185540:1 | +Ag.PRS |
| 27 | 334 | 1184141:1 | +At.CaMK4/At.CRK3 |
| 28 | 335 | 1183499:1 | +At.GLN1; 1 |
| 29 | 336 | 1184141:1 | +At.CaMK4/At.CRK3 |
| 30 | 337 | 1183676:1 | +Gm.Myc:Dam |
| 31 | 338 | Mnom001401 | +Gm.At.Hy5:Myc:Dam |
| 32 | 339 | 1184600:1 | +Gm.At.G1988:Myc:DAM |
| 33 | 340 | Mnom001436 | +Rp.indole-3-pyruvate |
| | | | decarboxylase |
| 34 | 341 | Mnom001439 | +At.tryptophane |
| | | | monooxygenase |
| 35 | 342 | Mnom001440 | +Pf.tryptophane |
| | | | monooxygenase |
| 36 | 343 | Mnom001453 | +Ta.Fd Nitrite reductase |
| 37 | 344 | 1181413:1 | −Zm.14-3-3 corn homolog |
| | | | (Chi) |
| 38 | 345 | Mnom001471 | +Zm.PMH1 |
| 39 | 346 | 1193022:1 | −Zm.NAC6-like |
| 40 | 347 | Mnom001521 | +Gm.ERL2 |
| 41 | 348 | 1168947:1 | +At.G1543-native-delta |
| | | | 1-21 |
| 42 | 349 | 1170457:1 | +At.G1543 Kozak Variant |
| | | | (aa 1-4, changed MIKL to |
| | | | MASS) |
| 43 | 350 | PHE0001187 | −Zm.spa1-like |
| 44 | 351 | Mnom001594 | −Zm.SLAC1 |
| 45 | 352 | 1171516:1 | +St.Pox1 |
| 46 | 353 | PHE0022652 | +St.Pox1 |
| 47 | 354 | Mnom001612 | +Zm.HOS15 |
| 48 | 355 | Mnom001615 | −Zm.ABI5 |
| 49 | 356 | Mnom001618 | +Le.SlGalLDH |
| 50 | 357 | Mnom001628 | −Zm.NAC-like |
| 51 | 358 | Mnom001629 | +Zm.NFD1 |
| 52 | 359 | Mnom001636 | +Zm.POZ domain protein |
| 53 | 360 | Mnom001641 | −Zm.Putative nodule |
| | | | inception protein |
| 54 | 361 | Mnom001642 | −Zm.ammonium |
| | | | transporter |
| 55 | 362 | Mnom001643 | −Zm.POZ domain protein |
| 56 | 363 | Mnom001644 | −Zm.isp4, oligopeptide |
| | | | transport |
| 57 | 364 | Mnom001645 | −Zm.unknown |
| 58 | 365 | Mnom001647 | −Zm.RAD-like |
| 59 | 366 | Mnom001651 | +Pp.Formiminotransferase |
| | | | cyclodeaminase |
| 60 | 367 | Mnom001652 | +Zm.Formiminotransferase |
| | | | cyclodeaminase |
| 61 | 368 | Mnom001653 | +Cr.Na/glucose |
| | | | cotransporter |
| 62 | 369 | Mnom001661 | +Cr.Cysteine sulfinate |
| | | | desulfinase/cysteine |
| | | | desulfurase, NifS |
| 63 | 370 | 1194860:1 | +Cr.Cysteine desulfurase |
| | | | (seleno-cysteine lyase) |
| 64 | 371 | Mnom001665 | +Ne.ammonium |
| | | | monooxygenase subunit A |
| 65 | 372 | 1180851:1 | +PMEI1 |
| 66 | 373 | Mnom001747 | −Gm.MBD6 Soy homolog |
| 67 | 374 | 1170440:1 | +Gm.Col9 |
| | | | MRT3847_269613C |
| 68 | 375 | 1174098:1 | +GITI.PHD_SET |
| 69 | 376 | 1183497:1 | +Gm.G2512-like_AP2 |
| | | | domain transcriptional |
| | | | regulator |
| 70 | 377 | 1188159:1 | +Gm.G565-like_bZIP |
| | | | domain transcriptional |
| | | | regulator |
| 71 | 378 | 1170356:1 | +Gm.G2113-like_AP2 |
| | | | domain transcriptional |
| | | | regulator |
| 72 | 379 | 1170348:1 | +Gm.CGPG3920_expressed |
| | | | protein |
| 73 | 380 | 1170352:1 | +Gm.CGPG7836_ethylene |
| | | | responsive element |
| | | | binding factor_G1005-like |
| 74 | 381 | 1170996:1 | +At.Hsp101 |
| 75 | 382 | 1170342:1 | +At.PEL1 |
| 76 | 383 | 1170354:1 | +Gm.Unknown G1988 |
| | | | soybean heat induced |
| | | | gene |
| 77 | 384 | PHE0024001 | −Zm.phosphoglucomutase |
| 78 | 385 | 1190095:1 | −Zm.phosphoglucomutase |
| 79 | 386 | Mnom001839 | −Zm.ATHB16 HD-Zip I |
| | | | transcription factor |
| 80 | 387 | Mnom001840 | −Zm.SOMNUS CCCH-type |
| | | | zinc finger protein |
| 81 | 388 | Mnom001841 | −Zm.zinc finger protein 10 |
| 82 | 389 | 1174488:1 | +Zm.basic helix-loop-helix |
| | | | (bHLH) family protein |
| | | | (AT5G50915-like) |
| 83 | 390 | Mnom001851 | +At.Arginosuccinate |
| | | | synthase |
| 84 | 391 | Mnom001869 | +Zm.Glutaredoxin domain |
| | | | containing protein |
| 85 | 392 | 1175917:1 | +Ec.AsnA |
| 86 | 393 | Mnom001877 | −Zm.FNR |
| 87 | 394 | Mnom001880 | +Ss.ntcA |
| 88 | 395 | Mnom001900 | −Gm.FW2.2 like, PLAC8 |
| 89 | 396 | Mnom001903 | +Mt.MTP_Ac160241 |
| 90 | 397 | Mnom001915 | −Zm.MYB74 |
| 91 | 398 | Mnom001921 | −Zm.ASA1 |
| 92 | 399 | Mnom001922 | −Zm.WRKY72 |
| 93 | 400 | Mnom001925 | −Zm.CND41 homolog |
| 94 | 401 | Mnom001930 | +Cr.Glutamine synthetase |
| | | | (GLN4) |
| 95 | 402 | Mnom001933 | +Cr.Ferredoxin-dependent |
| | | | glutamate synthase (GSF1) |
| 96 | 403 | Mnom001936 | +Cr.nitrite transporter |
| | | | NAR1 |
| 97 | 404 | Mnom001937 | −Zm.Initiator-binding |
| | | | protein; IBP2 |
| 98 | 405 | 1173274:1 | +At.Rif1 (Resistant to |
| | | | inhibition by FSM) |
| 99 | 406 | 1175406:1 | +At.Glyoxylate |
| | | | Reductase 2 |
| 100 | 407 | 1178997:1 | +At.Glyoxylate |
| | | | Reductase 1 |
| 101 | 408 | 1194803:1 | +Cr.CPYC type (GRX1) |
| 102 | 409 | 1194791:1 | +Cr.CGFS type (GRX3) |
| 103 | 410 | 1178100:1 | +At.wt G1543 |
| 104 | 411 | 1184554:1 | +At.G1543 (R138, 142A) |
| 105 | 412 | 1178103:1 | +At.G1543 NNO |
| 106 | 413 | 1170936:1 | −Zm.TAA1 |
| 107 | 414 | Mnom001989 | +Zm.Ghd7-2 |
| 108 | 415 | Mnom001991 | −Zm.MBD6 |
| 109 | 416 | Mnom001992 | −Zm.MBD10 |
| 110 | 417 | 1175891:1 | +Zm.CGPG9187_Putative |
| | | | aurora-related kinase |
| 111 | 418 | 1174476:1 | +At.CGPG4567_Hypothetical |
| | | | protein AT4g11250 |
| 112 | 419 | 1181001:1 | +At.CGPG3781_MADS-box |
| | | | protein-like |
| 113 | 420 | 1158841:1 | +Pd.PHE0017217_SCF5A8 |
| 114 | 421 | 1178370:1 | +At.HDG11 |
| 115 | 422 | 1178370:1 | +At.HDG11 |
| 116 | 423 | PHE0024161 | +At.HDG11 |
| 117 | 424 | PHE0024161 | +At.HDG11 |
| 118 | 425 | Mnom002088 | −Zm.Arginase (EC 3.5.3.1) |
| 119 | 426 | 1195680:1 | −Zm.KINB1 |
| 120 | 427 | Mnom002104 | +Gm.Nuclear factor Y |
| | | | NFYA5 |
| 121 | 428 | Mnom002105 | +Gm.Nuclear factor Y |
| | | | NFYA5 |
| 122 | 429 | Mnom002106 | +Gm.Nuclear factor Y |
| | | | NFYA5 |
| 123 | 430 | Mnom002107 | +Gm.Nuclear factor Y |
| | | | NFYA5 |
| 124 | 431 | Mnom002108 | +Gm.Nuclear factor Y |
| | | | NFYA5 |
| 125 | 432 | Mnom002115 | +At.phyB |
| 126 | 433 | 1176524:1 | +Gm.CGPG5320_HMG1/ |
| | | | 2-like protein |
| 127 | 434 | 1183504:1 | +Zm.SPRY |
| 128 | 435 | 1183504:1 | +Zm.SPRY |
| 129 | 436 | 1180104:1 | +Zm.RRM1 |
| 130 | 437 | 1180104:1 | +Zm.RRM1 |
| 131 | 438 | 1182647:1 | +Zm.RRM2 |
| 132 | 439 | 1182647:1 | +Zm.RRM2 |
| 133 | 440 | 1182645:1 | +Zm.RRM3 |
| 134 | 441 | 1182645:1 | +Zm.RRM3 |
| 135 | 442 | 1182640:1 | +Zm.KH2/ERA-like |
| 136 | 443 | 1182640:1 | +Zm.KH2/ERA-like |
| 137 | 444 | 1182640:1 | +Zm.KH2/ERA-like |
| 138 | 445 | 1180839:1 | +Os.BIRF1 |
| | | | (Benzathiodiazole induced |
| | | | ring finger protein 1) |
| 139 | 446 | PHE0024283 | +Os.BIRF1 |
| | | | (Benzathiodiazole induced |
| | | | ring finger protein 1) |
| 140 | 447 | PHE0024283 | +Os.BIRF1 |
| | | | (Benzathiodiazole induced |
| | | | ring finger protein 1) |
| 141 | 448 | 1180839:1 | +Os.BIRF1 |
| | | | (Benzathiodiazole induced |
| | | | ring finger protein 1) |
| 142 | 449 | Mnom002224 | +Zm.CTR2 |
| 143 | 450 | 1188219:1 | +Rs.glyoxylate reductase |
| 144 | 451 | 1187645:1 | +Bh.alanine dehydrogenase |
| 145 | 452 | 1187645:1 | +Bh.alanine dehydrogenase |
| 146 | 453 | 1147250:1 | +Zm.PHE0010151_glutaminyl-tRNA |
| | | | synthetase |
| 147 | 454 | PHE0024437 | −Bn.MAX1 |
| 148 | 455 | PHE0019867 | +At.GLN1; 1 |
| 149 | 456 | 1185568:1 | +At.HYH (HY5 Homolog) |
| | | | S24A mutant |
| 150 | 457 | PHE0024430 | +At.HYH (HY5 Homolog) |
| 151 | 458 | 1184541:1 | +Ss.high-affinity |
| | | | branched-chain amino |
| | | | acid transport protein; |
| | | | BraE |
| 152 | 459 | 1195300:1 | +Ss.high-affinity |
| | | | branched-chain amino |
| | | | acid transport protein; |
| | | | BraE |
| 153 | 460 | 1183470:1 | +Ss.aspartate |
| | | | 1-decarboxylase |
| 154 | 461 | 1183502:1 | +Ss.Bifunctional |
| | | | imidazoleglycerol- |
| | | | phosphate dehydratase; |
| | | | histidinol-phosphatase |
| 155 | 462 | 1184961:1 | +Ss.Glutamate permease |
| 156 | 463 | PHE0024436 | +Ss.Glutamate permease |
| 157 | 464 | 1184550:1 | +Ss.glutamine ABC |
| | | | transporter |
| 158 | 465 | PHE0024437 | +Ss.glutamine ABC |
| | | | transporter |
| 159 | 466 | Mnom002329 | +Ns.Nitrite/Nitrate |
| | | | permease |
| 160 | 467 | 1183533:1 | +Ss.alanine |
| | | | dehydrogenase |
| 161 | 468 | 1181792:1 | +Cg.PHE0007792_nitroreductase |
| 162 | 469 | Mnom002337 | +Zm.Putative acid |
| | | | phosphatase |
| 163 | 470 | 1183509:1 | +At.HYH (HY5 Homolog) |
| 164 | 471 | 1194207:1 | +Bn.SUM2 |
| 165 | 472 | 1184951:1 | +At.da1-1 |
| 166 | 473 | 1184947:1 | +Zm.da1-1 |
| 167 | 474 | Mnom002354 | −Zm.BB |
| 168 | 475 | Mnom002372 | +Zm.Putative |
| | | | UVB-resistance |
| | | | protein UVR8 |
| 169 | 476 | Mnom002381 | −Zm.Putative sulfate |
| | | | transporter |
| 170 | 477 | Mnom002382 | −Zm.Putative |
| | | | UVB-resistance |
| | | | protein UVR8 |
| 171 | 478 | Mnom002383 | −Zm.Putative transfactor |
| 172 | 479 | Mnom002384 | −Zm.Putative trehalose-6- |
| | | | phosphate synthase |
| 173 | 480 | Mnom002385 | −Zm.Putative |
| | | | polypyrimidine tract- |
| | | | binding protein homolog |
| 174 | 481 | Mnom002387 | −Zm.Putative aspartic |
| | | | proteinase nepenthesin I |
| 175 | 482 | Mnom002388 | −Zm.Os08g0542700 protein |
| 176 | 483 | Mnom002389 | −Zm.unknown protein |
| 177 | 484 | Mnom002390 | −Zm.Dual-specificity |
| | | | protein-like phosphatase 3 |
| 178 | 485 | Mnom002391 | −Zm.Hypothetical protein |
| 179 | 486 | Mnom002404 | −Zm.PUB22/23-like protein1 |
| 180 | 487 | Mnom002405 | −Zm.PUB22/23-like protein2 |
| 181 | 488 | Mnom002408 | +At.CLV3 |
| 182 | 489 | Mnom002410 | +At.mCLV3 |
| 183 | 490 | 1183506:1 | +At.CLE-like AT5G59305.1 |
| | | | precursor |
| 184 | 491 | 1183506:1 | +At.CLE-like AT5G59305.1 |
| | | | precursor |
| 185 | 492 | Mnom002414 | +At.CLE-like AT5G59305.1 |
| | | | mature |
| 186 | 493 | Mnom002426 | +Gh.Skd1 (DQ444282) |
| 187 | 494 | 1163080:1 | +At.ERF1 |
| 188 | 495 | 1179434:1 | +Zm.homolog Basic |
| | | | Transcription Factor 2 |
| | | | (CGPG1840) |
| 189 | 496 | Mnom002481 | +Zm.ATH1-1 |
| 190 | 497 | 1187102:1 | +Zm.ATH1-2 |
| 191 | 498 | 1186139:1 | +Zm.ZRP4 |
| 192 | 499 | Mnom002490 | +Zm.LSH1 |
| 193 | 500 | Mnom002494 | +Zm.Putative cytochrome |
| | | | P450 |
| 194 | 501 | 1183523:1 | +At.NRT1.5 (nitrate |
| | | | transporter 1.5) |
| 195 | 502 | Mnom002497 | −Zm.homolog of CIPK8 |
| | | | (Calcineurin B-like (CBL)- |
| | | | interacting protein kinase 8) |
| 196 | 503 | 1184967:1 | +Zm.RINUE8 |
| 197 | 504 | 1188565:1 | +At.phyA |
| 198 | 505 | Mnom002520 | +At.phyA Y242H |
| 199 | 506 | 1184536:1 | +At.phyA F389A |
| 200 | 507 | PHE0024283 | +Os.BIRF1 |
| | | | (Benzathiodiazole induced |
| | | | ring finger protein 1) |
| 201 | 508 | PHE0024283 | +Os.BIRF1 |
| | | | (Benzathiodiazole induced |
| | | | ring finger protein 1) |
| 202 | 509 | 1181806:1 | +Ec. cspG - D63344 |
| 203 | 510 | 1181808:1 | +At.PHE0007559_OXI1 |
| | | | (OXIDATIVE SIGNAL- |
| | | | INDUCIBLE1) |
| 204 | 511 | 1183495:1 | +Zm.PHE0007208_Putative |
| | | | polysialic acid capsule |
| | | | expression protein |
| 205 | 512 | PHE0007541 | +Zm.PHE0007541_A1ZM008639_at |
| 206 | 513 | 1181787:1 | +Dh.PHE0007554_BCCT |
| | | | transporter |
| 207 | 514 | 1181764:1 | +Ta.PHE0007755_Td25a |
| 208 | 515 | 1181792:1 | +Cg.PHE0007792_nitroreductase |
| 209 | 516 | 1181783:1 | +At.PHE0009197_PF02519- |
| | | | Auxin_inducible-9 |
| 210 | 517 | 1184534:1 | +At.TZP, At5g43630 |
| 211 | 518 | Mnom002555 | −Gm.PUB22/PUB23 like |
| 212 | 519 | 1181848:1 | +Os.bZIP23(Basic leucine |
| | | | zipper 23) |
| 213 | 520 | 1185549:1 | +At.ZEP (Zeaxanthin |
| | | | epoxidase) |
| 214 | 521 | 1184133:1 | +Gm.Transcription factor |
| | | | LIM |
| 215 | 522 | 1181771:1 | +Gm.Putative pollen |
| | | | specific protein |
| 216 | 523 | Mnom002564 | +Gm.unknown protein |
| | | | contain domains similar to |
| | | | protein serine/threonine |
| | | | kinase |
| 217 | 524 | Mnom002565 | +Gm.MADS-box protein |
| 218 | 525 | 1181802:1 | +Gm.Seven in absentia |
| | | | protein family putative |
| 219 | 526 | 1183468:1 | +At.bHLH047 |
| 220 | 527 | Mnom002582 | +At.MYB114 |
| 221 | 528 | Mnom002603 | +At.G2930 |
| 222 | 529 | Mnom002604 | +At.G2784 |
| 223 | 530 | 1087245:1 | +At.G154 MADS-box |
| | | | protein |
| 224 | 531 | Mnom002645 | +At.Twin Sister of FT (TSF) |
| | | | protein |
| 225 | 532 | Mnom002652 | −Zm.homolog of |
| | | | Arabidopsis peptide |
| | | | transporter 5(PTR5) |
| 226 | 533 | Mnom002653 | +At.Transport protein |
| | | | associated with antigen |
| | | | processing2 (TAP2)/ |
| | | | Aluminium sensitive1 |
| | | | (ALS1) |
| 227 | 534 | Mnom002662 | +Cc.Asparagine |
| | | | synthetase codon |
| | | | optimized |
| 228 | 535 | Mnom002667 | +Cr.Acetate kinase |
| 229 | 536 | Mnom002668 | +Cr.phosphate |
| | | | acetyltransferase |
| 230 | 537 | Mnom002669 | +Cr.Lysine decarboxylase |
| 231 | 538 | 1066582:1 | +At.Aromatic and neutral |
| | | | amino acid transporter 1 |
| | | | (ANT1) |
| 232 | 539 | PHE0009168 | +Le.PHE0009168_JERF3 |
| | | | (Jasmonate and ethylene |
| | | | responsive factor 3) |
| 233 | 540 | Mnom002676 | +Gm.CHI |
| 234 | 541 | Mnom002706 | −Zm.RAA1 |
| 235 | 542 | Mnom002708 | +Zm.Albino3 |
| 236 | 543 | 1186548:1 | +At.LPA2 (Low PSII |
| | | | Accumulation2) |
| 237 | 544 | Mnom002714 | −Zm.Lew1 (leaf wilting1) |
| 238 | 545 | Mnom002716 | −Zm.eIF-5A |
| 239 | 546 | 1190083:1 | +Vf.Amino acid permease |
| | | | 1 (AAP1) |
| 240 | 547 | Mnom002721 | +Gm.G1481 a homolog of |
| | | | AtG1481 |
| 241 | 548 | PHE0024832 | +At.Nitrate Transporter |
| | | | 1.5 |
| 242 | 549 | 1190115:1 | +Pp.HB4 |
| 243 | 550 | 1187139:1 | +At.HB1 |
| 244 | 551 | 1187139:1 | +At.HB1 |
| 245 | 552 | 1187139:1 | +At.HB1 |
| 246 | 553 | 1188214:1 | +Zm.PP2C |
| 247 | 554 | 1189232:1 | +Zm.PP2C |
| 248 | 555 | Mnom002750 | +Zm.PP2C |
| 249 | 556 | 1188212:1 | +Os.PP2C |
| 250 | 557 | PHE0006743 | +At.CGPG151 putative |
| | | | two-component response |
| | | | regulator protein |
| 251 | 558 | PHE0004397 | +Os.G3848 |
| 252 | 559 | PHE0006386 | +At.CGPG1137 expressed |
| | | | protein |
| 253 | 560 | 1187105:1 | +Zm.tubby 4 |
| 254 | 561 | Mnom002784 | +At.Cationic amino acid |
| | | | transporter1 (CAT1) |
| 255 | 562 | 1194701:1 | +At.Cationic amino acid |
| | | | transporter6 (CAT6) |
| 256 | 563 | 1194425:1 | +Cr.fructose 6 phosphate |
| | | | aldolase |
| 257 | 564 | PHE0025040 | +Cr.Acetate kinase |
| 258 | 565 | PHE0025042 | +Cr.Lysine decarboxylase |
| 259 | 566 | 1190117:1 | +At.galactose-binding |
| | | | lectin family protein |
| 260 | 567 | 1190127:1 | +At.zinc finger (C3HC4- |
| | | | type RING finger) family |
| | | | protein |
| 261 | 568 | 1191292:1 | +At.putative amino acid |
| | | | transport protein |
| 262 | 569 | 1190123:1 | +At.zinc finger (C3HC4- |
| | | | type RING finger) |
| | | | family protein |
| 263 | 570 | 1194201:1 | +At.lesion inducing |
| | | | protein-related |
| 264 | 571 | PHE0025430 | +Ta.DNA binding with one |
| | | | finger (TaDOF1) |
| 265 | 572 | 1187623:1 | +Ta.DNA binding with one |
| | | | finger (TaDOF1) |
| 266 | 573 | 1191310:1 | +Ta.DNA binding with one |
| | | | finger (TaDOF1) |
| 267 | 574 | Mnom002849 | +Bn.G1988 homologue |
| 268 | 575 | PHE0025389 | +At.phyA S602A |
| 269 | 576 | 1192540:1 | +At.phyA S602A |
| 270 | 577 | 1192540:1 | +At.phyA S602A |
| 271 | 578 | Mnom002860 | +At.ammonium |
| | | | transporter At Amt1; 5 |
| 272 | 579 | Mnom002864 | +At.TF At5g56860 |
| 273 | 580 | Mnom002882 | +At.Gene 5160 |
| 274 | 581 | Mnom002886 | +At.RUS1 |
| 275 | 582 | Mnom002887 | +Sl.TERF1 (Tomato |
| | | | ethylene response factor |
| | | | 1) |
| 276 | 583 | 1195225:1 | +Ca.Pathogen-induced |
| | | | membrane protein 1 |
| 277 | 584 | Mnom002889 | +Zm.DEK1 (Defective |
| | | | Kernel 1) |
| 278 | 585 | Mnom002891 | +Gm.BiPD(Endoplasmic |
| | | | reticulum HSC70-cognate |
| | | | binding protein) |
| 279 | 586 | 1191787:1 | +Ta.W55a |
| 280 | 587 | 1194731:1 | +At.WRKY25 |
| 281 | 588 | Mnom002895 | +At.WRKY33 |
| 282 | 589 | Mnom002896 | +Ps.Sym37 |
| 283 | 590 | Mnom002898 | +Mt.DMI2 |
| 284 | 591 | Mnom002899 | +Pv.NAS2 (asparagines |
| | | | synthetase 2) |
| 285 | 592 | 1187621:1 | +Os.Gene 179_MonNom |
| 286 | 593 | 1187788:1 | +Vv.Gene 197 |
| 287 | 594 | 1194056:1 | +Os.CGFS type |
| | | | glutaredoxin |
| | | | (mitochondrial form) |
| 288 | 595 | Mnom002995 | +Os.Multidomian |
| | | | Monothiol glutaredoxin |
| 289 | 596 | 1194054:1 | +At.Monothiol |
| | | | glutaredoxin |
| 290 | 597 | Mnom003001 | +At.GTPase ObgE family |
| 291 | 598 | Mnom003035 | +At.RGE1(RETARDED |
| | | | GROWTH OF EMBRYO1) |
| 292 | 599 | 1192245:1 | +Zm.Hypothetical protein |
| | | | specific to corn |
| | | | endosperm |
| 293 | 600 | 1194063:1 | +At.leucine-rich repeat |
| | | | family protein |
| 294 | 601 | PHE0021991 | +At.1g03190 (AtXPD) |
| 295 | 602 | 1193217:1 | +At.CoAse3(Coenzyme A |
| | | | pyrophosphatase 3) |
| 296 | 603 | Mnom003125 | +At.MRB1 (Membrane |
| | | | Related Bigger 1) |
| 297 | 604 | 1194048:1 | +Ca.RZFP1 (RING Zinc |
| | | | Finger Protein 1) |
| 298 | 605 | Mnom003151 | +Gm.G154 homologue3 |
| 299 | 606 | Mnom003152 | +Gh.G154 homologue |
| 300 | 607 | Mnom003153 | +Gm.AT1G47670 - LYS/HIS |
| | | | TRANSPORTER 7 |
| 301 | 608 | Mnom003154 | +Gm.AT5G23810 - AAP7 |
| 302 | 609 | Mnom003157 | +Gm.GXGXG domain |
| | | | containing protein |
| 303 | 610 | Mnom003158 | +Gm.DUF_domain |
| | | | containing protein |
| 304 | 611 | Mnom003159 | +Gm.AT5G65660 - |
| | | | hydroxyproline-rich |
| | | | glycoprotein family |
| | | | protein |
| 305 | 612 | Mnom003178 | +Le.AIM1(Abscisic acid- |
| | | | induced myb1) |
| 306 | 613 | Mnom003189 | +Ta.Wheat hemoglobin |
| | | | HMP reductase fusion |
| 307 | 614 | Mnom003190 | +Pp.Flavohemoglobin |
| | | | (codon optimized) |

Selection Methods for Transgenic Plants with Enhanced Agronomic Trait

Within a population of transgenic plants each regenerated from a plant cell having a nucleus with recombinant DNA many plants that survive to fertile transgenic plants that produce seeds and progeny plants will not exhibit an enhanced agronomic trait. Selection from the population is necessary to identify one or more transgenic plant cells having a transgenic nucleus that can provide plants with the enhanced trait. Transgenic plants having enhanced traits are selected from populations of plants regenerated or derived from plant cells transformed as described herein by evaluating the plants in a variety of assays to detect an enhanced trait, e.g. enhanced water use efficiency, enhanced cold tolerance, increased yield, enhanced nitrogen use efficiency, enhanced seed protein and enhanced seed oil. These assays also may take many forms including, but not limited to, direct screening for the trait in a greenhouse or field trial or by screening for a surrogate trait. Such analyses can be directed to detecting changes in the chemical composition, biomass, physiological properties, morphology of the plant. Changes in chemical compositions such as nutritional composition of grain can be detected by analysis of the seed composition and content of protein, free amino acids, oil, free fatty acids, starch or tocopherols. Changes in biomass characteristics can be made on greenhouse or field grown plants and can include plant height, stem diameter, root and shoot dry weights; and, for corn plants, ear length and diameter. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in morphology can be measured by visual observation of tendency of a transformed plant with an enhanced agronomic trait to also appear to be a normal plant as compared to changes toward bushy, taller, thicker, narrower leaves, striped leaves, knotted trait, chlorosis, albino, anthocyanin production, or altered tassels, ears or roots. Other selection properties include days to pollen shed, days to silking, leaf extension rate, chlorophyll content, leaf temperature, stand, seedling vigor, internode length, plant height, leaf number, leaf area, tillering, brace roots, stay green, stalk lodging, root lodging, plant health, barreness/prolificacy, green snap, and pest resistance. In addition, phenotypic characteristics of harvested grain may be evaluated, including number of kernels per row on the ear, number of rows of kernels on the ear, kernel abortion, kernel weight, kernel size, kernel density and physical grain quality.

Assays for screening for a desired trait are readily designed by those practicing in the art. The following illustrates useful screening assays for corn traits using hybrid corn plants. The assays can be readily adapted for screening other plants such as canola, cotton and soybean either as hybrids or inbreds.

Transgenic corn plants having nitrogen use efficiency are identified by screening in fields with three levels of nitrogen (N) fertilizer being applied, e.g. low level (0 N), medium level (80 lb/ac) and high level (180 lb/ac). Plants with enhanced nitrogen use efficiency provide higher yield as compared to control plants.

Transgenic corn plants having enhanced yield are identified by screening using progeny of the transgenic plants over multiple locations with plants grown under optimal production management practices and maximum weed and pest control. A useful target for improved yield is a 5% to 10% increase in yield as compared to yield produced by plants grown from seed for a control plant. Selection methods may be applied in multiple and diverse geographic locations, for example up to 16 or more locations, over one or more planting seasons, for example at least two planting seasons, to statistically distinguish yield improvement from natural environmental effects.

Transgenic corn plants having enhanced water use efficiency are identified by screening plants in an assay where water is withheld for a period to induce stress followed by watering to revive the plants. For example, a useful selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method are the changes in plant growth rate as determined by height and biomass during a vegetative drought treatment.

Transgenic corn plants having enhanced cold tolerance are identified by screening plants in a cold germination assay and/or a cold tolerance field trial. In a cold germination assay trays of transgenic and control seeds are placed in a growth chamber at 9.7° C. for 24 days (no light). Seeds having higher germination rates as compared to the control are identified as having enhanced cold tolerance. In a cold tolerance field trial plants with enhanced cold tolerance are identified from field planting at an earlier date than conventional Spring planting for the field location. For example, seeds are planted into the ground around two weeks before local farmers begin to plant corn so that a significant cold stress is exerted onto the crop, named as cold treatment. Seeds also are planted under local optimal planting conditions such that the crop has little or no exposure to cold condition, named as normal treatment. At each location, seeds are planted under both cold and normal conditions preferably with multiple repetitions per treatment.

Transgenic corn plants having seeds with increased protein and/or oil levels are identified by analyzing progeny seed for protein and/or oil. Near-infrared transmittance spectrometry is a non-destructive, high-throughput method that is useful to determine the composition of a bulk seed sample for properties listed in table 2.

TABLE 2

| | |
|---|---|
| Typical sample(s): | Whole grain corn and soybean seeds |
| Typical analytical range: | Corn - moisture 5-15%, oil 5-20%, |
| | protein 5-30%, starch 50-75%, and density |
| | 1.0-1.3%. |
| | Soybean - moisture 5-15%, oil 15-25%, |
| | and protein 35-50%. |

Although the plant cells and methods of this invention can be applied to any plant cell, plant, seed or pollen, e.g. any fruit, vegetable, grass, tree or ornamental plant, the various aspects of the invention are preferably applied to corn, soybean, cotton, canola, alfalfa, wheat, rice, sugarcane, and sugar beet plants. In many cases the invention is applied to corn plants that are inherently resistant to disease from the Mal de Rio Cuarto virus or the Puccina sorghi fungus or both.

The following examples are included to demonstrate aspects of the invention, those of skill in the art should, in light of the present disclosure, appreciate that many changes can be made in the specific aspects which are disclosed and still obtain a like or similar results without departing from the spirit and scope of the invention.

### Example 1

### Plant Expression Constructs

This example illustrates the construction of plasmids for transferring recombinant DNA into a plant cell nucleus that can be regenerated into transgenic plants.

### A. Plant Expression Constructs for Corn Transformation

A base corn transformation vector pMON93039, as set forth in SEQ ID NO:36443, illustrated in Table 3, is fabricated for use in preparing recombinant DNA for Agrobacterium-mediated transformation into corn tissue.

TABLE 3

| | | | Coordinates of |
|---|---|---|---|
| Function | Name | Annotation | SEQ ID NO: 36443 |
| Agrobacterium | B-AGRtu.right border | Agro right border sequence, | 11364-11720 |
| T-DNA transfer | | essential for transfer of T- | |
| | | DNA. | |
| Gene of interest | E-Os.Act1 | Upstream promoter region of | 19-775 |
| expression | | the rice actin 1 gene | |
| cassette | E-CaMV.35S.2xA1-B3 | Duplicated35S A1-B3 | 788-1120 |
| | | domain without TATA box | |
| | P-Os.Act1 | Promoter region of the rice | 1125-1204 |
| | | actin 1 gene | |
| | L-Ta.Lhcb1 | 5′ untranslated leader of | 1210-1270 |
| | | wheat major chlorophyll a/b | |
| | | binding protein | |
| | I-Os.Act1 | First intron and flanking | 1287-1766 |
| | | UTR exon sequences from | |
| | | the rice actin 1 gene | |
| | T-St.Pis4 | 3′ non-translated region of | 1838-2780 |
| | | the potato proteinase | |
| | | inhibitor II gene which | |
| | | functions to direct | |
| | | polyadenylation of the | |
| | | mRNA | |
| Plant selectable | P-Os.Act1 | Promoter from the rice actin | 2830-3670 |
| marker | | 1 gene | |
| expression | L-Os.Act1 | First exon of the rice actin 1 | 3671-3750 |
| cassette | | gene | |
| | I-Os.Act1 | First intron and flanking | 3751-4228 |
| | | UTR exon sequences from | |
| | | the rice actin 1 gene | |
| | TS-At.ShkG-CTP2 | Transit peptide region of | 4238-4465 |
| | | Arabidopsis EPSPS | |
| | CR-AGRtu.aroA-CP4.nat | Coding region for bacterial | 4466-5833 |
| | | strain CP4 native aroA gene. | |
| | T-AGRtu.nos | A 3′ non-translated region of | 5849-6101 |
| | | the nopaline synthase gene | |
| | | of Agrobacterium | |
| | | tumefaciens Ti plasmid | |
| | | which functions to direct | |
| | | polyadenylation of the | |
| | | mRNA. | |
| Agrobacterium | B-AGRtu.left border | Agro left border sequence, | 6168-6609 |
| T-DNA transfer | | essential for transfer of T- | |
| | | DNA. | |
| Maintenance in | OR-Ec.oriV-RK2 | The vegetative origin of | 6696-7092 |
| E. coli | | replication from plasmid | |
| | | RK2. | |
| | CR-Ec.rop | Coding region for repressor | 8601-8792 |
| | | of primer from the ColE1 | |
| | | plasmid. Expression of this | |
| | | gene product interferes with | |
| | | primer binding at the origin | |
| | | of replication, keeping | |
| | | plasmid copy number low. | |
| | OR-Ec.ori-ColE1 | The minimal origin of | 9220-9808 |
| | | replication from the E. coli | |
| | | plasmid ColE1. | |
| | P-Ec.aadA-SPC/STR | Promoter for Tn7 | 10339-10380 |
| | | adenylyltransferase | |
| | | (AAD(3″)) | |
| | CR-Ec.aadA-SPC/STR | Coding region for Tn7 | 10381-11169 |
| | | adenylyltransferase | |
| | | (AAD(3″)) conferring | |
| | | spectinomycin and | |
| | | streptomycin resistance. | |
| | T-Ec.aadA-SPC/STR | 3′ UTR from the Tn7 | 11170-11227 |
| | | adenylyltransferase | |
| | | (AAD(3″)) gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the intron element (coordinates 1287-1766) and the polyadenylation element (coordinates 1838-2780).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, the sense and anti-sense DNA is derived from an endogenous corn gene that expresses a corn protein with an amino acid sequence of SEQ ID NO: 316, 344, 346, 350-351, 355, 357, 360-365, 384-388, 393, 397-400, 404, 413, 415-416, 425-426, 474, 476-487, 502, 532, 541, or 544-545 or the corn homolog of SEQ ID NOs:309, 311, 324, 373, 395, 454, or 518.

### B. Plant Expression Constructs for Soy and Canola Transformation

Vectors for use in transformation of soybean and canola tissue are prepared having the elements of expression vector pMON82053 (SEQ ID NO: 36444) as shown in Table 4 below.

TABLE 4

| | | | Coordinates of |
|---|---|---|---|
| Function | Name | Annotation | SEQ ID NO: 36444 |
| Agrobacterium T- | B-AGRtu.left border | Agro left border sequence, essential for | 6144-6585 |
| DNA transfer | | transfer of T-DNA. | |
| Plant selectable | P-At.Act7 | Promoter from the Arabidopsis actin 7 gene | 6624-7861 |
| marker expression | L-At.Act7 | 5′UTR of Arabidopsis Act7 gene | |
| cassette | I-At.Act7 | Intron from the Arabidopsis actin7 gene | |
| | TS-At.ShkG-CTP2 | Transit peptide region of Arabidopsis | 7864-8091 |
| | | EPSPS | |
| | CR-AGRtu.aroA- | Synthetic CP4 coding region with dicot | 8092-9459 |
| | CP4.nno_At | preferred codon usage. | |
| | T-AGRtu.nos | A 3′ non-translated region of the nopaline | 9466-9718 |
| | | synthase gene of Agrobacterium | |
| | | tumefaciens Ti plasmid which functions to | |
| | | direct polyadenylation of the mRNA. | |
| Gene of interest | P-CaMV.35S-enh | Promoter for 35S RNA from CaMV | 1-613 |
| expression cassette | | containing a duplication of the −90 to −350 | |
| | | region. | |
| | T-Gb.E6-3b | 3′ untranslated region from the fiber protein | 688-1002 |
| | | E6 gene of sea-island cotton. | |
| Agrobacterium T- | B-AGRtu.right | Agro right border sequence, essential for | 1033-1389 |
| DNA transfer | border | transfer of T-DNA. | |
| Maintenance in E. coli | OR-Ec.oriV-RK2 | The vegetative origin of replication from | 5661-6057 |
| | | plasmid RK2. | |
| | CR-Ec.rop | Coding region for repressor of primer from | 3961-4152 |
| | | the ColE1 plasmid. Expression of this gene | |
| | | product interferes with primer binding at the | |
| | | origin of replication, keeping plasmid copy | |
| | | number low. | |
| | OR-Ec.ori-ColE1 | The minimal origin of replication from the | 2945-3533 |
| | | E. coli plasmid ColE1. | |
| | P-Ec.aadA-SPC/STR | Promoter for Tn7 adenylyltransferase | 2373-2414 |
| | | (AAD(3″)) | |
| | CR-Ec.aadA- | Coding region for Tn7 adenylyltransferase | 1584-2372 |
| | SPC/STR | (AAD(3″)) conferring spectinomycin and | |
| | | streptomycin resistance. | |
| | T-Ec.aadA-SPC/STR | 3′ UTR from the Tn7 adenylyltransferase | 1526-1583 |
| | | (AAD(3″)) gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 1-613) and the polyadenylation element (coordinates 688-1002).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, for soybean the sense and anti-sense DNA is derived from an endogenous soybean gene that expresses a soybean protein with an amino acid sequence of SEQ ID NOs: 309, 324, 373, 395, 518 or is a soybean homolog of SEQ ID NOs: 311, 316, 344, 346, 350-351, 355, 357, 360-365, 384-388, 393, 397-400, 404, 413, 415-416, 425-426, 454, 474, 476-487, 502, 532, 541, or 544-545, and for canola the sense and anti-sense DNA is derived from an endogenous canola gene that encodes the canola homolog of SEQ ID NOs: 309, 311, 316, 324, 344, 346, 350-351, 355, 357, 360-365, 373, 384-388, 393, 395, 397-400, 404, 413, 415-416, 425-426, 454, 474, 476-487, 502, 518, 532, 541, or 544-545.

### C. Cotton Transformation Vector

Plasmids for use in transformation of cotton tissue are prepared with elements of expression vector pMON99053 (SEQ ID NO: 36445) as shown in Table 5 below.

TABLE 5

| | | | Coordinates of |
|---|---|---|---|
| Function | Name | Annotation | SEQ ID NO: 36445 |
| Agrobacterium | B-AGRtu.right border | Agro right border sequence, | 1-357 |
| T-DNA transfer | | essential for transfer of T-DNA. | |
| Gene of interest | Exp-CaMV.35S- | Enhanced version of the 35S | 388-1091 |
| expression | enh + Ph.DnaK | RNA promoter from CaMV plus | |
| cassette | | the petunia hsp70 5′ untranslated | |
| | | region | |
| | T-Ps.RbcS2-E9 | The 3′ non-translated region of | 1165-1797 |
| | | the pea RbcS2 gene which | |
| | | functions to direct | |
| | | polyadenylation of the mRNA. | |
| Plant selectable | Exp-CaMV.35S | Promoter and 5′ untranslated | 1828-2151 |
| marker | | region from the 35S RNA of | |
| expression | | CaMV | |
| cassette | CR-Ec.nptII-Tn5 | Coding region for neomycin | 2185-2979 |
| | | phosphotransferase gene from | |
| | | transposon Tn5 which confers | |
| | | resistance to neomycin and | |
| | | kanamycin | |
| | T-AGRtu.nos | A 3′ non-translated region of the | 3011-3263 |
| | | nopaline synthase gene of | |
| | | Agrobacterium tumefaciens Ti | |
| | | plasmid which functions to | |
| | | direct polyadenylation of the | |
| | | mRNA. | |
| Agrobacterium | B-AGRtu.left border | Agro left border sequence, | 3309-3750 |
| T-DNA transfer | | essential for transfer of T-DNA. | |
| Maintenance in | OR-Ec.oriV-RK2 | The vegetative origin of | 3837-4233 |
| E. coli | | replication from plasmid RK2. | |
| | CR-Ec.rop | Coding region for repressor of | 5742-5933 |
| | | primer from the ColE1 plasmid. | |
| | | Expression of this gene product | |
| | | interferes with primer binding at | |
| | | the origin of replication, keeping | |
| | | plasmid copy number low. | |
| | OR-Ec.ori-ColE1 | The minimal origin of | 6361-6949 |
| | | replication from the E. coli | |
| | | plasmid ColE1. | |
| | P-Ec.aadA-SPC/STR | Promoter for Tn7 | 7480-7521 |
| | | adenylyltransferase (AAD(3″)) | |
| | CR-Ec.aadA-SPC/STR | Coding region for Tn7 | 7522-8310 |
| | | adenylyltransferase (AAD(3″)) | |
| | | conferring spectinomycin and | |
| | | streptomycin resistance. | |
| | T-Ec.aadA-SPC/STR | 3′ UTR from the Tn7 | 8311-8368 |
| | | adenylyltransferase (AAD(3″)) | |
| | | gene of E. coli. | |

To construct transformation vectors for expressing a protein identified in Table 1, primers for PCR amplification of the protein coding nucleotides are designed at or near the start and stop codons of the coding sequence, in order to eliminate most of the 5′ and 3′ untranslated regions. The protein coding nucleotides are inserted into the base vector in the gene of interest expression cassette at an insertion site, i.e. between the promoter element (coordinates 388-1091) and the polyadenylation element (coordinates 1165-1797).

To construct transformation vectors for suppressing a protein identified in Table 1, the amplified protein coding nucleotides are assembled in a sense and antisense arrangement and inserted into the base vector at the insertion site in the gene of interest expression cassette to provide transcribed RNA that will form a double-stranded RNA for RNA interference suppression of the protein. More specifically, the sense and anti-sense DNA is derived from an endogenous cotton gene that encodes the cotton homolog of SEQ ID NO: 309, 311, 316, 324, 344, 346, 350-351, 355, 357, 360-365, 373, 384-388, 393, 395, 397-400, 404, 413, 415-416, 425-426, 454, 474, 476-487, 502, 518, 532, 541, or 544-545.

### D. Plant Expression Constructs for Gene Stacking in Corn.

A base corn transformation vector pMON96782, as set forth in SEQ ID NO: 36446, illustrated in Table 6, is fabricated for use in preparing recombinant DNA for Agrobacterium-mediated transformation into corn tissue.

TABLE 6

| | | | Coordinates of |
|---|---|---|---|
| Function | Name | Annotation | SEQ ID NO: 36446 |
| Agrobacterium | B-AGRtu.right border | Agro right border sequence, | 1-357 |
| T-DNA transfer | | essential for transfer of T-DNA. | |
| Gene of interest | P-Os.Act1 | Promoter region of the rice actin 1 | 403-1243 |
| expression | | gene | |
| cassette 1 | L-Os.Act1 | 5′ untranslated leader of rice actin 1 | 1244-1323 |
| | | gene | |
| | I-Os.Act1 | First intron and flanking UTR exon | 1324-1801 |
| | | sequences from the rice actin 1 | |
| | | gene | |
| | T-Ta.Hsp17 | 3′ un-translated region of wheat low | 1834-2043 |
| | | molecular weight heat shock | |
| | | protein gene | |
| Gene of interest | E-Os.Act1 | Upstream Promoter region of rice | 2136-2892 |
| expression | | actin 1 | |
| cassette 2 | | | |
| | E-CaMV.35S.2xA1- | 35S A1-B3 Domain | 2905-2937 |
| | B3 | | |
| | P-Os.Act1 | Promoter from rice actin gene | 3242-3321 |
| | L-Ta.Lhcb1 | 5′ untranslated leader from wheat | 3327-3387 |
| | | chlorophyll protein | |
| | I-Os.Act1 | Intron and 5′ untranslated region | 3404-3883 |
| | | from rice actin 1 gene | |
| | T-AGRtu.tr7 | 3′ untranslated region from | 3918-4425 |
| | | “transcript 7” of Agrobacterium | |
| Plant selectable | P.Os.TubA | Promoter of alpha-tubulin gene of | 4452-5650 |
| marker | | rice | |
| expression | | | |
| cassette | | | |
| | L.Os.TubA | 5′ untranslated region of an alpha | 5651-5736 |
| | | tubulin from rice | |
| | I.Os.TubA | Intron 1 of an alpha tubulin from | 5737-6632 |
| | | rice. | |
| | Ts.Ta.waxy.nno_Zm | Chloroplast transit peptide from | 6637-6846 |
| | | wheat starch synthase | |
| | Cr.AGRtu.aroA- | CP4 EPSPS gene | 6847-8214 |
| | CP4.nno_Zm | | |
| | T.Os.TubA | 3′ untranslated region of alpha | 8219-8800 |
| | | tubulin from rice | |
| Agrobacterium | B-AGRtu.left border | Left border sequence for T-DNA | 8828-9269 |
| T-DNA transfer | | transfer | |
| Maintenance in | OR-Ec.oriV-RK2 | Origin of replication from the | 9356-9752 |
| E. coli | | E. coli plasmid RK2. | |
| | Cr-Ec.rop | Coding region for represser of | 11261-11452 |
| | | primer from ColE1 plasmid | |
| | OR-Ec.ori-ColE1 | Minimum origin of replication from | 11880-12468 |
| | | E. coli colE1 plasmid. | |
| | P-Ec.aadA-SPC/STR | Promoter for Tn7 | 12999-13040 |
| | | adenylyltransferase gene | |
| | CR-Ec.aadA- | Coding region for Tn7 | 13041-13829 |
| | SPC/STR | adenylyltransferase gene | |
| | T-Ec.aadA-SPC/STR | 3′ untranslated region from Tn7 | 13830-13887 |
| | | adenylyltransferase gene | |

Primers for PCR amplification of protein coding nucleotides of the genes of interest are designed at or near the start and stop codons of the coding sequence, in order to eliminate most of the 5′ and 3′ untranslated regions. Protein coding regions of genes encoding a first and second protein of interest are amplified. The amplified region from the first gene of interest is cloned between nucleotides 1801 and 1834 of the base vector and the amplified region from the second gene of interest is cloned between nucleotides 3883 and 3918 of the base vector.

### Example 2

### Corn Transformation

This example illustrates transformation methods useful in producing a transgenic nucleus in a corn plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil. A plasmid vector is prepared by cloning the DNA of SEQ ID NO:1 into the gene of interest expression cassette in the base vector for use in corn transformation of corn tissue provided in Example 1, Table 3.

For Agrobacterium-mediated transformation of corn embryo cells corn plants of a readily transformable line are grown in the greenhouse and ears are harvested when the embryos are 1.5 to 2.0 mm in length. Ears are surface sterilized by spraying or soaking the ears in 80% ethanol, followed by air drying Immature embryos are isolated from individual kernels on surface sterilized ears. Prior to inoculation of maize cells, Agrobacterium cells are grown overnight at room temperature Immature maize embryo cells are inoculated with Agrobacterium shortly after excision, and incubated at room temperature with Agrobacterium for 5-20 minutes. Immature embryo plant cells are then co-cultured with Agrobacterium for 1 to 3 days at 23° C. in the dark. Co-cultured embryos are transferred to selection media and cultured for approximately two weeks to allow embryogenic callus to develop. Embryogenic callus is transferred to culture medium containing 100 mg/L paromomycin and subcultured at about two week intervals. Transformed plant cells are recovered 6 to 8 weeks after initiation of selection.

For Agrobacterium-mediated transformation of maize callus immature embryos are cultured for approximately 8-21 days after excision to allow callus to develop. Callus is then incubated for about 30 minutes at room temperature with the Agrobacterium suspension, followed by removal of the liquid by aspiration. The callus and Agrobacterium are co-cultured without selection for 3-6 days followed by selection on paromomycin for approximately 6 weeks, with biweekly transfers to fresh media. Paromomycin resistant calli are identified about 6-8 weeks after initiation of selection.

To regenerate transgenic corn plants a callus of transgenic plant cells resulting from transformation and selection is placed on media to initiate shoot development into plantlets which are transferred to potting soil for initial growth in a growth chamber at 26° C. followed by a mist bench before transplanting to 5 inch pots where plants are grown to maturity. The regenerated plants are self-fertilized and seed is harvested for use in one or more methods to select seeds, seedlings or progeny second generation transgenic plants (R2 plants) or hybrids, e.g. by selecting transgenic plants exhibiting an enhanced trait as compared to a control plant.

The above process is repeated to produce multiple events of transgenic corn plant cells that are transformed with recombinant DNA from each of the genes identified in Table 1. Events are designed to produce in the transgenic cells one of the proteins identified in Table 1, except the proteins of SEQ ID NOs: 316, 344, 346, 350-351, 355, 357, 360-365, 384-388, 393, 397-400, 404, 413, 415-416, 425-426, 474, 476-487, 502, 532, 541, and 544-545, and the corn homolog of SEQ ID NOs: 309, 311, 324, 373, 395, 454, and 518 which are suppressed. Progeny transgenic plants and seed of the transformed plant cells are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 3

### Soybean Transformation

This example illustrates plant transformation useful in producing a transgenic nucleus in a soybean plant cell, and the plants, seeds and pollen produced from a transgenic cell with such a nucleus having an enhanced trait, i.e. enhanced water use efficiency, enhanced cold tolerance, increased yield, enhanced nitrogen use efficiency, enhanced seed protein and enhanced seed oil.

For Agrobacterium mediated transformation, soybean seeds are imbided overnight and the meristem explants excised. The explants are placed in a wounding vessel. Soybean explants and induced Agrobacterium cells from a strain containing plasmid DNA with the gene of interest cassette and a plant selectable marker cassette are mixed no later than 14 hours from the time of initiation of seed imbibition, and wounded using sonication. Following wounding, explants are placed in co-culture for 2-5 days at which point they are transferred to selection media for 6-8 weeks to allow selection and growth of transgenic shoots. Resistant shoots are harvested approximately 6-8 weeks and placed into selective rooting media for 2-3 weeks. Shoots producing roots are transferred to the greenhouse and potted in soil. Shoots that remain healthy on selection, but do not produce roots are transferred to non-selective rooting media for an additional two weeks. Roots from any shoots that produce roots off selection are tested for expression of the plant selectable marker before they are transferred to the greenhouse and potted in soil.

The above process is repeated to produce multiple events of transgenic soybean plant cells that are transformed with recombinant DNA from each of the genes identified in Table 1. Events are designed to produce in the transgenic cells one of the proteins identified in Table 1, except the proteins of SEQ ID NOs: 309, 324, 373, 395, and 518 and the soybean homologs of SEQ ID NOs: 311, 316, 344, 346, 350-351, 355, 357, 360-365, 384-388, 393, 397-400, 404, 413, 415-416, 425-426, 454, 474, 476-487, 502, 532, 541, and 544-545, which are suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 4

### Cotton Transgenic Plants with Enhanced Agronomic Traits

This example illustrates plant transformation useful in producing a transgenic nucleus in a cotton plant cell, and the plants, seeds and pollen produced from a transgenic cell with such a nucleus having an enhanced trait, i.e. enhanced water use efficiency, increased yield, enhanced nitrogen use efficiency and enhanced seed oil.

Transgenic cotton plants containing each recombinant DNA having a sequence of SEQ ID NO: 1 through SEQ ID NO: 307 are obtained by transforming with recombinant DNA from each of the genes identified in Table 1 using Agrobacterium-mediated transformation. The above process is repeated to produce multiple events of transgenic cotton plant cells that are transformed with recombinant DNA from each of the genes identified in Table 1. Events are designed to produce in the transgenic cells one of the proteins identified in Table 1, except the cotton homologs of the proteins of SEQ ID NOs: 309, 311, 316, 324, 344, 346, 350-351, 355, 357, 360-365, 373, 384-388, 393, 395, 397-400, 404, 413, 415-416, 425-426, 454, 474, 476-487, 502, 518, 532, 541, and 544-545 which are suppressed.

From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

Progeny transgenic plants are selected from a population of transgenic cotton events under specified growing conditions and are compared with control cotton plants. Control cotton plants are substantially the same cotton genotype but without the recombinant DNA, for example, either a parental cotton plant of the same genotype that was not transformed with the identical recombinant DNA or a negative isoline of the transformed plant. Additionally, a commercial cotton cultivar adapted to the geographical region and cultivation conditions, i.e. cotton variety ST474, cotton variety FM 958, and cotton variety Siokra L-23, are used to compare the relative performance of the transgenic cotton plants containing the recombinant DNA.

Transgenic cotton plants with enhanced yield and water use efficiency are identified by growing under variable water conditions. Specific conditions for cotton include growing a first set of transgenic and control plants under “wet” conditions, i.e. irrigated in the range of 85 to 100 percent of evapotranspiration to provide leaf water potential of −14 to −18 bars, and growing a second set of transgenic and control plants under “dry” conditions, i.e. irrigated in the range of 40 to 60 percent of evapotranspiration to provide a leaf water potential of −21 to −25 bars. Pest control, such as weed and insect control is applied equally to both wet and dry treatments as needed. Data gathered during the trial includes weather records throughout the growing season including detailed records of rainfall; soil characterization information; any herbicide or insecticide applications; any gross agronomic differences observed such as leaf morphology, branching habit, leaf color, time to flowering, and fruiting pattern; plant height at various points during the trial; stand density; node and fruit number including node above white flower and node above crack boll measurements; and visual wilt scoring. Cotton boll samples are taken and analyzed for lint fraction and fiber quality. The cotton is harvested at the normal harvest timeframe for the trial area. Enhanced water use efficiency is indicated by increased yield, improved relative water content, enhanced leaf water potential, increased biomass, enhanced leaf extension rates, and improved fiber parameters.

### Example 5

### Canola Transformation

This example illustrates plant transformation useful in producing the transgenic canola plants of this invention and the production and identification of transgenic seed for transgenic canola having enhanced water use efficiency, enhanced cold tolerance, increased yield, enhanced nitrogen use efficiency, enhanced seed protein and enhanced seed oil.

Tissues from in vitro grown canola seedlings are prepared and inoculated with overnight-grown Agrobacterium cells containing plasmid DNA with the gene of interest cassette and a plant selectable marker cassette. Following co-cultivation with Agrobacterium, the infected tissues are allowed to grow on selection to promote growth of transgenic shoots, followed by growth of roots from the transgenic shoots. The selected plantlets are then transferred to the greenhouse and potted in soil. Molecular characterizations are performed to confirm the presence of the gene of interest, and its expression in transgenic plants and progenies. Progeny transgenic plants are selected from a population of transgenic canola events under specified growing conditions and are compared with control canola plants. Control canola plants are substantially the same canola genotype but without the recombinant DNA, for example, either a parental canola plant of the same genotype that is not transformed with the identical recombinant DNA or a negative isoline of the transformed plant.

Transgenic canola plant cells are transformed with each of the recombinant DNA identified in Table 1. The above process is repeated to produce multiple events of transgenic canola plant cells that are transformed with recombinant DNA from each of the genes identified in Table 1. Events are designed to produce in the transgenic cells one of the proteins identified in Table 1, except the canola homologs of the proteins of SEQ ID NOs: 309, 311, 316, 324, 344, 346, 350-351, 355, 357, 360-365, 373, 384-388, 393, 395, 397-400, 404, 413, 415-416, 425-426, 454, 474, 476-487, 502, 518, 532, 541, and 544-545 which are suppressed. Progeny transgenic plants and seed of the transformed plant cells are screened for enhanced water use efficiency, enhanced cold tolerance, increased yield, enhanced seed protein and enhanced seed oil. From each group of multiple events of transgenic plants with a specific recombinant DNA from Table 1 the event that produces the greatest enhancement in yield, water use efficiency, nitrogen use efficiency, enhanced cold tolerance, enhanced seed protein and enhanced seed oil is identified and progeny seed is selected for commercial development.

### Example 6

### Homolog Identification

This example illustrates the identification of homologs of proteins encoded by the DNA identified in Table 1 which is used to provide transgenic seed and plants having enhanced agronomic traits. From the sequence of the homologs, homologous DNA sequence can be identified for preparing additional transgenic seeds and plants of this invention with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided herein as SEQ ID NO: 308 through SEQ ID NO: 614 using NCBI “blastp” program with E-value cutoff of 1e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided herein as SEQ ID NO: 308 through SEQ ID NO: 614 using NCBI “blastp” program with E-value cutoff of 1e-4. Up to 1000 top hits were kept. A BLAST searchable database was constructed based on these hits, and is referred to as “SubDB”. SubDB is queried with each sequence in the Hit List using NCBI “blastp” program with E-value cutoff of 1e-8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. For proteins of table 1 having an identified Pfam domain module, homology to reported homologs was further confirmed by searching the respective identified homologs for conservation of the Pfam domain module identified in the protein from table 1. Homologs from a large number of distinct organisms were identified and are reported below in table 7 with the SEQ ID NO of the DNA sequence corresponding to original protein query sequence and the identified homologs as [SEQ ID NO]: [Homolog SEQ ID NOs].

TABLE 7
Protein Sequences and their Homologs

1: 636 819 1097 1350 1889 1995 2165 2787 2937 3028 3574 3736 3793 3928 4331 4566 4851 5066 5207 5357
5398 5663 5664 6013 6476 6570 6701 6787 6959 7007 7057 7258 7401 7447 8148 8257 8679 8816 8900 8924
9039 9090 9392 9851 10181 10329 10383 10613 10783 10976 11004 11066 11131 11286 11480 11647 11722
11765 11832 12171 12412 12447 12798 12834 12898 12940 13177 13451 13570 13719 13950 14299 14438
14548 14592 14935 15659 15902 16223 16224 16225 16226 16227 16228 16274 16317 16550 16814 16848
17067 17087 17201 17262 17352 17689 18175 18176 18185 18519 18676 18855 18874 19060 19276 19656
19751 19922 20349 20723 21061 21081 21283 21397 21735 21976 22091 22248 22532 22808 23081 23178
23387 23483 23707 23922 23947 24001 24337 24585 24850 25090 25237 25290 25466 25663 25684 25788
25908 26445 26583 26823 27032 27073 27318 27324 27326 28043 28105 28199 28277 28306 28415 28508
28742 28774 29005 29247 29511 29652 30073 30230 30408 30456 30761 30816 30999 31063 31352 31385
31512 31559 31695 31898 32035 32178 32232 32337 32385 32394 32445 32691 32878 33131 33247 33249
33364 33479 34197 34394 34855 35042 35135 35209 35494 35573 35615 35629 35765 35871 35940 36015
36023 36173
2: 898 1260 1265 1375 1421 1533 1551 2768 3382 3404 3669 4434 5153 5179 5688 7146 7374 7885 8499
9364 11279 13414 13415 13416 13417 13418 13419 13420 13421 13422 16455 16779 16780 16781 16782
16783 18179 18180 18532 19184 19230 19766 20402 20438 21437 22781 23681 27774 27990 27999 28285
33033 34259 35255 35282 35285 35288 35289 36420
3: 456 457 470 712 881 1142 1361 2336 2631 2853 2989 3462 3491 3834 3927 4028 4048 4060 4127 4140
4160 4191 4241 4250 4302 5192 5679 5712 5811 5914 7872 7874 9042 9469 13359 13901 13913 16134
16173 17389 17790 17866 18577 18938 19220 19851 20421 20992 22719 23039 23691 24702 27436 27600
29693 31156 33797 36004
4: 344 385 4194
5: 741 1095 1126 1282 1869 2005 2063 2201 2860 3039 3267 3269 3340 3524 4426 6056 7596 8593 9054
9182 9183 9360 9694 9714 10214 11228 11363 11572 12507 13329 13330 13331 13332 13333 13334 13335
13347 13348 13349 13350 13351 13352 13425 13973 14651 15098 15291 15709 17686 17750 18147 18555
18750 18786 18813 19370 19457 19479 19838 19839 20150 20152 20184 20693 21241 21595 21596 22542
22643 22644 22645 22646 22798 23714 25078 25487 26417 26441 26498 26499 26500 27004 27025 28214
29144 30518 30709 30758 31167 31344 31394 31692 31734 32109 32138 32578 32959 33935 36167 36426
6: 775 1376 1383 1619 1972 2004 2037 2825 2866 4396 6439 9718 11312 11422 12356 12359 12365 12381
12402 12407 12443 12450 12455 12467 12468 12470 12472 12495 12498 12500 12501 16180 17480 17889
17895 18236 18975 19053 19298 19392 19761 19783 20005 20053 21580 21704 22589 22648 30963 30964
31241 31242 31243 31244 31245 31246 31247 31248 31249 31250 31251 33709 36349
7: 653 714 763 991 1289 2076 2340 2710 2905 3406 7933 11367 13848 15284 17753 19310 19456 19919
22778 31068 36330
8: 648 691 832 889 950 1035 1049 1085 1555 1747 1952 2074 2235 3471 4715 7929 9413 10227 10570 11271
13763 13766 13911 15477 17962 18934 18990 18991 20092 21463 22102 22819 25479 26945 28548 32656
33989 36086 36325
9: 1767 2508 2758 2873 2995 3173 3272 3961 5849 13687 13715 17896 18662 22881
10: 12216 13118 14734 14862 15330 17937 17977 18728 22144 22171 25713 30281 31805 33005
11: 733 1167 1803 1947 2134 2187 2226 2258 2325 2473 2532 3031 3165 3758 3934 3971 4005 4310 4453
4496 4523 4677 4710 4788 4797 4806 4827 4846 4847 4902 4910 4916 4942 4963 4971 5030 5063 5175 5235
5259 5269 5307 5446 5448 5512 5525 5543 5576 5639 5651 5654 5695 5848 5957 5967 5972 5994 6049 6098
6115 6126 6145 6172 6203 6209 6210 6211 6231 6234 6270 6287 6288 6289 6294 6295 6296 6301 6314 6330
6343 6377 6602 6607 6631 6661 6671 6696 6725 7028 7208 7233 7283 7428 7627 7655 7691 7695 7721 7899
8093 8095 8098 8121 8254 8278 8289 8307 8326 8352 8384 8523 8649 8664 8684 8737 8741 8750 8775 8778
8799 8800 8831 8835 8907 8946 8953 9003 9004 9013 9153 91 61 9288 9340 9345 951 8 9553 9559 9574 9678
9702 9719 9766 9792 9919 9922 10063 10084 10101 10113 10209 10298 10304 10312 10349 10440 10442
10486 10497 10509 10628 10646 10654 10721 10754 10773 10776 10840 10857 10861 10876 10882 10899
11004 11025 11041 11051 11072 11096 11110 11194 11255 11258 11450 11504 11527 11553 11597 11599
11603 11685 11734 11737 11825 11938 12012 12036 12056 12069 12073 12077 12080 12112 12136 12156
12161 12171 12187 12233 12250 12269 12291 12318 12405 12521 12522 12541 12561 12576 12615 12623
12649 12666 12683 12698 12728 12740 12850 12921 13038 13108 13184 13442 13465 13511 13546 13620
13718 13743 13786 13815 13860 13863 13868 13921 13981 13992 14021 14160 14167 14171 14180 14194
14255 14470 14632 14681 14688 14730 14749 14809 14819 14846 14882 14925 14933 14943 14976 14977
14983 15012 15031 15089 15090 15129 15139 15144 15168 15199 15327 15329 15335 15511 15527 15537
15698 15758 15772 15886 15910 15978 16079 16149 16158 16210 16267 16359 16363 16368 16371 16519
16557 16571 16633 16656 16674 16712 16720 16734 16736 16752 16875 17052 17064 17079 17105 17141
17146 17150 17186 17203 17225 17247 17402 17443 17452 17453 17454 17455 17456 17479 17508 17512
17536 17587 17592 17715 17894 17908 17922 17958 17997 18095 18102 18116 18126 18153 18258 18336
18338 18367 18383 18412 18421 18429 18452 18455 18469 18493 18502 18661 18676 18736 18762 18777
18784 18913 19019 19093 19105 19153 19465 19619 19699 19700 19733 19741 19894 20086 20089 20168
20172 20189 20213 20230 20256 20295 20307 20308 20379 20394 20556 20558 20590 20591 20611 20637
20638 20729 20741 20750 20752 20764 20772 20869 20874 20888 20962 20978 21024 21073 21252 21287
21374 21426 21623 21678 21684 21763 21779 21818 21852 22027 22032 22034 22094 22125 22164 22205
22224 22253 22301 22307 22320 22337 22359 22378 22389 22420 22435 22450 22486 22519 22627 22652
22818 22890 22891 22895 22966 23069 23075 23102 23161 23176 23253 23298 23356 23408 23424 23442
23541 23542 23547 23554 23592 23619 23736 23779 23780 23811 23815 23824 23833 23838 23851 23879
23907 23934 23942 23958 24097 24101 24106 24125 24137 24145 24369 24376 24450 24479 24495 24558
24612 24638 24651 24698 24850 24899 25020 25090 25149 25154 25189 25200 25208 25218 25237 25258
25265 25271 25290 25303 25309 25337 25358 25361 25407 25584 25612 25629 25647 25648 25732 25741
26255 26339 26407 26445 26552 26694 26717 26723 26858 26880 26891 27003 27069 27124 27213 27324
27331 27374 27377 27379 27409 27493 27526 27546 27558 27567 27606 27626 27649 27650 27660 27695
27698 27707 27802 27866 27889 27912 27959 28049 28068 28113 28132 28174 28269 28377 28443 28478
28679 28719 28801 28856 28866 28881 28906 28914 29014 29022 29033 29106 29139 29160 29168 29242
29266 29285 29316 29337 29339 29404 29408 29454 29538 29566 29575 29598 29713 29716 29735 29739
29767 29773 29789 29793 29809 29826 29933 29935 29949 29968 30016 30057 30102 30125 30141 30162
30173 30188 30226 30230 30234 30277 30309 30368 30408 30429 30456 30472 30489 30540 30551 30561
30563 30609 30638 30639 30654 30683 30792 30793 30866 30878 30884 30904 30924 30967 31063 31134
31215 31216 31219 31375 31482 31490 31602 31733 31810 32008 32013 32023 32049 32051 32055 32234
32346 32358 32381 32398 32422 32462 32494 32521 32529 32653 32664 32669 32722 32753 32762 32772
32776 32789 32798 32855 32875 32889 32986 33002 33059 33119 33146 33152 33197 33219 33286 33300
33316 33329 33351 33442 33460 33486 33526 33621 33643 33663 33690 33697 33699 33772 33845 33846
33847 33848 33849 33850 33929 33930 33947 34024 34031 34038 34063 34085 34161 34164 34199 34216
34238 34251 34286 34288 34317 34334 34348 34399 34545 34598 34688 34697 34778 34808 34832 34879
34895 34900 34936 34972 34996 35057 35083 35111 35181 35229 35263 35268 35362 35377 35428 35470
35671 35679 35704 35708 35720 35733 35738 35770 35806 35842 35865 35892 35946 35949 36060 36083
36102 36124 36180 36206 36250 36267 36275 36398
12: 3551 4778 5210 5604 7474 8618 9196 10382 11155 11645 12560 12587 12810 12835 13066 13172 13568
13630 14570 15055 15890 16081 16563 17858 18083 18323 18839 20636 21261 21978 22393 23236 23580
24043 24809 25035 25102 25138 25306 25786 26437 27283 28010 28108 28725 29361 29646 29878 30100
30657 31040 31866 32676 34201 35216 35792
13: 4912 5245 5316 5329 6070 6515 6660 7632 7661 7690 7728 7864 8199 8295 8840 8957 8999 9165 9520
10348 10398 10414 10433 10463 10996 11052 11091 11132 11174 11457 11488 11625 11824 12140 12165
12225 12277 12548 12565 12715 12851 12866 12922 12930 12937 13095 13545 13595 13619 13647 13657
13778 13983 14473 14643 14733 14788 14801 15116 15379 15490 15541 15697 16023 16275 16387 16414
16443 16659 17092 17113 17899 17965 18161 18274 18275 18398 18438 18731 18765 19738 19755 20260
20850 20912 21251 21770 21778 22001 22022 22077 22099 22172 22267 22368 22461 23199 23240 23252
23609 23765 23993 24126 24132 24152 24664 25024 25343 25362 25404 25715 25803 25839 25961 26610
26670 27096 27557 27617 27641 27796 28079 28087 28101 28802 28841 28848 28965 29889 29907 30283
30294 30329 30335 30546 31591 31792 31986 32520 32522 32536 32619 32737 32965 33281 33532 33778
33832 34013 34236 34673 34791 34998 35123 35245 35604 35728
14: 451 452 467 2486 2567 4431 4518 4563 4590 4660 4684 4686 4727 4786 4809 4862 4864 5005 5019 5036
5055 5141 5201 5266 5284 5313 5324 5355 5371 5387 5479 5538 5551 5573 5609 5908 6074 6079 6132 6156
6173 6204 6233 6341 6360 6361 6497 6558 6568 6592 6620 6689 6733 6742 6747 6806 6874 6875 6916 7019
7069 7123 7128 7157 7177 7239 7457 7473 7636 7664 7680 7684 7731 8166 8224 8235 8299 8419 8506 8609
8637 8730 8771 8797 8869 9241 9325 9524 9529 9610 9759 9814 9830 9849 9912 9931 9972 9992 9999
10052 10334 10363 10467 10667 10677 10700 10738 10772 10782 10813 10850 10885 10894 10975 11031
11154 11460 11528 11569 11636 11704 11752 11766 11886 11930 11934 12016 12050 12201 12210 12211
12222 12323 12331 12352 12471 12530 12555 12586 12639 12691 12759 12763 12843 12916 13061 13076
13083 13145 13176 13195 13211 13429 13557 13625 13759 13967 14169 14281 14304 14359 14360 14402
14481 14512 14564 14634 14709 14715 14726 14826 14855 14864 14957 14979 15057 15207 15228 15342
15370 15517 15914 15936 15969 16069 16293 16441 16575 16593 16692 16718 16823 16851 16861 17036
17102 17228 17337 17351 17418 17421 17516 17582 17583 17727 17728 17912 17931 17966 17987 17988
18082 18106 18125 18309 18358 18363 18391 18476 18484 18707 18732 18843 18885 19620 19666 19703
19759 20284 20560 20573 20596 20602 20626 20696 20883 20911 20966 21004 21160 21210 21671 21753
21802 21987 22121 22145 22177 22180 22238 22278 22303 22326 22332 22341 22382 22416 22463 22504
22526 23086 23138 23263 23434 23481 23488 23520 23536 23588 23631 23760 24003 24041 24091 24304
24323 24392 24433 24490 24523 24549 24559 24647 24717 24761 24796 24797 24819 24827 24828 24829
24879 24915 25003 25007 25029 25033 25176 25231 25346 25368 25372 25396 25398 25492 25520 25747
25775 25827 25907 26249 26301 26359 26396 26571 26582 26738 26779 26785 26830 27074 27273 27355
27368 27369 27382 27398 27471 27495 27553 27621 27676 27751 27769 27772 27963 27968 28061 28092
28483 28502 28538 28555 28601 28602 28623 28696 28738 28806 28882 28901 28947 28994 29118 29119
29177 29272 29298 29401 29431 29456 29464 29547 29548 29582 29609 29637 29645 29660 29745 29813
29835 29883 29963 29980 29989 30054 30068 30155 30195 30256 30292 30332 30643 30876 30991 31019
31049 31161 31530 31809 31812 31840 31841 31842 31843 32072 32174 32238 32289 32377 32433 32443
32458 32501 32540 32564 32565 32567 32568 32621 32631 32639 32646 32673 32706 32872 32967 32999
33082 33185 33195 33293 33332 33381 33417 33456 33537 33540 33572 33635 33782 33833 34086 34104
34109 34131 34169 34247 34258 34287 34304 34308 34322 34423 34476 34512 34544 34615 34677 34685
34714 34716 34765 34781 34794 34796 34813 34826 34881 34942 34953 34979 35002 35046 35100 35103
35113 35121 35136 35142 35182 35395 35400 35427 35489 35496 35525 35526 35527 35546 35552 35572
35694 35829 35849 35955 35978 36111 36129 36187 36224 36289 36367 36383
15: 323 793 812 880 904 916 1016 1377 1453 1492 1516 1626 1764 1790 1791 1949 1950 2042 2154 2458
2616 2703 2819 2877 2911 2914 2948 3015 3134 3154 3260 3261 3270 3297 3303 3329 3365 3451 3852 3855
3915 4116 4129 4142 4167 4207 4246 4279 4370 4393 4460 4471 4500 4505 4547 4583 4595 4611 4612 4613
4616 4617 4663 4670 4673 4675 4811 4824 4858 4955 4983 5001 5003 5006 5009 5015 5018 5020 5025 5028
5029 5031 5032 5044 5046 5057 5071 5078 5108 5232 5335 5364 5382 5406 5441 5465 5477 5558 5585 5589
5599 5632 5676 5840 5906 5939 5948 5952 5961 5982 6164 6167 6176 6207 6363 6365 6537 6538 6555 6579
6608 6676 6680 6690 6692 6697 6703 6714 6722 6724 6755 6758 6770 6775 6779 6819 6913 6970 6979 7021
7034 7043 7045 7060 7114 7117 7135 7166 7173 7183 7206 7222 7225 7261 7266 7272 7305 7405 7406 7407
7413 7429 7432 7455 7456 7586 7853 7854 7863 8128 8139 8161 8169 8181 8182 8185 8186 8226 8229 8230
8252 8261 8268 8319 8348 8418 8492 8513 8542 8608 8610 8628 8631 8658 8677 8680 8696 8756 8764 8828
8844 8848 8849 8850 8899 8901 8974 9118 9121 9140 9145 9179 9192 9226 9227 9234 9235 9258 9465 9575
9595 9614 9615 9623 9624 9751 9769 9772 9778 9780 9805 9808 9813 9835 9920 9924 9991 10020 10056
10115 10154 10362 10364 10366 10371 10396 10444 10449 10450 10489 10505 10510 10515 10552 10553
10566 10599 10610 10617 10621 10632 10678 10733 10799 10824 10841 10853 10878 10880 10901 10905
10986 11026 11092 11106 11129 11203 11231 11385 11531 11537 11567 11694 11700 11705 11709 11715
11747 11784 11794 11801 11806 11810 11812 11816 11817 11828 11923 11942 11947 11948 11966 11997
12019 12081 12125 12213 12263 12271 12280 12303 12305 12404 12559 12625 12641 12657 12663 12725
12732 12733 12750 12774 12809 12885 12924 12947 13019 13036 13051 13088 13126 13130 13147 13214
13215 13258 13259 13343 13455 13457 13461 13474 13506 13509 13581 13640 13692 13748 13946 14012
14195 14253 14256 14264 14267 14286 14392 14430 14589 14787 14800 14821 14831 14885 14907 14917
14921 14939 14940 14949 15024 15062 15151 15162 15221 15244 15245 15248 15249 15252 15417 15470
15476 15480 15484 15679 15893 15894 15900 15927 15937 16057 16147 16198 16249 16263 16273 16320
16477 16527 16532 16533 16548 16552 16610 16660 16723 16838 16879 17022 17031 17072 17089 17093
17164 17172 17184 17224 17234 17245 17332 17367 17406 17408 17437 17438 17439 17440 17539 17658
17709 17711 17721 17755 17959 18151 18159 18198 18211 18218 18361 18439 18490 18516 18522 18769
18866 18869 18918 19129 19194 19350 19485 19494 19623 19640 19654 19984 20147 20248 20294 20300
20414 20473 20534 20579 20628 20635 20718 20721 20728 20739 20753 20815 20816 20819 20834 20845
20860 20862 20892 20898 20899 20907 20998 21000 21031 21052 21059 21063 21124 21141 21173 21174
21175 21176 21177 21178 21179 21180 21181 21182 21183 21217 21673 21717 21728 21872 21874 21875
22002 22039 22045 22069 22089 22225 22242 22250 22259 22330 22336 22357 22383 22390 22401 22437
22527 22534 22547 22754 22861 22892 22923 22946 22959 23018 23089 23100 23120 23144 23183 23202
23237 23268 23374 23388 23392 23393 23404 23411 23422 23435 23436 23437 23458 23492 23496 23508
23515 23544 23548 23597 23655 23759 23933 23960 23963 23971 23990 24037 24046 24048 24066 24133
24302 24335 24351 24389 24396 24480 24498 24503 24528 24569 24582 24639 24654 24659 24714 24733
24963 25131 25134 25168 25175 25199 25217 25230 25234 25248 25256 25264 25278 25280 25284 25298
25307 25446 25485 25523 25526 25527 25650 25670 25755 25831 25834 25838 26020 26186 26201 26202
26212 26217 26240 26286 26392 26418 26439 26492 26545 26548 26549 26566 26567 26569 26578 26615
26697 26722 26730 26735 26894 26898 26993 27047 27240 27247 27292 27340 27344 27346 27348 27387
27477 27478 27550 27696 27709 27742 27765 27813 27945 27949 27951 28014 28029 28034 28036 28037
28047 28082 28201 28246 28273 28286 28310 28347 28395 28432 28438 28519 28655 28691 28757 28795
28796 28798 28816 28818 28885 28894 28905 28913 28969 28970 29130 29151 29217 29218 29224 29227
29246 29249 29250 29276 29289 29310 29311 29313 29327 29436 29437 29452 29458 29483 29498 29512
29559 29611 29617 29622 29683 29736 29819 29823 29825 29828 29893 29938 29944 29948 29965 29972
29977 29982 29995 30041 30046 30055 30078 30080 30084 30126 30154 30157 30168 30208 30237 30252
30308 30314 30370 30377 30578 30580 30581 30695 30696 30733 30787 30845 30860 30868 30899 30900
31141 31252 31407 31481 31505 31508 31519 31551 31552 31553 31554 31577 31687 31693 31719 31737
31844 31856 31879 31907 31967 32000 32006 32010 32018 32037 32056 32115 32120 32124 32170 32264
32269 32292 32334 32341 32364 32365 32416 32420 32435 32453 32500 32503 32506 32516 32561 32605
32618 32622 32636 32648 32655 32679 32680 32681 32687 32938 32943 32948 32952 33016 33099 33106
33117 33127 33133 33196 33211 33216 33221 33236 33237 33288 33306 33311 33319 33326 33337 33349
33372 33375 33379 33380 33382 33390 33451 33476 33477 33481 33501 33502 33578 33588 33589 33593
33611 33804 33873 33990 34025 34052 34069 34073 34082 34111 34150 34170 34173 34175 34178 34203
34218 34220 34265 34266 34293 34303 34316 34326 34351 34353 34400 34402 34468 34469 34487 34508
34520 34540 34616 34626 34636 34702 34704 34708 34711 34721 34825 34847 34859 34876 34950 34967
35036 35050 35068 35099 35119 35130 35189 35193 35195 35227 35230 35242 35392 35402 35406 35421
35431 35434 35440 35452 35453 35501 35518 35548 35551 35700 35791 35813 35817 35827 35830 35835
35844 35847 35873 35874 35875 35919 35925 35938 35972 35974 36078 36085 36109 36110 36139 36151
36185 36205 36208 36270 36279 36357 36370 36376 36417 36425
16: 322 793 812 880 904 916 1016 1377 1453 1492 1516 1626 1764 1790 1791 1949 1950 2042 2154 2616
2703 2819 2877 2911 2914 2948 3015 3134 3154 3260 3261 3270 3297 3303 3329 3365 3451 3852 3855 3915
4116 4129 4142 4167 4207 4246 4279 4370 4393 4460 4471 4500 4505 4524 4547 4583 4595 4611 4613 4616
4617 4663 4670 4673 4675 4811 4824 4858 4955 4983 5001 5003 5006 5009 5015 5018 5020 5028 5029 5031
5032 5044 5057 5071 5108 5232 5335 5364 5382 5406 5441 5465 5477 5558 5585 5589 5599 5632 5676 5840
5906 5939 5948 5952 5961 5982 6031 6164 6167 6176 6207 6363 6365 6537 6538 6555 6579 6608 6676 6680
6692 6697 6703 6714 6722 6724 6755 6758 6767 6770 6775 6779 6819 6913 6970 6979 7021 7034 7043 7045
7060 7114 7117 7135 7166 7173 7183 7206 7222 7225 7261 7266 7272 7305 7405 7406 7407 7413 7429 7432
7455 7456 7586 7853 7854 7863 8128 8139 8161 8169 8181 8182 8185 8186 8226 8229 8230 8252 8261 8268
8319 8348 8418 8492 8513 8542 8559 8608 8610 8628 8631 8658 8677 8680 8696 8720 8756 8764 8828 8848
8849 8850 8899 8901 8974 9118 9121 9140 9145 9179 9192 9193 9194 9226 9227 9234 9235 9258 9465 9575
9595 9614 9615 9623 9624 9751 9769 9772 9778 9780 9805 9808 9813 9835 9914 9920 9924 9991 10020
10056 10115 10154 10362 10364 10366 10371 10396 10416 10444 10449 10450 10454 10489 10505 10510
10515 10552 10553 10566 10599 10610 10617 10632 10678 10733 10799 10824 10841 10853 10878 10880
10901 10905 10986 11026 11092 11106 11129 11203 11231 11385 11531 11537 11567 11694 11700 11705
11709 11715 11747 11784 11794 11801 11806 11810 11812 11816 11817 11828 11923 11942 11947 11948
11966 11997 12019 12081 12125 12213 12263 12271 12280 12303 12305 12404 12434 12559 12625 12641
12663 12725 12730 12732 12733 12750 12774 12809 12885 12924 12934 12947 13019 13036 13051 13088
13126 13130 13147 13214 13215 13258 13259 13343 13455 13457 13461 13474 13506 13509 13581 13590
13640 13692 13748 13946 14012 14195 14256 14264 14267 14286 14392 14430 14589 14787 14800 14821
14831 14885 14907 14917 14921 14939 14940 14949 15024 15062 15151 15162 15221 15244 15245 15248
15249 15252 15417 15470 15476 15480 15484 15679 15893 15894 15900 15927 15937 16057 16147 16198
16249 16263 16273 16320 16477 16527 16532 16533 16548 16552 16596 16610 16660 16723 16838 16879
17022 17031 17072 17089 17093 17164 17172 17184 17224 17234 17245 17332 17367 17408 17440 17539
17658 17709 17711 17721 17755 17959 18151 18159 18198 18211 18218 18361 18439 18490 18516 18522
18769 18866 18869 18918 19129 19350 19485 19494 19623 19640 19654 19984 20147 20248 20294 20300
20414 20473 20534 20579 20628 20635 20718 20721 20728 20739 20815 20816 20819 20834 20845 20860
20862 20892 20898 20899 20907 20998 21000 21031 21052 21063 21124 21141 21155 21173 21178 21217
21673 21717 21728 21872 21874 21875 22002 22039 22045 22069 22089 22207 22225 22242 22250 22259
22330 22336 22357 22383 22390 22401 22437 22527 22534 22547 22754 22861 22892 22923 22946 22959
23018 23089 23100 23120 23144 23183 23202 23237 23268 23374 23388 23392 23393 23404 23411 23422
23435 23436 23437 23458 23492 23496 23508 23515 23544 23548 23597 23598 23655 23759 23933 23960
23963 23971 23990 24037 24046 24048 24066 24133 24302 24335 24351 24389 24396 24480 24498 24503
24528 24569 24582 24639 24654 24659 24714 24733 24963 25131 25134 25168 25175 25199 25217 25230
25234 25248 25256 25264 25278 25280 25284 25298 25307 25446 25485 25523 25527 25549 25650 25670
25755 25831 25834 25838 26020 26186 26201 26202 26212 26217 26240 26286 26392 26418 26439 26492
26545 26548 26549 26566 26567 26569 26578 26615 26697 26722 26730 26735 26894 26898 26993 27015
27047 27240 27247 27292 27340 27344 27346 27348 27387 27477 27478 27550 27696 27709 27742 27765
27813 27945 27951 28014 28029 28032 28034 28036 28037 28047 28073 28082 28163 28201 28246 28273
28286 28310 28347 28395 28432 28519 28655 28691 28757 28795 28796 28798 28816 28818 28885 28894
28905 28913 28969 28970 29130 29151 29217 29218 29224 29227 29246 29249 29250 29276 29289 29310
29311 29313 29327 29436 29437 29452 29458 29471 29483 29512 29559 29611 29617 29622 29683 29736
29819 29823 29825 29828 29893 29938 29944 29948 29965 29972 29977 29982 29995 30041 30046 30055
30078 30080 30084 30126 30154 30157 30168 30208 30252 30308 30314 30370 30377 30580 30581 30695
30696 30733 30787 30845 30860 30868 30899 30900 31141 31252 31407 31481 31505 31508 31519 31577
31687 31693 31719 31737 31844 31856 31879 31907 31967 32000 32006 32010 32018 32056 32115 32120
32124 32170 32264 32269 32292 32334 32341 32364 32365 32416 32420 32435 32453 32500 32503 32506
32516 32561 32605 32618 32622 32636 32648 32655 32679 32680 32681 32685 32687 32938 32943 32948
32952 32954 33016 33099 33106 33117 33127 33196 33211 33216 33221 33236 33237 33280 33288 33306
33311 33319 33326 33337 33349 33372 33375 33379 33380 33382 33390 33451 33476 33477 33481 33501
33502 33578 33588 33589 33593 33611 33804 33873 33990 34025 34052 34069 34073 34082 34111 34150
34170 34173 34175 34178 34203 34218 34220 34265 34266 34293 34303 34316 34326 34351 34353 34400
34468 34469 34487 34508 34520 34540 34616 34626 34702 34708 34711 34721 34825 34847 34859 34950
34967 35036 35050 35068 35099 35119 35130 35189 35193 35195 35227 35230 35242 35392 35402 35406
35421 35431 35434 35440 35452 35453 35501 35518 35548 35551 35700 35791 35813 35817 35827 35830
35835 35844 35847 35873 35874 35875 35901 35919 35925 35938 35974 36078 36085 36109 36110 36139
36151 36185 36205 36208 36270 36279 36357 36370 36376 36417 36425
17: 1716 1932 2155 2504 2792 2832 2969 3152 3606 3896 4029 4150 4278 4288 4303 6431 9439 11428
12366 16161 18992 19782 20155 20403 21107 21405 22832 22907 22972
18: 326 640 685 986 987 1562 3256 4450 4631 4636 4653 4744 4815 4881 4903 5050 5224 5233 5337 5414
5456 5511 5584 5698 5911 6040 6158 6223 6232 6284 6450 6505 6606 6665 6718 6789 6911 7038 7093 7127
7158 7274 7295 7425 7479 7641 7669 7736 7773 8106 8259 8303 8325 8336 8526 8586 8681 8754 8873 8939
8945 9008 9074 9154 9212 9228 9307 9622 9781 9797 9811 9938 9966 10005 10017 10068 10098 10205
10297 10417 10484 10579 10705 10736 10741 10762 10830 10847 10872 10886 10893 10919 10938 10997
11011 11030 11082 11160 11179 11469 11490 11506 11522 11565 11587 11589 11590 11591 11593 11677
11699 11762 11829 11845 11857 11925 12049 12054 12072 12111 12141 12166 12228 12268 12292 12319
12320 12448 12549 12591 12604 12633 12702 12731 12793 12821 12863 12936 13116 13163 13188 13242
13243 13244 13245 13246 13247 13326 13493 13531 13607 13660 13731 13749 13824 13933 13948 14014
14030 14094 14175 14246 14444 14454 14489 14529 14541 14547 14583 14653 14695 14770 14790 14869
14934 15022 15140 15172 15323 15372 15489 15677 15889 15992 16074 16323 16555 16630 16652 16678
16737 17193 17254 17380 17417 17493 17565 17573 17731 17777 17811 17832 17907 17978 18016 18071
18089 18101 18143 18279 18339 18397 18414 18485 18497 18679 18726 18775 18879 19010 19366 19657
19739 20183 20205 20237 20243 20621 20682 20685 20758 20871 20876 21035 21083 21139 21273 21304
21342 21370 21444 21675 21756 21784 21808 21855 21868 22005 22064 22115 22198 22240 22277 22308
22428 22446 22525 22618 23122 23293 23420 23449 23507 23620 23622 23724 23778 23792 23828 23845
23878 23906 23949 24024 24090 24121 24303 24414 24454 24477 24580 24616 24636 24820 24838 24903
24959 24995 25165 25229 25350 25476 25500 25576 25601 25616 25692 25696 25805 25941 26204 26347
26389 26395 26540 26718 26862 26878 26928 26991 27068 27263 27338 27383 27517 27548 27551 27568
27615 27625 27630 27638 27644 27658 27692 27736 27755 27807 27854 27877 27934 27960 27962 28023
28123 28173 28283 28315 28421 28499 28530 28694 28754 28803 28805 28844 28859 28876 28916 28932
28999 29019 29021 29128 29185 29258 29391 29446 29531 29557 29590 29664 29730 29770 29791 29817
29909 29921 29937 29974 30009 30031 30118 30200 30263 30352 30358 30413 30426 30464 30484 30490
30510 30555 30571 30610 30633 30675 30770 30791 30801 30827 30852 30917 30978 31041 31209 31217
31360 31456 31510 31772 31808 31920 31965 32040 32096 32193 32229 32273 32371 32390 32406 32448
32482 32545 32672 32734 32801 32883 33001 33135 33169 33302 33307 33356 33484 33583 33639 33696
33789 33829 33946 34022 34028 34059 34189 34257 34311 34429 34499 34538 34594 34738 34770 34839
34880 34912 35059 35095 35158 35188 35396 35442 35497 35587 35652 35736 35753 35881 35948 36044
36106 36212 36403 36429
19: 325 640 685 986 987 1562 3256 3841 3847 4003 4068 4286 4450 4492 4511 4522 4546 4548 4631 4636
4653 4815 4881 4903 5050 5191 5224 5233 5288 5308 5337 5414 5456 5511 5584 5698 5911 6040 6092 6158
6223 6232 6284 6450 6505 6606 6665 6718 6789 6911 7038 7093 7127 7158 7274 7295 7425 7479 7641 7669
7676 7736 7773 8106 8143 8259 8303 8325 8336 8526 8586 8681 8754 8873 8939 8945 9007 9008 9074 9154
9212 9228 9303 9307 9501 9622 9781 9797 9811 9938 9966 10068 10093 10098 10205 10297 10417 10484
10579 10705 10736 10741 10762 10830 10847 10872 10886 10893 10919 10938 10997 11011 11030 11082
11160 11179 11313 11427 11469 11490 11506 11522 11565 11587 11589 11590 11591 11593 11677 11699
11762 11829 11845 11857 11925 12049 12054 12072 12111 12141 12166 12228 12268 12292 12319 12320
12448 12549 12591 12604 12633 12660 12702 12731 12793 12821 12863 13041 13116 13163 13188 13242
13243 13244 13245 13246 13247 13326 13493 13531 13607 13660 13731 13749 13824 13933 13948 14014
14030 14094 14175 14246 14444 14454 14489 14529 14541 14547 14583 14653 14695 14707 14770 14790
14869 14934 15022 15140 15172 15264 15323 15372 15489 15533 15549 15677 15889 15992 16016 16037
16074 16323 16555 16630 16652 16678 16737 16832 17051 17193 17254 17380 17417 17493 17565 17573
17601 17602 17603 17731 17777 17811 17832 17907 17978 18016 18071 18089 18101 18143 18279 18339
18397 18414 18485 18497 18570 18587 18679 18726 18775 18879 19010 19366 19657 19739 19781 20007
20161 20167 20183 20205 20237 20243 20621 20682 20685 20758 20871 20876 20942 21035 21083 21090
21095 21139 21273 21304 21342 21370 21444 21675 21756 21784 21808 21855 21868 22005 22064 22115
22198 22240 22277 22308 22428 22446 22525 22618 23122 23160 23220 23293 23420 23449 23507 23620
23622 23666 23699 23724 23778 23792 23828 23845 23878 23906 23949 24024 24090 24121 24303 24370
24414 24454 24477 24513 24580 24616 24636 24820 24838 24903 24959 24995 25036 25109 25165 25229
25350 25354 25389 25476 25500 25576 25601 25616 25692 25696 25722 25805 25941 26204 26347 26389
26395 26400 26540 26715 26718 26862 26878 26928 26991 27068 27089 27263 27287 27338 27383 27466
27517 27548 27551 27568 27615 27625 27630 27638 27644 27658 27692 27736 27755 27807 27854 27877
27934 27960 27962 28023 28123 28173 28283 28315 28421 28499 28530 28694 28754 28803 28805 28844
28859 28876 28916 28932 28999 29019 29021 29128 29185 29258 29357 29391 29446 29531 29557 29590
29664 29730 29770 29791 29817 29909 29921 29937 29974 30009 30031 30118 30200 30263 30352 30358
30413 30426 30464 30484 30490 30510 30555 30571 30610 30611 30633 30675 30770 30791 30801 30827
30852 30893 30917 30978 31041 31078 31209 31217 31360 31456 31510 31772 31808 31846 31920 31965
32040 32096 32193 32229 32273 32371 32390 32406 32448 32482 32545 32672 32734 32801 32883 33001
33135 33169 33302 33307 33356 33484 33514 33583 33639 33696 33789 33829 33946 34022 34028 34059
34189 34257 34311 34429 34499 34538 34594 34609 34653 34738 34770 34839 34880 34912 35013 35059
35095 35158 35188 35396 35442 35497 35587 35652 35736 35753 35881 35948 36044 36106 36212 36403
36429
20: 1444 3299 4511 4548 4744 4838 5186 5224 5288 5308 5375 5385 5556 5627 6092 6174 6311 6450 6588
6606 6647 6649 6665 6812 6928 7022 7172 7295 7676 8422 8945 9095 9154 9501 9797 9908 9909 10017
10135 10309 10413 10498 10688 10741 10769 10886 10893 10919 10938 10985 11011 11048 11185 11506
11674 11699 11733 11783 11845 12054 12109 12111 12411 12461 12532 12599 12633 12793 12867 12936
13041 13159 13212 13531 13596 13656 13731 13749 13933 13948 14398 14541 14547 14583 14619 14636
14637 14638 14639 14640 14641 14743 15172 15489 15674 15962 16308 16352 16376 16402 16652 16759
16835 17129 17152 17382 17565 17597 17601 17602 17603 17687 17955 18143 18205 18286 18339 18381
18414 18876 20563 20677 20871 21083 21125 21163 21273 21758 22012 22078 22115 22434 23290 23348
23461 23699 23828 24027 24096 24303 24352 24414 24477 24513 24586 24636 24959 24995 25036 25109
25354 25576 25811 25861 25951 26400 26718 26878 26974 27089 27287 27289 27315 27338 27466 27548
27625 27638 27658 27692 27736 27755 27934 27989 28127 28202 28217 28345 28412 28488 28532 28630
28714 28803 28859 28916 28932 29001 29019 29021 29128 29169 29357 29557 29590 29623 29817 29906
30025 30358 30556 30705 30827 31004 31102 31126 31209 31217 31378 31456 31707 31778 31846 31881
32106 32280 32572 32747 32760 33302 33307 33514 33946 34022 34028 34534 34627 34653 34949 34975
35013 35053 35154 35383 35385 35396 35442 35458 35596 35736 36044
21: 329 672 1271 1349 1648 1894 2345 2728 2729 3215 3423 3521 3774 4123 4263 5686 5777 6442 9126
13871 13878 13907 13994 17507 17765 17845 18562 18754 21092 21106 21604 22614 22650 23750 26807
29621 30298 30962 34748
22: 328 672 1271 1349 1894 2345 2728 2729 3215 3423 3521 3774 4123 4263 5686 5777 6442 9126 13871
13878 13907 13994 17507 17765 17845 18562 18754 21092 21106 21604 22614 22650 23750 26807 29621
30298 30962 34748
23: 1271 1349 1779 1904 2396 2479 3473 4171 4489 6442 9126 9127 9128 11335 11729 13871 13878 13907
13994 15798 17829 18529 18602 18754 19017 19097 19976 20396 22735 24706 25079 30962
24: 1271 1349 1779 1904 2396 2479 3473 4171 4489 6442 9126 9127 9128 11335 11729 13871 13878 13907
13994 15798 17829 18529 18602 18754 19017 19097 19976 20396 22735 24706 25079 30962
25: 595 836 837 943 962 1191 1392 1579 2461 2477 2727 2836 3226 4061 4080 4120 4184 4238 4256 4301
4521 4784 5220 5241 5350 5438 5500 6039 6595 6836 6938 6962 7012 7091 7113 7238 7419 7494 7634 7663
7742 7900 8221 8271 8297 8544 8567 8726 9091 9215 9282 9603 9696 9825 9873 10019 10180 10186 10207
10267 10370 10436 10625 10701 10794 10795 10796 10918 10977 11149 11237 11440 11459 11566 11596
11637 11871 12047 12178 12509 12580 12699 12844 13160 13314 13501 13558 13841 13899 13966 14429
14472 14577 14962 15040 15072 15402 15678 15701 15919 16700 16824 16987 17191 17252 17355 17564
17656 17720 18249 18307 18795 18917 19081 19320 19632 19953 19954 20440 20714 20818 21306 21683
21742 21983 22252 22356 22442 22514 22710 23101 23362 23482 23698 23734 23742 23968 24016 24576
24729 24953 24984 25116 25139 25196 25345 25536 25744 25780 25847 26710 26742 26992 27033 27150
27211 27276 27306 28022 28090 28158 28198 28204 28220 28239 28338 28396 28462 28739 28993 29146
29352 29393 29415 29571 29651 29714 29911 30017 30124 30180 30216 30734 30848 30873 30961 30992
31033 31351 31363 31406 31516 31724 31759 31859 31891 32046 32132 32188 32221 32230 32271 32380
32393 32472 32710 32898 32920 33142 33182 33246 33252 33256 33365 33678 33762 33780 34058 34291
34350 34514 34726 34889 34907 35024 35138 35221 35511 35570 35626 35746 35991 36133 36181
26: 617 854 1058 1192 1342 1416 1435 1733 1910 1942 1980 2036 2054 2136 2352 2399 2400 2448 2476
2492 2499 2547 2570 2651 2869 2885 3112 3171 3460 3582 3851 3981 4023 4177 4287 4456 4532 4552 4618
4651 4696 4726 4800 4868 4970 5076 5139 5216 5239 5297 5372 5435 5461 5554 5603 6072 6146 6200 6235
6252 6281 6306 6370 6524 6525 6553 6603 6776 6927 6944 6964 7040 7083 7137 7164 7230 7309 7421 7630
7659 7688 7847 7936 8037 8060 8118 8222 8293 8356 8426 8543 8582 8698 8747 8806 9041 9078 9105 9110
9171 9207 9289 9331 9473 9522 9558 9789 9846 9905 9956 10061 10206 10217 10244 10291 10305 10375
10482 10556 10644 10689 10767 10797 10842 10852 10949 10978 11012 11027 11083 11097 11104 11115
11153 11178 11320 11337 11455 11485 11633 11701 11741 11768 11781 11870 11958 12090 12119 12135
12151 12160 12207 12258 12288 12308 12398 12543 12583 12603 12686 12724 12892 13065 13113 13173
13191 13534 13535 13561 13671 13676 13827 13861 13891 13958 13998 14058 14142 14183 14262 14367
14450 14509 14538 14630 14650 14669 14727 14777 14878 14919 15029 15078 15079 15080 15081 15082
15155 15250 15353 15421 15473 15637 15664 15699 15752 15979 16021 16043 16082 16222 16270 16325
16342 16416 16531 16636 16647 16673 16757 16808 17026 17047 17148 17166 17229 17373 17504 17561
17591 17708 17785 17928 17930 17994 18010 18105 18156 18311 18353 18368 18409 18472 18492 18508
18575 18689 18719 18759 18827 18892 18951 19327 19354 19416 19495 19613 19712 19732 19744 19800
20026 20169 20186 20234 20289 20362 20553 20613 20706 20837 20847 20961 20970 21026 21053 21076
21131 21147 21232 21365 21476 21661 21693 21754 21762 21776 21792 21793 21794 21795 21877 21926
21939 21988 22127 22179 22220 22275 22345 22418 22456 22529 22784 22800 22801 22951 23088 23156
23210 23261 23324 23351 23446 23451 23593 23652 23695 23741 23755 23816 23842 23871 23936 23998
24063 24087 24144 24153 24315 24357 24402 24412 24425 24520 24575 24662 24663 24774 24822 24849
24873 24896 24954 25001 25026 25055 25098 25114 25178 25204 25341 25364 25406 25548 25572 25599
25633 25704 25711 25774 25844 25949 25973 26019 26256 26332 26510 26519 26554 26672 26675 26704
26741 26773 26780 26792 26821 26997 27005 27027 27040 27118 27241 27279 27291 27307 27328 27359
27361 27448 27452 27473 27513 27538 27633 27685 27798 27855 27890 28054 28091 28144 28145 28168
28182 28216 28233 28266 28281 28289 28305 28317 28329 28358 28390 28397 28446 28464 28482 28511
28515 28546 28567 28650 28733 28760 28822 28828 28839 28997 29112 29158 29181 29225 29342 29422
29442 29500 29580 29657 29766 29780 29788 29799 29858 29881 29922 29996 30039 30050 30115 30204
30223 30284 30310 30315 30319 30372 30440 30457 30485 30494 30522 30562 30651 30762 30764 30775
30777 30875 30887 30918 30923 30985 31035 31129 31220 31221 31361 31371 31379 31399 31444 31484
31572 31696 31712 31745 31799 31862 31874 31877 31913 31927 31970 32087 32121 32154 32167 32220
32254 32408 32473 32491 32538 32651 32735 32740 32787 32870 32887 32984 33009 33056 33150 33175
33314 33333 33504 33530 33603 33641 33674 33776 33799 33840 33876 34003 34006 34057 34128 34193
34230 34237 34360 34419 34464 34505 34561 34585 34586 34587 34588 34589 34590 34613 34629 34675
34706 34789 34798 34887 34904 34931 35000 35060 35086 35134 35226 35270 35384 35413 35520 35684
35757 35845 35921 36037 36054 36081 36190 36293 36306 36355 36405
27: 1233 2200 2321 2633 3114 3208 3558 3572 3730 3763 3809 4009 4368 6383 6468 6940 6968 7316 8411
8595 8597 8598 8599 8600 8847 8876 8950 8978 9097 9442 9663 10168 10537 10539 10780 10867 11206
11207 11208 12363 12369 12393 12397 12464 12473 12508 12974 12993 13120 13363 14220 14411 14550
14772 15006 15007 15008 15009 15010 15083 15188 15287 15399 15694 16117 16132 16259 16260 16489
16490 18642 18814 18826 18830 18862 18865 18872 18873 18878 18884 18887 18888 18889 18896 18899
18906 18907 18909 18919 19028 19363 20032 20040 20924 21109 21645 22831 23325 24755 24970 24975
25665 25666 25890 26295 26923 26949 26966 26967 26972 27108 27198 27260 27671 28152 28188 28378
28445 28767 28768 29388 29845 30766 30774 30972 30973 31400 31557 31671 31704 31710 32145 32153
32209 32210 32308 32309 32598 33760 33808 33812 34383 35064 35065 35368 35997 36353
28: 401 4474 4856 4885 5515 6148 6880 6897 7356 7360 7365 8124 8729 9065 9073 9143 9587 10054 10456
10818 10819 10820 10821 10934 10935 11543 11731 11732 11792 11905 11906 11907 11909 11911 11912
11913 11967 11968 11969 11970 11971 11972 11973 11974 11975 11976 11977 11978 11979 11980 11982
11983 11984 11987 11988 11989 11990 11991 11993 11994 12236 12259 12385 12386 12388 12465 12600
12790 13035 13315 14123 14313 14315 14317 14319 14323 14325 14326 14431 14663 14690 14834 15074
15279 15487 15488 15582 15600 15692 15743 15783 15785 15786 15789 15790 15791 16005 16493 16494
16787 16788 16789 17040 17450 17459 17543 17544 17545 17546 17547 18231 18232 18233 18234 18268
18269 18270 18271 18272 18475 18911 20157 20159 20327 20333 20339 20578 20649 20686 20688 20689
20793 20794 20795 20947 21184 21185 21186 21187 21188 21189 21830 21831 23058 23059 23060 23061
23062 23146 23332 23553 23886 24294 24387 24805 24897 25706 25707 25924 26028 26032 26034 26035
26052 26055 26084 26104 26250 26289 26526 26612 26619 26620 26621 26622 26623 26624 26625 26626
26627 26628 26629 26630 26631 26632 26633 26634 26635 26636 26637 26638 26641 26642 26643 26644
26646 26647 26648 26650 26651 26652 26985 27100 27153 27154 27155 27156 27157 27158 27159 27160
27161 27162 27163 27164 27165 27166 27168 27169 27170 27171 27172 27302 27414 27442 27443 27444
27445 27446 27821 27829 27995 28184 28253 28365 28448 28587 28588 28589 29150 29765 30218 30392
30686 30694 31166 31257 31285 31286 31287 31288 31289 31290 31409 31410 31535 31536 31537 31538
31539 31540 31541 31542 31543 31544 31545 31546 31547 31548 31549 31550 31596 31667 31718 31957
31958 32123 32955 33069 33070 33810 34116 34844 34959 35280 35544 36001 36002 36234
29: 1233 2200 2321 2633 3114 3208 3558 3572 3730 3763 3809 4009 4368 6383 6468 6940 6968 7316 8411
8595 8597 8598 8599 8600 8847 8876 8950 8978 9097 9442 9663 10168 10537 10539 10780 10867 11206
11207 11208 12363 12369 12393 12397 12464 12473 12508 12974 12993 13120 13363 14220 14411 14550
14772 15006 15007 15008 15009 15010 15083 15188 15287 15399 15694 16117 16132 16259 16260 16489
16490 18642 18814 18826 18830 18862 18865 18872 18873 18878 18884 18887 18888 18889 18896 18899
18906 18907 18909 18919 19028 19363 20032 20040 20924 21109 21645 22831 23325 24755 24970 24975
25665 25666 25890 26295 26923 26949 26966 26967 26972 27108 27198 27260 27671 28152 28188 28378
28445 28767 28768 29388 29845 30766 30774 30972 30973 31400 31557 31671 31704 31710 32145 32153
32209 32210 32308 32309 32598 33760 33808 33812 34383 35064 35065 35368 35997 36353
30: 338 339 4338 4454 4666 4714 5221 5273 5377 5383 6511 6590 6610 6885 6992 7023 7307 7335 7336
7337 7467 8108 8116 8198 8370 8376 8417 8721 8832 8960 9156 9197 9641 9666 9973 10081 10157 10288
10405 10709 10911 10956 10983 11049 11666 11926 12096 12200 12202 12684 12701 12875 12914 13044
13086 13168 13317 13533 13552 13592 13605 13609 13639 14308 14492 14566 14714 14742 14838 15091
15293 15336 15419 15552 15558 15771 16219 16237 16645 16834 16885 17211 17213 17349 17929 17985
18194 18328 18720 18721 18761 20197 20249 21268 21719 21720 21771 21955 22013 22090 22122 22152
22170 23255 23284 23311 23463 23761 24025 24834 24875 24985 25524 25639 25736 25754 25796 25859
25998 26122 26410 26520 26818 26825 27065 27425 27481 27681 27992 28099 28124 28732 29007 29023
29114 29133 29340 29445 29529 29545 29581 29625 29865 29892 29898 30026 30291 30452 30530 30950
31005 31140 31672 31791 31848 31928 32272 32990 33166 33173 33224 33226 33320 33607 33807 33834
33851 34033 34535 34591 35052 35156 35417 35594 35782 35958 35960 35968 36158 36256 36396
31: 337 339 982 1313 1510 2295 2336 2440 2631 2853 2989 3491 3834 3927 4048 4060 4127 4140 4160 4191
4241 4302 4338 4454 4666 4714 5221 5273 5377 5383 5679 5712 5811 5899 5900 5914 6447 6485 6511 6590
6610 6885 6907 6992 7023 7307 7335 7336 7337 7467 7872 7874 8108 8116 8198 8370 8376 8417 8721 8832
8960 9156 9197 9457 9641 9666 9973 10081 10157 10288 10405 10709 10911 10956 10983 11049 11666
11926 12096 12200 12202 12227 12684 12701 12875 12914 13044 13086 13168 13317 13359 13533 13552
13592 13605 13609 13639 13901 13913 14308 14492 14566 14714 14742 14838 15091 15293 15336 15419
15552 15558 15771 16173 16469 16645 16834 16885 16892 17211 17213 17349 17389 17471 17790 17866
17929 17985 18194 18328 18577 18720 18721 18761 19128 19441 20197 20249 20421 20992 21268 21409
21479 21642 21719 21720 21771 21955 22013 22090 22122 22152 22170 22654 23039 23255 23284 23311
23313 23463 23691 23761 24025 24834 24875 24985 25491 25524 25639 25736 25754 25796 25859 25998
26122 26410 26520 26818 26825 27065 27425 27436 27600 27681 27992 28099 28124 28732 29007 29023
29114 29133 29340 29445 29529 29545 29581 29625 29693 29806 29865 29892 29898 30026 30291 30452
30530 30950 31005 31140 31156 31672 31774 31791 31848 31928 32272 32990 33166 33173 33224 33226
33320 33607 33807 33834 33851 34033 34535 34591 35052 35156 35417 35594 35782 35958 35960 35968
36158 36256 36396
32: 337 338 574 725 1386 2240 2304 2609 2881 3222 4338 4454 4666 4714 5221 5273 5377 5383 5709 6463
6511 6590 6610 6885 6992 7023 7307 7335 7336 7337 7467 7922 8108 8116 8198 8370 8376 8417 8721 8832
8960 9156 9197 9641 9666 9973 10081 10157 10288 10405 10709 10911 10956 10983 11049 11666 11926
12096 12200 12202 12684 12701 12875 12914 13044 13086 13168 13317 13533 13552 13592 13605 13609
13639 14308 14492 14566 14714 14742 14838 15091 15293 15336 15419 15552 15558 15771 16645 16834
16885 17211 17213 17349 17929 17985 18194 18328 18720 18721 18761 20197 20249 21268 21644 21719
21720 21771 21955 22013 22090 22122 22152 22170 23255 23284 23311 23463 23761 24025 24834 24875
24985 25524 25639 25736 25754 25796 25859 25998 26122 26410 26520 26818 26825 27065 27425 27681
27992 28099 28124 28732 29007 29023 29114 29133 29340 29445 29529 29545 29581 29625 29865 29892
29898 30026 30291 30452 30530 30950 31005 31140 31672 31791 31848 31928 32272 32990 33166 33173
33224 33226 33320 33607 33807 33834 33851 34033 34535 34591 35052 35156 35417 35594 35782 35958
35960 35968 36158 36256 36396
33: 784 964 973 974 1033 1256 1300 1330 1332 1697 1719 1723 1785 1914 2057 2205 2207 2394 2436 2539
2558 2663 2679 2838 2858 2887 2960 3130 3420 3508 3636 3790 3818 3864 3990 3991 4033 4126 4187 4190
4245 4272 4333 4342 4407 4409 4468 4469 4519 4586 4593 4599 4619 4681 4688 4722 4723 4770 4789 4999
5037 5056 5075 5090 5121 5135 5176 5254 5260 5291 5319 5336 5352 5396 5415 5495 5557 5597 5602 5641
5645 5648 5650 5678 5747 5790 5828 5932 5963 5970 5986 5997 6099 6110 6114 6182 6227 6228 6229 6241
6254 6268 6269 6307 6331 6548 6549 6556 6572 6604 6651 6700 6741 6751 6752 6777 6788 6798 6810 6813
6884 6926 6933 7024 7073 7132 7155 7198 7241 7275 7338 7344 7345 7346 7347 7482 7580 7625 7653 7683
7719 7794 7795 7796 7797 7859 7871 7956 7976 8117 8119 8168 8172 8187 8243 8262 8287 8330 8410 8494
8525 8591 8629 8651 8685 8738 8772 8788 8915 8916 8941 8989 9015 9172 9184 9244 9276 9297 9305 9342
9410 9509 9511 9547 9554 9561 9649 9650 9680 9685 9734 9810 9845 9895 9899 9900 9978 10055 10097
10099 10121 10122 10123 10134 10136 10172 10177 10276 10289 10292 10311 10337 10372 10386 10409
10459 10513 10551 10588 10636 10645 10651 10655 10704 10715 10792 10908 10942 10960 10998 11020
11037 11045 11086 11087 11089 11162 11172 11187 11188 11220 11227 11387 11401 11448 11535 11586
11669 11672 11702 11754 11757 11758 11786 11807 11808 11877 11888 11897 11899 11902 11944 12088
12099 12107 12115 12132 12142 12167 12174 12182 12194 12256 12295 12302 12506 12523 12540 12575
12605 12669 12708 12745 12800 12812 12832 12858 12869 12872 12906 12911 12932 12933 12941 12942
12943 12983 12989 12991 12992 12993 12994 12995 12999 13024 13032 13064 13089 13090 13179 13213
13253 13460 13464 13472 13505 13510 13522 13523 13538 13573 13591 13601 13613 13633 13651 13654
13688 13773 13793 13794 13795 13820 13854 13999 14000 14002 14024 14121 14245 14273 14284 14399
14400 14419 14511 14513 14520 14521 14522 14528 14553 14554 14555 14556 14557 14616 14757 14762
14769 14773 14785 14818 14835 14866 14877 14910 14912 14944 14999 15000 15003 15034 15225 15238
15251 15305 15320 15343 15348 15373 15439 15440 15441 15442 15443 15444 15445 15446 15447 15448
15449 15450 15451 15452 15453 15454 15455 15456 15457 15458 15459 15460 15461 15462 15463 15464
15465 15466 15467 15495 15496 15531 15662 15683 15706 15718 15734 15766 15885 15899 15956 15957
16027 16030 16089 16128 16191 16245 16296 16298 16302 16306 16314 16331 16348 16357 16367 16388
16406 16439 16472 16521 16558 16569 16588 16632 16681 16715 16843 17075 17084 17114 17149 17154
17161 17200 17221 17334 17335 17356 17405 17458 17486 17531 17552 17553 17703 17738 17904 17918
17943 17961 17999 18002 18092 18096 18100 18122 18203 18238 18247 18308 18345 18355 18379 18389
18396 18443 18444 18537 18596 18714 18724 18771 19079 19117 19262 19263 19391 19418 19504 19505
19506 19507 19508 19509 19510 19511 19598 19608 19672 19697 19719 19864 19903 19948 20113 20185
20212 20270 20309 20316 20317 20319 20387 20557 20592 20610 20676 20719 20771 20887 20896 20913
20960 21021 21047 21049 21050 21066 21068 21088 21098 21126 21151 21153 21166 21169 21203 21204
21205 21206 21207 21220 21270 21272 21296 21298 21333 21410 21609 21687 21712 21733 21764 21812
21864 21886 21968 22040 22076 22084 22095 22120 22147 22162 22204 22210 22232 22273 22276 22344
22369 22371 22391 22415 22422 22464 22468 22479 22490 22499 22522 22590 22637 22638 22752 22835
23070 23072 23151 23187 23194 23200 23229 23233 23288 23322 23367 23375 23423 23582 23584 23650
23685 23700 23822 23862 23872 23905 23921 23935 23992 24052 24053 24054 24055 24070 24094 24116
24131 24330 24347 24381 24440 24455 24463 24466 24509 24532 24602 24607 24617 24625 24720 24777
24798 24821 24841 24917 24968 24977 24979 25016 25047 25144 25183 25205 25207 25269 25273 25335
25369 25414 25458 25493 25542 25553 25581 25701 25702 25703 25724 25738 25742 25798 25911 25914
25915 25930 25953 26008 26114 26188 26214 26228 26244 26276 26287 26315 26351 26438 26486 26505
26517 26518 26551 26573 26609 26614 26689 26828 26853 26863 26871 26920 26931 26960 26969 26979
26986 26995 27058 27064 27112 27130 27207 27229 27297 27304 27314 27332 27335 27351 27492 27511
27528 27561 27562 27579 27580 27581 27582 27583 27584 27585 27651 27652 27674 27693 27700 27745
27760 27763 27771 27801 27812 27824 27837 27887 27899 27906 27935 27948 28062 28085 28117 28120
28121 28126 28140 28153 28195 28205 28225 28251 28262 28302 28327 28364 28416 28420 28492 28540
28611 28643 28652 28697 28707 28710 28715 28737 28780 28800 28891 28923 28925 28931 28937 28995
29008 29025 29090 29123 29189 29231 29241 29321 29338 29363 29426 29480 29491 29502 29551 29584
29628 29703 29707 29746 29787 29803 29831 29856 29857 29869 29875 29904 29926 29962 30029 30071
30088 30095 30105 30110 30111 30197 30240 30245 30273 30356 30364 30414 30437 30566 30652 30653
30706 30847 30880 31045 31075 31076 31077 31119 31120 31348 31354 31367 31384 31387 31419 31434
31435 31483 31555 31556 31588 31729 31731 31767 31782 31785 31820 31931 31961 31990 32012 32025
32054 32128 32183 32249 32260 32262 32335 32437 32456 32496 32558 32573 32608 32668 32682 32717
32736 32742 32750 32792 32891 32923 32924 32931 32991 33008 33114 33140 33151 33289 33310 33322
33373 33444 33487 33498 33523 33573 33620 33642 33645 33670 33680 33703 33770 33824 33868 33878
33975 33976 33977 33978 33979 33980 33981 33982 33983 33984 33985 33986 33987 33988 34042 34043
34053 34056 34090 34160 34167 34210 34245 34355 34393 34412 34478 34484 34493 34537 34549 34624
34652 34664 34690 34720 34724 34782 34802 34818 34846 34873 34952 34982 35011 35018 35034 35102
35163 35212 35235 35430 35463 35555 35568 35592 35622 35659 35710 35717 35724 35788 35819 35826
35831 35860 35863 35869 35944 35947 36036 36101 36112 36114 36191 36214 36277 36348 36363 36371
36372 36373 36374 36394 36402
34: 342 4708 5034 5156 5419 6539 7341 8635 8858 8859 10313 13433 14254 15794 17033 18390 20776
20777 21807 21842 21931 23134 23344 23928 24395 24459 24619 26867 27670 27820 27901 28928 31152
31266 31452 31492 31525 31600 32062 32359 33157 35838
35: 341 4711 4713 5034 5156 5249 6539 7341 8635 8858 8859 10087 10313 12712 13433 14254 15550 15794
17033 18390 20776 20777 21842 22114 23134 23344 23928 24395 26057 27186 27670 27777 27778 27820
28331 29096 29476 31152 31153 31266 31492 31525 32359 33157 35838
36: 1144 1860 1896 1915 1997 2391 2438 2523 2752 2870 3495 3723 4159 4299 4607 4652 4833 4869 4922
4938 4951 4987 4998 5125 5246 5298 5449 5539 5588 5620 5687 5734 5962 5985 6043 6088 6135 6368 6691
6698 6794 6844 7058 7097 7145 7175 7202 7226 7709 7738 8127 8133 8178 8260 8344 8380 8381 8535 8615
8673 8691 8752 8929 9238 9266 9536 9585 9613 9673 9674 9675 9822 9882 9897 10102 10242 10429 10458
10479 10598 10615 10635 10752 10810 11039 11714 11723 11826 11884 12000 12009 12053 12068 12226
12229 12424 12529 12734 12772 13110 13458 13482 13513 14043 14061 14270 14497 14508 14693 14731
14841 14942 14952 15035 15046 15123 15170 15239 15242 15340 15391 15409 15571 15911 16108 16129
16301 16361 16378 16379 16382 16454 16545 16565 16598 16773 17082 17107 17108 17143 17158 17404
17710 17740 17766 17806 17925 17969 18144 18145 18220 18462 18584 18716 18844 19308 19631 19816
20221 20227 20303 20478 20479 20480 20481 20482 20483 20484 20485 20486 20487 20488 20489 20490
20491 20492 20493 20494 20495 20496 20497 20498 20499 20500 20501 20502 20503 20504 20505 20506
20507 20508 20509 20510 20511 20512 20513 20514 20515 20516 20517 20518 20519 20520 20521 20522
20523 20524 20525 20526 20600 20705 20735 20949 20950 21010 21020 21130 21495 21843 21903 22151
22194 22212 22302 22317 22438 22503 22706 23026 23073 23193 23211 23394 23426 23518 23560 23956
23974 24340 24447 24609 24767 24768 24783 24872 24877 25182 25197 25262 25385 25424 25535 25592
25656 25657 25717 25876 26239 26251 26362 26374 26521 26560 26564 26855 26940 26941 26942 26963
27343 27416 27431 27432 27433 27702 27933 28004 28008 28059 28590 28935 29026 29234 29244 29253
29309 29322 29407 29462 29729 29733 29822 30044 30193 30260 30290 30656 30837 31223 31225 31500
31523 31665 31796 32015 32027 32081 32186 32369 32372 32451 32509 32547 32609 32703 32755 32770
32778 32992 33012 33088 33111 33123 33170 33232 33233 33234 33235 33472 33494 33548 33598 33610
33881 34039 34081 34179 341 86 34292 34342 34408 34472 34569 34570 34597 34657 34727 34756 34875
34974 35007 35074 35197 35253 35444 35507 35723 35772 35805 35810 35853 35945 36020 36125 36223
36233 36269 36400 36431
37: 311 385
38: 732 1187 1222 2716 2766 3147 4169 4515 4582 4614 4758 4785 4793 4867 5081 5087 5088 5100 5208
5271 5310 5374 5386 5418 5458 5464 5514 5548 5601 5608 5984 6024 6069 6379 6510 6587 6616 6677 6688
6757 6763 6768 6867 7014 7168 7212 7213 7223 7240 7260 7287 7438 7633 7662 7687 7727 8160 8211 8223
8296 8496 8511 8532 8547 8694 8727 9059 9203 9237 9259 9519 9693 9749 9763 9796 9832 9854 9859 9877
9954 10001 10003 10095 10302 10307 10308 10571 10614 10679 10798 10815 11458 11562 11713 11745
11782 11818 11903 11952 12044 12337 12432 12581 12658 12894 12919 13045 13112 13128 13357 13450
13549 14187 14266 14287 14385 14474 14567 14602 14623 14624 14626 14768 14848 14879 14923 14928
14948 15222 15246 15255 15365 15479 15518 15907 15908 16024 16086 16093 16182 16236 16389 16535
16554 16560 16858 17054 17058 17066 17070 17216 17381 17589 17946 18086 18183 18366 18739 18851
19130 19424 20228 20296 20433 20722 20774 20780 20932 20969 21009 21016 21019 21111 21133 21144
21254 21279 21280 21388 21695 21757 21883 21993 22006 22050 22057 22061 22067 22129 22200 22226
22227 22298 22312 22328 22331 22334 22395 22433 22459 22524 22540 23020 23142 23165 23169 23181
23254 23386 23484 23517 23570 23638 23946 24035 24355 24361 24363 24574 24578 24793 24799 24851
24999 25023 25344 25365 25403 25528 25556 25598 25619 25682 25730 25769 25824 25833 25946 25950
26221 26235 26247 26266 26333 26406 26577 26822 26826 26990 27092 27259 27322 27372 27680 27744
27886 27905 27973 28318 28490 28495 28615 28628 28713 28724 28804 28982 29161 29178 29230 29239
29257 29425 29494 29497 29608 29811 29888 29925 30011 30014 30019 30053 30056 30069 30085 30215
30236 30445 30620 30640 31013 31039 31124 31154 31232 31513 31780 31849 31918 32036 32060 32065
32107 32187 32237 32261 32263 32288 32300 32343 32348 32360 32461 32481 32493 32505 32535 32647
32658 32666 32667 32784 32884 32939 32975 32983 33132 33155 33160 33164 33198 33483 33489 33499
33533 33779 34070 34089 34158 34162 34187 34255 34314 34337 34382 34397 34405 34446 34483 34490
34532 34553 34628 34672 34696 34728 34747 34779 34969 34997 35198 35205 35217 35275 35517 35636
35706 35735 35768 35769 35861 35880 35897 35963 36088 36122 36123
39: 426 946
40: 761 821 978 1165 2748 2749 2816 3174 3561 3822 4144 4247 7326 8364 8365 8366 8861 8862 9122 9733
11408 13752 13758 13862 13953 13963 14127 16775 17809 17859 18634 18748 18785 18799 19284 19401
20045 20444 21091 21631 21827 22579 23219 23713 24157 24158 25649 25909 27451 28836 32951 34582
34857
41: 309 411 898 905 915 1533 1595 1792 2768 3382 3404 3669 4434 5153 5688 6982 7146 7374 7885 9364
10152 10153 11279 15067 16455 16779 16780 16781 16782 16783 18532 19184 19230 19766 20402 21437
22781 24696 25892 26049 27990 33033 34259 35288 35289 36420
42: 309 348 410 411 412 630 898 905 915 1533 1595 1792 2768 3382 3404 3669 4434 5153 5688 6982 7146
7374 7885 9364 10152 10153 11279 15067 16455 16779 16780 16781 16782 16783 18532 19184 19230
19766 20402 21437 22781 24696 25892 26049 27990 33033 34259 35288 35289 36420
43: 762 1070 1190 1272 1505 1924 2220 4883 5276 5689 6016 6047 6906 6947 6976 9448 10058 10194
11275 13784 13851 14391 14392 15431 15638 15639 15640 15641 15642 15643 15644 15645 15646 15647
15648 15685 15686 16312 17056 17771 18566 19255 19343 19527 19971 20097 21418 22878 24160 24742
25052 25867 26091 26288 26489 26595 27761 28272 28299 28387 30624 30753 31744 31903 32119 32941
33063 33064 34566 36008 36422
44: 1671 2465 3294 5450 5774 6626 7964 8053 10369 10714 11634 12842 12915 13048 13556 13626 13764
13780 13897 15202 15975 19324 19790 21532 22559 23091 23262 23566 24522 24842 25250 25589 26597
28971 29367 29373 29612 29882 31853 32615 32701 35206 35411
45: 977 1038 1039 1148 1382 1602 1639 1690 1709 1732 1793 1844 1856 1946 2035 2047 2144 2145 2151
2152 2199 2209 2261 2280 2283 2300 2316 2318 2341 2361 2364 2383 2389 2405 2435 2469 2530 2602 2648
2659 2664 2670 2705 2731 2735 2776 2780 2786 2933 2986 3020 3024 3025 3057 3103 3132 3150 3157 3179
3181 3201 3280 3309 3313 3341 3485 3511 3519 3529 3571 3673 3804 3805 3833 3932 3992 3993 4032 4096
4221 4483 5285 5286 5287 5290 5685 5716 5717 5718 5719 5751 5805 5806 5823 5824 5825 5826 5855 5864
6405 6410 6424 6437 6483 6646 7882 7896 7902 7924 7925 7926 7988 7989 7999 8000 8059 8389 8390 8391
8912 9441 9724 9898 10159 10236 10268 11263 11398 11399 11407 11901 12024 12025 12997 13104 13776
14238 14994 14995 14996 15077 15298 15299 15493 15505 15520 15737 15738 15739 15740 15741 16001
16045 16168 16187 16190 16457 16507 16614 16701 17136 17482 17762 17820 17844 17880 17881 17882
18171 18172 18523 18560 18608 18615 18653 18782 18926 18927 18932 19118 19162 19183 19192 19317
19318 19332 19333 19347 19348 19421 19422 19423 19463 19497 19596 19785 19786 19861 19862 19906
19907 19908 19974 19975 19990 20079 20080 20081 20082 20346 20351 20447 21331 21357 21378 21381
21382 21383 21384 21401 21413 21414 21432 21455 21456 21457 21480 21506 21507 21508 21557 21564
21582 21654 21655 21656 21710 21911 21941 21943 22474 22561 22562 22563 22564 22696 22718 22729
22740 22743 22744 22745 22746 22747 22846 22847 22937 22941 22975 22989 22997 22999 23000 23009
23037 23607 23711 23712 23866 23867 23868 24159 24854 25103 25104 25456 25457 25472 25885 25886
25887 26184 26185 26411 27456 27757 27758 28002 28136 28137 28834 28835 29153 29433 29674 29690
29751 29752 29753 29754 29755 29756 30400 30534 30535 30536 30583 30839 31064 31065 31066 31142
31178 31277 31411 31436 31578 31579 31580 31581 31871 32593 32594 32913 32914 32915 33034 34921
35302 35535 36228 36239 36421
46: 977 1038 1039 1148 1382 1602 1639 1690 1709 1732 1793 1844 1856 1946 2035 2047 2144 2145 2151
2152 2199 2209 2261 2280 2283 2300 2316 2318 2341 2361 2364 2383 2389 2405 2435 2469 2530 2602 2648
2659 2664 2670 2705 2731 2735 2776 2780 2786 2933 2986 3020 3024 3025 3057 3103 3132 3150 3157 3179
3181 3201 3280 3309 3313 3341 3485 3511 3519 3529 3571 3673 3804 3805 3833 3932 3992 3993 4032 4096
4221 4483 5285 5286 5287 5290 5685 5716 5717 5718 5719 5751 5805 5806 5823 5824 5825 5826 5855 5864
6405 6410 6424 6437 6483 6646 7882 7896 7902 7924 7925 7926 7988 7989 7999 8000 8059 8389 8390 8391
8912 9441 9724 9898 10159 10236 10268 11263 11398 11399 11407 11901 12024 12025 12997 13104 13776
14238 14994 14995 14996 15077 15298 15299 15493 15505 15520 15737 15738 15739 15740 15741 16001
16045 16168 16187 16190 16457 16507 16614 16701 17136 17482 17762 17820 17844 17880 17881 17882
18171 18172 18523 18560 18608 18615 18653 18782 18926 18927 18932 19118 19162 19183 19192 19317
19318 19332 19333 19347 19348 19421 19422 19423 19463 19497 19596 19785 19786 19861 19862 19906
19907 19908 19974 19975 19990 20079 20080 20081 20082 20346 20351 20447 21331 21357 21378 21381
21382 21383 21384 21401 21413 21414 21432 21455 21456 21457 21480 21506 21507 21508 21557 21564
21582 21654 21655 21656 21710 21911 21941 21943 22474 22561 22562 22563 22564 22696 22718 22729
22740 22743 22744 22745 22746 22747 22846 22847 22937 22941 22975 22989 22997 22999 23000 23009
23037 23607 23711 23712 23866 23867 23868 24159 24854 25103 25104 25456 25457 25472 25885 25886
25887 26184 26185 26411 27456 27757 27758 28002 28136 28137 28834 28835 29153 29433 29674 29690
29751 29752 29753 29754 29755 29756 30400 30534 30535 30536 30583 30839 31064 31065 31066 31142
31178 31277 31411 31436 31578 31579 31580 31581 31871 32593 32594 32913 32914 32915 33034 34921
35302 35535 36228 36239 36421
47: 847 874 1056 1405 1721 1857 1873 1977 1978 2077 2496 2730 3014 3244 3458 4438 4499 4520 4879
5815 6048 6366 7764 7765 7766 7767 7768 7769 7770 8572 9077 9113 9419 9452 10167 10218 11251 11252
11346 11347 12429 12960 14034 14174 14176 14237 15631 15985 17874 18646 18704 18819 19090 19256
19527 19897 19898 20574 21541 22772 23157 23568 24160 25478 25686 26275 26282 26309 26435 26501
26989 27104 27754 28138 28190 28258 28454 30401 30743 31060 31224 31342 31935 32095 32471 34066
34595 34820
48: 1195 1211 1244 1485 1528 1574 1684 2268 2574 2963 2975 3097 3127 3468 5113 5694 6534 8877 9135
9190 11352 13667 16174 16496 17477 17600 17776 18568 18696 19114 19190 20373 21425 31775 35992
35993 35994 35995 35996 36227
49: 782 1561 1934 1936 2192 2257 2271 2393 2446 2700 2953 3957 3959 4405 5080 5110 5703 5856 5866
5977 6006 6019 6465 6923 6994 7054 7506 8794 9466 10141 10216 10662 10804 11365 11500 12525 12736
13025 13251 13804 13805 13919 14038 14244 14410 14611 14763 14797 14840 15234 15304 15314 15363
15695 16189 16456 16719 17077 17083 17435 17468 17739 17744 17747 18012 18337 18432 18781 18841
19408 19587 19706 19754 19916 20338 20471 20587 20872 21120 21145 21161 21353 22239 22348 22498
22852 23191 23304 23335 24339 24344 24427 24437 24590 24598 25501 26187 26298 26836 26844 27018
27270 27415 27731 27750 27835 27916 27928 27997 28162 28237 28295 28389 28467 28944 28952 28959
29493 29530 30244 30718 31391 31440 31532 31683 31899 32009 32029 32090 32510 32933 33014 33073
33110 33277 33437 33440 34596 35990 36128 36197
50: 1046 1074 1712 2251 4422 5157 5665 5666 5667 6545 7675 7784 8018 10526 13837 14070 14097 14116
19294 19832 22811 30631 31410 33883 33884 33885 33936
51: 621 958 1220 1646 2101 2241 3051 4562 4826 4843 5267 5304 5444 5504 5544 5583 5915 6077 6165
6247 6292 6494 6634 6653 6656 6989 7095 7276 7417 7497 7498 7499 7666 7733 8088 8247 8320 8541 8762
8807 9209 9224 9527 9546 9804 9833 9881 9907 10079 10401 10473 10641 10735 10892 10932 10955 11009
11085 11113 11140 11238 11466 11661 11867 11918 12020 12048 12061 12153 12329 12367 12400 12557
12721 12819 12890 12902 12904 13586 13682 13757 13962 14540 14696 14816 14839 14961 15018 15150
15387 15754 15857 16044 16208 16240 16676 16728 16802 17167 17230 17399 17490 17725 17934 18113
18182 18369 18416 18489 18506 18699 18894 19376 19610 19718 20132 20137 20464 20632 20808 20831
20882 21034 21286 21629 21707 21837 22134 22189 22218 22365 22454 22476 22516 22711 23110 23318
23510 23629 23749 23836 23930 24111 24129 24406 24489 24565 24867 25031 25042 25106 25142 25480
25496 25710 26117 26195 26373 26696 26705 26748 26767 26888 27363 27502 27661 27850 27874 28057
28647 28693 28711 28788 28875 29173 29275 29758 29864 29895 29961 30033 30184 30363 30396 30477
30637 30804 30855 30896 30910 30930 30976 31053 31202 31478 31794 32059 32311 32468 32542 32709
32727 32881 32953 32979 33036 33212 33299 33452 33538 33608 33649 33671 33786 33858 34014 34065
34215 34283 34346 34409 34477 34662 34705 34886 34941 34981 35094 35114 35214 35379 35451 35618
35781 35900 35915 36058 36132 36217 36388
52: 634 644 1346 1724 1911 1912 2420 2421 2929 2974 3107 3346 4395 5829 13866 13908 14076 17883
18654 18971 20099 21581 21936 22871 34584 34852
53: 620 645 689 765 780 897 969 1145 1486 1500 1745 1877 2369 2846 3375 9356 9431 11305 11423 17897
18663 19013 19102 19193 19206 19207 19208 19368 19409 19585 19810 20066 21039 21040 21041 21042
21934 22575 22796 24722 25100 26488 28472 31422
54: 1198 2135 2339 2826 4820 5022 5314 5552 5614 5615 5616 5953 6032 6163 6226 6282 6373 6542 6914
6956 7194 7292 7370 7376 7624 7856 7954 8193 8427 8428 8502 8556 8837 9115 9715 10128 10129 10200
10474 10749 11043 11062 11093 11175 11341 11342 11614 12120 12284 12681 12737 13702 13735 13744
13850 13952 14077 14102 14178 14369 14389 14448 14449 14833 15227 15504 15636 15693 15704 15770
16095 16297 16463 16492 17160 17220 17425 17706 18295 18487 18751 18987 19137 19429 19748 19889
19890 19891 20199 20239 20474 20631 20680 20827 20935 21343 21862 21891 21893 22072 22475 22755
23106 24123 24781 25054 25585 25622 25800 26269 26305 26810 26914 26973 27012 27200 27201 27218
27230 27373 27720 27784 27809 28146 28171 28176 28223 28243 28244 28322 28353 28357 28381 28388
28398 28441 28456 28458 28534 28535 28563 28957 29692 29697 29810 30048 30186 30520 30691 30704
30782 30882 31164 31368 31396 31413 31449 31674 31676 31690 31756 31878 31880 31936 31943 32222
32290 32306 32397 32414 32868 33057 33199 35531 35727 35910 36258
55: 634 644 1346 1724 1911 1912 2420 2421 2929 2974 3107 3346 4395 5829 13866 13908 14076 17883
18654 18971 20099 21581 21936 22871 34584 34852
56: 746 849 856 1030 1031 1266 1367 1461 1523 1992 2086 2247 2299 2505 2594 2665 2709 4295 5796 5797
6945 6963 8579 8592 9069 9082 9087 9098 9102 9108 9111 9114 10189 10208 10273 11402 11404 11415
13918 14364 14374 14375 14412 14443 14452 15109 15665 15715 16321 16327 16339 16458 16887 17855
18014 19048 19051 19267 19789 19841 20041 21054 21648 22658 22708 22830 24719 26272 26290 26977
26978 26987 27030 27232 27233 27234 27258 27262 27269 27290 27296 27305 28247 28249 28250 28311
28321 28341 28380 28382 28391 28393 28394 28405 28407 28409 28411 28437 28439 28452 28453 28457
28459 28465 28569 28570 30702 30712 30741 30751 30755 31319 31374 31416 31651 31743 31750 31760
31763 31766 31875 31908 31910 31939 31940 31942 31944 31966 32112 32129 32134 32137 32151 33815
34643 34644 34645 34646 34647 35295
57: 961 1158 1364 1424 1746 1881 1987 2020 2091 2174 2175 2176 2236 2249 2293 2362 2366 3203 3206
4038 4044 5691 6474 7771 7888 9391 9422 11353 13664 15269 16171 17849 18625 19080 19297 19321
19335 19390 19455 19972 20090 20127 21420 21626 22555 22699 22964 22981 30623 34743
58: 2444 3083 3094 3096 3696 4203 5817 6866 7994 18922 21080 21613 29761
59: 367 1159 1294 1840 2015 2099 2313 2488 2542 2579 2707 2715 2763 2823 2925 2940 2964 3170 3229
35073771 3808 4069 4104 4271 4366 4388 5753 6038 6359 7947 8820 8910 10105 10114 11000 11135
11371 11372 12419 12679 14093 14526 15270 16172 16203 16213 16277 16648 16721 17137 18163 18239
18427 18633 18859 19329 19330 19334 19980 19981 20268 20411 21352 21647 22700 22701 22901 23008
23789 24099 25562 26591 26753 27806 28522 28644 28819 29193 30312 31136 33298 33928 35669 36265
60: 366 1159 1294 1840 2763 3229 3771 4104 6038 6359 8820 8910 10105 10114 11000 11135 12419 12679
14093 14526 15270 16203 16213 16277 16648 16721 18163 18427 18859 19330 19981 20268 21647 22701
23008 23789 24099 25562 26753 27806 28522 28644 28819 29193 30312 31136 33298 33928 35669 36265
61: 4462 4615 4689 4923 5023 5058 5061 5204 5206 5261 5264 5312 5331 5380 5532 5619 5622 5631 5633
5634 6054 6080 6171 6664 6748 7087 7161 7214 7229 7444 7459 8184 8518 8621 8687 8843 9160 9332 9333
9334 9335 9336 9337 9799 9871 9883 9955 9985 10359 10404 10418 10567 10684 10695 10706 10777 10891
11193 11201 11493 11511 11541 11601 11611 11632 12123 12127 12283 12285 12336 12608 12616 12621
12659 12807 12877 12895 12929 13053 13141 13589 13645 14485 14575 14753 14807 14997 15076 15114
15482 16087 16350 16354 16561 17071 17122 17138 18001 18019 18181 18201 18313 18333 18672 18673
18753 20198 20584 20702 20856 20886 20902 21119 21281 21884 21929 21999 22036 22105 22111 22167
22260 22327 22485 23051 23071 23082 23139 23190 23239 23260 23383 23429 23495 23498 23575 23637
23773 24051 24354 24379 24397 24439 24444 24471 24474 24530 24600 24605 24630 24632 24815 24888
24964 24987 24989 25171 25242 25293 25681 25832 25836 25947 26335 26388 26846 26851 26872 26874
27060 27061 27077 27175 27272 27320 27396 27474 27488 27722 27732 27733 27738 27800 27841 27898
27908 27909 27926 27972 28035 28067 28075 28125 28598 28749 28810 28864 28897 28898 28920 28938
28943 28983 29004 29093 29094 29201 29238 29291 29478 29619 29638 29666 29719 29981 29992 30328
30342 30467 30619 31023 31788 31857 32192 32255 32616 33021 33423 33430 33490 33565 33606 33627
33660 34118 34176 34233 34241 34511 34542 34611 34814 34829 34831 34990 35026 35039 35159 35172
35492 35561 35598 35692 35702 35719 35804 35812 35859 36201 36247
62: 739 1054 1563 1824 2541 2576 2581 2661 3077 3516 4195 4414 4437 4557 4561 4585 4637 4659 4753
4795 4874 4908 4928 4930 4960 4968 5089 5140 5203 5253 5275 5278 5293 5294 5351 5395 5440 5518 5572
5621 5624 5941 6012 6084 6097 6138 6253 6255 6316 6324 6613 6627 6628 6683 6791 6802 6894 6895 6952
7003 7009 7065 7126 7191 7199 7228 7256 7257 7433 7477 7615 7618 7628 7648 7657 7705 7715 7725 8097
8103 8125 8132 8163 8218 8273 8282 8291 8313 8324 8375 8376 8377 8378 8533 8573 8614 8630 8683 8713
8714 8715 8716 8717 8724 8777 8905 8906 8928 8938 8940 9021 9047 9217 9264 9283 9350 9514 9533 9582
9604 9757 9803 9827 9828 9861 9876 9901 9933 9968 10073 10075 10078 10082 10118 10131 10310 10327
10389 10435 10507 10601 10629 10690 10728 10729 10757 10760 10814 10835 10864 10865 10897 10923
10928 10962 11014 11021 11080 11098 11112 11114 11158 11246 11331 11443 11453 11520 11560 11561
11584 11653 11683 11755 11785 11793 11830 11924 11949 12042 12043 12074 12084 12145 12150 12177
12215 12224 12254 12274 12327 12333 12534 12570 12578 12631 12636 12744 12829 13071 13105 13152
13185 13320 13441 13459 13577 13636 13754 13761 13801 13831 13955 13956 13970 14005 14017 14172
14191 14196 14260 14285 14305 14401 14476 14479 14537 14549 14578 14610 14697 14711 14713 14717
14718 14723 14751 14752 14753 14754 14755 14756 14757 14758 14759 14760 14813 14814 14849 14858
14861 14930 14970 15039 15135 15143 15158 15224 15331 15356 15390 15668 15809 15888 15986 16010
16050 16083 16088 16138 16195 16269 16343 16362 16377 16380 16556 16580 16584 16620 16646 16683
16687 16713 16741 16753 16763 16807 17018 17039 17074 17104 17144 17333 17342 17383 17420 17423
17457 17569 17595 17733 17734 17905 17913 17941 17951 17952 17970 17971 18025 18026 18087 18132
18155 18302 18310 18371 18373 18378 18411 18465 18505 18507 18691 18698 18712 18760 18780 18820
18838 19151 19616 19694 19745 19747 20050 20146 20162 20204 20209 20216 20232 20252 20254 20281
20299 20306 20390 20603 20727 20765 20800 20813 20880 20885 20939 20945 20979 21118 21152 21276
21290 21732 21774 21777 21819 21820 21821 21859 21861 21879 21885 21956 21972 22010 22055 22100
22109 22119 22132 22133 22154 22160 22174 22261 22287 22291 22311 22412 22440 22444 22489 22521
22858 23111 23145 23186 23355 23395 23417 23454 23521 23556 23627 23633 23705 23775 23781 23843
23847 23875 23903 23926 23941 23961 23995 24122 24143 24305 24307 24348 24353 24388 24410 24486
24487 24493 24527 24550 24644 24645 24650 24728 24735 24782 24811 24837 24898 24906 24913 24949
24991 25011 25019 25119 25162 25224 25328 25339 25357 25381 25409 25419 25502 25605 25620 25621
25751 25765 25791 25793 25809 25843 25952 26016 26219 26259 26304 26354 26364 26369 26404 26405
26574 26686 26687 26713 26716 26725 26733 26752 26885 26886 26930 26933 26970 27301 27337 27404
27460 27464 27497 27552 27555 27563 27619 27629 27653 27655 27703 27704 27842 27859 27883 27922
27940 27965 27970 28028 28103 28109 28133 28160 28242 28360 28379 28408 28477 28487 28513 28514
28543 28545 28572 28641 28686 28687 28734 28746 28794 28811 28872 28873 28878 28908 28910 28911
28940 29002 29127 29202 29259 29305 29314 29364 29370 29394 29395 29399 29492 29556 29589 29632
29771 29781 29816 29914 29915 29958 29959 29967 29999 30020 30086 30087 30106 30107 30123 30231
30276 30287 30345 30346 30367 30416 30443 30453 30474 30475 30525 30572 30617 30658 30659 30744
30790 30799 30836 30841 30885 30886 30907 30908 30922 30926 30927 30934 30982 31038 31206 31212
31426 31460 31472 31489 31506 31705 31800 31811 31815 31850 31895 32033 32050 32148 32194 32207
32247 32284 32344 32354 32434 32438 32439 32495 32532 32553 32566 32671 32724 32725 32756 32768
32774 32785 32886 32949 32950 32960 32971 33007 33010 33015 33037 33053 33060 33129 33147 33190
33191 33305 33428 33436 33485 33527 33543 33567 33569 33653 33765 33774 33860 33861 34011 34032
34061 34084 34202 34221 34277 34344 34421 34474 34518 34552 34559 34617 34625 34638 34660 34669
34732 34733 34761 34783 34811 34824 34882 34884 34888 34898 34902 34916 34935 34962 34991 35012
35032 35040 35085 35125 35234 35267 35394 35441 35498 35579 35614 35642 35650 35668 35677 35714
35718 35762 35763 35800 35801 35882 35939 36022 36065 36066 36104 36156 36210 36249 36252 36268
36390
63: 680 700 1755 1849 2137 2269 3141 3178 3251 3332 3401 4244 4267 4537 4596 4602 4638 4735 4794
4850 5099 5136 5195 5281 5299 5356 5399 5433 5442 5487 5492 5501 5505 5508 5517 5567 5653 6015 6082
6137 6215 6242 6308 6380 6388 6489 6569 6652 6655 6759 6771 6815 7064 7179 7443 7617 7646 7678 7713
8175 8231 8270 8281 8331 8360 8725 8739 8796 8845 8996 9001 9006 9019 9149 9155 9163 9177 9205 9225
9265 9270 9284 9450 9531 9583 9589 9592 9605 9736 9784 9800 9815 9852 9904 10247 10321 10373 10434
10485 10506 10531 10560 10710 10745 10800 10884 10900 10903 10933 10953 11005 11008 11042 11060
11095 11107 11184 11441 11452 11530 11598 11638 11706 11735 11736 11743 11796 11841 11860 11937
12011 12093 12144 12183 12197 12198 12289 12314 12533 12568 12640 12656 12695 12743 12845 13111
13180 13560 13623 13830 13872 14033 14079 14280 14496 14539 14543 14625 14672 14677 14724 14820
14822 14857 15038 15053 15054 15058 15165 15175 15231 15375 15497 15525 15535 15539 15918 15974
16018 16094 16215 16216 16264 16364 16395 16415 16621 16627 16641 16675 16686 16761 17037 17120
17147 17195 17256 17488 17732 17901 17973 17995 18110 18187 18296 18357 18417 18420 18431 18466
18477 18498 18729 18774 19160 19473 19617 19673 19692 19735 20105 20244 20451 20595 20604 20749
20801 20812 20891 20968 20993 21003 21007 21015 21114 21259 21292 21688 21765 21890 21982 22019
22033 22042 22123 22188 22217 22294 22400 22455 22457 22536 22762 22945 23121 23140 23264 23323
23543 23557 23599 23615 23727 23767 23770 23834 23874 23904 24059 24103 24146 24162 24311 24391
24405 24441 24456 24497 24568 24603 24653 24679 24770 24911 24955 25009 25115 25194 25227 25327
25374 25395 25560 25641 25714 25781 25956 26189 26220 26555 26690 26720 26758 26848 26864 26893
27070 27325 27364 27461 27505 27727 27795 27803 27860 27882 27897 28086 28089 28493 28537 28580
28740 28764 28809 28884 28939 29113 29164 29260 29335 29427 29484 29544 29722 29768 29841 29971
29975 29984 30103 30185 30227 30254 30293 30369 30435 30438 30495 30544 30557 30636 30850 31121
31200 31817 32028 32216 32389 32511 32551 32749 32775 32791 32921 32970 33189 33358 33541 33632
33676 33763 33866 34088 34099 34155 34227 34278 34343 34386 34422 34463 34485 34506 34517 34606
34683 34695 34767 34835 34934 34977 35084 35273 35382 35499 35619 35696 35722 35839 35976 36209
36238 36391
64: 4475 7743 7744 7745 7746 7747 7748 7749 7750 7751 7752 7753 7754 7755 7756 7757 7758 8130 15292
15959 16515 21803 22539 23299 24561 27662 27663 29400 31148 31168 32386 32480 33992 33993 35367
65: 616 624 632 659 674 692 694 695 717 718 751 755 756 776 778 814 816 817 860 864 872 896 934 935
1023 1029 1047 1048 1072 1079 1092 1124 1200 1203 1215 1239 1280 1293 1327 1343 1356 1357 1358 1362
1374 1389 1404 1434 1440 1451 1457 1476 1478 1487 1495 1512 1615 1677 1737 1768 1798 1878 1884 1893
1895 1900 1938 1943 1979 2018 2028 2033 2052 2061 2079 2083 2098 2119 2142 2172 2173 2222 2337 2363
2623 2626 2653 3011 3125 3359 3362 3415 3444 3656 3657 3658 3737 3752 3765 3786 3832 3839 3877 3917
3923 3925 3929 3946 4001 4066 4114 4145 4146 4237 4337 4443 4720 4736 4891 5122 5170 5748 5760 5827
5832 6192 6266 6267 6387 6422 6427 6464 6466 6499 6828 6832 6854 7138 7609 7785 7897 7898 7901 8001
8064 8067 8070 9142 9370 9417 9482 10146 10229 10245 11285 11321 11322 11357 11388 11389 11392
11395 11400 11433 12704 13356 13576 15275 15277 15326 15404 15651 15652 15653 15654 16039 16125
16136 16872 17500 17503 17527 17532 17638 17643 17693 17869 18548 18605 18960 19026 19033 19034
19035 19036 19061 19062 19065 19066 19068 19186 19221 19229 19265 19277 19278 19279 19280 19285
19301 19304 19311 19319 19358 19365 19379 19380 19385 19396 19397 19398 19399 19400 19414 19439
19445 19484 19488 19490 19768 19794 19795 19796 19797 19850 19880 19920 19921 19956 19957 19958
20001 20002 20037 20043 20060 20110 20114 20115 20116 20117 20118 20119 20120 20340 20410 20417
20431 20796 21322 21323 21356 21385 21400 21430 21502 21537 21543 21666 21705 22478 22556 22557
22558 22581 22582 22583 22584 22596 22600 22601 22612 22613 22675 22676 22677 22678 22679 22680
22683 22756 22757 22806 22813 22829 22867 22868 22902 22915 22928 22929 22992 24064 24115 24168
24942 27139 29386 29620 30391 31107 31143 31144 31181 31441 31589 31605 33811 34592 34848 35532
36324
66: 1045 1819 1960 7774 7775 19202 19869 22869 22938
67: 388 697 833 1644 2386 2442 2611 2618 2880 3058 3227 3557 3750 4192 4235 4401 4459 5802 6453 7921
7986 9940 11242 11384 13775 13811 14075 15797 17661 17802 18582 19031 19369 19983 20133 20134
20404 21489 21651 22769 35370 35371 35372 35373 35374 35375
68: 699 1121 1778 2376 2377 2604 2688 3398 3416 3500 3509 3897 4156 4363 4501 5668 5684 6007 6374
7458 8500 8548 8607 8648 9043 9051 9277 9598 11181 11861 12387 12502 13008 14067 14671 14966 15781
15923 16518 17194 17255 17763 17769 18561 18565 19729 20265 21404 21417 22759 23117 25135 25257
25308 25475 26243 27264 28777 29286 29524 29525 30132 30133 30134 30135 30851 32215 32714 33357
33441 34182 35816 35837 35908 35981 36137 36266
69: 494 582 637 866 1080 1184 1381 1627 1759 2673 2719 2726 2865 3050 3120 3279 3352 3369 3970 4375
5816 5903 8045 8074 8142 8985 9498 10143 10248 10586 10941 11998 12454 13342 13554 14889 15759
16295 17678 17835 18266 18952 20337 20957 21231 21311 21367 21371 21390 21514 23041 23228 24741
25451 25971 25977 25979 25981 25983 26936 28837 30395 33957 33996 35369
70: 698 851 1163 1531 1880 2243 2445 2687 2897 3360 3383 3461 3501 3502 3533 3555 3563 3564 3569
4013 4369 4466 5193 5739 5839 5851 7937 8009 8010 8038 9712 10241 11248 11302 15201 15810 17392
17681 18659 18948 19074 19212 19807 20070 20422 21104 21328 21364 21376 21578 22572 22620 23043
25462 26663 26808 27187 29861
71: 1057 1071 1104 1344 1378 1580 1585 1586 1748 2058 2094 2194 2692 2772 2896 2907 2936 3149 3186
3205 3249 3357 3787 3933 5420 6020 6184 6406 6871 7304 8055 8491 8985 9195 9415 9459 9651 10550
10879 11038 13960 13969 14221 14222 14223 15295 16470 17614 18260 18623 19254 19342 19584 19973
20098 20337 20647 21423 21533 21625 22862 23803 24686 24966 25818 25972 25986 26430 26495 26496
29743 29747 29804 30136 30385 30388 31103 31237 31238 31239 31463 31597 31776 34437 34438 34439
34440 34441 34442 34443
72: 676 807 903 1274 1373 2096 2395 2654 2855 3535 3719 4079 5913 7781 7782 11417 12401 17868 19132
19425 19915 20372 21449 21665 22865 26664 29387 31115 32853
73: 719 1133 1278 1341 1388 1449 1642 1854 1973 1998 2223 2723 2922 2942 2978 3064 3098 3148 3211
3522 3541 3620 4062 4251 4398 5706 5711 5814 6020 6406 6871 7967 8063 8491 8985 9195 10550 10879
11038 13707 13714 13767 13915 13930 13960 13969 13995 14120 14657 16470 18260 18533 18970 20093
20337 20393 20452 21478 22842 22965 23803 24686 25818 25972 25977 25981 25983 26430 29748 29862
30388 30390 30395 31103 31238 31597 31776 32803 33793 34448 35537
74: 1216 1429 1524 2073 2180 2225 3063 3184 3351 3820 4233 4446 4733 4746 4772 4773 4774 4775 4873
5070 5093 5369 5472 5490 5496 5503 5506 5905 6026 6206 6273 6428 6507 6593 6654 6921 7017 7018 7094
7131 7209 7249 7293 7323 7446 7478 7626 7654 7720 7852 8212 8244 8245 8288 8405 8430 8531 8583 8655
8678 8809 8961 8962 8986 9208 9414 9510 9534 9721 9735 9802 9929 9957 9959 10004 10182 10392 10536
10639 10671 10719 10739 10768 10785 10836 10926 10945 10961 10988 11006 11022 11090 11159 11222
11223 11449 11479 11486 11502 11693 11763 11776 11843 11850 11854 11896 12034 12176 12179 12317
12452 12579 12689 12923 13143 13151 13435 13477 13532 13616 14104 14297 14368 14608 14936 14975
15001 15212 15676 15735 15751 15901 15963 15989 15991 16013 16026 16107 16220 16332 16338 16525
16549 16665 16684 16806 16815 16817 16867 17374 17416 17489 17572 17574 17599 17850 18088 18212
18513 18545 18681 18773 18778 18808 18831 19359 19638 19662 19731 19756 19757 20064 20163 20196
20226 20298 20465 20645 20708 20782 21062 21233 21282 21419 21627 21680 21708 21709 21749 21772
21781 21961 21995 22003 22004 22044 22088 22343 22448 22501 22509 22639 22985 23021 23112 23162
23277 23317 23346 23455 23491 23618 23738 23880 23908 23915 23932 23954 23981 23994 24007 24044
24113 24167 24291 24413 24587 24681 24695 24736 24773 24817 24824 24831 24904 24986 25021 25040
25111 25155 25201 25219 25267 25287 25336 25517 25545 25568 25588 25614 25625 25655 25760 25767
25804 25829 25842 25967 26056 26071 26192 26313 26384 26493 26508 26522 26678 26706 26770 26791
26937 27002 27029 27095 27098 27127 27188 27191 27205 27327 27403 27428 27429 27465 27683 27868
27892 27958 27986 27988 28154 28206 28263 28276 28292 28330 28340 28442 28455 28497 28556 28561
28577 28634 28681 28706 29175 29204 29248 29279 29287 29293 29304 29312 29320 29375 29396 29441
29507 29515 29516 29517 29518 29519 29520 29630 29659 29920 30002 30089 30146 30243 30465 30481
30505 30524 30526 30608 30693 30763 30780 30843 30889 30914 30921 30929 30981 30993 31043 31062
31080 31128 31190 31208 31291 31353 31382 31450 31480 31529 31684 31688 31887 31924 31925 32057
32066 32156 32181 32236 32282 32357 32383 32409 32444 32449 32488 32528 32690 32731 32854 32876
33125 33153 33162 33279 33478 33615 33771 33890 34010 34129 34136 34138 34141 34156 34177 34235
34521 34567 34836 34878 34894 34897 34957 35025 35118 35152 35261 35397 35432 35500 35512 35554
35590 35623 35741 35779 35872 35942 36021 36052 36092 36117 36171 36319
75: 1498 1606 1683 1882 2544 2599 2781 2796 2971 3079 3122 3440 3592 3900 4109 4249 4298 4354 4476
5629 6050 6191 6668 6939 8824 8998 9066 9166 9447 9939 10202 11044 11190 11607 11890 12101 12297
12406 12674 13353 14068 14432 15403 15540 15633 15807 16311 16360 16398 17142 18752 18898 19096
19152 19840 19913 22031 22616 22967 23033 24165 26971 27057 27228 27788 28142 28161 28213 28298
28401 29712 30550 30689 31398 31728 31945 32103 32754 35729
76: 19052 19780 22647
77: 1093 1852 1939 2277 2454 2485 2554 2593 3298 3431 11280 17862 18647 18935 19185 19767 21372
22807 29154 29521
78: 311 344
79: 783 971 1168 1384 1428 1471 1556 10265 26959 29149 33844
80: 955 1156 1264 1308 1519 1643 1704 1707 2143 2161 2162 2329 2994 3337 3453 3454 3455 3886 4189
4326 6822 7878 9477 9495 9703 12391 12413 14029 14078 14101 14146 19142 19820 19965 21407 22698
23005 25449 25450 25927 25974 25976 25978 25980 25982 30138 31108
81: 374 697 729 827 833 870 1052 1088 1101 1181 1276 1312 1354 1644 1673 1698 1827 1832 2039 2442
2611 2880 3058 3227 4459 4755 4756 4757 5919 5920 6453 6860 10231 10258 11242 11272 11373 11384
13775 13811 14595 14596 15435 15814 15815 15816 15817 15818 15819 15820 15821 15822 15823 15824
15825 15826 15827 15828 15829 15830 15831 15832 15833 15834 15835 15836 15837 15838 15839 15840
15841 15842 15843 15844 15845 15846 15847 15848 15849 15850 15851 15852 15853 16886 17485 17556
17661 17662 17802 18070 18582 18694 18742 19031 19145 19369 19502 19599 19982 19983 20094 20133
20134 20658 20659 20660 20661 20662 20663 20664 20665 20666 20667 20668 20669 20670 20671 20672
20673 20674 20675 21805 22656 22769 22998 23303 26809 27105 27106 27107 35370 35371 35372 35373
35374 35375 35608 36310
82: 878 1247 1468 1605 1845 2641 2835 2931 3788 4423 5776 6452 6454 7147 7783 8080 9697 11729 12511
13716 13781 13874 13875 13879 13991 14041 14089 14092 14106 14134 15798 18755 19300 19394 20055
20359 22587 29741 30938 30962 35281
83: 682 931 1277 1481 1666 1734 1811 1812 1874 1922 2118 2218 2592 2601 2736 2997 3108 3168 3542
3584 3629 3882 4027 4176 4275 4377 4473 4503 4601 4682 4823 4871 4913 4993 5137 5174 5190 5226 5243
5301 5378 5425 5426 5439 5533 5598 5652 5999 6091 6144 6162 6212 6298 6349 6477 6551 6637 6711 6743
6986 6987 6991 6997 7090 7116 7176 7263 7441 7489 7644 7672 7712 7740 7851 7911 8090 8144 8174 8220
8306 8334 8521 8577 8633 8765 8833 8865 8954 9052 9106 9107 9222 9539 9544 9729 9783 9806 9870 9935
10016 10064 10299 10399 10469 10624 10702 10761 10801 10895 10913 10984 11016 11047 11167 11186
11332 11472 11523 11612 11659 11688 11750 11791 11856 11954 12060 12108 12163 12223 12260 12296
12316 12422 12558 12594 12634 12665 12718 12799 12817 12824 12881 12926 12971 13068 13119 13167
13198 13218 13248 13249 13250 13500 13806 13807 14009 14128 14190 14230 14274 14384 14514 14534
14582 14613 14701 14766 14789 14881 14955 15014 15048 15127 15173 15233 15383 15523 15951 15972
16092 16159 16374 16413 16585 16631 16688 16754 16774 16836 17095 17187 17248 17348 17415 17558
17590 17611 17612 17613 17947 17956 18073 18097 18199 18241 18316 18387 18418 18474 18486 18520
18682 18708 18779 18904 19161 19236 19637 19709 19734 20052 20194 20238 20304 20399 20634 20715
20802 20833 20910 20974 21008 21065 21103 21159 21218 21265 21294 21587 21608 21668 21698 21718
21766 21853 21889 22009 22011 22079 22113 22186 22251 22280 22338 22360 22453 22518 22552 22736
22815 22950 23078 23126 23294 23363 23462 23501 23512 23617 23693 23723 23764 23819 23967 24026
24072 24078 24080 24081 24107 24327 24393 24491 24507 24540 24567 24648 24844 24902 24969 24993
25039 25185 25232 25353 25388 25427 25519 25602 25740 25808 25810 25822 25860 25955 26087 26198
26325 26556 26816 26832 26889 26955 26956 27055 27134 27217 27256 27312 27333 27366 27417 27508
27564 27642 27688 27791 27938 27957 28020 28072 28234 28300 28500 28553 28597 28730 28815 28877
28909 28961 28996 29020 29024 29037 29087 29111 29167 29223 29252 29421 29469 29543 29579 29624
29668 29731 29759 29814 29964 29988 30075 30108 30149 30275 30365 30404 30430 30448 30473 30502
30513 30642 30748 30869 30906 31006 31055 31125 31461 31515 31825 31869 31906 32045 32139 32196
32225 32239 32368 32410 32455 32513 32550 32629 32723 32767 32797 32851 32985 33024 33058 33141
33206 33315 33352 33427 33551 33559 33647 33683 33792 33943 34045 34106 34165 34212 34271 34324
34424 34491 34526 34619 34654 34710 34769 34805 34913 34943 35004 35031 35072 35155 35222 35399
35423 35510 35595 35638 35703 35726 35773 35896 35927 36049 36164 36199 36287 36414
84: 829 975 1398 1464 1467 1647 2034 3506 4148 7961 8043 9449 10280 11421 13354 18599 20103 21320
22634 23305 29152 35616
85: 4725 4915 4964 5227 6259 6369 7039 8087 8349 8415 8429 8439 8902 10117 10411 10713 10952 11127
11671 12158 12235 12870 13049 13599 13653 14586 14792 15146 16204 16272 16664 18278 18515 18895
19740 21271 21856 21954 23312 23768 23827 24095 24453 24615 24775 25006 25636 25753 26532 26700
26793 26861 27316 27691 28071 28518 28973 29190 29368 29374 29769 29790 29903 29991 30808 31451
31854 34541 35055 36251
86: 706 957 1115 1193 1246 1470 1970 2030 2889 3009 3012 3022 3044 3151 3207 3252 3317 4224 4447
4768 4896 5166 5270 5325 6003 6073 6397 6418 6533 6650 7139 7271 7296 7297 7298 7629 7658 7685 7730
8084 8292 8409 8853 8933 8979 8980 8981 9035 9036 9048 9390 9496 9523 9607 10196 10274 11247 11454
11570 11574 11575 11577 11578 11724 11725 11726 11727 11728 12133 12134 12169 12437 12584 13098
13972 14028 14231 14477 14518 14562 14729 14873 15013 15092 15358 15671 15892 15942 16020 16051
16233 16234 16281 16282 16283 16284 16285 16286 16287 16288 16442 16444 16445 16467 16882 17697
17926 18003 18526 18531 18713 18857 18870 18915 19064 19377 19843 19844 20068 20288 20530 20544
20954 21445 21699 21839 21840 22156 22279 22451 22574 22880 23232 23708 23709 23795 25027 25340
25367 25399 25658 25728 25900 25905 25906 26068 26078 26281 26383 26509 26524 26737 26968 27038
27135 27375 27435 27665 27666 27844 27845 27846 28775 29848 30045 30212 30264 30506 30600 30828
30835 30897 30898 30919 31162 31186 31187 31189 31201 31292 31653 31654 31655 31657 31678 31797
31974 32155 32539 32581 32582 32590 32599 32678 32716 32823 32824 32944 32977 33536 33775 33888
33889 34387 34676 34788 34925 35001 35061 35225 35478 35834 35883 35928 36009 36108 36254
87: 4543 4630 4650 4656 4658 4665 4671 4680 4749 4803 4853 4861 4911 4917 4939 4952 4959 5051 5251
5256 5306 5447 5489 5497 5563 6104 6130 6325 6336 6339 6367 6597 6745 6790 7159 7169 7170 7171 7248
7251 7426 7622 7651 7701 7718 8120 8214 8239 8285 8497 8719 8891 9137 9242 9280 9506 9548 9593 9601
9609 9817 9980 10069 10104 10144 10296 10431 10472 10573 10829 10854 11128 11446 11690 11703
11708 11761 11779 11945 12059 12212 12270 12573 12646 12957 13018 13091 14236 14247 14277 14493
14495 14722 14876 14913 15064 15130 15132 15133 15263 15341 15364 15371 15382 15485 15933 16075
16305 16618 16638 16679 16740 16762 17050 17718 17730 17916 17957 18023 18111 18118 18196 18202
18214 18251 18511 18538 18722 19625 19670 20235 20258 20598 20938 20946 20976 20996 21045 21138
21869 22150 22193 22262 22300 22460 22520 23159 23241 23309 23537 23574 23578 23587 23679 23784
23985 24371 24429 24460 24620 25014 25212 25282 25332 25377 25743 25942 26246 26248 26353 26378
26537 26541 26669 26838 27176 27177 27353 27354 27360 27400 27402 27519 27531 27706 27839 27896
27902 28479 28639 28817 28861 29448 29834 29839 29928 29929 29930 30077 30094 30274 30442 30685
31293 31294 31295 31296 31297 31298 31299 31300 31301 31302 31303 31304 31305 31306 31307 31308
31309 31310 31311 31312 31313 31314 31453 31752 31822 31969 31985 32061 32430 32484 32556 32786
32833 32834 32835 32836 32837 32838 32839 32840 32841 32865 32901 32972 32989 32996 33186 33518
33556 33566 33648 33768 33841 33891 33892 33893 33894 33895 33896 33897 33898 33899 33900 33901
33902 33903 33904 33905 33906 33907 33908 33909 33910 33911 33912 33913 33914 33915 33916 33917
33918 33919 33920 33921 33922 33923 33924 33925 33995 34020 34510 34631 34680 34717 34775 34822
34922 34929 34985 35388 35398 35419 35436 35455 35464 35624 35681 35951 36062 36144 36174 36243
36248
88: 743 752 753 779 1089 1248 1321 1340 1359 1514 1539 1953 2439 2675 2682 2774 2777 2814 3067 3253
3282 3287 3290 3320 3338 3345 3530 3597 3731 3759 3880 4012 4228 4320 4327 5722 5723 5758 5766 6444
6492 6842 6900 7951 7952 7953 9363 9471 9472 11277 11281 11282 12403 12457 12469 12484 13684 13770
13779 13809 13873 14039 14040 14111 14446 14447 17794 17795 19112 19143 19168 19169 19219 19241
19242 19244 19769 19770 19771 19772 19773 20367 20391 20407 20427 20453 21315 21316 21484 22804
22805 22821 22912 22927 23024 23029 26293 26513 27203 27223 29381 31680 31747 31755 32163 32164
34742 35610
89: 984 985 1073 1122 1331 1387 1447 1538 1547 1663 1736 1770 1892 2084 2087 2275 2378 2543 2677
2902 2932 2979 2985 3019 3030 3037 3047 3065 3071 3095 3102 3159 3190 3193 3198 3218 3230 3412 3414
3580 3627 3755 3762 3795 3853 3905 3942 3964 4193 4201 4208 4230 4261 4280 5165 5646 5713 5737 6002
6401 6408 6423 6425 6438 6446 6449 6471 6520 6531 6675 6888 7611 8804 8922 8975 8991 9326 9327 9328
9329 9368 9377 9393 9394 9398 9399 9427 9444 9445 9446 9458 9639 9701 10150 10514 10546 10993
11283 11284 11581 12002 12003 12004 12241 12479 12481 12945 12973 13000 13099 13101 13729 14232
14706 14830 14990 15394 15395 15396 15397 15398 15548 16002 16156 16162 16165 16890 16893 16983
17001 17534 17810 18206 18402 18805 18954 19045 19125 19492 19823 19824 19825 19826 19827 19828
19829 19830 19831 19924 20472 20922 20923 21471 21910 22671 22672 22673 22822 22896 22916 22922
23010 23307 23603 23604 24306 24688 24859 25667 25672 25863 25864 25928 26080 26447 26515 26616
26762 26912 27045 27759 28001 28471 29082 29135 29680 30140 32934 33067 33971 35457
90: 673 838 914 953 1227 1408 1418 1548 1702 1917 2106 2215 3543 4743 4979 5185 5818 6385 7777 7846
7866 7996 8065 8984 9022 9489 10137 10260 11274 14211 14560 14712 15282 18542 19078 19253 19258
19303 19316 19986 20096 20138 20141 20642 20981 20982 20983 20988 20989 21194 21195 21196 21197
21198 21199 21200 21201 21394 22473 22702 22771 23687 23911 23912 23914 24691 24721 24804 25459
26120 26604 27042 27991 32159 32526 32570 35066 35290 35297
91: 947 1242 1455 1821 2380 2954 5130 5321 5640 6041 6105 6197 6666 6693 6843 7086 7099 7621 7650
7700 7717 7955 8284 8437 9012 9094 9174 9493 9507 9886 11445 11928 11929 11996 12124 12125 12131
12300 12389 12462 12572 14019 14117 15015 15093 15171 15247 15536 15811 16017 16453 16625 17473
17499 17513 17585 18099 18893 18994 19111 20069 21540 22236 22306 22347 22466 22573 24855 24980
25013 25331 25376 25394 25453 25991 26568 26781 27342 27480 27510 27576 28255 28324 28625 29532
29821 30255 30296 30330 31427 31469 32555 32830 32831 32832 32918 33038 33039 33519 33644 33767
34357 34679 34774 34984 35108 36047 36381
92: 873 988 1655 1708 2157 2184 2254 2319 2884 5143 5482 5617 5671 5681 6543 6861 7301 7778 7786
7875 8126 8860 9691 10162 10163 10164 11383 11839 13369 13370 13371 13372 13373 13374 13375 13376
13377 13378 13379 13380 13381 13382 13383 13384 13385 13386 13387 13388 13389 13390 13391 13392
13393 13394 13395 13396 13397 13398 13399 13400 13401 13402 13403 13404 13405 13406 13407 13408
13409 13410 13666 13696 13728 13741 13839 14008 14082 14658 15099 15204 15205 15427 15795 15812
16258 16478 16793 17856 18559 19312 19432 19778 20020 20059 20692 21433 22497 22595 22693 22725
24420 24703 24869 25461 25488 26385 26657 26658 26659 27043 27441 27781 28591 29694 30955 31214
31659 34571 34572 34575 34576 34577 34578 35999
93: 1393 1744 1890 2166 2463 2500 6456 8017 9124 9125 9688 12360 13321 15649 15650 17680 19728
21375 21798 22035 31150 31151 34380 34581 34861 34862 34863 34864 34865 34866 34867 34868 34869
34870 34871 34872
94: 335 455 679 723 745 785 797 798 989 1000 1060 1081 1105 1232 1241 1306 1307 1318 1323 1420 1426
1437 1445 1535 1546 1557 1624 1631 1634 1652 1659 1661 1739 1781 1788 1799 1808 1814 1891 1916 1948
2019 2041 2170 2181 2188 2189 2267 2272 2308 2449 2457 2517 2559 2572 2575 2587 2590 2628 2639 2690
2794 2812 2821 2845 2927 2957 2958 2959 2961 2965 2999 3003 3021 3023 3052 3076 3088 3090 3217 3233
3254 3259 3276 3307 3325 3333 3347 3348 3356 3384 3392 3393 3400 3472 3503 3642 3675 3683 3684 3743
3744 3745 3746 3766 3770 3781 3785 3829 3868 3876 3883 3911 3943 3945 3985 3994 4041 4042 4043 4052
4083 4098 4105 4122 4236 4277 4305 4317 4355 4474 4745 4856 4885 4886 4895 5118 5177 5289 5515 5732
6148 6395 6443 6517 6518 6550 6829 6837 6851 6880 6897 7150 7353 7354 7355 7356 7357 7358 7359 7361
7362 7363 7364 7365 7366 7367 7372 7857 8190 8374 8387 8413 8729 8857 8892 8934 8935 8936 9065 9073
9143 9262 9309 9382 9400 9587 9937 10054 10225 10284 10285 10286 10426 10456 10779 10934 10935
11792 11968 11969 11970 11971 11972 11973 11974 11975 11976 11977 11978 11979 11980 11988 11990
11991 11993 11994 12236 12237 12238 12239 12240 12259 12790 12961 12995 13001 13035 13315 13361
13755 14122 14123 14227 14233 14431 14517 14644 14645 14646 14663 14690 14834 14893 15074 15106
15190 15191 15279 15283 15559 15600 15692 15768 15806 15955 16005 16034 16055 16059 16139 16146
16150 16699 16794 16795 16796 17040 17459 17495 17502 17543 17544 17545 17546 17547 17665 17804
17826 18231 18232 18233 18234 18268 18269 18270 18271 18272 18350 18351 18475 18789 18911 18950
19009 19082 19089 19154 19188 19222 19223 19224 19286 19323 19478 19711 19763 19854 19878 20004
20051 20339 20459 20531 20532 20549 20578 20649 20650 20651 20947 21184 21185 21186 21187 21189
21190 21213 21335 21804 21829 21830 21922 21923 22685 22794 22925 22952 22957 22994 23013 23146
23330 23331 23332 23333 23553 23710 23863 23917 24294 24387 24707 24708 24709 24710 24731 24852
24897 25089 25880 25881 25924 26052 26055 26084 26104 26105 26113 26250 26289 26421 26422 26423
26424 26425 26433 26434 26526 26527 26611 26612 26833 26950 26951 26985 27049 27100 27302 27442
27443 27444 27445 27446 27673 27767 27821 27828 27829 27995 28184 28253 28365 28448 29150 29527
29749 29765 30142 30143 30144 30145 30218 30392 30512 30676 30694 30830 30940 31072 31073 31081
31092 31093 31138 31166 31222 31227 31409 31410 31465 31535 31536 31546 31547 31596 31661 31662
31667 31718 31957 31958 31979 32123 32301 32302 32303 32304 32305 32583 32955 33069 33070 33880
33969 33970 34618 34854 34858 34959 35280 35303 35304 35544 36014 36234 36432
95: 846 1166 1207 1409 1473 1494 1520 1575 1850 2259 2810 3199 3291 3796 4463 4995 5007 5008 5021
5041 5079 5103 5104 5146 5237 5292 5368 5408 5478 5491 5499 5581 5630 5820 5974 5988 6106 6286 6441
6581 6681 6723 6731 6732 6784 6988 7013 7027 7130 7151 7152 7317 7328 7329 7330 7331 7332 7333 7343
7600 7601 7640 7668 7699 7735 8085 8086 8091 8196 8197 8248 8302 8314 8871 8937 8949 9186 9187 9188
9343 9508 9762 9847 9866 9910 9934 9994 10175 10403 10421 10657 10766 10831 10943 10994 11063
11088 11169 11170 11191 11416 11468 11492 11507 11663 11738 11872 11895 11950 12110 12190 12298
12306 12346 12590 12764 12803 12808 12813 12822 12878 12896 12928 12954 13117 13187 13196 13197
13327 13336 13337 13338 13339 13340 13341 13518 13588 13644 13777 13829 13982 14059 14243 14276
14358 14502 14524 14703 14764 14806 14875 14896 14911 15180 15218 15266 15367 15516 15522 15554
15767 16029 16073 16347 16393 16407 16622 16670 16696 16726 16845 17118 17159 17218 17372 17409
17432 17879 17938 17967 18107 18130 18142 18365 18400 18524 18650 18718 18900 19131 19407 19614
19914 20187 20222 20302 20701 20937 20965 21202 21244 21399 21677 21700 21725 21768 21867 21997
22073 22135 22175 22214 22284 22340 22462 22508 22791 23238 23334 23432 23645 23654 23678 23743
23955 23977 24308 24419 24431 24475 24512 24579 24592 24633 24680 24718 24802 25034 25349 25375
25417 25546 25593 25680 25683 25716 26051 26338 26380 26391 26539 26592 26613 26736 26840 26875
26924 26932 26996 27041 27056 27062 27115 27129 27356 27516 27527 27711 27715 27723 27764 27810
27836 27923 27947 27952 27982 27994 28065 28191 28265 28375 28536 28581 28599 28600 28648 28709
28785 28919 28950 28960 29017 29107 29148 29539 29576 29669 29673 29691 29704 29836 29896 30001
30028 30109 30178 30206 30214 30267 30431 30446 30519 30529 30567 30577 30666 30684 30707 30813
30814 30966 31198 31199 31347 31447 31474 31498 31558 31784 31904 31905 32078 32202 32228 32328
32347 32401 32402 32412 32544 32584 32744 32795 32906 32922 32945 32978 33074 33085 33202 33313
33321 33520 33557 33581 33637 33693 33702 33788 33828 33998 33999 34026 34050 34211 34269 34273
34321 34328 34341 34347 34426 34548 34560 34678 34691 34752 34773 34930 34945 34983 35023 35067
35190 35211 35265 35403 35513 35625 35699 35707 35716 35752 35783 36057 36077 36194 36236 36237
36377 36412
96: 1162 4573 4730 4929 4936 4943 5149 5222 5315 5348 5393 5513 5577 6089 6297 6326 6565 6694 6804
7245 7710 8152 8340 8355 8509 8944 9210 9323 9324 9537 9982 10002 10147 10195 10317 10354 10425
10676 10687 10973 11084 11126 11583 11628 11652 11680 11927 12067 12364 12396 12629 12650 12847
12876 12918 13043 13134 13171 13550 13606 13646 13706 14467 14565 14791 15115 15136 15481 16209
16542 16716 16809 16825 17346 18158 18197 18320 18329 18384 18512 18832 19659 20257 20863 20973
21285 21743 21991 22083 22305 22517 23257 23283 23321 23479 23487 23489 23528 23772 23793 23986
24005 24040 24042 24118 24135 24445 24472 24476 24518 24543 24556 24606 24631 24635 24810 24988
25120 25386 25505 25558 25771 25826 26010 26229 26328 26558 26760 26819 26852 26873 26877 27028
27071 27103 27231 27288 27323 27544 27610 27679 27699 27734 27739 27766 27911 27914 27921 28098
28183 28282 28349 28433 28517 28826 28851 28900 28917 28979 29369 29639 29653 29656 29670 29672
29887 29897 30159 30246 30304 30336 30559 30986 31014 31022 31163 31372 31455 31524 31769 31914
32091 32201 32382 32425 32442 32548 32890 33292 33549 34002 34040 34054 34656 34807 35006 35129
35187 35247 35420 35515 35574 35639 35648 35660 35661 35682 35683 35748 36189
97: 628 687 705 716 738 774 795 799 831 855 888 928 1040 1129 1297 1480 1503 1511 1636 1679 1694 1710
2164 2502 2555 3164 3477 4047 4767 4897 5745 5991 6648 6887 7320 9033 9045 9046 9383 9891 11267
11334 11349 13924 13925 13974 15316 15958 16291 16292 16452 16468 17817 18349 18525 18593 18981
19115 19163 19200 19246 19867 19910 19992 20533 21051 21360 22854 22888 24967 25875 26103 26601
26801 27776 29678 33959 34853 36005
98: 777 1309 1988 2183 2322 2556 2837 2861 3066 3116 3953 4328 4385 4697 4698 4699 4729 5700 6018
6246 6300 7920 8432 9704 9794 10868 11077 11108 11390 11518 11613 12157 12234 12326 12478 12566
12628 13693 14795 16207 16265 16870 17419 18425 18499 18573 18756 18791 19419 19750 20121 20245
20879 21238 21447 21785 21863 22487 22585 23626 23835 24149 24485 24643 25634 26698 26790 26802
27964 28291 28512 28871 29351 29740 29784 29802 29957 30311 30349 30806 31264 33872 34750
99: 1568 1676 2334 2642 3166 3417 3814 3935 4421 4494 4556 4644 4750 4792 4931 5004 5065 5197 5263
5302 5360 5403 5473 5527 5579 5586 5690 5926 5934 5943 5956 6021 6052 6085 6100 6202 6238 6310 6371
6575 6635 6644 6662 6699 6705 6734 6778 6780 6949 6975 6978 7046 7062 7105 7121 7210 7219 7284 7285
7410 7431 7483 7706 7723 7772 8255 8269 8350 8373 8503 8507 8557 8568 8594 8728 8732 8743 8822 9093
9263 9291 9294 9551 9586 9823 9887 9981 9986 9990 10320 10476 10530 10591 10604 10668 10748 10790
10839 10887 11036 11163 11289 11684 11809 12095 12173 12219 12267 12282 12374 12375 12442 12453
12585 12655 12738 12742 12935 13107 13124 13489 13498 13543 13598 13726 14259 14263 14265 14303
14306 14405 14491 14515 14579 14600 14648 14666 14684 14705 14721 14746 14824 14863 15037 15121
15217 15378 15528 15878 15897 15935 16358 16551 16553 16586 16626 16820 16856 17027 17086 17162
17169 17222 17232 17246 17360 17366 17671 17676 17861 17910 17932 18129 18136 18282 18334 18435
18461 18641 18824 19384 19693 19714 19926 20233 20280 20725 21017 21157 21435 21682 21738 21984
22131 22140 22245 22249 22255 22270 22290 22321 22322 22355 22535 22739 23077 23105 23108 23170
23195 23296 23315 23366 23415 23503 23555 23579 23673 23929 24150 24325 24334 24338 24481 24555
24622 24792 24887 25028 25153 25158 25177 25213 25245 25247 25296 25297 25382 25420 25422 25463
25522 25541 25543 25746 25830 25897 25898 26213 26218 26233 26284 26285 26342 26343 26444 26550
26589 26761 27125 27195 27216 27250 27266 27367 27648 27804 28009 28033 28045 28060 28116 28449
28750 28799 29198 29344 29410 29457 29496 29647 29808 29890 29905 29942 29994 30007 30128 30169
30238 30289 30359 30375 30644 30662 30692 30708 30742 30823 30831 30857 30901 31010 31046 31458
31477 31677 31679 31702 31742 31790 31999 32004 32110 32114 32161 32257 32463 32464 32530 32635
32998 33098 33102 33179 33183 33215 33324 33335 33544 33546 33586 33605 33835 34078 34137 34281
34470 34496 34543 34599 34601 34665 34703 34709 34759 34801 34850 35078 35147 35233 35564 35631
35693 35695 35713 35814 36030 36039 36113 36145 36172 36215 36263 36271
100: 1390 2044 2320 2580 2603 2904 3143 3155 3189 4011 4494 4556 4644 5197 5263 5302 5360 5403 5579
5690 5943 6021 6052 6085 6100 6150 6202 6310 6371 6413 6490 6575 6635 6644 6662 6699 6705 6734 6774
6778 6780 6949 6975 6978 7062 7121 7210 7219 7410 7431 7483 7706 7723 8269 8350 8373 8557 8594 8652
8728 8732 8743 8822 9093 9274 9291 9586 9767 9823 9887 9981 9986 9990 10320 10422 10476 10530
10591 10604 10668 10711 10748 10790 10791 10839 10887 11036 11101 11163 11503 11623 11673 11684
11809 12018 12173 12219 12282 12374 12375 12442 12453 12585 12655 12742 12868 12882 12935 13055
13107 13543 13598 13717 13920 14147 14182 14370 14405 14515 14579 14600 14648 14665 14684 14705
14746 14824 14863 14965 15037 15097 15121 15217 15256 15378 15528 15878 15897 15922 15928 15935
15950 16358 16522 16626 16820 16854 16856 17027 17086 17103 17162 17222 17360 17673 17861 17932
17968 18282 18334 18435 18461 18824 19351 19655 19664 19693 19714 20139 20140 20233 20280 20457
20577 20607 20725 20858 21017 21461 21682 21738 22131 22140 22178 22290 22321 22322 22355 22535
22768 22986 23077 23105 23108 23170 23195 23250 23296 23315 23366 23415 23503 23527 23535 23673
23849 24334 24338 24555 24792 24887 25028 25153 25158 25177 25247 25297 25382 25420 25422 25463
25541 25543 25746 25897 25898 25966 26213 26218 26284 26285 26342 26343 26444 26550 26589 26761
27125 27195 27216 27250 27266 27367 27412 27545 27648 27804 27838 28009 28033 28045 28060 28449
28750 28799 28855 28862 28967 29198 29256 29282 29307 29344 29410 29457 29496 29647 29808 29890
29905 29934 29942 30128 30169 30189 30196 30238 30289 30375 30644 30662 30692 30708 30823 30831
30901 31010 31046 31458 31677 31679 31702 31741 31742 32004 32016 32110 32114 32161 32463 32464
32530 32610 32612 32635 32683 32713 32866 32998 33089 33098 33102 33179 33215 33324 33432 33445
33480 33482 33544 33546 33586 33605 33835 34078 34137 34151 34200 34281 34470 34496 34543 34599
34601 34620 34665 34703 34709 34759 34801 35058 35078 35147 35233 35564 35631 35693 35695 35713
35907 35912 35930 35953 35979 36030 36039 36113 36136 36145 36172 36215 36263 36271 36393 36404
101: 683 722 728 1099 1110 1157 1219 1251 1275 1292 1355 1394 1479 1515 1541 1549 1552 1564 1569
1572 1594 1611 1637 1718 1751 1774 1802 1817 1926 2121 2133 2230 2286 2344 2350 2490 2518 2548 2551
2591 2615 2662 2771 2804 2828 2830 2872 2926 2947 3004 3035 3068 3073 3075 3124 3128 3172 3182 3371
3372 3373 3512 3613 3628 3637 3646 3661 3666 3718 3739 3768 3769 3936 3976 3982 4020 4170 4188 4206
4225 4285 4353 4365 5726 5769 5990 6386 6392 6407 6419 6436 6479 6484 6826 6830 6931 6990 7493 7598
8011 8561 8576 8880 8881 9116 9374 9706 9726 10235 10281 10944 11197 11198 11377 11379 11432 11889
12395 12761 13553 13859 13964 13971 13980 14035 14110 14372 14380 14890 15290 15322 15708 16004
16064 16070 16186 16328 16438 16880 16881 17215 17470 17481 17519 17615 17685 17796 18229 18243
18818 18821 18956 18967 19158 19296 19344 19355 19362 19389 19451 19779 19845 19853 19887 19949
19970 20006 20015 20061 20381 20412 21464 21493 21530 21577 21643 22591 22686 22720 22770 22803
22879 22904 22905 22919 22960 23025 23438 24418 24705 26268 27024 27209 27219 27819 28227 28323
28359 28404 29138 29565 29671 29843 30729 30749 31346 31358 31721 31884 31885 31902 32140 33707
36012 36327
102: 804 1466 1573 1715 2006 2775 3216 3395 3396 3397 3583 3705 3997 4255 4356 4362 4521 4784 5241
5334 5350 5500 5510 5606 6068 6139 6396 6595 6938 7012 7091 7113 7238 7419 7634 7663 7686 7742 8271
8297 8544 8567 8657 8726 9215 9254 9282 9515 9603 9825 9873 9941 10019 10257 10370 10502 10625
10701 10734 10794 10795 10796 10918 10977 11149 11314 11440 11459 11596 11637 11871 12047 12178
12255 12535 12580 12699 12844 13079 13160 13501 13558 14057 14143 14386 14472 14735 14842 14962
15040 15368 15919 16025 16465 16824 17191 17355 17564 17720 17833 17935 17991 18307 18460 18711
18880 18901 19116 19346 19632 20008 20378 20397 20419 20605 20714 21306 21341 21683 21742 21983
22098 22168 22252 22292 22356 22392 22442 22514 22617 22903 23101 23362 23482 23572 23680 23698
23734 23742 23968 24016 24296 24576 24729 24953 24984 25022 25116 25139 25196 25345 25366 25402
25536 25617 25744 25780 25847 26211 26292 26710 26742 26934 26992 27150 27211 27276 27306 27862
27884 28022 28090 28204 28220 28338 28692 28739 28993 29146 29211 29352 29415 29571 29651 29714
30017 30035 30124 30180 30216 30262 30483 30669 30726 30895 30916 30961 30992 31033 31406 31516
31724 31818 31859 31959 32046 32117 32188 32221 32230 32271 32380 32393 32472 32534 32710 32733
32898 32920 32974 33142 33182 33246 33252 33450 33534 33678 33780 34188 34291 34350 34398 34514
34671 34726 34780 34907 34993 35024 35110 35138 35221 35511 35570 35626 35746 35902 35916 35991
36133 36181 36386
103: 309 348 349 411 630 898 905 915 1533 1595 1792 2768 3382 3404 3669 4434 5153 5688 6982 7146
7374 7885 9364 10152 10153 11279 15067 16455 16779 16780 16781 16782 16783 18532 19184 19230
19766 20402 21437 22781 24696 25892 26049 27990 33033 34259 35288 35289 36420
104: 309 348 349 410 412 630 898 905 915 1533 1595 1792 2768 3382 3404 3669 4434 5153 5688 6982 7146
7374 7885 9364 10152 10153 11279 15067 16455 16779 16780 16781 16782 16783 18532 19184 19230
19766 20402 21437 22781 24696 25892 26049 27990 33033 34259 35288 35289 36420
105: 309 348 349 411 630 898 905 915 1533 1595 1792 2768 3382 3404 3669 4434 5153 5688 6982 7146
7374 7885 9364 10152 10153 11279 15067 16455 16779 16780 16781 16782 16783 18532 19184 19230
19766 20402 21437 22781 24696 25892 26049 27990 33033 34259 35288 35289 36420
106:
107: 2302 2317 2621 4459 9731 12383 12416 12445 14161 15400 16479 16480 16481 16482 16483 16484
16485 16486 17549 18273 18549 18645 21327 27051 27052 27078 27079 27080 27081 27082 27083 27084
27085 28573 36295 36296 36297 36298 36299 36300
108: 711 750 773 1291 1577 11268 17501 18982 19216 22866
109: 696 720 876 1113 1262 1263 1267 1397 1414 1641 1645 1662 1691 1984 2010 2029 2056 2159 2160
2434 2483 3236 3621 4292 4694 5872 6462 9349 9402 10256 12996 13997 14138 15203 17664 18556 18803
19133 19449 19481 19944 19967 21319 21791 22695 22978 23001 23007 35609
110: 622 1325 1507 1616 2282 2533 2805 3136 3861 4394 5907 6044 6859 6955 7877 8574 8988 9109 10132
12376 12377 12427 12440 12510 13228 13229 13230 13231 13232 13233 13234 13235 13236 13237 13238
13239 13240 13368 14350 14351 14352 14418 14649 15401 15509 16054 16120 16487 16488 17492 17555
18020 18174 18921 19135 20034 20356 20953 21345 21603 22603 24062 24676 24764 25877 26306 27010
27174 27224 27261 27479 27830 28149 28215 28314 28447 29675 30752 31404 31424 31740 31827 31953
32094 32591 32958 33027 33758 33759 33761 34385 35529
111: 713 715 737 844 875 885 887 895 924 994 1003 1004 1005 1006 1009 1051 1160 6501 10250 10270
10275 10277 15319 15321 18166 19176 19352 22642 22823 22849 26804 26805 26812 30509
112: 686 822 840 868 921 925 940 1002 1019 1020 1059 1076 1134 6502 18167 20313 22860 24866 28902
36333
113: 545 749 757 758 764 781 882 909 1090 1114 1123 1131 1229 1230 1259 1273 1336 1348 1395 1396
1430 1439 1463 1482 1491 1496 1590 1601 1608 1610 1630 1658 1717 1720 1726 1727 1763 1815 1823 1842
1846 1855 1888 1962 1963 1969 1971 2072 2169 2231 2273 2357 2413 2415 2470 2491 2510 2513 2516 2535
2606 2647 2667 2676 2789 2793 2799 2800 2802 2803 2815 2820 2829 2843 2854 2862 2915 3036 3041 3081
3091 3110 3126 3145 3160 3204 3322 3324 3330 3354 3363 3410 3421 3425 3427 3429 3480 3482 3496 3514
3520 3525 3549 3556 3585 3589 3594 3600 3604 3605 3608 3609 3610 3611 3612 3618 3622 3626 3631 3632
3633 3635 3639 3650 3651 3653 3654 3664 3667 3671 3672 3676 3685 3686 3687 3688 3689 3690 3691 3692
3693 3694 3695 3700 3702 3706 3713 3714 3724 3729 3732 3738 3751 3756 3772 3773 3775 3777 3779 3780
3782 3792 3794 3797 3798 3799 3800 3801 3802 3810 3812 3813 3856 3888 3889 3938 3947 3948 3952 3954
3986 3987 3988 3989 4016 4017 4018 4025 4035 4070 4093 4101 4103 4106 4112 4197 4200 4204 4282 4290
4300 4323 4330 4339 4344 4358 4364 4379 4487 4488 5147 5625 5757 5869 6412 6670 6825 6943 7348 7760
7761 8408 8825 8895 9002 9037 9070 9162 9420 10145 10234 11059 11209 11210 11571 11573 12114 12293
12463 12673 13006 14006 14408 15178 15538 15672 15711 15730 15731 15732 15805 16410 16429 16430
16431 16432 16433 16434 16435 16436 16440 16461 16888 17387 17436 17509 17647 17690 17691 17828
17872 17876 18008 18601 18644 18649 18836 19070 19124 19179 19233 19448 19833 20065 20131 21246
22607 22640 22825 22898 22906 22908 23040 24544 24545 24546 24547 25059 25060 25061 25062 25063
25064 25065 25066 26420 26485 26490 26798 27017 27485 27529 27794 28165 28267 28350 29376 30547
30745 31258 31259 31260 31261 31262 31359 31377 31656 31716 31726 31916 32113 32829 32846 32847
33028 33508 33512 33513 33960 33961 33962 33963 33964 33965 33968 34456 34849 35254 35301 36006
114: 1135 1540 1553 2328 2563 2620 2698 2875 3048 3161 3361 3464 3921 4721 4759 7315 7339 7776 9475
11418 15415 15626 16229 16230 19264 19325 20107 20122 20366 21470 21649 21790 22809 26079 26300
28568 28781 33075
115: 1135 1540 1553 2328 2563 2620 2698 2875 3048 3161 3361 3464 3921 4721 4759 7315 7339 7776 9475
11418 15415 15626 16229 16230 19264 19325 20107 20122 20366 21470 21649 21790 22809 26079 26300
28568 28781 33075
116: 1135 1540 1553 2328 2563 2620 2698 2875 3048 3161 3361 3464 3921 4721 4759 7315 7339 7776 9475
11418 15415 15626 16229 16230 19264 19325 20107 20122 20366 21470 21649 21790 22809 26079 26300
28568 28781 33075
117: 1135 1540 1553 2328 2563 2620 2698 2875 3048 3161 3361 3464 3921 4721 4759 7315 7339 7776 9475
11418 15415 15626 16229 16230 19264 19325 20107 20122 20366 21470 21649 21790 22809 26079 26300
28568 28781 33075
118: 842 843 910 1417 1581 1584 1591 1907 2255 2893 2952 4440 4457 4536 4584 4591 4597 4603 4604
4633 4657 4683 4712 4783 4831 4909 5033 5272 5328 5549 5626 5761 5933 6086 6129 6136 6160 6161 6169
6434 6491 6636 6659 6667 6750 6760 6781 6807 6858 6930 6953 6954 6999 7036 7110 7193 7196 7224 7409
7484 7642 7670 7707 7737 8096 8162 8170 8176 8216 8304 8316 8372 8575 8627 8641 8731 8768 8786 8795
8826 8930 8990 9100 9101 9147 9281 9295 9301 9535 9550 9602 9679 9771 9925 10083 10314 10393 10397
10496 10568 10589 10680 10712 10877 11046 11137 11143 11180 11375 11470 11487 11539 11610 11657
11687 11716 11823 12007 12106 12128 12199 12221 12266 12278 12339 12528 12547 12556 12592 12647
12651 12652 12672 12696 12884 12984 12985 12986 13072 13081 13169 13199 13456 13479 13582 13882
14371 14484 14494 14784 14854 14984 15021 15041 15060 15125 15169 15386 15547 15682 15684 15705
16014 16318 16322 16341 16390 16409 16540 16579 16602 16609 16677 16735 17116 17208 17266 17359
17426 17522 17667 17972 18004 18165 18193 18359 18360 18386 18437 18454 18458 18463 18467 18801
19001 19199 19336 19606 19618 20038 20353 20559 20562 20599 20617 20630 20679 20756 20810 20894
21001 21014 21036 21060 21149 21517 21838 21881 21967 22017 22047 22141 22143 22169 22293 22304
22319 22335 22346 22363 22381 22388 22396 22402 22409 22419 22441 22512 22602 22893 22979 23079
23096 23152 23279 23421 23475 24031 24298 24380 24504 24562 24634 24660 24756 24823 25037 25083
25112 25137 25159 25202 25235 25268 25351 25383 25412 25421 25799 26022 26060 26085 26721 26745
26876 27013 27037 27075 27101 27222 27252 27350 27358 27494 27603 27724 27783 27786 27793 27910
27975 28025 28268 28270 28284 28308 28312 28344 28356 28429 28524 28621 28783 28843 28895 28903
28918 29200 29292 29681 29682 29699 29724 29750 29830 29838 29978 30004 30163 30165 30187 30191
30192 30247 30248 30307 30326 30337 30378 30539 30548 30746 30844 31024 31050 31267 31338 31746
31757 31762 31777 31873 31915 31917 32002 32075 32240 32268 32447 32483 32546 32579 32638 32745
32864 33079 33100 33112 33177 33180 33225 33295 33369 33391 33405 33438 33467 33525 33545 33622
33790 33801 33974 34034 34126 34145 34280 34282 34312 34339 34340 34523 34600 34659 34753 34771
34932 34963 35010 35016 35020 35097 35101 35105 35168 35223 35386 35416 35422 35560 35651 35711
35734 35808 35856 35935 35980 36018 36045 36068 36141 36179 36188 36264 36384 36385 36387
119: 346 946 1022 4065
120: 1443 6972 9136 9655 9660 9661 10192 14378 15663 18250 21395 24694 24972 26308 27014 27199
27424 27672 28164 28209 28355 28463 30747 31364 31758 31919 31921 32104
121: 429 1465 1886 1887 2407 2487 2568 2720 2917 3008 3192 3862 4329 6535 6972 9136 9386 9656 9662
10192 11380 11995 11996 13826 14378 15663 15761 15763 17698 17825 18250 19197 19475 19995 22697
22988 23665 24694 24972 26308 27014 27178 27179 27180 27181 27182 27199 27424 27672 28164 28209
28355 28463 30747 31364 31758 31919 31921 32104
122: 428 1887 2032 2407 2487 2568 2720 2917 3862 3916 4329 6972 9136 9386 9656 9658 9662 10192
11380 13826 14378 15663 17698 17825 17877 18250 18983 19197 19475 19995 21601 22697 22988 23665
24694 24972 26308 27014 27199 27424 27672 28164 28209 28355 28463 30747 31364 31758 31919 31921
32104
123: 431 858 932 1098 1180 1252 1320 1443 1913 2104 2105 2113 2471 2565 3240 3437 3540 3866 4063
4064 4071 4378 4477 4478 5736 6972 9058 9136 9372 9655 9659 9660 9661 9662 10192 10230 10254 12564
14378 15663 15761 15763 16052 16124 16155 16163 17396 17698 17808 17823 18250 18586 18787 18923
19120 19443 19963 20380 21230 21339 21358 21395 22632 23014 23796 24694 24972 26308 26811 27014
27199 27424 27672 28164 28209 28355 28463 30747 31364 31758 31919 31921 32104 34851
124: 430 1252 1320 1443 1674 1913 1957 2104 2112 2331 2471 2678 3540 4063 4064 4071 4378 4477 4478
6972 9058 9136 9372 9657 9658 9659 9660 9661 9662 9732 10192 14378 15663 16163 17698 17823 18250
18787 19120 19443 19963 21339 22632 23014 23796 24694 24972 26308 27014 27199 27424 27672 28164
28209 28355 28463 30424 30747 31364 31758 31919 31921 32104
125: 1151 1928 2289 2296 2472 3878 4173 4707 5127 5167 5168 5749 6532 6881 6882 6883 7350 7507 7508
7509 7510 7511 7512 7513 7514 7515 7516 7517 7518 7519 7520 7521 7522 7523 7524 7525 7526 7527 7528
7529 7530 7531 7532 7533 7534 7535 7536 7537 7538 7539 7540 7541 7542 7543 7544 7545 7546 7547 7548
7549 7550 7551 7552 7553 7554 7555 7556 7557 7558 7559 7560 7561 7562 7563 7564 7565 7566 7567 7568
7569 7570 7571 7572 7573 7574 7575 7576 7780 8392 8393 8394 8395 8440 8441 8442 8443 8444 8445 8446
8447 8448 8449 8451 8452 8453 8454 8455 8456 8457 8458 8459 8460 8461 8462 8463 8464 8465 8466 8467
8468 8469 8470 8471 8472 8473 8474 8475 8476 8477 8478 8479 8480 8481 8701 8846 8884 9667 9668 9669
9670 9671 9672 10512 11122 11123 11124 11125 11393 11834 11835 11836 11900 12242 13254 13255
13256 13257 13264 13265 13266 13267 13268 13269 13270 13271 13272 13273 13274 13275 13276 13277
13278 13279 13280 13281 13282 13283 13284 13285 13286 13287 13288 13289 13290 13291 13292 13293
13294 13295 13296 13297 13298 13299 13300 13301 13302 13303 13304 13305 13306 13307 13308 13309
13310 13311 13312 13313 13364 13524 13739 13838 13904 13917 14015 14074 14115 14886 15088 15513
15514 15567 15568 15569 15570 15602 15603 15604 15605 15606 15607 15608 15609 15610 15611 15612
15613 15614 15615 15616 15617 15618 15619 15620 15621 15622 15623 15624 15861 15862 15863 15864
15865 15866 15867 15868 15869 15872 15873 16497 16498 16499 16500 16501 16502 16503 16504 16505
16506 16897 16898 16899 16900 16901 16902 16903 16904 16905 16906 16907 16908 16909 16910 16911
16912 16913 16914 16915 16916 16917 16918 16919 16920 16921 16922 16923 16924 16925 16926 16927
16928 16929 16930 16931 16932 16933 16934 16935 16936 16937 16938 16939 16940 16941 16942 16943
16944 16945 16946 16947 16948 16949 16950 16951 16952 16953 16954 16955 16956 16957 16958 16959
16960 16961 16962 16963 16964 16965 16966 16967 16968 16969 16970 16971 16972 16973 16974 16975
16976 16977 16978 16979 16980 16981 16982 17617 17618 17619 17620 17621 17622 17623 17624 17625
17626 17627 17628 17629 17630 17631 17632 17633 17634 17635 17636 18028 18029 18030 18031 18032
18033 18035 18036 18037 18038 18039 18040 18041 18042 18043 18044 18045 18046 18047 18048 18049
18050 18051 18055 18056 18057 18058 18059 18060 18061 18062 18063 18064 18065 18066 18067 18068
18069 18597 19181 19231 19483 19528 19529 19531 19532 19534 19537 19540 19541 19542 19543 19544
19545 19546 19547 19548 19549 19550 19551 19552 19553 19554 19555 19556 19557 19558 19559 19560
19561 19562 19564 19565 19566 19567 19571 19572 19573 19847 20537 21056 21359 21844 21845 21847
21913 22716 22982 22983 23222 23223 23224 23225 23226 23670 23882 24169 25048 25070 25700 25868
25902 25903 25916 25917 25918 26072 26092 26093 26094 26095 26096 26097 26098 26099 26100 26101
26102 26124 26125 26126 26127 26128 26129 26130 26131 26132 26133 26134 26135 26136 26137 26138
26139 26140 26141 26142 26143 26144 26145 26146 26147 26148 26149 26150 26151 26152 26153 26154
26155 26156 26157 26158 26159 26160 26161 26162 26163 26164 26165 26166 26167 26179 26180 26181
26182 26183 26267 26382 26412 26448 26449 26450 26451 26452 26453 26454 26455 26456 26457 26458
26459 26460 26461 26462 26463 26464 26465 26466 26467 26468 26469 26470 26471 26472 26473 26474
26475 26476 26477 26478 26479 27459 28656 28657 28658 28659 28660 28661 28662 28663 28664 28665
28666 28667 28668 28669 28670 28671 28674 29039 29040 29041 29042 29043 29044 29045 29046 29047
29048 29049 29050 29051 29052 29053 29054 29055 29056 29057 29058 29059 29060 29061 29062 29063
29064 29065 29066 29067 29068 29069 29070 29071 29072 29073 29074 29075 29076 29077 29078 29081
29684 30533 30585 30586 30587 30588 30589 30590 30591 30592 30593 30594 30595 30597 31027 31213
31322 31323 31324 31325 31326 31327 31328 31329 31330 31331 31332 31333 31334 31335 31336 31437
31607 31608 31609 31610 31611 31612 31613 31615 31616 31618 31619 31621 31622 31623 31624 31625
31626 31627 31628 31629 31630 31631 31632 31633 31634 31635 31636 31637 31638 31639 31640 31641
31642 31643 31644 31645 31646 31647 31648 31649 31650 32802 33270 33274 33710 33711 33712 33713
33714 33715 33716 33717 33718 33719 33720 33721 33722 33723 33724 33725 33726 33727 33728 33729
33730 33731 33732 33733 33734 33735 33736 33737 33738 33739 33740 33741 33742 33743 33744 33745
33746 33747 33748 33749 33750 33751 33752 33753 33754 33755 33756 33757 34465 35305 35306 35308
35309 35310 35311 35312 35313 35314 35315 35316 35317 35318 35319 35320 35321 35322 35323 35324
35325 35326 35327 35328 35329 35330 35331 35332 35333 35334 35335 35336 35337 35338 35339 35340
35341 35342 35343 35344 35345 35346 35347 35349 35351 35352 35353 35354 35355 35356 35357 35358
35538 35539 35540
126: 1224 1477 1625 1706 3735 3747 6929 10188 11224 11424 11435 11436 11437 12417 13914 13932
14435 15084 15110 15288 15691 16007 16185 17694 18190 19846 20067 21569 21703 22576 24057 26431
26432 26925 26948 28208 28224 28264 28328 31317 31341 31412 31421 32587 32919
127: 2203 4147 6030 9436 11226 12373 16170 20153 21556 25988 35530
128: 2203 4147 6030 9436 11226 12373 16170 20153 21556 25988 35530
129: 650 658 703 1217 1411 1489 2529 2598 3158 3908 4100 5184 6399 6433 6448 9096 9488 9711 10261
11374 12415 12426 13366 13988 14593 14906 15712 16046 17648 17651 17783 18574 18877 19004 19281
19361 19402 20033 20044 21475 22577 24715 26523 29762 31751 31932 32577 33029
130: 650 658 703 1217 1411 1489 2529 2598 3158 3908 4100 5184 6399 6433 6448 9096 9488 9711 10261
11374 12415 12426 13366 13988 14593 14906 15712 16046 17648 17651 17783 18574 18877 19004 19281
19361 19402 20033 20044 21475 22577 24715 26523 29762 31751 31932 32577 33029
131: 789 960 997 1172 1209 1324 1422 1526 1542 2237 2497 2519 3922 3972 4178 4179 5725 6459 6855
9428 10237 10511 11381 12423 12475 14053 14118 17652 17670 17675 19291 19293 19446 19454 19775
20108 22653 22886 23215 23216 25087 33816 33882 36019
132: 789 960 997 1172 1209 1324 1422 1526 1542 2237 2497 2519 3922 3972 4178 4179 5725 6459 6855
9428 10237 10511 11381 12423 12475 14053 14118 17652 17670 17675 19291 19293 19446 19454 19775
20108 22653 22886 23215 23216 25087 33816 33882 36019
133: 789 960 997 1172 1209 1324 1422 1526 1542 1730 1795 1796 1797 1901 2878 3311 4084 6459 6855
9428 10213 10214 10215 10216 10237 10511 11381 12423 12475 14053 14118 15675 17652 19291 19446
19454 19775 20108 22653 22886 22899 22971 23215 23216 36019
134: 789 960 997 1172 1209 1324 1422 1526 1542 1730 1795 1796 1797 1901 2878 3311 4084 6459 6855
9428 10213 10214 10215 10216 10237 10511 11381 12423 12475 14053 14118 15675 17652 19291 19446
19454 19775 20108 22653 22886 22899 22971 23215 23216 36019
135: 1055 1379 1560 2050 2696 2903 4412 4420 4555 4606 4661 4728 4780 4870 4906 4947 4997 5225 5262
5326 5367 5390 5452 5535 5605 5958 5987 6075 6128 6213 6250 6283 6381 6605 6721 6821 7031 7068 7112
7174 7250 7312 7450 7471 7591 7637 7665 7698 7732 7982 8104 8167 8177 8253 8300 8345 8434 8530 8668
8791 8908 8918 8919 8920 9009 9158 9214 9273 9525 9591 9798 9842 9857 9921 9989 10051 10089 10323
10391 10499 10559 10574 10686 10723 10746 10774 10802 10838 10870 10920 10936 10951 10979 11001
11024 11065 11078 11109 11148 11360 11465 11516 11555 11655 11686 11744 11772 11797 11864 11883
11933 12037 12064 12147 12188 12252 12324 12476 12527 12588 12626 12703 12741 12771 12816 12827
12908 13039 13102 13166 13503 13579 13638 13751 13951 14140 14197 14271 14471 14525 14591 14621
14691 14748 14796 14843 14932 15044 15085 15147 15241 15332 15424 15515 15529 15755 15859 15904
15984 16098 16197 16241 16266 16386 16397 16537 16642 16651 16691 16731 16831 17042 17170 17233
17361 17371 17403 17566 17578 17736 17927 17993 18079 18127 18152 18221 18300 18426 18456 18500
18632 18683 18697 18776 18875 18912 19157 19642 19749 19834 20206 20223 20247 20285 20597 20712
20877 20967 21011 21121 21236 21264 21630 21726 21783 21849 21895 21970 22092 22103 22184 22229
22285 22324 22414 22465 22513 22551 22741 23019 23074 23076 23147 23273 23316 23378 23466 23565
23624 23701 23731 23785 23840 23873 23969 24020 24108 24147 24312 24320 24384 24483 24526 24583
24641 24771 24813 24835 24870 24892 25000 25030 25068 25170 25347 25355 25413 25521 25573 25595
25610 25640 25693 25697 25752 25795 25913 25958 26223 26264 26356 26398 26403 26497 26563 26693
26734 26768 26789 26883 27285 27313 27334 27371 27389 27486 27682 27721 27857 27878 28050 28111
28290 28485 28544 28574 28627 28677 28745 28763 28792 28870 28977 29120 29191 29192 29262 29355
29420 29440 29463 29526 29549 29583 29661 29772 29792 29833 29871 29917 29955 29993 30081 30113
30166 30229 30272 30351 30403 30447 30479 30498 30613 30663 30671 30800 30858 30912 30936 31003
31032 31133 31135 31211 31233 31517 31562 31666 31803 31996 32172 32213 32274 32440 32498 32541
32693 32729 32739 32894 33011 33025 33095 33167 33318 33336 33464 33524 33594 33662 33681 33708
33785 33862 34017 34079 34174 34232 34290 34335 34416 34482 34556 34604 34686 34715 34740 34762
34800 34893 34909 34917 34944 35003 35027 35087 35153 35219 35241 35269 35409 35445 35508 35578
35641 35764 35888 35966 36050 36063 36119 36225 36283 36410
136: 1055 1379 1560 2050 2696 2903 4412 4420 4555 4606 4661 4728 4780 4870 4906 4947 4997 5225 5262
5326 5367 5390 5452 5535 5605 5958 5987 6075 6128 6213 6250 6283 6381 6605 6721 6821 7031 7068 7112
7174 7250 7312 7450 7471 7591 7637 7665 7698 7732 7982 8104 8167 8177 8253 8300 8345 8434 8530 8668
8791 8908 8918 8919 8920 9009 9158 9214 9273 9525 9591 9798 9842 9857 9921 9989 10051 10089 10323
10391 10499 10559 10574 10686 10723 10746 10774 10802 10838 10870 10920 10936 10951 10979 11001
11024 11065 11078 11109 11148 11360 11465 11516 11555 11655 11686 11744 11772 11797 11864 11883
11933 12037 12064 12147 12188 12252 12324 12476 12527 12588 12626 12703 12741 12771 12816 12827
12908 13039 13102 13166 13503 13579 13638 13751 13951 14140 14197 14271 14471 14525 14591 14621
14691 14748 14796 14843 14932 15044 15085 15147 15241 15332 15424 15515 15529 15755 15859 15904
15984 16098 16197 16241 16266 16386 16397 16537 16642 16651 16691 16731 16831 17042 17170 17233
17361 17371 17403 17566 17578 17736 17927 17993 18079 18127 18152 18221 18300 18426 18456 18500
18632 18683 18697 18776 18875 18912 19157 19642 19749 19834 20206 20223 20247 20285 20597 20712
20877 20967 21011 21121 21236 21264 21630 21726 21783 21849 21895 21970 22092 22103 22184 22229
22285 22324 22414 22465 22513 22551 22741 23019 23074 23076 23147 23273 23316 23378 23466 23565
23624 23701 23731 23785 23840 23873 23969 24020 24108 24147 24312 24320 24384 24483 24526 24583
24641 24771 24813 24835 24870 24892 25000 25030 25068 25170 25347 25355 25413 25521 25573 25595
25610 25640 25693 25697 25752 25795 25913 25958 26223 26264 26356 26398 26403 26497 26563 26693
26734 26768 26789 26883 27285 27313 27334 27371 27389 27486 27682 27721 27857 27878 28050 28111
28290 28485 28544 28574 28627 28677 28745 28763 28792 28870 28977 29120 29191 29192 29262 29355
29420 29440 29463 29526 29549 29583 29661 29772 29792 29833 29871 29917 29955 29993 30081 30113
30166 30229 30272 30351 30403 30447 30479 30498 30613 30663 30671 30800 30858 30912 30936 31003
31032 31133 31135 31211 31233 31517 31562 31666 31803 31996 32172 32213 32274 32440 32498 32541
32693 32729 32739 32894 33011 33025 33095 33167 33318 33336 33464 33524 33594 33662 33681 33708
33785 33862 34017 34079 34174 34232 34290 34335 34416 34482 34556 34604 34686 34715 34740 34762
34800 34893 34909 34917 34944 35003 35027 35087 35153 35219 35241 35269 35409 35445 35508 35578
35641 35764 35888 35966 36050 36063 36119 36225 36283 36410
137: 1055 1379 1560 1800 2050 2696 2903 4412 4420 4555 4606 4661 4728 4780 4870 4906 4947 4997 5225
5262 5326 5367 5390 5452 5535 5605 5958 5987 6075 6128 6213 6250 6283 6381 6605 6721 6821 7031 7068
7112 7174 7250 7312 7450 7471 7591 7637 7665 7698 7732 7982 8104 8167 8177 8253 8300 8345 8434 8530
8668 8791 8908 8918 8919 8920 9009 9158 9214 9273 9525 9591 9798 9842 9857 9921 9989 10051 10089
10323 10391 10499 10559 10574 10686 10723 10746 10774 10802 10838 10870 10920 10936 10951 10979
11001 11024 11065 11078 11109 11148 11360 11465 11516 11555 11655 11686 11744 11772 11797 11864
11883 11933 12037 12064 12147 12188 12252 12324 12476 12527 12588 12626 12703 12741 12771 12816
12827 12908 13039 13102 13166 13503 13579 13638 13751 13951 14140 14197 14271 14471 14525 14591
14621 14691 14748 14796 14843 14932 15044 15085 15147 15241 15332 15424 15515 15529 15755 15859
15904 15984 16098 16197 16241 16266 16386 16397 16537 16642 16651 16691 16731 16831 17042 17170
17233 17361 17371 17403 17566 17578 17736 17927 17993 18079 18127 18152 18221 18300 18426 18456
18500 18632 18683 18697 18776 18875 18912 19157 19642 19749 19834 20206 20223 20247 20285 20597
20712 20877 20967 21011 21121 21236 21264 21630 21726 21783 21849 21895 21970 22092 22103 22184
22229 22285 22324 22414 22465 22513 22551 22741 23004 23019 23074 23076 23147 23273 23316 23378
23466 23565 23624 23701 23731 23785 23840 23873 23969 24020 24108 24147 24312 24320 24384 24483
24526 24583 24641 24771 24813 24835 24870 24892 25000 25030 25068 25170 25347 25355 25413 25521
25573 25595 25610 25640 25693 25697 25752 25795 25913 25958 26223 26264 26356 26398 26403 26497
26563 26693 26734 26768 26789 26883 27285 27313 27334 27371 27389 27486 27682 27721 27857 27878
28050 28111 28290 28485 28544 28574 28627 28677 28745 28763 28778 28792 28870 28977 29120 29191
29192 29262 29355 29420 29440 29463 29526 29549 29583 29661 29772 29792 29833 29871 29917 29955
29993 30081 30113 30166 30229 30272 30351 30403 30447 30479 30498 30613 30663 30671 30800 30858
30912 30936 31003 31032 31133 31135 31211 31233 31517 31562 31666 31803 31996 32172 32213 32274
32440 32498 32541 32693 32729 32739 32894 33011 33025 33095 33167 33318 33336 33464 33524 33594
33662 33681 33708 33785 33862 34017 34079 34174 34232 34290 34335 34416 34482 34556 34604 34686
34715 34740 34762 34800 34893 34909 34917 34944 35003 35027 35087 35153 35219 35241 35269 35409
35445 35508 35578 35641 35764 35888 35966 36050 36063 36119 36225 36283 36410
138: 998 1007 1143 1433 1740 2067 3449 4211 5733 6872 9416 9480 13985 14018 16446 16777 17805 18966
19271 19375 19434 19777 20019 20063 20955 21488 22593 22692 22732 22969 23003 23884 34744
139: 998 1007 1143 1433 1740 2067 3449 4211 5733 6872 9416 9480 13985 14018 16446 16777 17805 18966
19271 19375 19434 19777 20019 20063 20955 21488 22593 22692 22732 22969 23003 23884 34744
140: 998 1007 1143 1433 1740 2067 3449 4211 5733 6872 9416 9480 13985 14018 16446 16777 17805 18966
19271 19375 19434 19777 20019 20063 20955 21488 22593 22692 22732 22969 23003 23884 34744
141: 998 1007 1143 1433 1740 2067 3449 4211 5733 6872 9416 9480 13985 14018 16446 16777 17805 18966
19271 19375 19434 19777 20019 20063 20955 21488 22593 22692 22732 22969 23003 23884 34744
142: 936 1146 1213 2385 2717 2778 2790 2908 2984 4143 5123 5124 5740 5850 6055 6058 7940 8039 8894
10037 10038 10039 10040 10041 10042 10043 10044 10045 10046 10535 11034 14153 17743 18924 19322
19364 20003 21086 21324 21363 22610 24806 24857 26027 36320 36338 36346
143: 656 667 668 787 835 890 965 1117 1281 1400 1438 1638 1682 1735 1742 1831 1835 1923 2097 2147
2193 2211 2245 2276 2285 2347 2358 2372 2381 2401 2702 2725 2756 2757 2785 2910 2912 2918 2928 2996
3231 3234 3277 3379 3478 3493 3596 3670 3726 3823 3837 3901 3918 3963 4008 4053 4086 4088 4135 4139
4152 4165 4166 4213 4234 4544 4545 4587 4588 4645 4649 4751 4769 4781 4782 4810 4816 4828 4829 4830
4920 4973 5027 5045 5062 5091 5107 5198 5236 5279 5322 5342 5410 5455 5484 5516 5521 5529 5537 5550
5562 5569 5590 5612 5628 5644 5775 5803 5804 5813 5945 5960 5966 6001 6078 6113 6117 6119 6152 6157
6170 6193 6194 6195 6199 6208 6276 6290 6327 6345 6352 6358 6393 6455 6473 6583 6611 6669 6674 6678
6685 6687 6740 6749 6765 6785 6800 6862 6876 6889 6899 6912 6920 6958 6965 7002 7052 7079 7106 7197
7203 7235 7273 7420 7430 7434 7437 7440 7445 7465 7593 7638 7667 7682 7692 7734 7850 7987 7990 8006
8058 8062 8164 8165 8232 8251 8274 8298 8301 8400 8401 8402 8416 8520 8546 8550 8560 8569 8570 8581
8602 8625 8663 8667 8676 8736 8753 8757 8770 8774 8839 8866 8879 8956 9000 9017 9018 9067 9150 9164
9173 9220 9231 9245 9257 9267 9268 9286 9287 9299 9300 9339 9406 9411 9528 9542 9560 9562 9584 9594
9620 9645 9676 9753 9791 9809 9841 9868 10015 10057 10065 10088 10103 10108 10199 10262 10282
10293 10316 10407 10419 10443 10457 10460 10478 10487 10503 10580 10606 10631 10642 10716 10756
10771 10874 10890 10898 10940 10980 10981 10992 11007 11054 11138 11182 11250 11304 11311 11364
11419 11467 11514 11529 11533 11585 11656 11698 11753 11787 11887 11957 11960 12005 12021 12023
12026 12030 12051 12052 12083 12181 12230 12273 12315 12334 12341 12358 12368 12379 12517 12545
12551 12589 12644 12667 12710 12746 12756 12767 12770 12806 12823 12931 12948 12981 13011 13014
13023 13026 13046 13087 13092 13164 13165 13182 13438 13448 13471 13490 13508 13519 13603 13649
13668 13669 13686 13691 13699 13730 13817 13853 13912 13978 14145 14177 14179 14184 14201 14202
14203 14219 14258 14283 14300 14363 14365 14377 14453 14488 14542 14558 14580 14617 14668 14674
14686 14710 14739 14747 14837 14852 14908 14931 14963 14972 14978 15027 15049 15056 15061 15112
15161 15177 15208 15226 15265 15300 15354 15362 15380 15524 15526 15542 15703 15877 15905 15929
15947 15996 15997 15998 16006 16032 16049 16060 16065 16091 16153 16164 16179 16250 16251 16324
16356 16366 16403 16404 16510 16536 16547 16572 16600 16607 16640 16671 16682 16695 16746 16747
16758 16844 17017 17048 17076 17124 17140 17183 17192 17214 17244 17253 17365 17377 17414 17424
17427 17460 17478 17540 17679 17764 17778 17836 17837 17838 17863 17898 17924 17933 17996 18015
18072 18121 18189 18209 18210 18213 18306 18340 18344 18362 18376 18377 18403 18419 18428 18430
18449 18479 18483 18528 18606 18607 18614 18658 18703 18944 18977 18978 18988 18998 19002 19003
19049 19058 19076 19119 19174 19213 19248 19337 19338 19349 19406 19467 19615 19624 19641 19663
19715 19716 19752 19791 19792 19809 19818 19941 19942 19946 20017 20035 20075 20208 20217 20220
20297 20368 20376 20400 20441 20443 20561 20581 20622 20627 20640 20683 20710 20730 20737 20744
20745 20746 20809 20822 20841 20844 20889 20890 20897 20901 20904 20905 20999 21005 21028 21038
21097 21154 21295 21403 21491 21518 21519 21520 21535 21538 21539 21575 21652 21663 21681 21750
21888 21969 22018 22028 22030 22065 22118 22124 22142 22163 22211 22219 22266 22283 22333 22380
22387 22429 22431 22458 22484 22496 22506 22560 22567 22604 22605 22668 22669 22670 22690 22704
22775 22855 22894 22943 22954 22963 23107 23188 23308 23352 23372 23381 23382 23405 23416 23464
23465 23505 23514 23559 23567 23586 23591 23600 23606 23616 23634 23639 23766 23769 23820 23938
23962 24021 24023 24088 24346 24403 24446 24496 24534 24570 24608 24652 24765 24787 24833 24894
24895 24939 24965 25032 25051 25129 25181 25186 25228 25233 25252 25255 25272 25277 25279 25304
25348 25370 25397 25540 25569 25574 25596 25606 25726 25797 25857 25858 25954 26026 26199 26200
26225 26230 26253 26283 26299 26337 26345 26538 26572 26662 26743 26764 26797 26854 26892 26981
27020 27076 27090 27126 27225 27238 27294 27321 27345 27347 27386 27406 27407 27491 27512 27533
27611 27636 27664 27668 27701 27708 27753 27808 27847 27931 27946 27956 27971 27977 27993 28027
28064 28102 28178 28231 28316 28319 28402 28425 28489 28501 28539 28698 28741 28756 28782 28821
28829 28868 28883 28888 28890 28907 28936 29003 29009 29109 29140 29179 29214 29251 29263 29267
29333 29365 29371 29419 29488 29541 29568 29578 29600 29627 29732 29824 29827 29900 29946 29951
29969 30021 30092 30098 30120 30131 30164 30170 30177 30190 30207 30295 30299 30300 30320 30322
30354 30409 30421 30439 30541 30543 30558 30573 30607 30621 30635 30641 30645 30699 30701 30731
30732 30739 30754 30794 30821 30874 31069 31117 31159 31226 31268 31395 31486 31507 31571 31681
31732 31736 31761 31783 31851 31882 31886 31926 31934 31950 31994 32011 32014 32039 32053 32098
32100 32102 32130 32259 32340 32351 32362 32424 32478 32497 32527 32543 32559 32560 32602 32620
32661 32689 32694 32751 32764 32788 33004 33061 33093 33107 33115 33118 33121 33124 33149 33176
33184 33188 33210 33217 33218 33220 33303 33348 33355 33387 33393 33410 33455 33459 33463 33475
33539 33596 33602 33636 33666 33679 33787 33994 34004 34037 34077 34107 34112 34185 34194 34250
34262 34264 34298 34299 34338 34349 34354 34359 34401 34473 34488 34527 34623 34661 34722 34730
34763 34803 34819 34840 34877 34954 34980 35029 35080 35098 35104 35246 35426 35433 35454 35460
35534 35612 35664 35705 35731 35744 35751 35759 35793 35796 35802 35840 35851 35867 35879 35889
35920 35931 35943 35952 35964 35969 35987 35988 35989 36055 36120 36130 36143 36146 36154 36157
36162 36244 36380 36382 36416 36427
144: 321 467 2486 2567 4431 4518 4563 4590 4660 4684 4686 4727 4786 4809 4862 4864 5005 5019 5036
5055 5141 5201 5266 5284 5313 5324 5355 5371 5387 5479 5538 5551 5573 5609 5908 6074 6079 6132 6156
6173 6204 6233 6341 6360 6361 6497 6558 6568 6592 6620 6689 6733 6742 6747 6806 6874 6875 6916 7019
7069 7123 7128 7157 7177 7239 7457 7473 7636 7664 7680 7684 7731 8166 8224 8235 8299 8419 8506 8609
8637 8730 8771 8797 8869 9241 9325 9524 9529 9610 9759 9814 9830 9849 9912 9931 9972 9992 9999
10052 10334 10363 10467 10667 10677 10700 10738 10782 10813 10850 10885 10894 10975 11031 11154
11460 11528 11569 11636 11704 11752 11766 11886 11930 11934 12016 12050 12201 12210 12211 12222
12323 12331 12352 12471 12530 12555 12586 12639 12691 12759 12763 12843 12916 13061 13076 13083
13145 13176 13195 13211 13429 13557 13625 13759 13967 14169 14281 14304 14359 14360 14402 14481
14512 14564 14634 14709 14715 14726 14826 14855 14864 14957 14979 15057 15207 15228 15342 15370
15517 15914 15936 15969 16069 16293 16441 16575 16593 16692 16718 16823 16851 16861 17036 17102
17228 17337 17351 17418 17421 17516 17582 17583 17727 17728 17912 17931 17966 17987 17988 18082
18106 18125 18309 18358 18363 18391 18476 18484 18707 18732 18843 18885 19620 19666 19703 19759
20284 20560 20573 20596 20602 20626 20696 20883 20911 20966 21004 21160 21210 21671 21753 21802
21987 22121 22145 22177 22180 22238 22278 22303 22326 22332 22341 22382 22416 22463 22504 22526
23086 23138 23263 23434 23481 23488 23520 23536 23588 23631 23760 24003 24041 24091 24304 24323
24392 24433 24490 24523 24549 24559 24647 24717 24761 24796 24797 24819 24827 24828 24829 24879
24915 25003 25007 25029 25033 25176 25231 25346 25368 25372 25396 25398 25492 25520 25747 25775
25827 25907 26249 26301 26359 26396 26571 26582 26738 26779 26785 26830 27074 27273 27355 27368
27369 27382 27398 27471 27495 27553 27621 27676 27751 27769 27772 27963 27968 28061 28092 28483
28502 28538 28555 28601 28602 28623 28696 28738 28806 28882 28901 28947 28994 29118 29119 29177
29272 29298 29401 29431 29456 29464 29547 29548 29582 29609 29637 29645 29745 29813 29835 29883
29963 29980 29989 30054 30068 30155 30195 30256 30292 30332 30643 30876 30991 31019 31049 31161
31530 31809 31812 31840 31841 31842 31843 32072 32174 32238 32289 32377 32433 32443 32458 32501
32540 32564 32565 32567 32568 32621 32631 32639 32646 32673 32706 32872 32967 32999 33082 33185
33195 33293 33332 33381 33417 33456 33537 33540 33572 33635 33782 33833 34086 34104 34109 34131
34169 34247 34258 34287 34304 34308 34322 34423 34476 34512 34544 34615 34677 34685 34714 34716
34765 34781 34794 34796 34813 34826 34881 34942 34953 34979 35002 35046 35100 35113 35121 35136
35142 35182 35395 35400 35427 35489 35496 35525 35526 35527 35546 35552 35572 35694 35829 35849
35955 35978 36111 36129 36187 36224 36289 36367 36383
145: 321 467 2486 2567 4431 4518 4563 4590 4660 4684 4686 4727 4786 4809 4862 4864 5005 5019 5036
5055 5141 5201 5266 5284 5313 5324 5355 5371 5387 5479 5538 5551 5573 5609 5908 6074 6079 6132 6156
6173 6204 6233 6341 6360 6361 6497 6558 6568 6592 6620 6689 6733 6742 6747 6806 6874 6875 6916 7019
7069 7123 7128 7157 7177 7239 7457 7473 7636 7664 7680 7684 7731 8166 8224 8235 8299 8419 8506 8609
8637 8730 8771 8797 8869 9241 9325 9524 9529 9610 9759 9814 9830 9849 9912 9931 9972 9992 9999
10052 10334 10363 10467 10667 10677 10700 10738 10782 10813 10850 10885 10894 10975 11031 11154
11460 11528 11569 11636 11704 11752 11766 11886 11930 11934 12016 12050 12201 12210 12211 12222
12323 12331 12352 12471 12530 12555 12586 12639 12691 12759 12763 12843 12916 13061 13076 13083
13145 13176 13195 13211 13429 13557 13625 13759 13967 14169 14281 14304 14359 14360 14402 14481
14512 14564 14634 14709 14715 14726 14826 14855 14864 14957 14979 15057 15207 15228 15342 15370
15517 15914 15936 15969 16069 16293 16441 16575 16593 16692 16718 16823 16851 16861 17036 17102
17228 17337 17351 17418 17421 17516 17582 17583 17727 17728 17912 17931 17966 17987 17988 18082
18106 18125 18309 18358 18363 18391 18476 18484 18707 18732 18843 18885 19620 19666 19703 19759
20284 20560 20573 20596 20602 20626 20696 20883 20911 20966 21004 21160 21210 21671 21753 21802
21987 22121 22145 22177 22180 22238 22278 22303 22326 22332 22341 22382 22416 22463 22504 22526
23086 23138 23263 23434 23481 23488 23520 23536 23588 23631 23760 24003 24041 24091 24304 24323
24392 24433 24490 24523 24549 24559 24647 24717 24761 24796 24797 24819 24827 24828 24829 24879
24915 25003 25007 25029 25033 25176 25231 25346 25368 25372 25396 25398 25492 25520 25747 25775
25827 25907 26249 26301 26359 26396 26571 26582 26738 26779 26785 26830 27074 27273 27355 27368
27369 27382 27398 27471 27495 27553 27621 27676 27751 27769 27772 27963 27968 28061 28092 28483
28502 28538 28555 28601 28602 28623 28696 28738 28806 28882 28901 28947 28994 29118 29119 29177
29272 29298 29401 29431 29456 29464 29547 29548 29582 29609 29637 29645 29745 29813 29835 29883
29963 29980 29989 30054 30068 30155 30195 30256 30292 30332 30643 30876 30991 31019 31049 31161
31530 31809 31812 31840 31841 31842 31843 32072 32174 32238 32289 32377 32433 32443 32458 32501
32540 32564 32565 32567 32568 32621 32631 32639 32646 32673 32706 32872 32967 32999 33082 33185
33195 33293 33332 33381 33417 33456 33537 33540 33572 33635 33782 33833 34086 34104 34109 34131
34169 34247 34258 34287 34304 34308 34322 34423 34476 34512 34544 34615 34677 34685 34714 34716
34765 34781 34794 34796 34813 34826 34881 34942 34953 34979 35002 35046 35100 35113 35121 35136
35142 35182 35395 35400 35427 35489 35496 35525 35526 35527 35546 35552 35572 35694 35829 35849
35955 35978 36111 36129 36187 36224 36289 36367 36383
146: 1197 1254 1290 1688 1787 1810 1865 2315 2419 2566 2582 2617 2856 2909 3043 4539 4724 4935 4965
5214 5362 5404 5623 5680 5910 5937 6357 6547 6577 6672 6951 7041 7089 7133 7236 7424 7481 8100 8263
8337 8514 8580 8671 8838 8997 9167 9202 9271 9298 9381 9590 9713 9903 9996 10107 10211 10351 10595
10694 10922 10966 11064 11168 11278 11338 11604 11712 11858 11939 12102 12294 12671 12773 12848
12901 12920 13060 13155 13478 13548 13622 13832 14436 14573 14694 14926 15126 15174 15545 15681
15854 15909 16242 16355 16411 16462 16543 16811 17139 17165 17227 17362 17568 17760 18091 18115
18184 18227 18563 18817 18867 19104 19182 19466 19651 19707 19765 20027 20748 21074 21289 21408
21618 21729 21850 21851 21928 21994 22026 22221 22315 22523 22793 23085 23400 23563 23704 23725
23964 24000 24089 24584 24670 24671 24672 24808 24880 24950 24996 25113 25180 25244 25295 25514
25571 25768 25846 25926 26076 26118 26224 26291 26375 26402 26415 26502 26599 26727 26829 27039
27067 27087 27226 27339 27521 27797 28041 28084 28192 28279 28351 28403 28496 28651 28722 28991
29029 29036 29126 29184 29255 29358 29554 29587 29635 29702 29818 30010 30060 30222 30554 30688
30877 30988 31044 31402 31493 31773 31864 31894 32032 32142 32177 32218 32277 32339 32403 32467
32665 32752 32879 33050 33126 33331 33469 33554 33624 33684 34172 34225 34529 34938 35048 35141
35232 35277 35381 35473 35505 35585 35674 35743 35776 35884 35959 36051 36064 36099 36183 36321
36418
147: 3965 4571 4752 4832 4986 5011 5038 5043 5095 5097 5255 5305 5540 5560 5944 5959 6067 6112 6175
6225 6240 6272 6305 6710 6744 6769 7051 7227 7247 7264 7368 7414 7635 7693 8151 8379 8420 8504 8672
8755 8921 9213 9230 9252 9269 9516 10315 10341 10353 10367 10437 10462 10471 10500 10605 10630
10637 10658 10683 10789 10846 10999 11029 11111 11519 11592 11627 11664 11742 11789 12008 12033
12094 12138 12143 12154 12162 12168 12172 12186 12192 12198 12231 12272 12550 12630 12709 12739
13010 13030 13073 13084 13094 13109 13133 13480 13507 13520 14294 14500 14527 14544 14612 14720
14741 14745 14750 14771 14865 14874 14915 14945 14950 14951 14982 15043 15059 15087 15101 15167
15345 15377 15469 15556 15736 15880 15934 15946 16096 16104 16247 16268 16544 16722 16859 17044
17906 17911 17917 17950 17954 17963 17974 17979 17981 18133 18134 18140 18154 18226 18292 18297
18331 18395 18457 18501 18504 18680 18757 18768 19671 19742 20255 20567 20580 20582 20616 20697
20698 20711 20733 20766 20781 20959 21002 21012 21156 21162 21267 21773 21782 21857 21892 21899
21958 22062 22108 22161 22173 22274 22421 22424 22432 23087 23150 23189 23196 23203 23256 23282
23373 23506 23533 23549 23558 23585 23635 23763 23825 23846 23939 23957 23959 23978 24039 24130
24336 24382 24435 24517 24531 24595 24840 24847 24946 25017 25128 25143 25164 25188 25195 25254
25283 25360 25400 25504 25547 25582 25583 25737 25757 25963 26222 26587 26594 26618 26681 26682
26692 26841 26843 27190 27329 27378 27395 27498 27499 27518 27725 27863 27885 28069 28074 28114
28131 28593 28618 28619 28718 28790 28949 29000 29254 29341 29486 29504 29614 29776 29782 29783
29795 29796 29800 29801 29947 30070 30160 30194 30201 30259 30271 30333 30419 30458 30460 30493
30523 30528 30681 30783 30784 30797 31015 31057 31149 31473 31487 31501 31503 31787 31793 31801
31804 31806 31835 31995 32073 32171 32333 32336 32355 32604 32611 32696 32957 32976 33000 33031
33068 33078 33083 33094 33122 33143 33161 33168 33470 33471 33535 33630 33631 33650 33652 33781
34157 34315 34610 34621 34670 34793 34994 35131 35244 35465 35469 35550 35566 35798 35823 35852
35870 35913 35924 35934 35970 35977 36094 36095 36147
148: 401 1323 1624 2267 2639 2690 2959 2965 2999 3003 3021 3052 3259 3472 3642 3683 3684 3744 3745
3770 3829 3911 3943 4042 4043 4105 4122 4236 4277 4317 4474 4745 4856 4885 4886 4899 4900 5131 5132
5177 5289 5515 5732 6148 6395 6443 6516 6517 6518 6540 6541 6550 6837 6851 6880 6897 7150 7353 7354
7356 7359 7360 7361 7362 7363 7365 7366 7367 7372 7857 8124 8190 8374 8413 8729 8892 8934 8935 8936
9065 9073 9143 9262 9400 9587 9937 10054 10286 10456 10778 10779 10818 10819 10820 10821 10934
10935 11543 11544 11731 11732 11792 11905 11906 11907 11908 11909 11910 11911 11912 11913 11967
11968 11969 11970 11971 11972 11973 11974 11975 11976 11977 11978 11979 11980 11981 11982 11983
11984 11987 11988 11989 11990 11991 11992 11993 11994 12236 12237 12238 12239 12240 12259 12385
12386 12388 12465 12600 12601 12790 12961 13001 13035 13315 13525 13526 13527 13528 13529 13530
13755 14122 14123 14227 14312 14313 14314 14315 14316 14317 14318 14319 14320 14321 14322 14323
14324 14325 14326 14431 14459 14460 14461 14462 14463 14517 14644 14645 14646 14663 14690 14834
15074 15190 15191 15279 15487 15488 15572 15573 15574 15575 15576 15577 15578 15579 15580 15581
15582 15583 15600 15692 15742 15743 15744 15745 15746 15747 15748 15783 15784 15785 15786 15787
15788 15789 15790 15791 16005 16034 16112 16113 16114 16115 16116 16146 16294 16493 16494 16699
16703 16704 16705 16785 16786 16787 16788 16789 16795 16796 17040 17156 17447 17448 17449 17450
17451 17459 17543 17544 17545 17546 17547 17804 17826 18231 18232 18233 18234 18268 18269 18270
18271 18272 18350 18351 18475 18789 18911 19009 19082 19089 19154 19286 19323 19478 19711 19763
20004 20051 20156 20157 20158 20159 20160 20320 20321 20322 20323 20324 20325 20326 20327 20328
20329 20330 20332 20333 20339 20549 20578 20649 20654 20655 20656 20686 20687 20688 20689 20690
20691 20792 20793 20794 20795 20947 21184 21185 21186 21187 21188 21189 21190 21335 21701 21702
21829 21830 21831 22794 23058 23059 23060 23061 23062 23146 23332 23553 23710 23885 23886 23887
23888 24294 24387 24707 24708 24709 24710 24805 24852 24897 25706 25707 25924 26028 26029 26030
26031 26032 26033 26034 26035 26036 26052 26055 26084 26104 26105 26113 26250 26289 26422 26423
26424 26425 26433 26526 26527 26611 26612 26619 26620 26621 26622 26623 26624 26625 26626 26627
26628 26629 26630 26631 26632 26633 26634 26635 26636 26637 26638 26639 26640 26641 26642 26643
26644 26645 26646 26647 26648 26649 26650 26651 26652 26833 26985 27100 27153 27154 27155 27156
27157 27158 27159 27160 27161 27162 27163 27164 27165 27166 27167 27168 27169 27170 27171 27172
27302 27414 27442 27443 27444 27445 27446 27574 27673 27767 27821 27829 27995 28184 28253 28365
28448 28582 28583 28584 28585 28586 28587 28588 28589 29150 29527 29749 29765 30142 30143 30144
30218 30392 30676 30686 30694 30830 31072 31073 31081 31166 31255 31256 31257 31285 31286 31287
31288 31289 31290 31409 31410 31535 31536 31537 31538 31539 31540 31541 31542 31543 31544 31545
31546 31547 31548 31549 31550 31596 31661 31662 31667 31718 31957 31958 32123 32301 32302 32583
32955 33069 33070 33810 33880 34116 34364 34618 34843 34844 34858 34959 35280 35303 35544 36000
36001 36002 36003 36014 36234
149: 310 457 470 712 881 1361 1493 1772 2295 2336 2440 2630 2631 2708 2853 2989 3462 3491 3834 3927
4048 4060 4127 4140 4160 4191 4241 4250 4302 5192 5679 5712 5811 5914 6447 6485 7872 7874 9469
13359 13901 13913 16134 16173 16892 17389 17790 17866 18577 18938 19220 19851 20421 20992 21409
21479 21642 22719 23039 23691 24702 27436 27600 29693 31156
150: 310 456 1361 1772 2295 2336 2440 2630 2631 2708 2853 2989 3462 3491 3834 3927 4048 4060 4127
4140 4160 4191 4241 4250 4302 5192 5679 5712 5811 5914 6447 6485 7872 7874 9469 13359 13901 13913
16134 16173 16892 17389 17790 17866 18577 18938 19220 19851 20421 20992 21409 21479 21642 22719
23039 23691 27436 27600 29693 31156
151: 4669 4822 4994 5636 5930 6141 6149 6598 6663 6764 7001 7156 7189 7291 7449 7453 7708 8109 8279
8622 8760 8789 9255 9306 9643 9785 10106 10322 10342 10376 10406 10582 10653 11003 11061 11667
12015 12130 12185 12546 12874 13178 13604 13648 14170 14499 14545 14606 14754 14847 14953 15011
15164 15166 15179 15369 15906 16605 16635 16650 17038 17368 17705 17707 17839 17900 18000 18149
18228 18287 18422 18478 18686 18705 18715 19653 20583 20615 20704 20717 20861 21269 22000 22043
22202 22216 22223 22403 22404 22430 23141 23285 23365 23854 23877 23901 23975 24047 24390 24457
24618 24677 24724 25157 25226 25236 25276 25285 25384 25597 25735 25802 26003 26242 26377 26744
26865 26961 27393 27728 27881 27936 28128 28930 28955 29034 29329 29466 29711 29727 29738 29899
29970 29973 30049 30181 30278 30288 30420 30434 30461 30496 31272 31476 31824 31987 32080 32283
32329 32502 32504 32695 32793 33003 33087 33181 33296 33396 33582 33617 33623 33836 34047 34076
34097 34105 34134 34285 34319 34392 34658 35009 35112 35210 35475 35485 35932 36121 36392
152: 458 4669 4822 4994 5636 5930 6141 6149 6598 6663 6764 7001 7156 7189 7291 7449 7453 7708 8109
8279 8622 8760 8789 9255 9306 9643 9785 10106 10322 10342 10376 10406 10582 10653 11003 11061
11667 12015 12130 12185 12546 12874 13100 13178 13604 13648 14170 14499 14545 14606 14754 14847
14953 15011 15164 15166 15179 15369 15906 16605 16635 16650 17038 17368 17705 17707 17839 17900
18000 18149 18228 18287 18422 18478 18686 18705 18715 19653 20583 20615 20704 20717 20861 21269
22000 22043 22202 22216 22223 22403 22404 22430 23141 23285 23365 23854 23877 23901 23975 24047
24390 24457 24618 24677 24724 25157 25226 25236 25276 25285 25384 25597 25735 25802 26003 26242
26377 26744 26865 26961 27393 27728 27881 27936 28128 28930 28955 29034 29466 29711 29727 29738
29899 29970 29973 30049 30181 30278 30288 30420 30434 30461 30496 31272 31476 31824 31987 32080
32283 32329 32502 32504 32695 32793 33003 33087 33181 33296 33396 33582 33617 33623 33836 34047
34076 34097 34105 34134 34285 34319 34392 35009 35112 35210 35475 35485 35932 36121 36392
153: 4509 4626 4709 4882 5054 5242 5437 5595 5968 6818 7056 7163 7319 7321 7454 7476 8099 8505 8690
8776 8798 9341 9644 9793 9862 9918 9995 10338 10475 10647 10755 10826 10833 10875 11002 11018
11157 11513 11618 11689 11740 11775 11999 12031 12082 12347 12624 12748 12856 13181 13241 13539
13787 13993 14159 14168 14251 14505 14546 14605 14682 14728 14810 14867 14958 15213 15236 15261
15355 15557 15915 15965 16071 16564 16639 16658 17029 17180 17241 17433 17576 17914 17990 18085
18291 18424 18552 19649 19704 20203 20236 20569 20825 20875 21135 21291 21679 21866 21965 22107
22257 23201 23523 23647 23721 23817 24110 24139 24293 24400 24449 24611 24836 24893 25163 25221
25509 25567 25600 25739 25748 25764 26002 26106 26107 26108 26109 26110 26111 26112 26543 26747
26857 27128 27341 27559 27627 27697 27983 28055 28112 28638 28717 28867 29145 29172 29235 29406
29451 29570 29602 29820 29950 30233 30357 30417 30436 30454 30491 30601 30822 31037 31663 31664
32160 32166 32235 32411 32459 32508 32790 32893 33244 33283 33345 33493 33695 34008 34184 34252
34420 34602 34634 34810 34940 35208 35266 35380 35415 35491 35627 35685 35754 35855 35917 35926
36072 36207
154: 4646 4855 5119 5228 5349 5354 5394 5400 5429 5564 5565 5611 5980 6315 6372 6566 6567 6612 6695
6712 6998 7025 7048 7103 7231 7252 7412 7487 7599 7677 7861 8249 8264 8423 8508 8611 8695 8742 8827
9201 9279 9302 9338 9600 9744 9745 9863 10006 10012 10070 10339 10381 10468 10562 10708 10717
10781 10843 10914 10964 10969 11028 11165 11462 11619 11874 11956 12086 12204 12209 12662 12693
12766 12814 12836 12854 13047 13050 13137 13183 13427 13541 13567 13615 13629 14193 14198 14215
14498 14585 14633 14708 14716 14922 15478 15551 15876 15982 15987 16214 16534 16617 16667 16813
16816 17085 17347 17358 17559 17596 17713 17921 18075 18139 18318 18324 18669 18674 18733 19085
19627 20210 20224 20272 20274 20694 20839 20843 20868 21730 21741 21963 21992 22087 22246 22352
22528 23114 23130 23246 23340 23452 23480 23576 23925 23997 24004 24082 24297 24317 24461 24524
24563 24673 24785 24790 24818 24848 25004 25118 25127 25246 25511 25591 25635 25695 25698 25709
25762 25770 25813 25848 26245 26355 26416 26514 26553 26575 26817 26827 26868 27420 27476 27506
27747 27826 27904 28097 28115 28728 28926 28980 29006 29088 29089 29124 29131 29199 29228 29269
29306 29328 29347 29366 29372 29409 29475 29552 29560 29585 29592 29636 29658 30018 30037 30093
30355 30412 30444 30463 30469 30488 30499 30521 30660 30983 30994 31058 31470 31789 31813 31852
31855 32070 32297 32353 32470 32613 32657 32663 32874 32897 33080 33500 33568 33633 33673 34103
34181 34551 34568 34815 35017 35030 35160 35165 35192 35240 35586 35597 35633 35640 35756 35848
35975 36034 36087 36226 36286
155: 5200 5345 5391 5954 6334 6528 6562 7118 7436 7466 7697 8333 9878 9969 9970 10067 10408 10446
10618 10669 10849 11033 11139 11668 12208 12344 12873 13063 13142 13446 13602 13650 14587 14756
15945 16196 16205 16217 16862 16863 17369 17386 17430 17548 18280 18684 19468 21746 21960 22037
22038 22254 22386 23286 23360 23499 23500 23642 23790 23791 24029 24030 24074 25005 25130 25249
25299 25401 25503 25638 25642 25643 25759 25821 25997 26216 26740 26754 26820 27280 27317 27980
28015 28972 29613 29643 29901 30175 31017 31018 31054 31863 32076 32285 34792 35133 35567 36307
156: 5200 5345 5391 5954 6334 6528 6562 7118 7436 7466 7697 8333 9878 9969 9970 10067 10408 10446
10618 10669 10849 11033 11139 11668 12208 12344 12873 13063 13142 13446 13602 13650 14587 14756
15945 16196 16205 16217 16862 16863 17369 17386 17430 17548 18280 18684 19468 21746 21960 22037
22038 22254 22386 23286 23360 23499 23500 23642 23790 23791 24029 24030 24074 25005 25130 25249
25299 25401 25503 25638 25642 25643 25759 25821 25997 26216 26740 26754 26820 27280 27317 27980
28015 28972 29613 29643 29901 30175 31017 31018 31054 31863 32076 32285 34792 35133 35567 36307
157: 454 3965 4571 4752 4986 5011 5038 5043 5095 5097 5255 5305 6067 6240 6272 6305 6744 7051 7247
7264 7368 7414 7635 7693 8379 8420 8755 8921 9230 9252 9269 9516 10683 10846 11029 11519 11592
11664 11789 12143 12168 12192 12198 12630 12709 13030 13480 13507 13520 14294 14500 14612 14771
14865 14915 14945 14950 14951 14982 15043 15059 15087 15377 15556 15736 15880 15946 16104 16859
17044 17906 17954 18226 18457 18501 18680 18768 19671 19742 20567 20580 20616 20697 20711 20733
20766 20781 20959 21002 21012 21156 21267 21782 21857 21899 22108 22161 22173 22421 22424 23087
23196 23282 23373 23549 23825 23939 23957 23959 23978 24130 24336 24382 24435 24531 24595 24840
24847 25017 25195 25254 25360 25400 25737 25963 26222 26618 26681 26682 26692 26841 26843 27190
27329 27725 28069 28593 28718 28790 28949 29000 29341 29486 29504 29614 29776 29783 29795 29801
29947 30070 30194 30201 30259 30271 30333 30419 30458 30460 30493 30523 30528 30784 31057 31149
31473 31487 31501 31503 31806 31995 32073 32333 32336 32355 32611 32696 32957 33000 33078 33083
33094 33122 33161 33535 33781 34315 34670 34793 34994 35131 35244 35465 35469 35550 35870 35913
35924 36094 36095 36147
158: 454 3965 4571 4752 4986 5011 5038 5043 5095 5097 5255 5305 6067 6240 6272 6305 6744 7051 7247
7264 7368 7414 7635 7693 8379 8420 8755 8921 9230 9252 9269 9516 10683 10846 11029 11519 11592
11664 11789 12143 12168 12192 12198 12630 12709 13030 13480 13507 13520 14294 14500 14612 14771
14865 14915 14945 14950 14951 14982 15043 15059 15087 15377 15556 15736 15880 15946 16104 16859
17044 17906 17954 18226 18457 18501 18680 18768 19671 19742 20567 20580 20616 20697 20711 20733
20766 20781 20959 21002 21012 21156 21267 21782 21857 21899 22108 22161 22173 22421 22424 23087
23196 23282 23373 23549 23825 23939 23957 23959 23978 24130 24336 24382 24435 24531 24595 24840
24847 25017 25195 25254 25360 25400 25737 25963 26222 26618 26681 26682 26692 26841 26843 27190
27329 27725 28069 28593 28718 28790 28949 29000 29341 29486 29504 29614 29776 29783 29795 29801
29947 30070 30194 30201 30259 30271 30333 30419 30458 30460 30493 30523 30528 30784 31057 31149
31473 31487 31501 31503 31806 31995 32073 32333 32336 32355 32611 32696 32957 33000 33078 33083
33094 33122 33161 33535 33781 34315 34670 34793 34994 35131 35244 35465 35469 35550 35870 35913
35924 36094 36095 36147
159: 4045 4470 4787 4860 5164 5460 5530 5531 5575 5936 5946 6168 6786 7071 7075 7237 7442 8122 8493
8510 8640 8749 9249 9253 9569 9597 9869 9872 10374 10452 10453 10481 10594 10607 11697 12001 12195
12265 12726 13015 13082 13452 13469 14275 14327 14328 14329 14330 14331 14332 14333 14334 14335
14336 14337 14338 14339 14340 14341 14342 14343 14344 14345 14346 14347 14348 14349 14466 14478
14487 14692 14719 14918 15352 15472 16090 16188 16244 16351 16408 16599 17123 17185 17476 17554
17949 17984 18380 18436 18717 19469 19639 20572 20757 20836 21150 21887 22128 22197 22235 22241
22405 22423 23300 23389 23407 23490 23529 23573 23706 23952 24045 24876 25095 25334 25425 25718
25725 25828 26215 26237 27434 27602 28005 28040 28104 29027 29028 29206 29299 29430 29470 30261
31183 31496 32020 32295 32350 32426 32650 32895 33013 33103 33350 33411 33585 33669 33688 33689
33831 33838 34117 34274 34275 34435 34734 34757 34833 35117 35201 35202 35509 35549 35740 35747
35799 35807 35822 35911 35950 35957 35998 36028 36080 36211
160: 321 451 452 4518 4590 4660 4684 4727 4786 4862 4864 5036 5055 5141 5266 5313 5355 5479 5538
5573 6079 6132 6204 6233 6360 6361 6497 6568 6592 6689 6742 6874 6875 6916 7019 7123 7177 7239 7473
7636 7664 7680 7731 8166 8224 8299 8637 8730 8797 8869 9529 9759 9814 9849 9912 9931 9992 10052
10334 10467 10738 10813 10850 10885 10894 10975 11031 11154 11460 11528 11569 11704 11752 11886
11930 11934 12050 12210 12211 12222 12323 12331 12352 12530 12586 12639 12691 12763 13145 13195
13759 13967 14169 14281 14359 14360 14402 14512 14634 14709 14715 14826 14855 15228 15370 15517
15969 16069 16293 16441 16593 16692 16718 16823 17036 17228 17351 17418 17421 17582 17583 17727
17728 17912 17988 18106 18125 18363 18476 18732 19620 19703 19759 20284 20602 20883 20911 20966
21160 21210 21671 22121 22177 22238 22303 22332 22382 22463 22526 23138 23481 23520 23536 23631
23760 24003 24091 24304 24392 24433 24490 24549 24647 24717 24761 24796 24797 24827 24828 24829
24879 25007 25033 25372 25396 25520 25747 26359 26396 26571 26738 26785 27074 27355 27368 27382
27471 27495 27553 27621 27676 27751 27769 27772 27963 27968 28502 28538 28601 28602 28623 28696
28806 28882 28901 28947 29119 29177 29298 29431 29464 29548 29582 29637 29645 29745 29813 29835
29963 29980 29989 30054 30155 30292 30332 30643 30876 30991 31049 31161 31809 31840 31841 31842
31843 32174 32238 32443 32501 32540 32646 32673 32872 32967 33195 33293 33417 33540 33572 33635
33782 33833 34104 34109 34131 34247 34304 34322 34423 34512 34544 34685 34714 34765 34813 34881
34942 34953 34979 35046 35100 35121 35136 35182 35395 35400 35427 35546 35572 35694 35955 36224
36289 36367
161: 4512 4991 5026 5593 6766 7594 8519 9844 10326 10358 10439 11510 11631 12017 12717 14301 14929
15887 16111 16491 16852 17068 17098 17205 17475 19652 21168 21256 21908 22407 22426 23084 23179
23259 24324 24356 25156 25220 25286 25516 25940 26252 27381 29213 30647 31504 32519 32670 32686
32947 33253 33327 33403 33422 34125 34144 34492 35795 36103 36155
162: 2213 2242 2292 2573 2697 2742 2850 2876 2977 3029 3087 3093 3214 3258 3283 3284 3288 3293 3295
3308 3335 4400 5779 5780 5781 5782 6846 7876 7913 7973 7974 7995 8404 9461 9462 13665 13931 13941
14126 14129 15100 16450 18616 18617 18979 19939 19940 19945 20386 21619 22666 23297 26534 26535
26536 26593 26943 29380 33072
163: 310 456 1361 1772 2295 2336 2440 2630 2631 2708 2853 2989 3462 3491 3834 3927 4048 4060 4127
4140 4160 4191 4241 4250 4302 5192 5679 5712 5811 5914 6447 6485 7872 7874 9469 13359 13901 13913
16134 16173 16892 17389 17790 17866 18577 18938 19220 19851 20421 20992 21409 21479 21642 22719
23039 23691 27436 27600 29693 31156
164: 747 951 1034 1729 1754 1935 1999 2081 2528 2540 2578 2607 2797 2806 2808 2924 3017 3100 3121
3153 3286 3386 4091 5148 6411 6430 7763 8028 9485 9892 10278 12414 13857 14107 14442 14594 15670
16475 16889 17465 17642 19006 19007 19367 19371 19978 20144 20151 21172 21799 22733 22826 22918
23052 26271 27023 28186 28228 28335 29805 30730 31357 31420 31691 31938 32149 32162 34467
165: 473 913 949 1743 3998 6028 10232 13705 13813 13816 13940 14073 14086 14133 14139 17134 18245
18842 18999 20072 22569 35366
166: 472 913 949 1670 1675 1743 2252 2253 3016 3998 5799 6028 9723 10232 11361 11362 13705 13813
13816 13940 14073 14086 14133 14139 14559 17134 17857 18245 18635 18842 18999 19149 19234 19378
19482 19836 19837 20072 21242 21636 22569 22874 35366
167: 850 1119 1725 1762 1839 2043 2198 3049 4435 6046 6878 7941 9717 11269 12362 18589 18973 18984
19055 19196 19871 19996 20529 22856 22940 22942 22996
168: 643 815 830 1155 1206 1329 2100 2291 2760 2892 3899 4168 4495 4527 4842 4848 4852 4863 5475
5476 5917 5996 6017 6908 7314 7779 8092 9085 9709 10139 11582 12496 13774 14071 14081 14381 14702
15669 16310 17002 17483 17885 18216 18578 18834 18853 18905 19339 19698 20014 20355 20423 20552
21525 21686 22684 23870 26011 26307 26340 26358 26482 27011 27123 27268 28175 28271 28296 28385
28550 30757 30834 31748 32136 33071 33796 35606 35646 35649 35657 35672 35678
169: 702 980 1513 1589 1738 2309 2625 2888 4479 4506 4558 4664 4957 5042 5049 5158 5353 5397 5451
5459 5741 5935 5981 6142 6375 6571 6615 6617 6621 6896 6942 6950 6960 7044 7072 7100 7143 7144 7186
7270 7278 7435 7490 8078 8227 8371 8501 8522 8551 8554 8565 8566 8620 8734 8856 9068 9071 9084 9198
9285 9388 9526 9606 9681 9820 9867 9888 9952 10171 10187 10493 10563 10593 10670 10747 10763 10803
10937 11071 11144 11254 11345 11532 11842 11851 11879 11951 12206 12433 12643 13005 13074 13346
13428 13437 13504 13677 13703 13734 13762 13835 13893 14214 14302 14406 14437 14654 14683 15103
15357 15627 15630 15667 15716 15891 15932 15971 16313 16337 16373 16464 16577 16601 17584 17701
17723 17726 17814 17815 17919 18007 18117 18124 18591 19020 19088 19147 19607 19650 19701 19720
19895 20085 20609 20695 20925 20933 21085 21215 21293 21336 21337 21714 21715 21734 21787 21796
21797 21878 21933 21985 22053 22208 22271 22286 22358 22367 22500 22531 22629 22817 23341 23350
23377 23445 23470 23497 23531 23540 23583 23924 24017 24319 24345 24415 24535 25096 25167 25173
25211 25281 25288 25513 25590 25689 25779 25835 25854 26254 26279 26294 26360 26401 26426 27031
27036 27063 27137 27206 27221 27239 27309 27310 27470 27487 27490 27539 27628 27730 27782 27815
27816 28196 28200 28261 28278 28301 28339 28354 28400 28434 28609 28612 28735 28889 29116 29196
29308 29546 30065 30067 30182 30258 30646 30687 30737 30738 30920 31028 31210 31231 31345 31386
31408 31464 31471 31603 31706 31711 31892 31897 31954 32017 32071 32150 32198 32241 32338 32421
32499 32569 32662 32675 32677 32769 32862 32892 32902 32903 32905 33081 33409 33626 33664 33820
33821 33958 34146 34253 34294 34297 34404 34411 34479 34480 34500 34754 34755 34939 35109 35186
35435 35775 35794 35864 35983 36091 36107 36140 36177 36246 36294 36368 36423
170: 643 815 830 1155 1206 1329 2100 2291 2760 2892 3899 4168 4495 4527 4842 4848 4852 4863 5475
5476 5917 5996 6017 6908 7314 7779 8092 9085 9709 10139 11582 12496 13774 14071 14081 14381 14702
15669 16310 17002 17483 17885 18216 18578 18834 18853 18905 19339 19698 20014 20355 20423 20552
21525 21686 22684 23870 26011 26307 26340 26358 26482 27011 27123 27268 28175 28271 28296 28385
28550 30757 30834 31748 32136 33071 33796 35606 35646 35649 35657 35672 35678
171: 2586 3587 4703 5835 7148 17886 18657 19722 19723 19724 19725 19726 19727 23042 29855
172: 735 894 927 1091 1214 1236 1270 1432 1518 1786 2085 2767 3010 4427 4533 4624 4687 4700 4761
4762 4763 4764 4765 4766 4790 4841 4892 4984 5060 5318 5428 5528 5592 5833 6057 6065 6096 6124 6322
6488 6713 6753 6890 6902 6966 6969 7204 7411 8189 8209 8388 8638 8669 8688 8733 8803 8842 8959 8983
9062 9083 9119 9296 9484 9513 9549 9626 9843 10179 10212 10215 10303 10379 10464 10770 10822 11192
11382 11642 11759 11774 11819 11893 11963 12444 12677 12840 12959 13013 13096 13220 13224 13319
13563 14186 14199 14204 14293 14310 14311 14425 14516 14615 14659 14660 14744 14888 14909 14974
14981 15163 15258 15325 15475 15560 15702 15707 15710 15713 15714 16103 16232 16345 16459 16513
16538 16562 16595 16849 16989 17059 17199 17261 17463 17464 17616 17640 17641 17884 18356 18394
18450 18656 18812 18829 18833 19295 19388 19776 20062 20211 20301 21117 21123 21574 21841 21898
22110 22158 22265 22592 22731 23090 23171 23266 23509 24060 24360 24416 24766 24784 24801 25088
25410 25468 25661 25962 26115 26260 26361 26480 26503 26546 26654 26660 26988 27007 27035 27253
27255 27267 27426 27427 27675 27773 27805 28038 28156 28159 28177 28180 28219 28256 28257 28342
28346 28362 28422 28436 28444 28486 28541 28675 28776 29016 29030 29031 29032 29035 29155 29237
29261 29331 29453 29705 29760 29763 29764 30032 30253 30655 30727 30768 30769 30854 31100 31180
31343 31350 31390 31393 31713 31722 31735 31768 31798 31883 31911 31955 32489 32654 32904 32942
33200 33250 33308 33362 33414 33465 33491 33528 33640 33809 33843 34030 34087 34149 34205 34214
34362 34434 34444 34481 34605 34635 34668 34712 34760 34920 34968 34992 35062 35075 35296 35365
35833 35857 35876 35886 35971 36010 36011 36084
173: 633 826 1027 1028 1132 1279 1460 1769 1868 2024 2065 2636 6451 6848 6849 9404 11429 13905
14114 15313 16784 17887 19386 19435 20021 20022 20101 21567 22870 22947 22993 24061 25448 33243
33818
174: 766 2115 2732 5837 6478 8016 12360 13321 13713 14007 14027 15649 15650 16447 18265 18946
19487 19728 21373 21798 22035 33263 34380 34581 34864 34868 34870 34871 34872
175: 1075 1188 1319 1527 1530 1543 1558 2117 2307 2782 3200 5838 6614 8008 8682 10228 10957 11396
13888 14150 14383 14413 14414 14416 14440 14455 14916 19144 19433 19860 20382 21101 21579 21696
22477 22820 23728 24629 26661 26708 26772 27133 27840 28193 29115 30892 30905 32400 33600 34901
176:
177: 681 1668 2263 2652 2919 5865 6985 8029 11386 12408 12998 16169 17518 17812 18553 18783 19453
19991 21583 22730
178: 1353 1758 1902 2163 2747 3265 3343 3390 3624 4351 4361 4372 4574 5789 5922 5923 5924 6420 6426
6870 7165 8153 9652 11074 12129 13360 13680 13858 13906 14141 14420 17135 17136 17506 17807 18200
18738 21438 25050 25734 27501 30721 30722 32746 32777 33275 35467
179: 518 684 792 1194 1317 1407 1448 1848 1959 2003 2048 2239 2738 3123 4283 4424 5112 5720 5763
6037 6189 6475 7373 7927 8034 8818 9467 9487 11327 11328 11411 11412 11413 11580 12503 13227 16874
18527 18962 18980 19005 19121 19122 19204 19218 19340 19426 19427 19464 19874 19875 19900 19901
19902 20398 21481 21482 21542 22624 22625 22765 22766 22944 22973 24423 24704 26962 32479 32932
33065 34741
180: 518 684 792 1194 1237 1317 1403 1407 1448 1459 1848 1959 2003 2048 2466 3399 3497 5112 6037
6475 7373 8818 9138 9139 9467 9487 11327 11328 11411 11412 11413 12503 18527 18962 18980 19005
19121 19122 19204 19218 19340 19426 19427 19464 19874 19875 19900 19901 19902 21558 22624 22625
22765 22766 22944 22973 24423 24704 26962 32479 32932 33065 34741 35533
181: 726 871 15310 27530
182:
183: 1760 6834
184: 1760 6834
185:
186: 1174 1221 1257 1288 1570 1578 1805 1806 2026 2158 2202 2311 2355 2450 2557 2595 2608 2967 2980
3210 3292 3941 4074 4076 4321 4389 4449 4510 4516 4627 4641 4817 4845 4933 4966 5010 5209 5258 5268
5283 5323 5344 5389 5463 5494 5546 5568 5635 5647 5649 5677 5708 6042 6095 6153 6201 6495 6560 6596
6673 6754 6863 6967 7029 7115 7162 7472 7620 7656 7696 7724 7787 7791 7792 7869 7870 8021 8114 8290
8312 8358 8488 8584 8587 8670 8758 8823 8834 8992 8995 9005 9016 9134 9148 9151 9159 9170 9175 9176
9178 9216 9240 9387 9453 9512 9611 9637 9642 9795 9819 9836 9911 10176 10185 10198 10203 10204
10648 10758 11057 11058 11070 11150 11177 11189 11451 11606 11609 11692 11795 11891 12089 12103
12104 12105 12113 12126 12170 12193 12203 12214 12264 12276 12279 12281 12290 12301 12309 12485
12552 12577 12670 12676 12685 12713 12757 13004 13037 13106 13190 13316 13584 13672 13864 13894
14046 14192 14217 14250 14362 14468 14568 14732 14736 14778 14853 14868 14880 14937 15026 15154
15160 15176 15223 15338 15344 15347 15349 15521 15532 15546 15689 15903 15961 15983 16011 16053
16140 16184 16221 16334 16370 16383 16391 16394 16396 16405 16412 16541 16624 16751 16765 16766
16805 16868 17106 17109 17115 17119 17121 17179 17240 17341 17461 17525 17695 17787 17903 17915
17980 18080 18098 18192 18246 18276 18289 18354 18480 18688 18709 18725 18772 18890 18920 19077
19177 19178 19210 19251 19702 19721 19876 20106 20207 20218 20439 20456 20623 20736 20848 20917
21029 21136 21167 21214 21229 21297 21411 21724 21811 21815 22007 22016 22021 22023 22024 22025
22097 22148 22155 22187 22231 22299 22373 22452 22507 22763 22774 23094 23589 23651 23688 23702
23716 23757 24109 24119 24310 24426 24589 24687 24732 24886 24938 24947 25018 25108 25151 25225
25275 25330 25356 25408 25411 25555 25637 25708 25723 25731 25806 25893 25920 25929 26005 26082
26090 26208 26302 26348 26598 26600 26677 26732 26756 26835 26922 27000 27059 27308 27385 27419
27455 27463 27509 27535 27656 27748 27790 27869 27893 28053 28078 28169 28221 28333 28399 28549
28578 28654 28712 28769 28772 28820 28986 29141 29171 29245 29362 29414 29534 29535 29629 29709
29710 29717 29721 29725 29737 29924 29997 30119 30202 30266 30321 30410 30441 30462 30527 30542
30552 30553 30560 30569 30661 30771 30779 30871 30980 31034 31415 31593 31709 31795 31826 31833
31889 31890 32105 32197 32531 32692 32741 32743 32748 32761 32766 32771 32843 32966 32988 32997
33194 33301 33344 33468 33529 33584 33634 33773 33875 34007 34062 34154 34243 34302 34313 34381
34384 34415 34498 34501 34555 34667 34693 34698 34784 34799 34970 35076 35407 35414 35621 35732
35737 35739 35742 35885 36118 36218 36242 36358 36408
187: 582 637 866 1365 1731 2080 2179 2387 2673 2719 2726 2865 3050 3352 3940 3970 4155 5816 7302
7993 8019 8045 8074 8142 8985 9365 9409 9470 9498 10143 10549 10586 10941 11434 11998 12454 12492
13554 14085 14889 14895 15295 15759 16295 17133 17835 18266 18535 18536 18621 18952 19015 19016
19099 19100 19239 19261 19309 19452 19584 19857 19882 19883 19884 19885 19886 20104 20337 20957
21311 21371 21814 22749 22853 22864 22926 23041 23228 24741 24966 25971 25979 25981 25986 28837
29010 29862 30389 30395 31029 31253 32936 33996 35283 35287 35369
188: 1069 1371 1804 2645 2646 2801 3273 3323 3376 3858 4894 5770 6027 6910 9403 9486 10170 12504
13885 14376 15632 16309 17842 18846 19175 19205 19272 19870 21546 22783 22939 23032 24941 25043
26314 26999 27254 28155 28254 28294 28426 28565 30772 30773 31383 31723 31876 32143
189: 942 963 1316 1499 1632 2761 2987 5809 6973 8874 10219 14062 14083 15289 17865 18637 19127
19444 19822 20370 21089 21658 22953 23038 24758 24780 29382 32571 32574 34378
190: 942 963 1316 1632 2484 2754 3895 5715 6973 10219 14062 14083 15289 16127 17792 19127 19444
19822 21453 22764 22953 24758 24780 27532 31697 32571 32574 34378
191: 825 966 1210 1462 2221 2913 3722 4186 4600 4702 5161 5162 5163 5791 5792 5904 6111 6506 6508
6536 6735 7218 7793 7977 8173 8792 10158 11195 11730 13849 14181 14662 14891 14892 14991 15389
15412 15413 15414 16252 16253 16254 16255 16256 16778 17541 17542 18146 18624 18749 20466 20467
20468 20469 20470 21212 21509 23213 23595 23660 23718 23919 23920 24404 24665 24666 24667 24668
24669 25044 25045 25046 25091 25092 25093 25878 25879 25891 25919 26952 26953 26954 27048 27142
27573 27599 27817 27818 28830 28831 30269 30508 30515 31182 31234 31273 31274 31275 31587 32849
33826 33942 34361 34363 34804 34856 35479 35480 35481 35482 35483 35542
192: 734 995 1107 1183 1234 1302 1338 2475 6398 9727 13722 13725 13834 13887 15273 17871 19491
20341 21387 22750
193: 979 1628 1686 1841 2871 2886 3387 4265 4425 4737 4738 4760 4974 5178 5672 5755 5921 6461 6527
6852 6877 7318 7340 7577 7578 7579 7616 8007 8014 8015 8490 8914 8923 8947 8969 9435 9455 9456 9699
9722 9728 10423 11221 11288 11323 11545 11546 11547 11548 11549 11550 11551 12116 12117 12118
13679 13923 14987 14988 15104 15195 16009 16303 16304 18541 19032 19043 19155 19273 19274 19438
19476 19477 19586 19604 19605 19925 19959 19960 19961 19962 20392 20436 20437 20551 21389 22541
22667 22681 22738 23028 23048 25053 25691 26088 26957 27430 27453 27454 27577 27578 27768 28003
30507 30517 30957 30958 30959 31114 31284 31991 32719 32861 32940 33020 33241 33242 33509 33667
33934 33937 33938 34206 34388 34389 34390 34449 34450 34451 34452 34453 34454 34455 35294 35363
35690
194: 1633 1669 2351 2503 2686 2783 2848 3101 3321 3456 3680 3817 4130 4266 5729 5876 5877 5878 5879
5880 5881 5882 5883 5884 5885 5886 5887 5888 5889 5891 5894 5896 5897 5898 9437 9478 10424 11319
11336 15802 17797 17799 19326 19353 19417 19799 20025 20798 21055 21368 21467 21468 21638 22723
22785 23310 24734 24737 26918 31534 34745 35256 35617 36232
195: 999 1235 1847 1975 2624 2643 3350 3538 3715 4436 4884 5151 6029 6060 6469 6859 6909 6915 6948
7463 7585 7597 8596 9099 9185 9321 9463 10174 10183 10193 10272 10528 11438 11588 12451 13002
13009 13233 13367 13368 14051 14055 14373 14382 14396 14407 14415 14451 14898 15401 15438 15499
15509 15635 15690 15700 15720 15801 16326 16335 16336 16346 17467 17492 17660 17672 17688 17867
18160 18639 18809 19113 19442 19685 19686 19966 20785 20790 20956 21208 21209 21660 21914 22767
23231 23663 23869 24421 24678 26413 26665 26975 26984 27001 27022 27298 27830 28185 28187 28203
28207 28210 28229 28275 28320 28336 28337 28363 30532 30710 30711 30723 30756 30767 31118 31146
31271 31355 31356 31381 31403 31405 31567 31689 31770 31888 31929 31930 31937 31941 32126 32141
32146 34385 34445 36013
196: 1559 2831 3898 4905 4919 4941 5918 6260 6274 7403 7404 8435 8436 8553 9359 11333 12098 12155
12232 12420 15156 15157 18114 18215 19299 19393 20054 20251 20445 21299 21369 21780 21806 22588
23754 23818 24120 26739 29777 30971 33287 33579 33628 33856 34041 34217 34395 34396 35437
197: 1173 1660 1863 1885 1928 2289 2296 2312 2472 2494 2515 2649 2650 3070 3570 3878 3966 3967 4095
4118 4149 4172 4173 4198 4276 4335 4455 4486 4635 4707 5126 5127 5128 5167 5168 5749 6532 6881 6882
6883 7300 7350 7507 7508 7509 7512 7517 7518 7520 7523 7524 7525 7526 7527 7529 7530 7533 7534 7535
7537 7538 7539 7540 7541 7543 7546 7547 7548 7549 7550 7551 7552 7553 7554 7555 7556 7557 7558 7559
7562 7563 7565 7569 7570 7571 7572 7574 7575 7576 7780 7950 8392 8393 8394 8395 8396 8397 8440 8441
8442 8443 8444 8445 8446 8447 8448 8449 8450 8451 8452 8453 8454 8455 8456 8457 8458 8459 8460 8461
8462 8463 8464 8465 8466 8467 8468 8469 8470 8471 8472 8473 8474 8475 8476 8477 8478 8479 8480 8481
8701 8846 8884 9366 9667 9668 9669 9670 9671 9672 10512 11117 11118 11119 11120 11121 11122 11123
11124 11125 11393 11834 11835 11836 11837 11898 11900 12242 13216 13217 1 3218 13264 13274 13275
13279 13280 13287 13288 13289 13300 13303 13305 13308 13364 13524 13704 13739 13838 13904 13917
14015 14074 14115 14523 14524 14525 14526 14886 14887 15088 15184 15185 15186 15187 15308 15513
15514 15564 15565 15566 15567 15568 15569 15570 15602 15603 15604 15605 15606 15607 15608 15609
15610 15611 15613 15614 15615 15616 15622 15623 15624 15861 15862 15863 15864 15865 15866 15867
15868 15869 15870 15871 15872 15873 15874 16040 16175 16497 16498 16499 16500 16501 16502 16503
16504 16505 16506 16702 16897 16898 16899 16900 16901 16902 16903 16904 16905 16906 16907 16908
16909 16910 16911 16912 16913 16914 16915 16916 16917 16918 16919 16920 16921 16922 16923 16924
16925 16926 16927 16928 16929 16930 16931 16932 16933 16934 16935 16936 16937 16938 16939 16940
16941 16942 16943 16944 16945 16946 16947 16948 16949 16950 16951 16952 16953 16954 16955 16956
16957 16958 16959 16960 16961 16962 16963 16964 16965 16966 16967 16968 16969 16970 16971 16972
16973 16974 16975 16976 16977 16978 16979 16980 16981 16982 17269 17270 17271 17272 17273 17274
17275 17276 17277 17278 17279 17280 17281 17282 17283 17284 17285 17286 17287 17288 17289 17290
17291 17292 17293 17294 17295 17296 17297 17298 17299 17300 17301 17302 17303 17304 17305 17306
17307 17308 17309 17310 17311 17312 17313 17314 17315 17316 17317 17318 17319 17320 17321 17322
17323 17324 17325 17326 17327 17328 17329 17393 17617 17618 17619 17620 17621 17622 17623 17624
17625 17626 17627 17629 17630 17631 17632 17633 17634 17635 17636 17819 17827 18028 18029 18030
18031 18032 18033 18034 18035 18036 18037 18038 18039 18040 18041 18042 18043 18044 18045 18046
18047 18048 18049 18050 18051 18052 18053 18054 18055 18056 18057 18058 18059 18060 18061 18062
18063 18064 18065 18066 18067 18068 18069 18597 19181 19231 19302 19483 19528 19529 19530 19531
19532 19533 19534 19535 19536 19537 19538 19539 19540 19541 19542 19543 19544 19545 19546 19547
19548 19549 19550 19551 19552 19553 19554 19555 19556 19557 19558 19559 19560 19561 19562 19563
19564 19565 19566 19567 19568 19569 19570 19571 19572 19573 19847 19985 20000 20102 20537 20545
20546 20547 21056 21318 21347 21359 21844 21845 21846 21847 21913 22615 22716 22721 22982 22983
22984 23221 23222 23223 23224 23225 23226 23670 23882 24068 24169 25048 25069 25070 25071 25072
25073 25074 25075 25076 25077 25652 25699 25700 25868 25882 25883 25884 25901 25902 25903 25904
25916 25917 25918 26072 26092 26093 26094 26095 26096 26097 26098 26099 26100 26101 26102 26124
26125 26126 26127 26128 26129 26130 26131 26132 26133 26134 26135 26136 26137 26138 26139 26140
26141 26142 26143 26144 26145 26146 26147 26148 26149 26150 26151 26152 26153 26154 26155 26156
26157 26158 26159 26160 26161 26162 26163 26164 26165 26166 26167 26168 26169 26170 26171 26172
26173 26174 26175 26176 26177 26178 26179 26180 26181 26182 26183 26316 26317 26318 26319 26320
26321 26322 26382 26412 26448 26449 26450 26451 26452 26453 26454 26455 26456 26457 26458 26459
26460 26461 26462 26463 26464 26465 26466 26467 26468 26469 26470 26471 26472 26473 26474 26475
26476 26477 26478 26479 27457 27458 27459 28656 28657 28658 28659 28660 28661 28662 28663 28664
28665 28666 28667 28668 28669 28670 28671 28672 28673 28674 28700 28701 28702 28703 28704 28705
29039 29040 29041 29042 29043 29044 29045 29046 29047 29048 29049 29050 29051 29052 29053 29054
29055 29056 29057 29058 29059 29060 29061 29062 29063 29064 29065 29067 29068 29069 29070 29071
29072 29073 29074 29075 29076 29077 29078 29079 29080 29081 29684 30503 30504 30533 30595 30596
30597 31027 31213 31218 31322 31323 31324 31325 31326 31327 31328 31329 31330 31331 31332 31333
31334 31335 31336 31437 31564 31604 31607 31608 31609 31610 31611 31612 31613 31614 31615 31616
31617 31618 31619 31620 31621 31622 31623 31624 31625 31626 31627 31628 31629 31630 31631 31632
31633 31634 31635 31636 31637 31638 31639 31640 31641 31642 31643 31644 31645 31646 31647 31648
31649 31650 32575 32802 32852 33030 33268 33269 33270 33271 33272 33273 33274 33710 33711 33712
33713 33714 33715 33716 33717 33718 33719 33720 33721 33722 33723 33724 33725 33726 33727 33728
33729 33730 33731 33732 33733 33734 33735 33736 33737 33738 33739 33740 33741 33742 33743 33744
33745 33746 33747 33748 33749 33750 33751 33752 33753 33754 33755 33756 33757 33926 34114 34465
34749 35305 35306 35307 35308 35309 35310 35311 35312 35313 35314 35315 35316 35317 35318 35319
35320 35321 35322 35323 35324 35325 35326 35327 35328 35329 35330 35331 35332 35333 35334 35335
35336 35337 35338 35339 35340 35341 35342 35343 35344 35345 35346 35347 35348 35349 35350 35351
35352 35353 35354 35355 35356 35357 35358 35538 35539 35540
198: 504 1173 1660 1863 1885 1928 2289 2296 2312 2472 2494 2515 2649 2650 3070 3570 3878 3966 3967
4095 4118 4149 4172 4173 4198 4276 4335 4455 4486 4635 4707 5126 5127 5128 5167 5168 5749 6532 6881
6882 6883 7300 7350 7507 7508 7509 7512 7517 7518 7520 7523 7524 7525 7526 7527 7529 7530 7533 7534
7535 7537 7538 7539 7540 7541 7543 7546 7547 7548 7549 7550 7551 7552 7553 7554 7555 7556 7557 7558
7559 7562 7563 7565 7569 7570 7571 7572 7574 7575 7576 7780 7950 8392 8393 8394 8395 8396 8397 8440
8441 8442 8443 8444 8445 8446 8447 8448 8449 8450 8451 8452 8453 8454 8455 8456 8457 8458 8459 8460
8461 8462 8463 8464 8465 8466 8467 8468 8469 8470 8471 8472 8473 8474 8475 8476 8477 8478 8479 8480
8481 8701 8846 8884 9366 9667 9668 9669 9670 9671 9672 10512 11117 11118 11119 11120 11121 11122
11123 11124 11125 11393 11834 11835 11836 11837 11898 11900 12242 13216 13217 13218 13264 13274
13275 13279 13280 13287 13288 13289 13300 13303 13305 13308 13364 13524 13704 13739 13838 13904
13917 14015 14074 14115 14523 14524 14525 14526 14886 14887 15088 15184 15185 15186 15187 15308
15513 15514 15564 15565 15566 15567 15568 15569 15570 15602 15603 15604 15605 15606 15607 15608
15609 15610 15611 15613 15614 15615 15616 15622 15623 15624 15861 15862 15863 15864 15865 15866
15867 15868 15869 15870 15871 15872 15873 15874 16040 16175 16497 16498 16499 16500 16501 16502
16503 16504 16505 16506 16702 16897 16898 16899 16900 16901 16902 16903 16904 16905 16906 16907
16908 16909 16910 16911 16912 16913 16914 16915 16916 16917 16918 16919 16920 16921 16922 16923
16924 16925 16926 16927 16928 16929 16930 16931 16932 16933 16934 16935 16936 16937 16938 16939
16940 16941 16942 16943 16944 16945 16946 16947 16948 16949 16950 16951 16952 16953 16954 16955
16956 16957 16958 16959 16960 16961 16962 16963 16964 16965 16966 16967 16968 16969 16970 16971
16972 16973 16974 16975 16976 16977 16978 16979 16980 16981 16982 17269 17270 17271 17272 17273
17274 17275 17276 17277 17278 17279 17280 17281 17282 17283 17284 17285 17286 17287 17288 17289
17290 17291 17292 17293 17294 17295 17296 17297 17298 17299 17300 17301 17302 17303 17304 17305
17306 17307 17308 17309 17310 17311 17312 17313 17314 17315 17316 17317 17318 17319 17320 17321
17322 17323 17324 17325 17326 17327 17328 17329 17393 17617 17618 17619 17620 17621 17622 17623
17624 17625 17626 17627 17629 17630 17631 17632 17633 17634 17635 17636 17819 17827 18028 18029
18030 18031 18032 18033 18034 18035 18036 18037 18038 18039 18040 18041 18042 18043 18044 18045
18046 18047 18048 18049 18050 18051 18052 18053 18054 18055 18056 18057 18058 18059 18060 18061
18062 18063 18064 18065 18066 18067 18068 18069 18597 19181 19231 19302 19483 19528 19529 19530
19531 19532 19533 19534 19535 19536 19537 19538 19539 19540 19541 19542 19543 19544 19545 19546
19547 19548 19549 19550 19551 19552 19553 19554 19555 19556 19557 19558 19559 19560 19561 19562
19563 19564 19565 19566 19567 19568 19569 19570 19571 19572 19573 19847 19985 20000 20102 20537
20545 20546 20547 21056 21318 21347 21359 21844 21845 21846 21847 21913 22615 22716 22721 22982
22983 22984 23221 23222 23223 23224 23225 23226 23670 23882 24068 24169 25048 25069 25070 25071
25072 25073 25074 25075 25076 25077 25652 25699 25700 25868 25882 25883 25884 25901 25902 25903
25904 25916 25917 25918 26072 26092 26093 26094 26095 26096 26097 26098 26099 26100 26101 26102
26124 26125 26126 26127 26128 26129 26130 26131 26132 26133 26134 26135 26136 26137 26138 26139
26140 26141 26142 26143 26144 26145 26146 26147 26148 26149 26150 26151 26152 26153 26154 26155
26156 26157 26158 26159 26160 26161 26162 26163 26164 26165 26166 26167 26168 26169 26170 26171
26172 26173 26174 26175 26176 26177 26178 26179 26180 26181 26182 26183 26316 26317 26318 26319
26320 26321 26322 26382 26412 26448 26449 26450 26451 26452 26453 26454 26455 26456 26457 26458
26459 26460 26461 26462 26463 26464 26465 26466 26467 26468 26469 26470 26471 26472 26473 26474
26475 26476 26477 26478 26479 27457 27458 27459 28656 28657 28658 28659 28660 28661 28662 28663
28664 28665 28666 28667 28668 28669 28670 28671 28672 28673 28674 28700 28701 28702 28703 28704
28705 29039 29040 29041 29042 29043 29044 29045 29046 29047 29048 29049 29050 29051 29052 29053
29054 29055 29056 29057 29058 29059 29060 29061 29062 29063 29064 29065 29067 29068 29069 29070
29071 29072 29073 29074 29075 29076 29077 29078 29079 29080 29081 29684 30503 30504 30533 30595
30596 30597 31027 31213 31218 31322 31323 31324 31325 31326 31327 31328 31329 31330 31331 31332
31333 31334 31335 31336 31437 31564 31604 31607 31608 31609 31610 31611 31612 31613 31614 31615
31616 31617 31618 31619 31620 31621 31622 31623 31624 31625 31626 31627 31628 31629 31630 31631
31632 31633 31634 31635 31636 31637 31638 31639 31640 31641 31642 31643 31644 31645 31646 31647
31648 31649 31650 32575 32802 32852 33030 33268 33269 33270 33271 33272 33273 33274 33710 33711
33712 33713 33714 33715 33716 33717 33718 33719 33720 33721 33722 33723 33724 33725 33726 33727
33728 33729 33730 33731 33732 33733 33734 33735 33736 33737 33738 33739 33740 33741 33742 33743
33744 33745 33746 33747 33748 33749 33750 33751 33752 33753 33754 33755 33756 33757 33926 34114
34465 34749 35305 35306 35307 35308 35309 35310 35311 35312 35313 35314 35315 35316 35317 35318
35319 35320 35321 35322 35323 35324 35325 35326 35327 35328 35329 35330 35331 35332 35333 35334
35335 35336 35337 35338 35339 35340 35341 35342 35343 35344 35345 35346 35347 35348 35349 35350
35351 35352 35353 35354 35355 35356 35357 35358 35538 35539 35540
199: 504 1173 1660 1863 1885 1928 2289 2296 2312 2472 2494 2515 2649 2650 3070 3570 3878 3966 3967
4095 4118 4149 4172 4173 4198 4276 4335 4455 4486 4635 4707 5126 5127 5128 5167 5168 5749 6532 6881
6882 6883 7300 7350 7507 7508 7509 7512 7517 7518 7520 7523 7524 7525 7526 7527 7529 7530 7533 7534
7535 7537 7538 7539 7540 7541 7543 7546 7547 7548 7549 7550 7551 7552 7553 7554 7555 7556 7557 7558
7559 7562 7563 7565 7569 7570 7571 7572 7574 7575 7576 7780 7950 8392 8393 8394 8395 8396 8397 8440
8441 8442 8443 8444 8445 8446 8447 8448 8449 8450 8451 8452 8453 8454 8455 8456 8457 8458 8459 8460
8461 8462 8463 8464 8465 8466 8467 8468 8469 8470 8471 8472 8473 8474 8475 8476 8477 8478 8479 8480
8481 8701 8846 8884 9366 9667 9668 9669 9670 9671 9672 10512 11117 11118 11119 11120 11121 11122
11123 11124 11125 11393 11834 11835 11836 11837 11898 11900 12242 13216 13217 13218 13264 13274
13275 13279 13280 13287 13288 13289 13300 13303 13305 13308 13364 13524 13704 13739 13838 13904
13917 14015 14074 14115 14523 14524 14525 14526 14886 14887 15088 15184 15185 15186 15187 15308
15513 15514 15564 15565 15566 15567 15568 15569 15570 15602 15603 15604 15605 15606 15607 15608
15609 15610 15611 15613 15614 15615 15616 15622 15623 15624 15861 15862 15863 15864 15865 15866
15867 15868 15869 15870 15871 15872 15873 15874 16040 16175 16497 16498 16499 16500 16501 16502
16503 16504 16505 16506 16702 16897 16898 16899 16900 16901 16902 16903 16904 16905 16906 16907
16908 16909 16910 16911 16912 16913 16914 16915 16916 16917 16918 16919 16920 16921 16922 16923
16924 16925 16926 16927 16928 16929 16930 16931 16932 16933 16934 16935 16936 16937 16938 16939
16940 16941 16942 16943 16944 16945 16946 16947 16948 16949 16950 16951 16952 16953 16954 16955
16956 16957 16958 16959 16960 16961 16962 16963 16964 16965 16966 16967 16968 16969 16970 16971
16972 16973 16974 16975 16976 16977 16978 16979 16980 16981 16982 17269 17270 17271 17272 17273
17274 17275 17276 17277 17278 17279 17280 17281 17282 17283 17284 17285 17286 17287 17288 17289
17290 17291 17292 17293 17294 17295 17296 17297 17298 17299 17300 17301 17302 17303 17304 17305
17306 17307 17308 17309 17310 17311 17312 17313 17314 17315 17316 17317 17318 17319 17320 17321
17322 17323 17324 17325 17326 17327 17328 17329 17393 17617 17618 17619 17620 17621 17622 17623
17624 17625 17626 17627 17629 17630 17631 17632 17633 17634 17635 17636 17819 17827 18028 18029
18030 18031 18032 18033 18034 18035 18036 18037 18038 18039 18040 18041 18042 18043 18044 18045
18046 18047 18048 18049 18050 18051 18052 18053 18054 18055 18056 18057 18058 18059 18060 18061
18062 18063 18064 18065 18066 18067 18068 18069 18597 19181 19231 19302 19483 19528 19529 19530
19531 19532 19533 19534 19535 19536 19537 19538 19539 19540 19541 19542 19543 19544 19545 19546
19547 19548 19549 19550 19551 19552 19553 19554 19555 19556 19557 19558 19559 19560 19561 19562
19563 19564 19565 19566 19567 19568 19569 19570 19571 19572 19573 19847 19985 20000 20102 20537
20545 20546 20547 21056 21318 21347 21359 21844 21845 21846 21847 21913 22615 22716 22721 22982
22983 22984 23221 23222 23223 23224 23225 23226 23670 23882 24068 24169 25048 25069 25070 25071
25072 25073 25074 25075 25076 25077 25652 25699 25700 25868 25882 25883 25884 25901 25902 25903
25904 25916 25917 25918 26072 26092 26093 26094 26095 26096 26097 26098 26099 26100 26101 26102
26124 26125 26126 26127 26128 26129 26130 26131 26132 26133 26134 26135 26136 26137 26138 26139
26140 26141 26142 26143 26144 26145 26146 26147 26148 26149 26150 26151 26152 26153 26154 26155
26156 26157 26158 26159 26160 26161 26162 26163 26164 26165 26166 26167 26168 26169 26170 26171
26172 26173 26174 26175 26176 26177 26178 26179 26180 26181 26182 26183 26316 26317 26318 26319
26320 26321 26322 26382 26412 26448 26449 26450 26451 26452 26453 26454 26455 26456 26457 26458
26459 26460 26461 26462 26463 26464 26465 26466 26467 26468 26469 26470 26471 26472 26473 26474
26475 26476 26477 26478 26479 27457 27458 27459 28656 28657 28658 28659 28660 28661 28662 28663
28664 28665 28666 28667 28668 28669 28670 28671 28672 28673 28674 28700 28701 28702 28703 28704
28705 29039 29040 29041 29042 29043 29044 29045 29046 29047 29048 29049 29050 29051 29052 29053
29054 29055 29056 29057 29058 29059 29060 29061 29062 29063 29064 29065 29067 29068 29069 29070
29071 29072 29073 29074 29075 29076 29077 29078 29079 29080 29081 29684 30503 30504 30533 30595
30596 30597 31027 31213 31218 31322 31323 31324 31325 31326 31327 31328 31329 31330 31331 31332
31333 31334 31335 31336 31437 31564 31604 31607 31608 31609 31610 31611 31612 31613 31614 31615
31616 31617 31618 31619 31620 31621 31622 31623 31624 31625 31626 31627 31628 31629 31630 31631
31632 31633 31634 31635 31636 31637 31638 31639 31640 31641 31642 31643 31644 31645 31646 31647
31648 31649 31650 32575 32802 32852 33030 33268 33269 33270 33271 33272 33273 33274 33710 33711
33712 33713 33714 33715 33716 33717 33718 33719 33720 33721 33722 33723 33724 33725 33726 33727
33728 33729 33730 33731 33732 33733 33734 33735 33736 33737 33738 33739 33740 33741 33742 33743
33744 33745 33746 33747 33748 33749 33750 33751 33752 33753 33754 33755 33756 33757 33926 34114
34465 34749 35305 35306 35307 35308 35309 35310 35311 35312 35313 35314 35315 35316 35317 35318
35319 35320 35321 35322 35323 35324 35325 35326 35327 35328 35329 35330 35331 35332 35333 35334
35335 35336 35337 35338 35339 35340 35341 35342 35343 35344 35345 35346 35347 35348 35349 35350
35351 35352 35353 35354 35355 35356 35357 35358 35538 35539 35540
200: 998 1007 1143 1433 1740 2067 3449 4211 5733 6872 9416 9480 13985 14018 16446 16777 17805 18966
19271 19375 19434 19777 20019 20063 20955 21488 22593 22692 22732 22969 23003 23884 34744
201: 998 1007 1143 1433 1740 2067 3449 4211 5733 6872 9416 9480 13985 14018 16446 16777 17805 18966
19271 19375 19434 19777 20019 20063 20955 21488 22593 22692 22732 22969 23003 23884 34744
202: 664 786 941 968 1106 1147 1161 1245 1285 1351 1368 1401 1474 1484 1506 1529 1604 1609 1612 1714
1728 1776 1801 1816 1871 1931 2027 2045 2062 2232 2526 2537 2583 2751 3405 3426 3428 3432 3534 3662
3710 3717 3720 3742 3761 3811 3939 3949 3950 4040 4085 4087 4110 4226 4242 4318 4334 4347 4502 4508
4517 4528 4550 4553 4594 4628 4639 4642 4796 4798 4799 4821 4985 4992 5053 5059 5074 5365 5417 5471
5474 5520 5522 5566 5702 5929 5955 61 18 6122 6125 6127 6133 6143 6178 6221 6258 6304 6318 6319 6376
6417 6509 6609 6618 6624 6625 6629 6772 6980 7011 7015 7016 7020 7055 7059 7063 7066 7067 7078 7101
7102 7107 7185 7207 7244 7259 7268 7342 7464 7884 8002 8025 8068 8069 8111 8210 8213 8272 8317 8327
8335 8341 8529 8534 8537 8639 8665 8740 8744 8745 8746 8780 8781 8793 8802 8864 8909 8925 9229 9236
9243 9260 9290 9389 9418 9474 9557 9580 9608 9621 9743 9748 9773 9774 9816 9821 9824 9848 9850 9893
9894 9906 9960 9963 9977 10009 10010 10071 10072 10240 10294 10328 10330 10331 10438 10461 10491
10504 10572 10581 10585 10611 10619 10672 10674 10682 10692 10696 10731 10764 10827 10902 10970
11013 11017 11076 11134 11152 11257 11303 11403 11491 11497 11512 11563 11630 11691 11739 11773
11780 11821 11866 11869 11943 11961 12013 12045 12057 12085 12247 12262 12313 12321 12350 12418
12514 12531 12537 12538 12539 12542 12606 12613 12622 12711 12727 12753 12754 12839 13012 13062
13127 13138 13139 13146 13148 13149 13153 13318 13445 13484 13487 13496 13499 13564 13627 13745
13785 13800 13822 13865 13927 14249 14353 14354 14355 14356 14357 14552 14642 14775 14804 14805
14823 14827 14884 14954 14964 14968 14980 15017 15030 15032 15045 15134 15189 15254 15268 15307
15483 15858 15912 15943 15949 15952 15967 16072 16077 16123 16167 16181 16201 16417 16466 16568
16637 16653 16661 16709 16710 16742 16800 16828 16860 16866 16876 16877 16878 17025 17053 17055
17060 17061 17062 17065 17078 17171 17174 17189 17250 17338 17580 17717 17719 17729 17782 18081
18237 18283 18284 18299 18408 18410 18434 18446 18464 18468 18470 18503 18518 18521 18546 18547
18594 18767 18802 18849 18860 18910 18942 18943 18996 19075 19110 19211 19621 19635 19660 19758
19808 19842 20088 20192 20231 20242 20262 20269 20282 20293 20305 20357 20424 20565 20575 20601
20608 20612 20716 20747 20751 20760 20761 20799 20804 20807 20811 20857 20865 20873 20919 20958
21023 21025 21105 21115 21132 21146 21158 21221 21474 21534 21711 21736 21739 21822 21823 21824
21825 21826 21871 21906 21966 22046 22233 22243 22323 22325 22361 22385 22397 22427 22492 22505
22533 22626 22885 22970 23104 23116 23124 23143 23164 23172 23173 23174 23205 23207 23274 23275
23359 23364 23369 23380 23396 23414 23469 23477 23493 23494 23504 23516 23522 23524 23534 23550
23551 23561 23590 23594 23602 23614 23623 23664 23733 23740 23804 23812 23830 23832 23940 23953
23965 23966 23988 24002 24012 24034 24049 24124 24134 24138 24141 24151 24328 24329 24358 24359
24362 24367 24373 24374 24442 24451 24464 24470 24494 24501 24519 24566 24571 24613 24623 24657
24743 24744 24745 24746 24747 24748 24749 24750 24812 24943 24990 24994 25132 25160 25161 25174
25190 25198 25222 25223 25259 25530 25537 25871 25872 25873 25932 25933 25934 25935 25936 25937
25944 25984 25985 26209 26227 26231 26236 26265 26326 26327 26330 26350 26393 26394 26547 26784
26849 26859 26896 26944 27099 27111 27365 27370 27384 27397 27399 27405 27418 27718 27865 27888
27985 27987 28026 28039 28042 28094 28521 28525 28547 28554 28559 28595 28613 28614 28616 28632
28640 28689 28747 28751 28752 28846 28850 28865 28956 28990 28992 29110 29122 29125 29134 29142
29143 29215 29232 29265 29278 29418 29424 29450 29479 29482 29489 29490 29542 29550 29553 29562
29569 29586 29596 29601 29604 29774 29775 29794 29812 29885 29940 29983 29990 30027 30051 30066
30101 30158 30174 30210 30221 30224 30306 30334 30371 30398 30399 30612 30626 30634 30648 30668
30853 30859 30870 31000 31009 31020 31026 31169 31235 31337 31488 31497 31977 31998 32007 32042
32043 32088 32173 32185 32200 32243 32244 32246 32251 32321 32322 32323 32324 32325 32327 32356
32432 32457 32465 32490 32562 32563 32580 32626 32627 32628 32632 32633 32704 32842 32860 32873
32880 33026 33032 33097 33104 33137 33138 33171 33172 33187 33193 33222 33223 33297 33304 33406
33413 33448 33461 33497 33560 33561 33574 33686 33698 33859 33863 33867 33874 34012 34075 34080
34098 34130 34219 34222 34289 34305 34306 34320 34336 34413 34427 34495 34503 34519 34531 34593
34637 34729 34735 34933 34951 35014 35015 35021 35038 35045 35049 35107 35122 35139 35143 35144
35148 35149 35169 35170 35174 35200 35203 35204 35207 35274 35471 35502 35503 35506 35522 35523
35524 35557 35591 35599 35601 35602 35632 35637 35670 35767 35771 35778 35785 35786 35868 35878
35891 35895 35904 36038 36071 36126 36142 36152 36163 36176 36186 36196 36198 36260 36262 36288
36290 36292 36397 36399 36428 36430
203: 861 1809 2718 2901 2956 3046 3528 3638 3828 4081 4346 4981 5724 5907 5998 6523 6898 7930 8035
8081 8562 8855 9072 9141 10264 11202 11230 12390 13328 13358 14433 15625 15680 15688 16319 16451
16474 16706 16707 17555 18173 18174 18612 19012 19498 20124 20148 20416 20653 21486 21527 22876
23657 23889 23892 23894 23895 23899 23916 24539 24676 25659 25660 26054 26116 26123 26921 26994
27144 27145 27146 27243 27479 28143 28240 28309 28383 28470 28766 29675 30511 30728 31082 31417
31424 31673 31949 31952 32147 32958 33027 35529 36352
204: 803 1311 1419 1550 2075 2424 3196 3213 3842 3871 4551 4598 4719 4808 4839 5215 5265 5338 5413
5466 5526 5571 5707 5965 6093 6155 6219 6285 6333 6526 6557 6632 6961 7004 7082 7124 7232 7461 7486
7703 7855 7862 8033 8171 8275 8353 8361 8545 8697 8751 9211 9292 9503 9555 9853 9874 9930 9997
10394 10402 10465 10638 10693 10730 10743 10765 10848 10930 10982 11032 11141 11559 11662 11847
11859 11946 12028 12041 12524 12688 12747 12768 12775 12801 12826 12879 12927 13028 13029 13067
13175 13217 13444 13580 13587 13643 13675 13890 14022 14100 14185 14574 14618 14687 14920 15051
15474 15931 15966 16206 16615 16804 16871 17196 17257 17336 17577 17714 18094 18128 18281 18285
18351 18445 18482 18539 18671 19014 19101 19471 19658 19696 19888 20188 20214 20283 20347 20593
20709 20805 20823 20995 21084 21219 21451 21670 21755 21998 22066 22071 22222 22282 22309 22398
22859 23095 23276 23278 23280 23281 23358 23385 23457 23596 23726 23771 23972 24022 24100 24299
24408 24510 24529 24814 24843 24871 24910 24952 24992 25117 25140 25379 25391 25532 25557 25580
25607 25626 25801 25840 25865 25866 25923 26086 26206 26349 26606 26724 26831 27019 27116 27300
27475 27489 27537 27632 27852 27876 28031 28076 28252 28419 28566 28608 28688 28748 28753 28987
29117 29162 29226 29268 29350 29353 29428 29663 29986 30036 30116 30152 30225 30282 30297 30301
30422 30466 30487 30665 30818 30833 30975 30979 31052 31204 31520 31694 31867 31988 31992 32116
32184 32217 32270 32376 32388 32466 32652 32871 33091 33203 33359 33383 33503 33515 33575 33619
33675 34049 34191 34192 34268 34327 34417 34509 34557 34562 34650 34701 34777 34797 34961 34988
35056 35069 35157 35215 35276 35391 35519 35588 35643 35697 35745 35846 35985 36069 36076 36082
36105 36161 36222 36255 36362
205: 791 900 1284 1649 1996 2294 3085 3753 5743 6391 7945 16036 17125 19123 19918 21361 22834 22897
23751 23752
206: 4579 4647 4718 4805 5073 5169 5333 5363 5405 5436 5443 5454 5519 5523 5534 5578 5674 5675 5978
6005 6035 6102 6261 6546 6578 6737 7032 7042 7061 7080 7104 7111 7119 7122 7140 7141 7142 7181 7451
7475 7639 8156 8215 8233 8234 8237 8238 8240 8250 8538 9504 9638 9746 9752 9754 9755 9756 9775 9838
9961 9962 9974 9998 10013 10100 10306 10347 10356 10420 10633 10661 10663 10718 10793 10871 10889
10890 10910 10947 10971 11156 11525 11602 11616 11620 11639 12244 12340 12349 12351 12354 12637
12794 12815 12825 12859 12955 12972 12978 13131 13135 13140 13144 13430 13467 13470 13517 13537
13612 13808 14010 14563 14609 14783 15096 15235 16080 16099 16100 16177 16591 16801 16812 16818
16819 16840 16857 17091 17202 17212 17226 17363 17364 17385 18084 18141 18186 18207 18208 18223
18224 18235 18343 18690 18847 18955 19472 19674 19675 19676 19677 19678 19679 19680 19681 19682
19683 19684 19746 20554 20619 20699 20726 20779 20852 20854 20859 20867 20893 20900 20903 21248
21860 21876 21896 21905 21907 22041 22049 22051 22052 22060 22070 22085 22215 22234 22256 22310
22314 22318 22354 22374 22399 22439 22480 23197 23244 23247 23342 23398 23406 23413 23431 23433
23448 23473 23610 23621 23649 23857 23883 23979 24008 24010 24013 24032 24170 24171 24172 24173
24174 24175 24176 24177 24178 24179 24180 24181 24182 24183 24184 24185 24186 24187 24188 24189
24190 24191 24192 24193 24194 24195 24196 24197 24198 24199 24200 24201 24202 24203 24204 24205
24206 24207 24208 24209 24210 24211 24212 24213 24214 24215 24216 24217 24218 24219 24220 24221
24222 24223 24224 24225 24226 24227 24228 24229 24230 24231 24232 24233 24234 24235 24236 24237
24238 24239 24240 24241 24242 24243 24244 24245 24246 24247 24248 24249 24250 24251 24252 24253
24254 24255 24256 24257 24258 24259 24260 24261 24262 24263 24264 24265 24266 24267 24268 24269
24270 24271 24272 24273 24274 24275 24276 24277 24278 24279 24280 24281 24282 24283 24284 24285
24286 24287 24288 24289 24290 24313 24432 24502 24506 24508 24593 24658 24661 24726 24730 24789
25145 25146 25240 25241 25392 25481 25482 25550 25577 25694 25852 25948 25959 25968 26533 26763
26842 26897 26899 26947 27093 27141 27284 27565 27575 27608 27643 27712 27729 27741 27762 27785
27924 27930 27937 27961 27976 28017 28018 28066 28088 28617 28620 28622 28629 28708 28723 28879
28948 28954 28966 28974 29100 29205 29301 29345 29354 29438 29501 29610 29616 29641 29642 29650
29700 29701 29863 29868 30008 30023 30171 30205 30323 30348 30361 30374 30537 30574 30995 30997
31008 31012 31036 31230 31442 31448 31527 31568 31601 31868 32064 32074 32085 32179 32182 32189
32267 32286 32299 32342 32485 32592 32779 32780 32781 33055 33084 33159 33214 33330 33419 33516
33658 33691 33701 33855 34133 34260 34296 34307 34333 34358 34403 34414 34515 34536 34539 34649
34987 35089 35120 35132 35146 35164 35175 35176 35224 35448 35449 35456 35474 35547 35553 35565
35569 35645 36042 36043 36067 36073 36182 36195 36365 36366 36379 36406
207: 4404 5859 5860 6839 6840 6841 6842 6843 6844 6845 6846 7371 9322 11985 11986 12969 18618 21634
25430 25431 25432 25433 25434 25435 25436 25437 25438 25439 25440 25441 25442 25443 25444 29866
36314
208: 4512 4991 5026 5593 6766 7594 8519 9844 10326 10358 10439 11510 11631 12017 12717 14301 14929
15887 16111 16491 16852 17068 17098 17205 17475 19652 21168 21256 21908 22407 22426 23084 23179
23259 24324 24356 25156 25220 25286 25516 25940 26252 27381 29213 30647 31504 32519 32670 32686
32947 33253 33327 33403 33422 34125 34144 34492 35795 36103 36155
209: 649 651 652 1208 1741 3649 3854 4415 4416 4417 7216 15075
210: 841 1011 1012 1587 1870 2197 2674 3144 4050 5693 11260 17773 19228 19855 22689 29389
211: 486 487 792 1194 1317 1407 1448 1848 1959 2239 2410 2466 2738 2972 3123 3197 3334 3399 3436
3441 3442 3467 3497 3906 4021 4283 4424 5112 5720 5763 6037 6189 6475 6873 7373 7890 7927 7966 7968
8027 8034 8054 8818 11412 12503 13227 16874 18580 18939 19122 19426 19464 19901 20365 20398 21440
21481 21482 21542 21558 22491 22625 22766 24704 26962 33065 35533
212: 1136 3162 3655 3778 5114 5735 7934 8877 9135 9190 9700 13667 14216 14661 15274 16496 17477
18585 18696 19307 20073 20420 21496 22814 24712 26796 31775 35992 35993 35994 35995 35996 36227
213: 852 1118 1565 1598 1958 2130 2131 2739 2798 2962 2991 3202 4055 4067 4444 4560 4609 4672 4678
4779 4814 4859 5040 5257 5300 5765 5949 5964 5993 6094 6134 6198 6820 6917 6918 6946 6984 7220 7265
7959 8047 8179 8421 8555 8585 8675 8718 9191 9256 9352 9378 9500 9596 9879 9913 9988 10011 10148
10477 10492 10620 11426 11495 11646 11805 11814 12010 12342 12487 12513 12609 12912 13485 13486
13569 13631 13916 14109 14434 14631 14897 15042 15065 15257 15351 15561 15926 16509 16590 16790
17032 17428 17940 18009 18433 18693 18822 18868 19107 19148 19480 19486 19647 19896 1999920314
20360 20543 20684 20916 21013 21079 21116 21865 22182 22372 22545 22546 22608 22761 23177 23412
23564 23571 23640 23983 24960 25166 25253 25302 25922 25989 26232 26270 26277 26278 26296 26507
26588 27235 27236 27237 27245 27246 27903 27978 28927 28941 29468 29860 29877 30038 30082 30211
30341 30516 30575 30622 30690 30698 30760 30951 30952 30953 30954 31574 31575 31686 31699 31765
31975 32242 32517 32642 32645 33062 33178 33400 33401 33408 33420 33473 33474 33795 33991 34279
34391 34573 34608 34630 34700 34751 35079 35258 35541 35787 35824 35836 35937 35941 35965 36350
36351
214: 867 1189 1335 1752 1905 1951 1961 2139 2156 2520 2923 3053 3268 6416 6440 6641 6642 6643 6827
13069 14212 14213 14836 15301 15302 15303 17645 17860 18969 19024 19357 19819 20076 22566 22910
22931 23327 26512 28469 30316 30968 30969 30970 31191 31428 32845 33794
215: 937 983 1036 1345 1360 1469 2126 2127 2128 2129 2256 2382 2414 2629 2813 2982 3194 3328 3344
3463 3483 3494 3552 3616 3641 3740 3845 3919 4119 4180 4216 4220 4229 4264 4314 4325 5682 5714 5783
5810 6063 7879 8023 8438 9026 9027 9028 9029 9030 9031 9032 9040 9689 10238 13984 14001 14063
14064 14099 14119 14137 16144 17390 17791 18638 18796 18800 18930 19141 19437 19821 20429 20535
21406 21452 21620 21659 21919 21920 22682 29757 31067 31145 32956 33927 33931 33932 33933
216: 1112 2306 4982 5756 6025 6482 6865 6904 7153 7608 7949 8578 10178 11256 11916 12380 12982
13003 13701 14105 14422 15416 15563 15757 15792 16315 17526 17550 18997 19109 19517 19518 20087
20678 21349 22228 22628 23715 24858 26273 26608 26998 27208 28181 28232 28303 28451 30759 31349
31708 31922 32144 33239 36442
217: 641 657 708 1283 1326 1696 1859 1955 2001 4274 4771 5188 5427 5902 6404 7334 7351 7673 8077
8363 8482 8483 8878 9348 9494 9654 10138 10155 11176 11276 11317 12988 13209 13324 13325 14087
14899 15111 15312 15318 15426 15436 15498 16033 16280 16473 16698 17330 17469 17644 18267 19083
19289 19760 20013 20056 20462 20920 20927 20928 20952 21087 21223 21224 21226 21227 21310 21836
22780 23677 23689 23809 23810 23861 23910 24161 24166 24538 24541 24739 24919 24920 24921 24923
24924 24925 24926 24927 24928 24929 24930 24931 24932 24933 24937 24961 24971 25084 25085 25314
25315 25316 25317 25318 25319 25321 25322 25323 25484 25653 25673 25674 25675 25912 26062 26063
26064 26065 26066 26667 26900 26904 27046 27586 27587 27588 27589 27590 27591 27592 27593 27594
27595 27596 28373 28773 29011 29012 29013 29846 29850 29851 29852 30584 30825 31085 31086 31087
31089 31090 31175 31176 31177 31196 31269 31321 31429 31431 31561 31576 31598 31828 31831 32089
32601 33018 33966 34207 34208 34462 34466 34640 34641 34642 34648 34845 34923 35177 35178 35248
35249 35293 35300 35605 35607 36303 36304 36305 36419
218: 813 952 1223 1228 1990 2111 2301 2634 2635 3060 3835 3975 4308 4428 4485 11914 11915 13807
13938 15278 15954 16418 17821 18651 18972 19056 20112 21330 21379 22622 27484 27601 30811 30812
31968
219: 1218 2342 2368 2467 2527 2743 3032 3603 3849 4015 4049 5861 6857 10518 10519 10520 10521 10522
10523 10524 10525 16141 17746 17878 19046 19383 20377 20383 20405 21398 21515 25469
220: 629 823 1082 1083 1084 1231 1607 4418 4419 4695 5421 6504 6639 6640 6891 6892 7581 7582 7583
7584 7789 8200 8201 8202 8203 8204 8205 8206 8207 8208 9023 9376 9395 9397 10124 10226 10881 11354
11355 11356 11409 11410 11439 11904 12777 12778 12779 12780 12781 12782 12783 12784 12785 12786
12787 12962 12963 12964 12965 12966 12967 12968 13121 13122 13663 15953 16495 16776 16873 17668
17699 18325 18544 18664 18665 18666 19037 19038 19039 19040 19041 19042 19512 19513 19514 19515
19516 19687 19688 19689 19690 19762 19801 19802 19803 19804 19805 19806 20023 20024 20334 20642
20783 20784 20931 21944 21945 21946 21947 21948 21949 21950 21951 21952 21953 22611 22884 22911
23912 23914 24691 24804 25428 26083 26120 26443 26602 26775 26776 26777 27147 27669 31170 31171
31172 31173 31174 31566 33042 33043 33044 33045 33046 33047 33048 33049 33264 33265 33266 33939
33940 33941 33944 33948 33949 33950 33951 33952 33953 33954 33955 34366 34367 34368 35290 35292
35297 35359 35360 35361 36336 36337
221: 853 1096 11376 19989 22827 22828
222: 1202 2046 2365 3221 6188 8026 9375 9476 11325 11326 19447 19968 19969 21529 22883 22977 25992
36331
223: 605 606 1622 1687 1820 2031 2416 2550 2657 2755 3038 3072 3117 3163 3195 3228 3554 3682 3741
3783 3884 3885 4117 4163 4196 4345 4392 4448 4465 4705 4717 4739 4754 5120 5187 5189 5483 5852 6107
6108 6404 6493 6983 8484 8485 8487 8498 9120 9123 9180 9181 10532 10592 10924 10925 11219 12705
12706 12707 12987 13322 13323 14025 14655 15436 15494 15510 15553 15793 15794 16008 16280 16289
16698 16791 17692 17780 18271 18572 18792 18947 19027 19360 20036 20318 20450 21087 21222 21223
21224 21226 21227 21340 21446 21788 21915 21916 21917 22469 23063 23674 23675 23690 23910 24161
24321 24536 24537 24538 24541 24689 24690 24727 24757 24803 24919 24922 24924 24930 24935 24936
25310 25314 25315 25320 25324 25325 25653 25938 26063 26064 26065 26440 26487 26491 26504 26529
26530 26655 26656 26667 26900 26902 26904 26905 26906 26907 26908 26913 26919 27046 27823 28280
28374 28562 29847 29849 30097 30629 30825 31088 31091 31122 31123 31157 31188 31192 31315 31316
31430 31431 31432 31533 31598 31753 31754 31829 31830 31978 31980 31981 31982 31983 32596 32597
32600 32826 33018 33822 33823 33966 34923 35248 35250 35605 35607 36301 36302 36315 36316 36317
36318
224: 1701 1703 1756 1757 2123 2177 2208 2233 2234 2356 2392 2538 2666 2712 2740 2750 2844 2906 3033
3301 3327 3367 3504 3553 3565 3588 3727 3728 3733 3791 3844 3914 3980 3999 4381 5194 5656 5657 5658
5659 5660 5661 5662 5669 5670 5683 5798 5807 6480 6971 7149 7327 7491 7492 7880 7903 7904 8024 8075
8076 8976 8977 9063 9076 9647 9739 9740 9896 10048 10049 10050 11478 11482 11964 11965 12243 12246
12596 12597 12598 13070 13200 13201 13202 13203 13204 13205 13206 13207 13225 13226 13252 13355
13413 13803 13870 13937 14080 14163 14164 14165 14210 14235 14441 14465 14667 14894 15182 15506
15507 15508 15773 15774 15775 15776 15777 15778 15779 15780 16231 16290 16329 16512 16767 16768
16769 16770 16771 16772 17751 17761 17772 17848 18178 18277 18554 18695 19164 19166 19519 19520
19521 19522 19523 19524 19525 19526 19601 20142 20176 20177 20178 20179 20180 20181 20182 20335
20336 21393 21421 21431 21454 21485 21522 21528 21597 21635 21657 21800 21942 22641 22812 23212
23661 23745 23746 23747 25676 25677 25678 25679 25970 25996 26018 26446 26525 27834 28325 28369
28392 29853 31197 31265 32157 32310 32320 33956 33967 33973 34001 34110 34458 34459 34460 34461
34927 35364
225: 801 972 1504 1700 1825 1864 2124 2305 2422 3176 3321 3614 3677 3678 3789 4890 5181 5182 5183
5873 5874 5875 5876 5877 5878 5879 5880 5881 5882 5883 5884 5885 5886 5887 5888 5889 5890 5891 5892
5893 5894 5895 5896 5897 5898 7991 10424 11266 11350 11351 13939 17505 17683 18790 18963 19136
19198 19428 19866 19911 19912 20797 20798 21055 21366 22727 22882 23310 23799 23800 23801 23802
24065 24734 26918 28376 31534 35617 36232 36312 36313
226: 1253 1689 2546 3133 3764 4257 4316 4357 4413 4514 4541 4621 4640 4679 4818 4844 4924 4925 4927
4932 4950 4969 5082 5133 5219 5296 5340 5361 5411 5453 5480 5541 5587 5931 5973 6066 6154 6220 6257
6262 6312 6332 6346 6354 6576 6584 6622 6738 6974 7035 7050 7074 7129 7180 7288 7289 7415 7422 7495
7496 7589 7590 7694 7883 7886 7887 7971 7981 8044 8123 8131 8141 8256 8265 8318 8328 8339 8351 8512
8524 8549 8589 8686 8759 8787 8868 8872 8993 8994 9060 9061 9075 9081 9103 9168 9169 9239 9324 9344
9396 9517 9612 9761 9764 9765 9768 9782 9812 10014 10076 10091 10116 10149 10169 10184 10318 10355
10447 10516 10517 10584 10656 10703 10732 10855 10856 10858 10862 10909 10974 10990 11081 11315
11414 11496 11508 11538 11564 11629 11718 11790 11798 11827 11868 11959 12022 12029 12046 12091
12312 12335 12431 12435 12516 12553 12611 12612 12619 12645 12697 12719 12758 12846 12917 12979
12980 13040 13132 13193 13194 13454 13515 13551 13670 13678 13683 13697 13846 13855 13886 13900
14050 14397 14421 14456 14531 14576 14597 14678 14793 14798 14799 14947 15025 15066 15120 15122
15137 15138 15296 15297 15381 15543 15544 15666 15769 15803 15804 15881 15883 15964 15980 16084
16176 16202 16316 16340 16559 16628 16672 16680 16689 16690 16693 16727 16755 16864 17030 17041
17094 17177 17217 17238 17350 17376 17413 17514 17575 17593 17770 17854 18104 18242 18244 18319
18335 18370 18405 18447 18481 18810 18902 18903 19313 19314 19315 19436 19609 19710 20009 20010
20057 20266 20271 20310 20442 20548 20566 20624 20639 20703 20720 20829 20853 20864 20870 20914
21030 21078 21099 21108 21113 21255 21346 21690 21691 21740 21752 21816 21990 22058 22063 22157
22272 22313 22339 22406 22417 22510 22538 22636 22877 23103 23132 23204 23243 23258 23326 23328
23329 23391 23428 23468 23486 23519 23612 23636 23739 23762 23783 23848 23853 23951 23976 24038
24102 24136 24148 24164 24343 24385 24398 24430 24458 24465 24500 24557 24560 24572 24591 24624
24656 24683 24738 24907 24948 25002 25049 25238 25291 25311 25359 25495 25618 25644 25705 25772
25825 25855 25921 25925 26207 26357 26408 26691 26709 26719 26769 26839 26866 26870 26982 27021
27094 27120 27121 27189 27215 27286 27352 27447 27449 27450 27515 27534 27540 27543 27572 27609
27612 27634 27640 27646 27714 27716 27743 27746 27870 27894 27900 27913 27917 27944 28046 28095
28139 28147 28230 28241 28304 28343 28428 28526 28527 28649 28690 28793 28813 28814 28825 28840
28849 28857 28863 28899 28924 28929 28951 28985 29102 29129 29163 29186 29240 29332 29343 29356
29513 29558 29591 29615 29648 29676 29677 29726 29832 29886 29912 29939 29943 30012 30058 30090
30121 30199 30217 30250 30251 30285 30324 30339 30340 30343 30373 30381 30432 30482 30598 30606
30618 30700 30713 30724 30785 30863 30891 30915 30925 30932 31016 31389 31446 31454 31475 31494
31563 31594 31595 31668 31669 31670 31701 31703 31715 31834 31845 31901 31909 31972 32005 32079
32111 32152 32190 32219 32296 32331 32366 32391 32417 32460 32507 32515 32698 32700 32732 32796
32899 32982 33066 33086 33128 33174 33342 33488 33595 33612 33638 33839 33854 33865 33972 34092
34102 34198 34209 34246 34248 34300 34310 34507 34607 34687 34736 34787 34860 34915 34966 34995
35047 35088 35150 35166 35183 35278 35299 35401 35425 35487 35581 35582 35658 35667 35749 35777
35862 35922 35956 36024 36035 36096 36098 36178 36184 36219 36259 36276 36285 36401 36434 36435
36436 36437 36438 36439 36440 36441
227: 591 922 926 981 1050 1333 1352 1483 1554 1571 1617 1620 1621 1623 1629 1640 1749 1807 2002 2011
2095 2190 2246 2284 2330 2524 2545 2569 2600 2791 2833 2863 2864 2966 2988 3113 3139 3146 3263 3296
3300 3305 3527 3576 3643 3644 3645 3708 3725 3831 3848 3893 3995 4030 4102 4108 4111 4218 4219 4324
4343 4359 4441 4442 4482 4589 4605 4804 4812 4836 4878 4996 5064 5129 5311 5359 5402 5423 5424 5432
5467 5524 5637 5746 5942 6009 6023 6087 6245 6313 6347 6351 6364 6487 6574 6594 6600 6601 6630 6657
6707 6715 6746 6795 6841 6937 6993 7033 7077 7281 7282 7602 8094 8258 8311 8332 8403 8643 8779 8784
8785 8801 8812 8813 8814 8893 9010 9011 9157 9233 9275 9278 9454 9578 9599 9616 9698 9705 9777 9932
9953 9976 10111 10130 10190 10324 10350 10529 10564 10603 10808 10873 10965 11055 11056 11079
11102 11151 11171 11235 11236 11243 11265 11464 11499 11526 11626 11746 11799 11853 11932 11936
11962 12075 12474 12494 12638 12694 12811 12849 12944 13027 13034 13156 13547 13621 13772 13833
13976 13989 14125 14144 14166 14272 14404 14673 14679 14680 14689 14760 14904 14989 14992 15237
15339 15346 15534 15660 15719 15733 15960 16041 16042 16097 16121 16183 16192 16193 16372 16401
16508 16603 16619 16711 16725 16810 17127 17128 17130 17153 17173 17175 17235 17343 17472 17637
17639 17682 17700 17818 18006 18024 18298 18348 18392 18595 18701 18845 18986 19087 19195 19499
19503 19622 19691 19865 20130 20143 20190 20276 20287 20568 20576 20643 20732 20820 20821 20828
20832 20838 20840 20842 20895 21070 21072 21112 21148 21253 21505 21650 21737 21747 21832 21894
21925 22014 22029 22074 22101 22104 22139 22192 22196 22425 22482 22488 22494 22495 22790 22845
23099 23113 23118 23127 23209 23234 23450 23577 23581 23656 23671 23672 23720 23829 23855 23864
23865 23999 24084 24117 24156 24300 24301 24443 24452 24462 24516 24573 24604 24614 24621 24674
24692 24711 24716 24760 24860 24890 24918 24958 25008 25057 25179 25191 25192 25193 25203 25260
25261 25512 25544 25554 25579 25603 25604 25688 25756 25784 25845 25931 25957 26009 26023 26024
26025 26037 26067 26191 26263 26280 26303 26352 26557 26559 26674 26850 26860 26869 27016 27066
27299 27311 27376 27391 27392 27522 27556 27624 27659 27690 27719 27726 27843 27920 27929 28083
28170 28222 28307 28386 28476 28551 28564 28592 28786 28787 28853 28880 28933 29018 29183 29197
29210 29229 29288 29295 29323 29390 29412 29432 29465 29634 29927 29931 30013 30061 30062 30079
30235 30353 30362 30402 30549 30667 30765 30776 30819 30861 30956 30987 31074 31110 31111 31131
31236 31318 31369 31414 31522 31592 31606 31658 31725 31771 31802 31814 31948 31951 31984 32092
32175 32176 32227 32250 32252 32404 32446 32525 32634 32718 32765 32863 32869 33035 33267 33284
33339 33402 33547 33592 33651 33659 33665 33677 33784 33802 33806 33813 33814 33857 34000 34027
34051 34083 34091 34095 34121 34142 34431 34489 34530 34614 34723 34828 34958 35037 35081 35140
35264 35408 35466 35484 35536 35584 35634 35721 35730 35973 36046 36424 36433
228: 3951 4570 4731 4802 4904 4907 4926 4949 4953 4962 5012 5212 5347 5468 5673 6131 6151 6217 6236
6263 6275 6291 6337 6340 6496 6564 6679 6805 6932 7005 7053 7125 7187 7290 7311 7324 7439 7480 7485
7674 7798 7803 7804 7805 7806 7807 7808 7809 7810 7811 7812 7813 7814 7815 7816 7817 7818 7819 7820
7821 7822 7823 7824 7825 7826 7827 7828 7829 7830 7831 7832 7833 7834 7835 7836 7837 7838 7839 7840
7841 7842 7843 7844 7845 8101 8150 8267 8308 8322 8343 8399 8424 8425 8433 8527 8616 8617 8619 8644
8782 8810 8851 8896 8898 8917 8926 9117 9206 9247 9293 9308 9310 9311 9312 9313 9314 9315 9316 9317
9318 9319 9320 9502 9564 9567 9618 9742 9758 9770 9975 9983 10085 10109 10110 10112 10300 10377
10384 10395 10495 10558 10577 10587 10675 10697 10722 10788 10844 10896 10916 10950 10968 10989
11015 11099 11100 11105 11142 11161 11481 11515 11524 11554 11648 11658 11695 11711 11770 11788
12071 12087 12159 12189 12332 12353 12371 12635 12802 12838 12883 12925 13031 13057 13080 13129
13162 13565 13571 13583 13628 13641 13724 13746 13760 13768 13928 13943 13968 14108 14200 14229
14295 14423 14532 14535 14536 14572 14599 14664 14698 14725 14774 14817 14859 15005 15063 15118
15141 15152 15215 15376 15423 15519 15756 15860 15896 15981 16105 16211 16235 16262 16384 16419
16514 16578 16654 16655 16669 16738 16748 16750 16826 16846 16865 17073 17110 17345 17422 17562
17594 17939 17983 18018 18022 18090 18093 18120 18217 18219 18305 18341 18406 18413 18494 18678
18734 18770 19103 19633 19644 19717 19736 20241 20292 20571 20884 20972 20997 21075 21129 21235
21305 21744 21786 21817 21848 21854 21900 21938 21975 22068 22081 22126 22199 22269 22408 22413
23098 23184 23287 23320 23472 23485 23526 23539 23632 23659 23696 23777 23794 23813 23814 23844
23945 24014 24036 24076 24077 24079 24098 24105 24140 24350 24482 24492 24511 24525 24581 24640
24649 24772 24845 24883 24909 24945 24997 25056 25148 25210 25390 25507 25538 25565 25611 25628
25712 25789 25874 25964 26021 26193 26257 26262 26368 26379 26586 26679 26685 26759 26782 26783
26787 26814 26882 26890 27143 27210 27278 27394 27469 27524 27547 27549 27554 27622 27623 27635
27654 27678 27689 27740 27825 27939 27969 28012 28106 28288 28468 28529 28605 28678 28743 28762
28779 28807 28808 28812 28869 28912 28989 29099 29137 29159 29166 29194 29207 29303 29349 29416
29443 29505 29564 29595 29605 29654 29786 29807 29876 29879 29894 29953 29966 30043 30074 30153
30241 30286 30313 30366 30406 30450 30500 30717 30781 30789 30805 30807 30826 30990 31021 31042
31051 31095 31096 31113 31116 31137 31459 31582 31730 31807 31861 32122 32169 32294 32428 32441
32452 32523 32659 32674 32758 32804 32805 32806 32807 32808 32809 32810 32811 32812 32813 32814
32815 32816 32817 32818 32819 32820 32821 32822 32877 32909 32973 33208 33285 33433 33510 33558
33570 33590 33591 33654 33657 33837 33853 33870 33877 34021 34060 34068 34071 34094 34135 34377
34622 34651 34785 34821 34838 34947 34989 35044 35092 35116 35127 35220 35243 35259 35439 35443
35563 35576 35760 35803 35818 35821 36090 36202
229: 4569 4701 4732 4791 4834 4835 4914 4956 5013 5035 5205 5211 5346 5392 5434 5561 5574 5928 6121
6140 6222 6237 6299 6320 6350 6384 6563 6720 6816 6879 7006 7188 7234 7279 7310 7322 7423 7460 7470
7799 7800 7801 7802 7848 7849 8102 8266 8412 8516 8528 8623 8645 8653 8660 8735 8773 8808 8852 8904
9199 9218 9261 9568 9581 9617 9677 9741 9788 9818 9917 9984 10053 10301 10385 10387 10388 10490
10494 10557 10576 10685 10698 10726 10787 10845 10906 10917 10946 10967 11094 11116 11147 11473
11474 11475 11476 11477 11540 11558 11608 11649 11650 11651 11681 11769 11855 12032 12040 12070
12152 12191 12253 12311 12372 12519 12536 12648 12752 12762 12830 12831 12833 12910 12939 12990
13033 13056 13059 13085 13161 13262 13439 13572 13574 13575 13632 13634 13635 13720 13810 14011
14218 14228 14241 14242 14296 14571 14588 14598 14699 14752 14765 14781 14946 14959 15036 15047
15142 15214 15422 15884 15895 15916 15938 16106 16212 16271 16385 16523 16576 16581 16587 16657
16739 16749 16798 16827 16847 16986 16990 17111 17344 17412 17563 17716 17722 17902 17936 17998
18017 18078 18119 18131 18148 18303 18304 18440 18441 18459 18509 18677 18758 19470 19643 19645
19668 19705 19743 20253 20267 20570 20588 20606 20707 20817 20835 20908 20943 20971 21018 21046
21048 21128 21216 21234 21262 21302 21303 21745 21775 21813 21858 21901 21974 21980 22080 22375
22410 23017 23097 23185 23214 23269 23270 23271 23319 23353 23471 23538 23545 23552 23611 23658
23697 23722 23776 23821 23937 23987 24015 24093 24104 24128 24349 24505 24551 24776 24791 24826
24846 24862 24863 24864 24865 24908 24944 24998 25150 25152 25184 25206 25209 25239 25270 25274
25292 25499 25506 25561 25566 25608 25627 25783 25790 25853 25869 25870 25965 26059 26226 26258
26346 26367 26585 26684 26729 26750 26786 26815 27152 27173 27183 27184 27277 27281 27390 27411
27496 27523 27566 27604 27677 27780 27831 27849 27858 27873 27880 27942 27955 28011 28044 28080
28107 28287 28509 28516 28528 28604 28653 28684 28744 28761 28896 28978 28988 29084 29085 29086
29136 29165 29188 29273 29281 29334 29348 29359 29444 29506 29522 29523 29563 29594 29655 29720
29779 29798 29873 29874 29910 29954 29979 30042 30059 30064 30127 30148 30198 30209 30242 30305
30379 30405 30449 30470 30501 30605 30678 30788 30817 30838 30902 30989 31031 31084 31485 31583
31584 31585 31586 31816 31860 31865 31973 31993 32168 32191 32226 32298 32312 32313 32314 32315
32316 32317 32318 32319 32370 32423 32429 32476 32589 32607 32660 32697 32707 32720 32759 32908
33054 33076 33092 33165 33207 33209 33251 33255 33257 33312 33363 33425 33434 33435 33446 33454
33571 33577 33655 33656 33871 34067 34072 34093 34100 34119 34152 34153 34261 34270 34325 34352
34356 34407 34502 34737 34874 34890 34899 34919 34928 34946 35043 35071 35082 35126 35260 35378
35438 35577 35611 35613 35701 35815 35858 35898 35906 35923 35982 36016 36100 36203 36395 36413
230: 1985 2721 2722 2973 3824 4185 4223 4445 4491 4564 4921 4946 4972 5105 5218 5247 5250 5320 5759
6071 6717 6808 7195 7631 7660 7689 7729 8112 8113 8146 8294 8315 8357 8647 8963 9521 9786 9801
10059 10060 10290 10596 10851 10888 11456 11484 11505 11811 12055 12076 12146 12196 12205 12307
12582 12602 12620 12855 12907 13075 13488 13540 13614 13842 14047 14090 14158 14278 14486 14767
14776 14812 14883 15124 15153 15333 15782 16022 16130 16199 16649 16717 16732 17099 17410 17669
17831 17923 17982 18191 18195 18372 18514 18604 18727 19268 19636 20428 20734 20846 21313 22146
22165 22281 22445 22483 22554 22782 23054 23129 23206 23653 23758 23788 23798 24424 24478 24588
24637 25025 25342 25363 25405 25518 25630 25745 26562 26834 26879 27570 27613 27639 27694 27749
27918 27919 27953 28823 28824 28838 28858 28915 28953 29092 29455 29870 29923 29936 30137 30268
30318 30360 30568 30603 31443 31457 31819 32022 32537 32987 33397 33531 33777 33827 34005 34044
34674 34790 34999 35412 35418
231: 666 1385 1618 2310 3106 3232 3314 3575 3590 3827 4014 4082 4181 5196 5213 5214 5215 5216 5217
5218 5219 5220 5221 5222 5223 5224 5225 5226 5227 5228 5229 5230 5730 6045 6934 8083 9079 9491 9936
12409 12788 12899 12953 12977 13877 14088 14124 14151 14366 14652 14905 15108 15434 15491 15661
15750 17800 18013 18259 18352 18388 18811 18974 18985 19054 19595 19872 19997 20536 20948 22623
22857 22948 23227 23686 23890 23891 23893 23896 23897 23898 23900 25080 25081 25082 25471 25662
26190 26938 27006 27138 28151 28259 28334 28384 28418 28460 28968 30715 30716 31061 31160 31339
31340 31392 31717 31912 32097 32859 32962 32963 33511 33616 33706 33817 33886 33887 36170
232: 709 944 1328 1339 1427 1501 1576 1783 1784 1967 2171 2274 2338 2367 2430 2451 2571 2577 2680
2753 2824 2874 2890 2891 2898 2930 2938 2976 3001 3002 3006 3042 3069 3105 3111 3137 3209 3242 3275
3278 3285 3312 3316 3318 3366 3407 3430 3435 3481 3545 3598 3625 3648 3679 3734 3748 3749 3806 3807
3860 3890 3937 3944 4006 4039 4097 4164 4199 4209 4260 4262 4289 4322 4390 4397 5420 5738 5819 5846
5916 6020 6470 6833 7304 7935 7942 7984 7998 8013 8056 8142 8965 8985 9038 9057 9195 9651 10143
10550 11287 12454 13788 13869 13945 14084 14221 14222 14223 14224 14225 15107 15271 15295 15393
15432 16067 16299 16792 17496 17529 17813 18248 18534 18590 18945 19059 19237 19275 19584 19923
20344 20395 20426 20646 20648 21231 21338 21396 21497 21568 21637 21814 21828 22872 22995 23797
24684 24685 24699 24700 24861 24966 25451 25979 25986 28370 28371 29742 29804 29859 30395 31030
31070 31101 31194 31195 31253 31590 34379 34739 35537
233:
234: 642 731 959 993 1130 1255 1366 1583 2462 3055 3237 4484 5752 5822 7881 13698 14020 14103 21057
21434 22688 22810 22850 23805 25995
235: 1175 1261 1301 1937 2108 2281 2431 2432 2701 2939 2946 3135 3955 4529 4634 4691 4776 4866 4934
4945 5039 5199 5303 5381 5416 5547 5580 6011 6022 6076 6123 6230 6264 6309 6321 6554 6638 6719 6797
7030 7047 7108 7215 7294 7306 7462 8022 8115 8195 8277 8362 8552 8700 8767 8870 9232 9619 9686 9690
9837 9860 9927 10077 10119 10243 10333 10335 10410 10466 10554 10707 10720 10740 10751 10832
10912 10927 10958 10959 11010 11035 11164 11461 11552 11670 11707 11767 11833 11844 11849 11873
11920 12035 12100 12180 12249 12338 12421 12520 12700 12729 12871 12886 12891 12900 13054 13093
13174 13186 13521 13600 13652 13944 14044 14060 14135 14188 14307 14482 14581 14604 14635 14675
14914 15020 15159 15267 15311 15418 15924 15994 16058 16110 16118 16194 16528 16634 16662 16685
16764 16799 17057 17210 17268 17331 17444 17445 17520 17557 17570 17737 17758 17964 18076 18109
18364 18415 18451 18675 18706 18825 18863 18964 19201 19667 19708 19868 20164 20193 20215 20290
20458 20589 20826 20915 20963 21006 21082 21127 21142 21300 21314 21672 21716 21767 21909 22015
22093 22288 22350 22384 22537 22848 23115 23167 23339 23459 23601 23692 23744 23753 23881 23909
23982 23989 24085 24155 24316 24365 24378 24411 24417 24533 24553 24701 24832 24900 24981 25041
25105 25123 25124 25136 25415 25552 25563 25594 25613 25632 25727 25814 25837 25969 25999 26000
26001 26012 26013 26014 26015 26205 26381 26390 26590 26703 26707 26749 26771 27117 27271 27275
27319 27330 27336 27468 27507 27620 27867 27891 27974 28130 28504 28560 28575 28610 28680 28758
28827 28981 29147 29182 29221 29326 29398 29514 29572 29603 29785 29815 29902 30030 30047 30096
30117 30161 30219 30257 30383 30433 30459 30471 30497 30627 30664 30810 30840 30883 30890 30903
30935 30965 30974 31059 31130 31147 31165 31207 31531 31573 31832 31858 32224 32278 32361 32413
32477 32486 32524 32715 32721 32794 32907 33201 33290 33371 33506 33522 33614 33646 33705 33783
33879 34048 34096 34168 34240 34263 34318 34433 34497 34550 34639 34663 34699 34786 34834 34892
34903 34910 34918 34964 35077 35191 35262 35404 35476 35662 35698 35784 35909 35986 36027 36138
36220 36245 36356 36378
236: 1024 1295 1600 2374 2443 2955 3156 3177 3212 3602 3913 4273 4893 5704 6445 9433 13709 17784
19381 20047 21448 22580 36326
237: 923 1125 1258 1699 1983 2012 2426 2441 2744 2795 2852 3220 3331 4480 4481 9034 9401 9683 11307
16137 17788 17789 17816 18530 18576 18592 19073 19812 20348 21096 21351 22712 22991
238: 420 764 1348 1717 1763 1842 1963 1971 2169 2491 2513 2802 2803 3091 3363 3609 3988 4487 4488
5147 5625 5757 5869 6412 6670 6825 7348 7760 8408 8825 9002 9070 9162 10145 11059 11209 11210
11571 11573 12114 12293 12463 12673 13006 14006 15178 15538 15730 15731 15732 15805 16410 16429
16430 16431 16432 16433 16434 16436 16440 16888 17436 17690 17828 17876 18008 18601 18649 18836
19070 19124 19179 19233 19448 19833 20065 20131 22607 22640 22825 22898 22906 22908 24545 24547
25059 25060 25061 25062 25063 25064 25065 26420 26490 26798 27485 27529 27794 28350 29376 30547
31656 31716 31726 31916 33508 33513 33960 33961 33962 33963 33964 33965 33968 34456 35254
239: 1782 1866 2038 2082 2214 2262 2264 2270 2279 2303 2323 2326 2346 2384 2388 2423 2429 2437 2464
2482 2511 2522 2531 2552 2553 2589 2614 2637 2644 2671 2672 2745 2764 2773 2817 2842 2879 2882 2899
2941 2951 2990 2993 3026 3045 3074 3084 3109 3131 3167 3223 3247 3255 3266 3274 3302 3339 3380 3394
3434 3447 3452 3489 3490 3492 3515 3517 3526 3536 3544 3566 3568 3578 3579 3581 3607 3615 3640 3660
3663 3681 3698 3699 3716 3826 3838 3867 3887 3910 3920 3956 3996 4007 4051 4075 4078 4092 4217 4254
4269 4281 4291 4311 4341 4350 4352 4371 4374 4376 4386 4693 5696 5697 5699 5731 5767 5768 5787 5800
5808 5858 5867 5868 6185 6187 6190 6458 7892 7893 7909 7910 7912 7919 7960 7985 7992 8031 8048 8057
9695 9707 9720 10220 10221 11068 12505 12975 13708 13892 13896 13898 13929 13935 13954 13956
13975 14045 14136 14149 14902 16047 16056 16894 16895 16896 17654 17659 17742 17749 17752 17767
17775 17779 17801 17824 17841 17843 17864 17870 17873 18168 18170 18551 18569 18581 18611 18636
18643 18793 18797 18798 18807 18940 18941 20342 20364 20415 20425 20454 21094 21354 21386 21428
21436 21439 21441 21442 21443 21472 21490 21494 21500 21501 21536 21545 21551 21559 21560 21561
21562 21563 21585 21586 21590 21591 21592 21593 21594 21598 21599 21606 21607 21614 21639 21640
21641 21653 21713 21940 23047 24675 25474 25990 26915 26916 26917 27044 27140 28427 29377 29378
29385 31185 34563 36235 36334
240: 638 663 839 869 1315 1425 1497 1534 1544 2406 3448 4031 4151 6463 6868 9351 9354 11273 13765
13819 13922 14132 15016 18931 19987 20095 21573 22753 22779
241: 1633 1669 2351 2503 2686 2783 2848 3101 3321 3456 3680 3817 4130 4266 5729 5876 5877 5878 5879
5880 5881 5882 5883 5884 5885 5886 5887 5888 5889 5891 5894 5896 5897 5898 9437 9478 10424 11319
11336 15802 17797 17799 19326 19353 19417 19799 20025 20798 21055 21368 21467 21468 21638 22723
22785 23310 24734 24737 26918 31534 34745 35256 35617 36232
242: 309 348 349 410 411 412 619 630 646 675 740 759 811 879 893 898 905 915 919 1015 1094 1169 1260
1265 1268 1375 1421 1533 1551 1593 1595 1654 1656 1693 1792 1826 1853 1861 1899 1919 1920 1921 1944
1956 1966 1968 1982 2009 2013 2017 2023 2049 2053 2060 2078 2140 2141 2186 2227 2260 2265 2290 2348
2408 2409 2459 2495 2507 2514 2525 2693 2741 2768 2770 2788 2841 2895 2934 2950 2983 3040 3062 3089
3129 3138 3246 3257 3342 3349 3382 3385 3403 3404 3408 3422 3433 3466 3469 3479 3488 3498 3499 3505
3523 3539 3619 3668 3669 3711 3767 3776 3850 3857 3859 3863 3865 3872 3891 3931 4036 4124 4138 4157
4182 4183 4222 4296 4307 4336 4367 4380 4384 4410 4434 5153 5618 5688 5721 5836 5847 5853 5925 6265
6390 6460 6472 6840 7146 7217 7303 7374 7504 7610 7885 8041 8052 8073 9129 9130 9131 9132 9133 9353
9358 9364 9369 9627 9628 9629 9630 9632 9633 9634 9635 9636 10239 10246 10255 10269 11233 11245
11279 11340 11366 11425 13345 13365 13414 13415 13416 13417 13418 13419 13420 13421 13422 13789
13792 13821 13828 13843 14048 14152 14206 14207 14208 14209 15067 15405 15407 15408 15410 16145
16455 16779 16780 16781 16783 17391 17523 17649 17663 17677 17888 18021 18179 18180 18263 18532
18579 18928 19000 19023 19047 19108 19134 19139 19159 19184 19217 19230 19290 19404 19410 19412
19450 19500 19501 19766 19917 19998 20031 20077 20123 20125 20128 20352 20354 20402 20550 20551
21100 21211 21325 21334 21355 21392 21437 21450 21483 21550 21576 21632 21633 21664 22565 22599
22609 22781 22802 22836 22851 22887 23002 23012 23015 23016 23068 23681 23682 24069 24696 24778
24779 25313 25445 25892 26041 26042 26048 26049 27110 27148 27149 27437 27990 27999 28006 28007
28285 29528 29685 29686 29687 29688 29689 32927 32928 32930 33033 33844 34259 35255 35282 35285
35288 35289 36007 36332 36420
243: 1304 1750 1906 1989 2000 2066 2102 2107 2109 2167 2287 2390 2452 2549 2605 2883 2945 3000 3018
3248 3510 3599 3665 3816 4002 4115 4153 4162 4202 4205 4212 4243 4284 4319 4373 4383 5179 5180 5834
6109 6457 6847 6981 7217 7303 8964 9481 9631 9692 10152 10153 11348 13700 13721 13791 13802 13867
13936 14003 14148 14206 14207 14208 14209 15069 15070 15071 15406 15411 15760 16048 16143 16160
16455 16994 17126 17674 18021 18179 18532 18804 18929 18953 19187 19270 19374 19500 19501 20018
20058 20361 20409 21391 22594 22691 22726 23045 23067 23068 23681 25313 25651 25654 25690 26043
26044 26045 26046 26047 26050 27109 27193 27423 27770 27774 31083 32856 32857 32937 33033
244: 1304 1750 1906 1989 2000 2066 2102 2107 2109 2167 2287 2390 2452 2549 2605 2883 2945 3000 3018
3248 3510 3599 3665 3816 4002 4115 4153 4162 4202 4205 4212 4243 4284 4319 4373 4383 5179 5180 5834
6109 6457 6847 6981 7217 7303 8964 9481 9631 9692 10152 10153 11348 13700 13721 13791 13802 13867
13936 14003 14148 14206 14207 14208 14209 15069 15070 15071 15406 15411 15760 16048 16143 16160
16455 16994 17126 17674 18021 18179 18532 18804 18929 18953 19187 19270 19374 19500 19501 20018
20058 20361 20409 21391 22594 22691 22726 23045 23067 23068 23681 25313 25651 25654 25690 26043
26044 26045 26046 26047 26050 27109 27193 27423 27770 27774 31083 32856 32857 32937 33033
245: 1304 1750 1906 1989 2000 2066 2102 2107 2109 2167 2287 2390 2452 2549 2605 2883 2945 3000 3018
3248 3510 3599 3665 3816 4002 4115 4153 4162 4202 4205 4212 4243 4284 4319 4373 4383 5179 5180 5834
6109 6457 6847 6981 7217 7303 8964 9481 9631 9692 10152 10153 11348 13700 13721 13791 13802 13867
13936 14003 14148 14206 14207 14208 14209 15069 15070 15071 15406 15411 15760 16048 16143 16160
16455 16994 17126 17674 18021 18179 18532 18804 18929 18953 19187 19270 19374 19500 19501 20018
20058 20361 20409 21391 22594 22691 22726 23045 23067 23068 23681 25313 25651 25654 25690 26043
26044 26045 26046 26047 26050 27109 27193 27423 27770 27774 31083 32856 32857 32937 33033
246: 1883 2489 2498 3870 5152 5154 5155 5179 5618 5925 6265 6850 6981 6982 7146 8499 9384 9490
10151 11232 11244 11253 13345 13414 13415 13416 13417 13418 13419 13420 13421 13422 15068 15760
16307 16455 16779 16780 16781 16782 16783 17741 18532 19022 19086 19091 19500 19501 20083 20129
20145 20438 22631 23681 25313 25654 25690 26043 26044 26045 26046 26047 26050 27774 27998 31139
31652 32929 33033 33844 35255 35282 35285 35288 35289
247: 1334 1813 2327 3007 3617 4304 5742 7943 8040 9330 10210 13957 14052 14309 15813 16993 17650
21043 21044 21362 23218 24422 26274 26980 27249 28236 28297 28417 31401 31433 31738 31923 32133
32858 32912 33238 36017 36335
248: 707 1334 1667 1981 2125 2398 2714 2935 3892 4490 6033 6935 7938 9429 12428 13732 13753 13957
14052 14091 15272 15813 16993 17484 18852 18856 18908 19180 19232 19848 21043 21044 21317 22717
26980 27427 28167 28179 28236 28297 31923 33040 33238 36017 36335
249: 1334 1650 1722 1851 1858 3078 3830 4000 4174 4215 4430 7895 10210 13732 13753 13957 14052
14091 15813 16131 16471 16993 17666 18882 20418 21043 21044 21667 22737 22777 22837 23218 24422
25671 26653 26980 27427 28167 28297 31401 31433 31738 31923 32858 32912 33238 36017 36335
250: 1240 2818 2949 2970 3059 3118 3180 3262 3289 4433 4439 4507 4534 4535 4540 4549 4554 4578 4643
4676 4807 4813 4840 5085 5240 5244 5280 5309 5379 5430 5485 5488 5498 5502 5507 5570 5979 5995 6047
6083 6279 6335 6348 6382 6529 6530 6561 6591 7026 7167 7286 7647 7704 7714 7726 7759 8110 8310 8359
8382 8383 8601 8689 9304 9532 9588 9738 9790 9880 9964 9965 10074 10094 10332 10412 10428 10483
10583 10666 10883 11442 11644 11676 11678 11710 11921 11931 11935 11940 11941 12137 12184 12248
12477 12554 12569 12760 12864 13097 13491 13593 13597 13655 13659 13727 13747 13790 13825 13996
14131 14289 14469 14480 14504 14507 14590 14607 14759 14870 14872 14924 15337 15360 15366 15384
15388 15392 15879 15970 16012 16437 16643 16733 16829 16842 17046 17384 17528 17735 17920 17986
18108 18123 18204 18327 18385 18471 18687 18737 20202 20259 20277 20625 20629 20738 20755 20762
20851 20918 21077 21274 21694 21748 21759 21957 21959 22130 22153 22191 22195 22247 22295 23154
23289 23292 23376 23418 23756 23782 23950 23970 24086 24552 24762 24885 24889 24957 25010 25338
25380 25418 25452 25465 25733 25758 25812 26323 26329 26331 26334 26336 26341 26344 26363 26365
26366 26370 26728 26746 26799 27097 27132 27388 27504 27520 27525 27560 27775 27856 28531 28576
28606 28624 28633 28635 28636 28637 28642 28797 29180 29195 29236 29330 29397 29403 29644 29908
29985 30003 30040 30156 30280 30382 30411 31001 31011 31509 31518 31786 31971 32003 32044 32205
32206 32253 32265 32307 32330 32352 32418 32436 32475 32518 32533 32552 32702 32867 32900 32981
33101 33105 33113 33139 33258 33309 33492 33542 33563 33576 33597 33599 33601 33604 33618 33764
33830 34018 34023 34204 34249 34329 34516 34522 34533 34682 34772 34795 34806 34823 34827 34973
34976 35033 35054 35096 35128 35161 35179 35194 35199 35410 35424 35447 35459 35462 35468 35488
35493 35575 35583 35647 35654 35655 35656 35663 35680 35758 35766 36025 36026 36029 36031 36033
36040 36056 36061 36070 36074 36093 36204 36261 36272
251: 618 671 810 883 1014 1269 1303 1603 1614 1680 1837 1867 2008 2068 2069 2920 3169 3188 3304 3601
3803 4154 4294 4399 5903 5916 6186 6400 6435 7302 7963 8051 8142 8489 8965 8987 9483 9492 9498 9499
10142 10549 10586 11225 11318 11339 11579 11998 13554 13689 13694 13710 13740 13771 13812 13814
13818 13844 13856 13884 13942 13987 14085 14098 14130 14657 14895 15393 15762 15995 16295 17517
17530 18261 18262 18535 18536 18959 19063 19069 19252 19403 19474 20028 20029 20030 20039 20154
20337 20363 20448 21231 21549 22586 22597 22598 22751 22863 22920 22955 25099 25451 25467 25685
25971 25975 25977 25979 25981 25983 26386 26387 26496 27597 29103 30384 30386 30387 30389 31029
31104 31105 31106 32935 33017 33957 34113 34564 35283 35287 35369 35537
252: 1226 1582 1651 1843 1862 1918 2481 2737 2857 3099 3115 3140 3336 3409 3445 3550 3567 3960 4348
4382 9371 13876 13949 19413 19798 21547 22722 22958
253: 647 1109 1250 1566 1954 2658 3924 4387 6051 6831 9423 9464 9684 10233 11391 12430 13926 14000
14056 14065 14066 15105 15655 15656 15657 15658 16035 16157 17398 18886 19018 19094 19095 19988
20091 21499 23717 30317 33022 36168
254: 911 2404 2684 3225 3402 3647 4452 4493 4531 4572 4577 4620 4632 4857 4937 4944 4967 5084 5138
5217 5229 5234 5376 5384 5596 5610 5638 5643 5927 5938 5976 5983 5989 6159 6205 6216 6224 6239 6243
6249 6256 6271 6278 6280 6302 6303 6317 6323 6328 6353 6356 6589 6658 6706 6782 6919 7070 7076 7081
7088 7595 7918 8159 8194 8309 8323 8329 8347 8354 8634 8636 8646 8650 8656 8699 8821 8836 8841 8897
8911 8955 8958 9357 9573 9577 9750 9760 9831 9839 9840 9884 10066 10343 10344 10345 10346 10352
10432 10508 10547 10565 10569 10575 10597 10602 10660 10725 10727 10806 10807 10809 10817 10859
11050 11073 11075 11103 11136 11173 11378 11557 11594 11605 11617 11621 11622 11624 11665 11679
11748 11749 11756 11800 11813 11862 11863 11880 11882 12039 12066 12079 12092 12121 12122 12148
12218 12287 12299 12330 12343 12345 12348 12357 12361 12563 12653 12654 12661 12668 12675 12682
12692 12714 12723 12735 12755 12852 12853 12861 12862 12938 13473 13481 13483 13492 13494 13502
13514 13516 13542 13544 13608 13610 13617 13618 13661 13681 13695 13723 13836 13840 13903 13910
14036 14173 14290 14417 14601 14603 14614 14627 14628 14629 14656 14704 14779 14780 14782 14786
14802 14811 14829 14850 14938 14993 15052 15113 15117 15119 15219 15285 15471 15898 15930 15940
15948 15976 16078 16200 16261 16278 16375 16381 16520 16526 16529 16539 16589 16592 16604 16839
16841 16853 16985 16988 17112 17155 17157 17181 17182 17190 17197 17198 17204 17206 17209 17242
17243 17249 17251 17258 17259 17264 17267 17429 17431 17434 17510 17511 17515 17684 18150 18164
18315 18322 18330 18491 18702 18763 18815 18854 18914 19283 19306 19382 19612 19628 19630 19950
19951 20166 20201 20240 20264 20275 20286 20311 20350 20700 20713 20743 20759 20769 20775 20855
20940 20964 20975 20980 20994 21134 21249 21250 21275 21321 21602 21674 21761 21964 21973 21977
21986 22096 22138 22366 22481 22511 22758 22786 22787 23065 23066 23080 23245 23248 23249 23251
23295 23301 23345 23347 23361 23371 23397 23419 23430 23569 23630 23641 23643 23644 23646 23648
23729 23730 23737 23831 23948 23980 23984 24071 24127 24142 24295 24314 24318 24514 24515 24554
24794 24878 24901 25531 25533 25631 25763 25773 25776 25787 25792 26203 26494 26561 26580 26617
26673 26676 26680 26683 26699 26701 26702 26731 26751 26755 26929 27131 27413 27467 27541 27542
27569 27607 27616 27637 27645 27657 27789 27799 27811 27814 27853 27864 27967 27979 27981 27984
28019 28024 28051 28056 28081 28096 28129 28406 28450 28481 28491 28494 28498 28506 28510 28523
28542 28557 28603 28645 28683 28685 28726 28731 28755 28842 28845 28847 28854 28860 28886 28887
29170 29208 29209 29219 29222 29243 29284 29290 29297 29300 29317 29318 29325 29447 29499 29698
29706 29708 29718 29778 29797 29891 30024 30112 30114 30122 30172 30220 30249 30331 30338 30576
30599 30602 30616 30632 30670 30725 30778 30786 30795 30798 30803 30809 30820 30824 30849 30864
30865 30872 30879 31127 31445 31495 31499 31528 31569 32001 32026 32030 32041 32047 32048 32067
32069 32077 32082 32084 32180 32211 32212 32214 32291 32332 32345 32363 32373 32395 32396 32405
32512 32625 32637 32643 32649 32738 32763 33108 33116 33136 33144 33145 33156 33245 33254 33291
33328 33338 33346 33347 33353 33354 33360 33361 33367 33370 33398 33399 33415 33418 33421 33424
33426 33443 33449 33466 33505 33687 33852 33869 34019 34035 34036 34120 34127 34132 34159 34166
34171 34223 34228 34234 34244 34256 34295 34436 3471 8 34758 34883 35137 35236 35429 35461 35571
35628 35653 35715 35750 35841 35843 35893 35903 36079 36150 36229 36230 36231 36241 36257 36278
36280 36282
255: 678 976 2324 2397 2585 3225 3979 4349 4530 4531 4568 4572 4577 4620 4622 4623 4632 4654 4667
4674 4685 4690 4857 4876 4937 4967 5138 5234 5536 5610 5638 5643 5772 5857 5927 5938 5940 5975 5983
5989 6159 6205 6216 6224 6239 6243 6244 6256 6271 6278 6302 6317 6323 6328 6329 6344 6353 6355 6356
6682 6706 6727 6729 6736 6756 6782 6783 6799 6811 6817 6903 6977 7070 7076 7081 7120 7184 7190 7205
7595 7962 8050 8149 8159 8188 8194 8276 8323 8329 8347 8368 8604 8605 8606 8650 8674 8693 8699 8790
8830 8836 8897 8911 8955 9497 9565 9573 9577 9750 9831 9839 9884 9926 10008 10066 10287 10319
10343 10344 10346 10352 10357 10360 10380 10427 10432 10508 10547 10569 10575 10578 10590 10600
10602 10626 10634 10643 10659 10665 10725 10807 10809 10817 10859 11050 11073 11075 11103 11136
11173 11489 11494 11501 11509 11534 11557 11594 11605 11617 11621 11635 11643 11665 11748 11749
11800 11813 11862 11863 11878 11881 11882 12027 12039 12058 12066 12078 12079 12092 12122 12148
12218 12257 12275 12286 12287 12310 12322 12330 12345 12348 12357 12361 12515 12563 12607 12610
12614 12617 12642 12653 12661 12678 12692 12714 12723 12735 12755 12837 12853 12857 12913 13432
13434 13443 13463 13473 13481 13483 13495 13502 13514 13516 13542 13544 13559 13566 13617 13624
13681 13695 13769 13836 13895 13903 13910 14036 14173 14261 14279 14290 14291 14426 14601 14603
14614 14627 14629 14656 14670 14704 14779 14780 14782 14786 14828 14938 14941 14993 15052 15113
15119 15197 15209 15216 15219 15230 15232 15240 15253 15471 15898 15930 15940 15948 15973 16085
16200 16243 16248 16261 16278 16353 16365 16375 16381 16520 16529 16539 16567 16594 16724 16744
16745 16756 16760 16839 16992 16995 17028 17034 17045 17063 17069 17080 17081 17090 17112 17132
17157 17181 17182 17188 17190 17198 17206 17209 17242 17243 17249 17260 17263 17264 17267 17411
17431 17434 17510 17515 17586 17684 17745 17748 17953 18150 18164 18230 18290 18294 18312 18317
18330 18332 18342 18347 18382 18491 18550 18763 18815 18854 18914 19249 19328 19411 19612 19628
19630 19669 20109 20149 20201 20240 20263 20264 20275 20279 20286 20311 20585 20740 20743 20775
20855 20934 20940 20975 20980 20994 21134 21249 21260 21278 21498 21548 21584 21589 21674 21761
21880 21882 21964 21973 21977 21979 21989 22048 22096 22138 22206 22511 22657 22844 23046 23049
23050 23065 23066 23125 23131 23135 23136 23137 23149 23153 23175 23180 23192 23198 23245 23248
23251 23267 23301 23345 23357 23361 23370 23397 23399 23409 23427 23430 23608 23630 23643 23644
23648 23729 23730 23737 23823 23826 23831 23837 23839 23852 23856 23948 23984 24127 24295 24326
24332 24333 24341 24342 24375 24377 24394 24401 24428 24434 24438 24467 24468 24473 24499 24514
24521 24554 24594 24596 24599 24601 24626 24627 24655 24795 24878 25101 25498 25531 25533 25551
25668 25720 25721 25729 25763 25773 25776 25785 25787 25792 25994 26203 26516 26561 26580 26617
26671 26673 26683 26699 26701 26702 26731 26751 26755 26765 26837 26845 26847 26881 26895 27131
27197 27220 27265 27413 27467 27541 27542 27569 27607 27616 27637 27645 27657 27686 27687 27710
27713 27717 27735 27787 27811 27814 27853 27861 27879 27907 27925 27927 27932 27954 27967 27981
27984 28019 28024 28030 28051 28056 28058 28081 28096 28119 28135 28148 28212 28313 28423 28424
28450 28481 28491 28494 28498 28503 28510 28523 28542 28552 28603 28683 28716 28721 28729 28755
28845 28847 28854 28887 28904 28921 28922 28942 28945 28946 29097 29104 29105 29108 29170 29208
29216 29220 29222 29243 29277 29283 29284 29296 29300 29315 29317 29318 29325 29392 29429 29447
29467 29473 29499 29536 29537 29540 29573 29574 29577 29698 29706 29708 29718 29778 29797 29884
29891 29932 29952 30006 30114 30122 30172 30228 30327 30331 30393 30564 30570 30576 30599 30602
30604 30616 30632 30670 30680 30725 30786 30795 30798 30803 30824 30846 30864 30865 30879 31094
31127 31263 31366 31511 31569 31570 32001 32021 32047 32048 32067 32069 32077 32082 32108 32180
32211 32212 32214 32258 32291 32326 32363 32395 32396 32405 32512 32606 32617 32625 32643 32649
32738 32763 33006 33108 33116 33144 33145 33229 33245 33346 33347 33353 33366 33367 33370 33384
33386 33389 33416 33443 33466 33496 33505 33687 33700 33852 33869 34009 34019 34029 34035 34036
34120 34127 34139 34140 34159 34171 34180 34190 34223 34228 34234 34244 34256 34295 34309 34428
34436 34525 34713 34718 34758 34883 35236 35238 35239 35272 35298 35429 35461 35628 35653 35715
35725 35750 35841 35843 35893 36079 36116 36150 36153 36229 36230 36231 36241 36257 36274 36280
36281 36282
256: 4542 4819 4865 4901 4958 5142 5238 5317 5594 6010 7681 7860 8338 8342 8763 8829 9779 9834 9928
10062 10415 10750 10904 10991 11053 11130 11133 11536 11675 11953 12065 12139 12164 12562 12664
12765 12776 12796 12804 12820 12865 12888 12893 13594 13658 13673 13674 13845 13889 13902 14049
14113 14506 14530 14761 14825 15023 15128 15993 16276 16616 16644 16743 17571 17944 17992 18103
18162 18188 18346 18404 18423 18495 18496 18517 18764 18850 19737 19753 20171 20195 20229 20246
20261 20814 20906 21032 21243 21685 21769 22056 22136 22183 22436 23217 23235 23291 23444 23748
23850 23858 23859 24050 24092 24154 24436 24469 24597 24628 25371 25525 25559 26371 26712 27072
27088 27349 27514 27536 27571 27614 27618 27631 27647 27792 27871 27895 27915 28118 28520 28596
28736 28892 28893 29101 29187 29829 29916 29976 30203 30376 30427 30476 30677 30679 30842 30909
30937 31132 31425 31821 32726 32800 33052 33192 33294 33552 33562 34046 34224 34239 34301 34418
34457 34494 34725 34764 34896 34908 34956 35091 35237 35390 35472 35635 35673 36048 36409
257: 3951 4570 4731 4802 4904 4907 4926 4949 4953 4962 5012 5212 5347 5468 5673 6131 6151 6217 6236
6263 6275 6291 6337 6340 6496 6564 6679 6805 6932 7005 7053 7125 7187 7290 7311 7324 7439 7480 7485
7674 7798 7803 7804 7805 7806 7807 7808 7809 7810 7811 7812 7813 7814 7815 7816 7817 7818 7819 7820
7821 7822 7823 7824 7825 7826 7827 7828 7829 7830 7831 7832 7833 7834 7835 7836 7837 7838 7839 7840
7841 7842 7843 7844 7845 8101 8150 8267 8308 8322 8343 8399 8424 8425 8433 8527 8616 8617 8619 8644
8782 8810 8851 8896 8898 8917 8926 9117 9206 9247 9293 9308 9310 9311 9312 9313 9314 9315 9316 9317
9318 9319 9320 9502 9564 9567 9618 9742 9758 9770 9975 9983 10085 10109 10110 10112 10300 10377
10384 10395 10495 10558 10577 10587 10675 10697 10722 10788 10844 10896 10916 10950 10968 10989
11015 11099 11100 11105 11142 11161 11481 11515 11524 11554 11648 11658 11695 11711 11770 11788
12071 12087 12159 12189 12332 12353 12371 12635 12802 12838 12883 12925 13031 13057 13080 13129
13162 13565 13571 13583 13628 13641 13724 13746 13760 13768 13928 13943 13968 14108 14200 14229
14295 14423 14532 14535 14536 14572 14599 14664 14698 14725 14774 14817 14859 15005 15063 15118
15141 15152 15215 15376 15423 15519 15756 15860 15896 15981 16105 16211 16235 16262 16384 16419
16514 16578 16654 16655 16669 16738 16748 16750 16826 16846 16865 17073 17110 17345 17422 17562
17594 17939 17983 18018 18022 18090 18093 18120 18217 18219 18305 18341 18406 18413 18494 18678
18734 18770 19103 19633 19644 19717 19736 20241 20292 20571 20884 20972 20997 21075 21129 21235
21305 21744 21786 21817 21848 21854 21900 21938 21975 22068 22081 22126 22199 22269 22408 22413
23098 23184 23287 23320 23472 23485 23526 23539 23632 23659 23696 23777 23794 23813 23814 23844
23945 24014 24036 24076 24077 24079 24098 24105 24140 24350 24482 24492 24511 24525 24581 24640
24649 24772 24845 24883 24909 24945 24997 25056 25148 25210 25390 25507 25538 25565 25611 25628
25712 25789 25874 25964 26021 26193 26257 26262 26368 26379 26586 26679 26685 26759 26782 26783
26787 26814 26882 26890 27143 27210 27278 27394 27469 27524 27547 27549 27554 27622 27623 27635
27654 27678 27689 27740 27825 27939 27969 28012 28106 28288 28468 28529 28605 28678 28743 28762
28779 28807 28808 28812 28869 28912 28989 29099 29137 29159 29166 29194 29207 29303 29349 29416
29443 29505 29564 29595 29605 29654 29786 29807 29876 29879 29894 29953 29966 30043 30074 30153
30241 30286 30313 30366 30406 30450 30500 30717 30781 30789 30805 30807 30826 30990 31021 31042
31051 31095 31096 31113 31116 31137 31459 31582 31730 31807 31861 32122 32169 32294 32428 32441
32452 32523 32659 32674 32758 32804 32805 32806 32807 32808 32809 32810 32811 32812 32813 32814
32815 32816 32817 32818 32819 32820 32821 32822 32877 32909 32973 33208 33285 33433 33510 33558
33570 33590 33591 33654 33657 33837 33853 33870 33877 34021 34060 34068 34071 34094 34135 34377
34622 34651 34785 34821 34838 34947 34989 35044 35092 35116 35127 35220 35243 35259 35439 35443
35563 35576 35760 35803 35818 35821 36090 36202
258: 1985 2721 2722 2973 3824 4185 4223 4445 4491 4564 4921 4946 4972 5105 5218 5247 5250 5320 5759
6071 6717 6808 7195 7631 7660 7689 7729 8112 8113 8146 8294 8315 8357 8647 8963 9521 9786 9801
10059 10060 10290 10596 10851 10888 11456 11484 11505 11811 12055 12076 12146 12196 12205 12307
12582 12602 12620 12855 12907 13075 13488 13540 13614 13842 14047 14090 14158 14278 14486 14767
14776 14812 14883 15124 15153 15333 15782 16022 16130 16199 16649 16717 16732 17099 17410 17669
17831 17923 17982 18191 18195 18372 18514 18604 18727 19268 19636 20428 20734 20846 21313 22146
22165 22281 22445 22483 22554 22782 23054 23129 23206 23653 23758 23788 23798 24424 24478 24588
24637 25025 25342 25363 25405 25518 25630 25745 26562 26834 26879 27570 27613 27639 27694 27749
27918 27919 27953 28823 28824 28838 28858 28915 28953 29092 29455 29870 29923 29936 30137 30268
30318 30360 30568 30603 31443 31457 31819 32022 32537 32987 33397 33531 33777 33827 34005 34044
34674 34790 34999 35412 35418
259: 721 1337 1372 1410 5422 6500 10253 15183 15315
260: 1322 2425 2562 2681 3364 3821 5701 5764 6421 6481 6552 9710 13426 13909 14037 14519 15764
17781 18610 18993 19259 19788 21458 21473 21523 22709 23031 24058 30394 34744 35284 35286
261: 661 688 1930 2266 2512 2534 2632 2724 3034 3368 3518 3703 3784 3819 3881 4128 6000 6183 6901
7932 8564 9064 9080 9407 9889 10156 10173 10201 11306 11343 12505 13742 14424 15696 15799 16330
16460 17491 18613 18816 19106 19189 19372 19811 19892 20408 20446 21102 21492 21552 21553 22742
22795 22924 23306 25993 26983 27009 27212 27248 28141 28150 28157 28235 28260 28326 28348 28431
30750 31380 31397 31727 31764 31933 31946 32118 32165
262: 884 1141 1314 1391 1509 1965 2055 3709 6864 7965 15673 19140 19440 19955 20955 21469 21554
21555 21572 21646 22674 22909 25894 30394
263: 1249 1450 1829 1875 2150 2168 2212 2584 2622 2669 2822 2859 2916 3238 3245 3264 3271 3306 3315
3319 3757 4058 4210 4214 4239 4340 4747 6180 6402 6486 6823 6824 6835 7939 11261 13219 13880 14042
14985 14986 17646 18588 19098 19240 19493 19856 20432 21521 22824 22900 22917 26058 26419
264: 572 573 918 1415 1653 1897 1933 2597 2713 3054 3235 3513 4132 4137 4898 5159 5160 5801 6414
8854 9640 11329 12425 13263 13733 14023 15181 15723 15724 15725 15726 15727 15728 15765 15796
16003 17003 17004 17005 17006 17007 17008 17009 17010 17011 17012 17013 17014 17015 17016 17400
17401 17498 17657 17803 18583 19269 19387 20048 20791 21466 21930 22635 25094 25687 26081 27598
28466 34692
265: 571 918 1415 1653 1897 1933 2597 2713 3054 3235 3513 4132 4137 4898 5159 5160 5801 6414 8854
9640 11329 12425 13263 13733 14023 15181 15723 15724 15725 15726 15727 15728 15765 15796 16003
17016 17400 17401 17498 17657 17803 18583 19269 19387 20048 20791 21466 21930 22635 25094 25687
26081 27598 28466 31700 34692
266: 571 918 1415 1653 1897 1933 2597 2713 3054 3235 3513 4132 4137 4898 5159 5160 5801 6414 8854
9640 11329 12425 13263 13733 14023 15181 15723 15724 15725 15726 15727 15728 15765 15796 16003
17016 17400 17401 17498 17657 17803 18583 19269 19387 20048 20791 21466 21930 22635 25094 25687
26081 27598 28466 31700 34692
267: 339 725 1789 2022 2240 2609 2881 3222 3532 3973 3977 3978 5709 6868 7922 19173 19247 19947
21573 22655
268: 504 1001 1151 1173 1660 1863 1885 1928 2289 2296 2312 2472 2494 2515 2649 2650 3070 3570 3878
3966 3967 4095 4118 4149 4172 4173 4198 4276 4335 4455 4486 4635 4707 5126 5127 5128 5167 5168 5749
6532 6881 6882 6883 7300 7350 7507 7508 7509 7512 7517 7518 7520 7523 7524 7525 7526 7527 7529 7530
7533 7534 7535 7537 7538 7539 7540 7541 7543 7546 7547 7548 7549 7550 7551 7552 7553 7554 7555 7556
7557 7558 7559 7562 7563 7565 7569 7570 7571 7572 7574 7575 7576 7780 7950 8392 8393 8394 8395 8396
8397 8440 8441 8442 8443 8444 8445 8446 8447 8448 8449 8450 8451 8452 8453 8454 8455 8456 8457 8458
8459 8460 8461 8462 8463 8464 8465 8466 8467 8468 8469 8470 8471 8472 8473 8474 8475 8476 8477 8478
8479 8480 8481 8701 8846 8884 9366 9667 9668 9669 9670 9671 9672 10512 11117 11118 11119 11120
11121 11122 11123 11124 11125 11393 11834 11835 11836 11837 11898 11900 12242 13216 13217 13218
13254 13255 13256 13264 13274 13275 13279 13280 13287 13288 13289 13300 13303 13305 13308 13364
13524 13704 13739 13838 13904 13917 14015 14074 14115 14523 14524 14525 14526 14886 14887 15088
15184 15185 15186 15187 15308 15513 15514 15564 15565 15566 15567 15568 15569 15570 15602 15603
15604 15605 15606 15607 15608 15609 15610 15611 15613 15614 15615 15616 15622 15623 15624 15861
15862 15863 15864 15865 15866 15867 15868 15869 15870 15871 15872 15873 15874 16040 16175 16497
16498 16499 16500 16501 16502 16503 16504 16505 16506 16702 16897 16898 16899 16900 16901 16902
16903 16904 16905 16906 16907 16908 16909 16910 16911 16912 16913 16914 16915 16916 16917 16918
16919 16920 16921 16922 16923 16924 16925 16926 16927 16928 16929 16930 16931 16932 16933 16934
16935 16936 16937 16938 16939 16940 16941 16942 16943 16944 16945 16946 16947 16948 16949 16950
16951 16952 16953 16954 16955 16956 16957 16958 16959 16960 16961 16962 16963 16964 16965 16966
16967 16968 16969 16970 16971 16972 16973 16974 16975 16976 16977 16978 16979 16980 16981 16982
17269 17270 17271 17272 17273 17274 17275 17276 17277 17278 17279 17280 17281 17282 17283 17284
17285 17286 17287 17288 17289 17290 17291 17292 17293 17294 17295 17296 17297 17298 17299 17300
17301 17302 17303 17304 17305 17306 17307 17308 17309 17310 17311 17312 17313 17314 17315 17316
17317 17318 17319 17320 17321 17322 17323 17324 17325 17326 17327 17328 17329 17393 17617 17618
17619 17620 17621 17622 17623 17624 17625 17626 17627 17629 17630 17631 17632 17633 17634 17635
17636 17819 17827 18028 18029 18030 18031 18032 18033 18034 18035 18036 18037 18038 18039 18040
18041 18042 18043 18044 18045 18046 18047 18048 18049 18050 18051 18052 18053 18054 18055 18056
18057 18058 18059 18060 18061 18062 18063 18064 18065 18066 18067 18068 18069 18597 19181 19231
19302 19483 19528 19529 19530 19531 19532 19533 19534 19535 19536 19537 19538 19539 19540 19541
19542 19543 19544 19545 19546 19547 19548 19549 19550 19551 19552 19553 19554 19555 19556 19557
19558 19559 19560 19561 19562 19563 19564 19565 19566 19567 19568 19569 19570 19571 19572 19573
19847 19985 20000 20102 20537 20545 20546 20547 21056 21318 21347 21359 21844 21845 21846 21847
21913 22615 22716 22721 22982 22983 22984 23221 23222 23223 23224 23225 23226 23670 23882 24068
24169 25048 25069 25070 25071 25072 25073 25074 25075 25076 25077 25652 25699 25700 25816 25868
25882 25883 25884 25901 25902 25903 25904 25916 25917 25918 26072 26092 26093 26094 26095 26096
26097 26098 26099 26100 26101 26102 26124 26125 26126 26127 26128 26129 26130 26131 26132 26133
26134 26135 26136 26137 26138 26139 26140 26141 26142 26143 26144 26145 26146 26147 26148 26149
26150 26151 26152 26153 26154 26155 26156 26157 26158 26159 26160 26161 26162 26163 26164 26165
26166 26167 26168 26169 26170 26171 26172 26173 26174 26175 26176 26177 26178 26179 26180 26181
26182 26183 26267 26316 26317 26318 26319 26320 26321 26322 26382 26412 26448 26449 26450 26451
26452 26453 26454 26455 26456 26457 26458 26459 26460 26461 26462 26463 26464 26465 26466 26467
26468 26469 26470 26471 26472 26473 26474 26475 26476 26477 26478 26479 27457 27458 27459 28656
28657 28658 28659 28660 28661 28662 28663 28664 28665 28666 28667 28668 28669 28670 28671 28672
28673 28674 28700 28701 28702 28703 28704 28705 29039 29040 29041 29042 29043 29044 29045 29046
29047 29048 29049 29050 29051 29052 29053 29054 29055 29056 29057 29058 29059 29060 29061 29062
29063 29064 29065 29067 29068 29069 29070 29071 29072 29073 29074 29075 29076 29077 29078 29079
29080 29081 29684 30503 30504 30533 30582 30585 30586 30587 30588 30589 30592 30593 30594 30595
30596 30597 31027 31213 31218 31322 31323 31324 31325 31326 31327 31328 31329 31330 31331 31332
31333 31334 31335 31336 31437 31564 31604 31607 31608 31609 31610 31611 31612 31613 31614 31615
31616 31617 31618 31619 31620 31621 31622 31623 31624 31625 31626 31627 31628 31629 31630 31631
31632 31633 31634 31635 31636 31637 31638 31639 31640 31641 31642 31643 31644 31645 31646 31647
31648 31649 31650 32575 32802 32852 33030 33268 33269 33270 33271 33272 33273 33274 33710 33711
33712 33713 33714 33715 33716 33717 33718 33719 33720 33721 33722 33723 33724 33725 33726 33727
33728 33729 33730 33731 33732 33733 33734 33735 33736 33737 33738 33739 33740 33741 33742 33743
33744 33745 33746 33747 33748 33749 33750 33751 33752 33753 33754 33755 33756 33757 33926 34114
34465 34749 35305 35306 35307 35308 35309 35310 35311 35312 35313 35314 35315 35316 35317 35318
35319 35320 35321 35322 35323 35324 35325 35326 35327 35328 35329 35330 35331 35332 35333 35334
35335 35336 35337 35338 35339 35340 35341 35342 35343 35344 35345 35346 35347 35348 35349 35350
35351 35352 35353 35354 35355 35356 35357 35358 35538 35539 35540
269: 504 1001 1151 1173 1660 1863 1885 1928 2289 2296 2312 2472 2494 2515 2649 2650 3070 3570 3878
3966 3967 4095 4118 4149 4172 4173 4198 4276 4335 4455 4486 4635 4707 5126 5127 5128 5167 5168 5749
6532 6881 6882 6883 7300 7350 7507 7508 7509 7512 7517 7518 7520 7523 7524 7525 7526 7527 7529 7530
7533 7534 7535 7537 7538 7539 7540 7541 7543 7546 7547 7548 7549 7550 7551 7552 7553 7554 7555 7556
7557 7558 7559 7562 7563 7565 7569 7570 7571 7572 7574 7575 7576 7780 7950 8392 8393 8394 8395 8396
8397 8440 8441 8442 8443 8444 8445 8446 8447 8448 8449 8450 8451 8452 8453 8454 8455 8456 8457 8458
8459 8460 8461 8462 8463 8464 8465 8466 8467 8468 8469 8470 8471 8472 8473 8474 8475 8476 8477 8478
8479 8480 8481 8701 8846 8884 9366 9667 9668 9669 9670 9671 9672 10512 11117 11118 11119 11120
11121 11122 11123 11124 11125 11393 11834 11835 11836 11837 11898 11900 12242 13216 13217 13218
13254 13255 13256 13264 13274 13275 13279 13280 13287 13288 13289 13300 13303 13305 13308 13364
13524 13704 13739 13838 13904 13917 14015 14074 14115 14523 14524 14525 14526 14886 14887 15088
15184 15185 15186 15187 15308 15513 15514 15564 15565 15566 15567 15568 15569 15570 15602 15603
15604 15605 15606 15607 15608 15609 15610 15611 15613 15614 15615 15616 15622 15623 15624 15861
15862 15863 15864 15865 15866 15867 15868 15869 15870 15871 15872 15873 15874 16040 16175 16497
16498 16499 16500 16501 16502 16503 16504 16505 16506 16702 16897 16898 16899 16900 16901 16902
16903 16904 16905 16906 16907 16908 16909 16910 16911 16912 16913 16914 16915 16916 16917 16918
16919 16920 16921 16922 16923 16924 16925 16926 16927 16928 16929 16930 16931 16932 16933 16934
16935 16936 16937 16938 16939 16940 16941 16942 16943 16944 16945 16946 16947 16948 16949 16950
16951 16952 16953 16954 16955 16956 16957 16958 16959 16960 16961 16962 16963 16964 16965 16966
16967 16968 16969 16970 16971 16972 16973 16974 16975 16976 16977 16978 16979 16980 16981 16982
17269 17270 17271 17272 17273 17274 17275 17276 17277 17278 17279 17280 17281 17282 17283 17284
17285 17286 17287 17288 17289 17290 17291 17292 17293 17294 17295 17296 17297 17298 17299 17300
17301 17302 17303 17304 17305 17306 17307 17308 17309 17310 17311 17312 17313 17314 17315 17316
17317 17318 17319 17320 17321 17322 17323 17324 17325 17326 17327 17328 17329 17393 17617 17618
17619 17620 17621 17622 17623 17624 17625 17626 17627 17629 17630 17631 17632 17633 17634 17635
17636 17819 17827 18028 18029 18030 18031 18032 18033 18034 18035 18036 18037 18038 18039 18040
18041 18042 18043 18044 18045 18046 18047 18048 18049 18050 18051 18052 18053 18054 18055 18056
18057 18058 18059 18060 18061 18062 18063 18064 18065 18066 18067 18068 18069 18597 19181 19231
19302 19483 19528 19529 19530 19531 19532 19533 19534 19535 19536 19537 19538 19539 19540 19541
19542 19543 19544 19545 19546 19547 19548 19549 19550 19551 19552 19553 19554 19555 19556 19557
19558 19559 19560 19561 19562 19563 19564 19565 19566 19567 19568 19569 19570 19571 19572 19573
19847 19985 20000 20102 20537 20545 20546 20547 21056 21318 21347 21359 21844 21845 21846 21847
21913 22615 22716 22721 22982 22983 22984 23221 23222 23223 23224 23225 23226 23670 23882 24068
24169 25048 25069 25070 25071 25072 25073 25074 25075 25076 25077 25652 25699 25700 25816 25868
25882 25883 25884 25901 25902 25903 25904 25916 25917 25918 26072 26092 26093 26094 26095 26096
26097 26098 26099 26100 26101 26102 26124 26125 26126 26127 26128 26129 26130 26131 26132 26133
26134 26135 26136 26137 26138 26139 26140 26141 26142 26143 26144 26145 26146 26147 26148 26149
26150 26151 26152 26153 26154 26155 26156 26157 26158 26159 26160 26161 26162 26163 26164 26165
26166 26167 26168 26169 26170 26171 26172 26173 26174 26175 26176 26177 26178 26179 26180 26181
26182 26183 26267 26316 26317 26318 26319 26320 26321 26322 26382 26412 26448 26449 26450 26451
26452 26453 26454 26455 26456 26457 26458 26459 26460 26461 26462 26463 26464 26465 26466 26467
26468 26469 26470 26471 26472 26473 26474 26475 26476 26477 26478 26479 27457 27458 27459 28656
28657 28658 28659 28660 28661 28662 28663 28664 28665 28666 28667 28668 28669 28670 28671 28672
28673 28674 28700 28701 28702 28703 28704 28705 29039 29040 29041 29042 29043 29044 29045 29046
29047 29048 29049 29050 29051 29052 29053 29054 29055 29056 29057 29058 29059 29060 29061 29062
29063 29064 29065 29067 29068 29069 29070 29071 29072 29073 29074 29075 29076 29077 29078 29079
29080 29081 29684 30503 30504 30533 30582 30585 30586 30587 30588 30589 30592 30593 30594 30595
30596 30597 31027 31213 31218 31322 31323 31324 31325 31326 31327 31328 31329 31330 31331 31332
31333 31334 31335 31336 31437 31564 31604 31607 31608 31609 31610 31611 31612 31613 31614 31615
31616 31617 31618 31619 31620 31621 31622 31623 31624 31625 31626 31627 31628 31629 31630 31631
31632 31633 31634 31635 31636 31637 31638 31639 31640 31641 31642 31643 31644 31645 31646 31647
31648 31649 31650 32575 32802 32852 33030 33268 33269 33270 33271 33272 33273 33274 33710 33711
33712 33713 33714 33715 33716 33717 33718 33719 33720 33721 33722 33723 33724 33725 33726 33727
33728 33729 33730 33731 33732 33733 33734 33735 33736 33737 33738 33739 33740 33741 33742 33743
33744 33745 33746 33747 33748 33749 33750 33751 33752 33753 33754 33755 33756 33757 33926 34114
34465 34749 35305 35306 35307 35308 35309 35310 35311 35312 35313 35314 35315 35316 35317 35318
35319 35320 35321 35322 35323 35324 35325 35326 35327 35328 35329 35330 35331 35332 35333 35334
35335 35336 35337 35338 35339 35340 35341 35342 35343 35344 35345 35346 35347 35348 35349 35350
35351 35352 35353 35354 35355 35356 35357 35358 35538 35539 35540
270: 504 1001 1151 1173 1660 1863 1885 1928 2289 2296 2312 2472 2494 2515 2649 2650 3070 3570 3878
3966 3967 4095 4118 4149 4172 4173 4198 4276 4335 4455 4486 4635 4707 5126 5127 5128 5167 5168 5749
6532 6881 6882 6883 7300 7350 7507 7508 7509 7512 7517 7518 7520 7523 7524 7525 7526 7527 7529 7530
7533 7534 7535 7537 7538 7539 7540 7541 7543 7546 7547 7548 7549 7550 7551 7552 7553 7554 7555 7556
7557 7558 7559 7562 7563 7565 7569 7570 7571 7572 7574 7575 7576 7780 7950 8392 8393 8394 8395 8396
8397 8440 8441 8442 8443 8444 8445 8446 8447 8448 8449 8450 8451 8452 8453 8454 8455 8456 8457 8458
8459 8460 8461 8462 8463 8464 8465 8466 8467 8468 8469 8470 8471 8472 8473 8474 8475 8476 8477 8478
8479 8480 8481 8701 8846 8884 9366 9667 9668 9669 9670 9671 9672 10512 11117 11118 11119 11120
11121 11122 11123 11124 11125 11393 11834 11835 11836 11837 11898 11900 12242 13216 13217 13218
13254 13255 13256 13264 13274 13275 13279 13280 13287 13288 13289 13300 13303 13305 13308 13364
13524 13704 13739 13838 13904 13917 14015 14074 14115 14523 14524 14525 14526 14886 14887 15088
15184 15185 15186 15187 15308 15513 15514 15564 15565 15566 15567 15568 15569 15570 15602 15603
15604 15605 15606 15607 15608 15609 15610 15611 15613 15614 15615 15616 15622 15623 15624 15861
15862 15863 15864 15865 15866 15867 15868 15869 15870 15871 15872 15873 15874 16040 16175 16497
16498 16499 16500 16501 16502 16503 16504 16505 16506 16702 16897 16898 16899 16900 16901 16902
16903 16904 16905 16906 16907 16908 16909 16910 16911 16912 16913 16914 16915 16916 16917 16918
16919 16920 16921 16922 16923 16924 16925 16926 16927 16928 16929 16930 16931 16932 16933 16934
16935 16936 16937 16938 16939 16940 16941 16942 16943 16944 16945 16946 16947 16948 16949 16950
16951 16952 16953 16954 16955 16956 16957 16958 16959 16960 16961 16962 16963 16964 16965 16966
16967 16968 16969 16970 16971 16972 16973 16974 16975 16976 16977 16978 16979 16980 16981 16982
17269 17270 17271 17272 17273 17274 17275 17276 17277 17278 17279 17280 17281 17282 17283 17284
17285 17286 17287 17288 17289 17290 17291 17292 17293 17294 17295 17296 17297 17298 17299 17300
17301 17302 17303 17304 17305 17306 17307 17308 17309 17310 17311 17312 17313 17314 17315 17316
17317 17318 17319 17320 17321 17322 17323 17324 17325 17326 17327 17328 17329 17393 17617 17618
17619 17620 17621 17622 17623 17624 17625 17626 17627 17629 17630 17631 17632 17633 17634 17635
17636 17819 17827 18028 18029 18030 18031 18032 18033 18034 18035 18036 18037 18038 18039 18040
18041 18042 18043 18044 18045 18046 18047 18048 18049 18050 18051 18052 18053 18054 18055 18056
18057 18058 18059 18060 18061 18062 18063 18064 18065 18066 18067 18068 18069 18597 19181 19231
19302 19483 19528 19529 19530 19531 19532 19533 19534 19535 19536 19537 19538 19539 19540 19541
19542 19543 19544 19545 19546 19547 19548 19549 19550 19551 19552 19553 19554 19555 19556 19557
19558 19559 19560 19561 19562 19563 19564 19565 19566 19567 19568 19569 19570 19571 19572 19573
19847 19985 20000 20102 20537 20545 20546 20547 21056 21318 21347 21359 21844 21845 21846 21847
21913 22615 22716 22721 22982 22983 22984 23221 23222 23223 23224 23225 23226 23670 23882 24068
24169 25048 25069 25070 25071 25072 25073 25074 25075 25076 25077 25652 25699 25700 25816 25868
25882 25883 25884 25901 25902 25903 25904 25916 25917 25918 26072 26092 26093 26094 26095 26096
26097 26098 26099 26100 26101 26102 26124 26125 26126 26127 26128 26129 26130 26131 26132 26133
26134 26135 26136 26137 26138 26139 26140 26141 26142 26143 26144 26145 26146 26147 26148 26149
26150 26151 26152 26153 26154 26155 26156 26157 26158 26159 26160 26161 26162 26163 26164 26165
26166 26167 26168 26169 26170 26171 26172 26173 26174 26175 26176 26177 26178 26179 26180 26181
26182 26183 26267 26316 26317 26318 26319 26320 26321 26322 26382 26412 26448 26449 26450 26451
26452 26453 26454 26455 26456 26457 26458 26459 26460 26461 26462 26463 26464 26465 26466 26467
26468 26469 26470 26471 26472 26473 26474 26475 26476 26477 26478 26479 27457 27458 27459 28656
28657 28658 28659 28660 28661 28662 28663 28664 28665 28666 28667 28668 28669 28670 28671 28672
28673 28674 28700 28701 28702 28703 28704 28705 29039 29040 29041 29042 29043 29044 29045 29046
29047 29048 29049 29050 29051 29052 29053 29054 29055 29056 29057 29058 29059 29060 29061 29062
29063 29064 29065 29067 29068 29069 29070 29071 29072 29073 29074 29075 29076 29077 29078 29079
29080 29081 29684 30503 30504 30533 30582 30585 30586 30587 30588 30589 30592 30593 30594 30595
30596 30597 31027 31213 31218 31322 31323 31324 31325 31326 31327 31328 31329 31330 31331 31332
31333 31334 31335 31336 31437 31564 31604 31607 31608 31609 31610 31611 31612 31613 31614 31615
31616 31617 31618 31619 31620 31621 31622 31623 31624 31625 31626 31627 31628 31629 31630 31631
31632 31633 31634 31635 31636 31637 31638 31639 31640 31641 31642 31643 31644 31645 31646 31647
31648 31649 31650 32575 32802 32852 33030 33268 33269 33270 33271 33272 33273 33274 33710 33711
33712 33713 33714 33715 33716 33717 33718 33719 33720 33721 33722 33723 33724 33725 33726 33727
33728 33729 33730 33731 33732 33733 33734 33735 33736 33737 33738 33739 33740 33741 33742 33743
33744 33745 33746 33747 33748 33749 33750 33751 33752 33753 33754 33755 33756 33757 33926 34114
34465 34749 35305 35306 35307 35308 35309 35310 35311 35312 35313 35314 35315 35316 35317 35318
35319 35320 35321 35322 35323 35324 35325 35326 35327 35328 35329 35330 35331 35332 35333 35334
35335 35336 35337 35338 35339 35340 35341 35342 35343 35344 35345 35346 35347 35348 35349 35350
35351 35352 35353 35354 35355 35356 35357 35358 35538 35539 35540
271: 736 1413 2148 2288 2359 2627 2656 2981 3281 3353 3476 3586 4056 4158 4258 4360 4451 4668 4706
4849 4875 4877 5252 5274 5282 5330 5373 5431 5553 5600 5614 5615 5616 5642 5727 5728 5771 5953 6004
6053 6103 6196 6362 6586 6619 6996 7049 7109 7269 7299 7369 7623 7652 7702 8036 8107 8236 8242 8286
8536 8837 8927 9405 9505 9715 9776 9864 9885 9987 10096 10133 10249 10474 10907 10995 11043 11062
11204 11205 11447 11922 12220 12284 12382 12384 12392 12410 12439 12459 12460 12482 12493 12574
12681 13078 13114 13115 13170 13453 13796 13797 13798 13799 14154 14155 14389 14483 14501 14510
14755 14794 14832 14844 14845 15374 15437 15555 15770 15977 16392 16399 16463 16666 16833 17019
17020 17117 17375 17579 17588 17909 17960 17989 18138 18399 18700 18730 18823 18828 18848 18858
18881 18883 19171 19226 19661 19748 19859 20278 20385 20474 20538 20539 20540 20541 20542 20680
20724 20866 21301 21465 21531 21689 21721 21789 21862 22072 22112 22149 22181 22190 22213 22230
22296 22351 22362 22364 22411 22449 23083 23390 23467 23476 24009 24019 24083 24123 24781 24856
24891 24912 25015 25333 25378 25393 25850 25856 26040 26774 26914 26965 27012 27053 27054 27122
27192 27401 27422 27720 27784 27809 27996 28223 28243 28244 28458 28594 28676 28784 28958 29038
29095 29417 29607 29662 29692 29697 29728 29842 29941 30022 30213 30270 30380 30939 31164 31462
31779 32034 32203 32275 32557 32773 32994 32995 33057 33130 33517 33564 33580 33661 33685 33692
33694 33704 33769 33825 34229 34276 34330 34332 34425 34432 34504 34528 34681 34776 34809 34812
34817 34960 34986 35028 35106 35145 35180 35231 35486 35531 35543 35589 35644 35675 35676 35691
35709 35712 35727 35774 35866 35984 36032 36175 36192 36221 36291 36407 36411
272: 1037 1298 1490 1567 1588 1711 1765 2685 3548 6845 7923 9725 11324 14903 19126 19964 22633
273: 828 1186 1822 2185 2250 2371 3027 3355 3701 3869 4312 4538 6922 9426 11183 11344 12394 13986
14427 15634 16400 16448 17145 17853 18011 18631 19008 19893 21170 21171 22116 22833 26806 26976
27136 27204 28172 28218 28352 28430 30740 31365 31682 31893 32093 32757 32917
274: 760 2612 2613 2968 3418 3962 4121 5762 6936 9086 9408 9687 11368 13750 17840 18609 18864 18897
18916 20100 20343 21429 22724 25987 27303 31739 32595
275: 376 494 637 866 1080 1184 1201 1381 1613 1627 1731 1759 1777 2080 2179 2387 2673 2719 2726 2865
3050 3120 3279 3352 3369 3419 3474 3940 3970 4155 4175 4375 5420 5816 7302 7993 8019 8045 8074 9195
10248 10549 10586 10941 11998 13342 13554 15393 15759 16295 17133 17678 17835 18260 18621 18952
20337 21311 21367 21371 21390 21503 21514 22926 22961 22962 23041 24741 25971 26495 26496 26936
28837 29010 29804 29862 31237 31238 31239 31240 31597 31776 33996 35369
276: 1199 1204 1205 1286 1310 1592 1903 2070 2480 2733 4019 5786 9367 9385 9430 9438 19250 19287
19420 19774 19904 19993 19994 21510 21511 21512 21513 21621 22748 22788 22839 22840 22932 22934
25121 34565
277: 754 945 1522 2064 2807 2834 3243 6036 6062 6101 6941 7607 9088 9092 9104 9479 11270 12436
12446 12458 12466 14026 14387 14388 14390 14393 14394 14395 14457 15196 15200 17537 17798 18840
18957 19057 19873 20681 22760 25778 26007 26312 27214 27227 27251 27257 31675 31720 32844
278: 654 948 1010 1108 1488 1766 2195 2219 2638 2655 2660 3080 3836 4231 4411 4432 4467 4525 4526
4567 4648 4704 4777 4872 4954 4975 5067 5094 5096 5106 5150 5172 5223 5248 5295 5332 5343 5388 5462
5486 5493 5509 5542 5582 5788 5831 6064 6090 6120 6218 6248 6293 6378 6512 6513 6514 6521 6522 6559
6599 6704 6801 6886 6957 7000 7092 7098 7242 7308 7402 7448 7468 7505 7612 7613 7614 7643 7671 7711
7739 7858 8082 8105 8129 8145 8217 8305 8346 8398 8539 8558 8571 8662 8702 8703 8704 8705 8706 8707
8708 8709 8710 8711 8712 8722 8723 8766 8805 8815 8867 8883 8885 8886 8887 8888 8889 8890 8966 8967
8970 8971 8972 8973 8982 9020 9055 9056 9204 9221 9346 9538 9543 9625 9664 9665 9730 9737 9787 9807
9865 9967 10047 10090 10336 10430 10441 10555 10649 10699 10724 10742 10759 10784 10834 10869
10915 10929 10948 10963 11019 11145 11358 11405 11471 11517 11556 11615 11764 11777 11846 11865
11875 11917 12006 12038 12063 12217 12325 12486 12491 12518 12593 12627 12687 12716 12860 12946
12992 13007 13058 13103 13154 13192 13423 13424 13512 13536 13611 13662 14031 14095 14162 14239
14240 14298 14379 14409 14475 14503 14584 14647 14676 14738 14851 14856 14956 15002 15050 15073
15086 15102 15148 15198 15210 15294 15328 15334 15359 15385 15420 15429 15433 15468 15492 15512
15562 15721 15729 15855 15913 15968 15999 16015 16031 16061 16238 16369 16449 16476 16511 16573
16623 16694 16714 16730 16822 16830 16855 16883 16884 17151 17176 17237 17340 17354 17446 17487
17551 17560 17581 17702 17942 17975 18077 18135 18222 18326 18374 18393 18401 18448 18600 18619
18620 18685 18710 18723 18835 18976 19050 19266 19305 19629 19695 19787 20042 20165 20170 20200
20225 20273 20331 20461 20463 20555 20641 20773 20824 20878 20921 20926 20941 20951 21037 21058
21067 21143 21191 21192 21193 21239 21247 21284 21348 21427 21669 21692 21723 21834 21835 21902
21912 21918 21924 21927 21935 21937 21962 22054 22117 22185 22258 22268 22349 22370 22467 22470
22471 22530 22707 23055 23056 23057 23064 23092 23163 23230 23242 23336 23337 23338 23425 23447
23453 23513 23625 23669 23694 23732 23786 23806 23973 23996 24114 24163 24309 24368 24399 24409
24484 24542 24577 24642 24682 24713 24725 24751 24752 24753 24754 24759 24788 24816 24825 24839
24905 24951 24976 24982 25038 25110 25172 25243 25294 25312 25352 25387 25426 25510 25570 25586
25609 25749 25761 25910 26004 26069 26119 26194 26197 26297 26324 26397 26409 26432 26436 26483
26484 26584 26596 26607 26666 26695 26714 26726 26757 26884 26909 26910 26911 26926 26939 27050
27114 27119 27194 27282 27293 27357 27472 27500 27752 27848 27872 28070 28122 28435 28474 28558
28579 28626 28682 28759 28791 28984 29091 29098 29132 29176 29270 29360 29413 29434 29435 29510
29561 29593 29631 29640 29734 29837 29844 29918 29956 29998 30072 30104 30129 30130 30151 30232
30279 30302 30350 30407 30428 30455 30480 30492 30538 30614 30628 30649 30672 30673 30674 30719
30720 30802 30832 30862 30894 30913 30931 30941 30942 30943 30944 30945 30946 30947 30948 30998
31002 31048 31112 31193 31205 31228 31376 31521 31599 31698 31781 31976 31989 32086 32099 32125
32204 32231 32276 32279 32375 32384 32392 32415 32427 32487 32549 32585 32586 32588 32705 32730
32799 32827 32828 32848 32888 32961 32968 32969 33023 33090 33204 33227 33317 33341 33458 33550
33613 33625 33668 33682 33791 33819 33842 34016 34101 34196 34226 34331 34406 34486 34554 34633
34655 34719 34768 34837 34891 34906 34914 34924 34926 34955 35005 35019 35070 35124 35151 35184
35252 35271 35405 35446 35514 35528 35556 35603 35620 35686 35687 35688 35689 35761 35850 35954
36053 36075 36127 36193 36253 36309 36311 36322 36323 36359 36375
279: 820 865 1225 1238 1305 1431 1441 1830 1836 1986 2759 3926 4436 5134 5151 5754 5912 6008 6859
6915 6948 7352 7585 8032 8596 9099 9112 9146 9185 9321 10174 10193 11588 11840 12438 12441 12449
12456 12483 12489 12490 12497 14373 14382 14396 14407 14415 15401 15792 15801 16066 16326 16336
17467 17535 17655 17822 18160 19021 19092 19146 20084 20449 20785 20786 20787 20788 20789 20790
20956 21208 21329 21914 22630 23869 24056 24678 26413 26481 26975 27001 27298 28185 28187 28207
28210 28274 28275 28320 28337 28413 28440 30486 30710 30723 30756 30767 31146 31270 31271 31355
31381 31660 31888 31929 31930 31941 31947 32126 32146 33019 34841 34842 36013
280: 5173 5481 5909 6543 9691 9890 10023 10024 10025 10026 10027 10028 10029 10030 10031 10032
10033 10034 10035 10036 12378 13344 13666 13979 14658 15099 15795 15812 16611 18177 25454 25455
25464 26511 27043 27667 27833 28591 34108 34575 34576 34577 34578 34580 34583 35999
281: 1532 1685 1753 1838 1940 2343 2734 2746 2921 3119 3142 3183 3219 3310 3443 3459 3487 3846 3930
4034 4077 4090 4125 4134 5144 5145 5481 5671 5785 5909 6838 7375 7867 7868 8951 9024 9025 9144 9691
10023 10024 10025 10026 10027 10028 10029 10030 10031 10032 10033 10034 10035 10036 10140 10165
10166 11264 11406 11839 12378 13344 13411 15099 15205 15428 15500 15501 15502 15503 15795 15808
15812 16119 16258 16612 16984 18622 19138 19331 19430 19462 19863 19905 20358 20401 21424 21624
22789 22875 24869 25454 25461 25464 25488 26039 26658 26659 27043 27440 27667 27756 27781 27833
28505 29157 30960 32825 34108 34571 34572 34575 34576 34577 34578 34580 34583 35291 35999
282: 3909 9355 9440 9950 9951 11369 11431 12567 13260 13823 15584 15585 15586 15587 15588 15589
15590 15591 15592 15593 15594 15595 15596 15597 15598 15599 19260 19952 19977 20434 25489 25490
25819 25820
283: 625 626 627 631 655 669 670 701 710 724 727 730 748 767 768 769 770 771 772 788 794 796 800 805
806 808 809 818 824 834 848 877 891 892 899 901 902 906 907 912 929 930 954 956 970 990 996 1017 1018
1032 1041 1042 1043 1044 1061 1062 1063 1064 1065 1066 1067 1068 1077 1078 1086 1100 1111 1120 1127
1128 1137 1138 1149 1150 1152 1153 1154 1164 1176 1177 1178 1179 1182 1196 1596 1665 1695 1761 1818
1834 1927 1993 2059 2196 2206 2229 2278 2297 2314 2332 2333 2354 2360 2373 2375 2379 2402 2403 2411
2417 2418 2427 2428 2453 2474 2501 2509 2560 2640 2668 2769 3061 3439 3559 3562 3721 3974 4059 4073
4313 4429 4692 4887 5231 5705 5793 5794 5795 5841 5842 5843 5844 5845 5854 5863 7377 7378 7379 7380
7381 7382 7383 7384 7385 7386 7387 7388 7389 7390 7391 7392 7393 7394 7395 7396 7397 7398 7399 7400
7603 7604 7605 7917 7975 7978 7979 7980 7997 8012 8066 8072 8414 9049 9050 9373 9425 9443 9451 9468
10223 10224 10251 10252 10259 10271 10540 10541 10542 10543 10544 10545 11040 11211 11212 11213
11214 11215 11217 11239 11240 11241 11290 11291 11292 11293 11294 11295 11296 11297 11298 11299
11300 11301 11359 11394 11397 12595 12994 13123 13221 13412 13736 13737 13738 13782 13783 13847
13852 13959 14013 14226 14361 15004 15192 15193 15194 15276 15309 15430 15601 16062 16166 17538
17604 17605 17606 17607 17608 17609 17610 17612 17613 17696 17756 17774 17786 17851 17852 17890
17891 17892 17893 18558 18571 18626 18627 18628 18629 18630 18660 18743 18744 18745 18746 18747
19011 19030 19150 19172 19209 19215 19227 19235 19415 19459 19460 19461 19588 19589 19590 19591
19592 19593 19594 19784 19909 19927 19928 19929 19930 19931 19932 19933 19934 19935 19936 19937
19938 20111 20126 20135 20136 20174 20175 20374 20435 20657 21240 21377 21504 21611 21612 21628
22619 22649 22659 22660 22661 22662 22663 22664 22665 22715 22734 22776 22797 23027 23034 23035
23036 23044 23683 24763 25473 25623 25645 25646 26061 26795 26800 26803 26927 27086 27779 27822
28000 28699 28962 28963 29383 29695 29696 29867 31179 31279 31280 31281 31282 31283 32208 33228
33231 33240 33800 33805 35257 36328 36329 36339 36340 36341 36342 36343 36344 36345 36347
284: 534 926 1333 1352 1483 1554 1571 1620 1621 2863 2864 3113 3146 3263 3708 3725 3848 4441 4442
4482 4589 4605 4804 4812 4836 4878 4880 4996 5064 5129 5311 5359 5402 5423 5424 5432 5467 5524 5637
5746 5942 6009 6023 6087 6245 6313 6347 6351 6364 6574 6594 6600 6601 6630 6645 6657 6707 6715 6746
6795 6937 6993 7033 7077 7280 7281 7282 7602 7722 8094 8258 8311 8332 8643 8779 8784 8785 8812 8813
8814 8893 9010 9011 9157 9233 9275 9278 9578 9599 9616 9698 9705 9777 9932 9953 9976 10111 10130
10190 10324 10350 10529 10564 10603 10622 10808 10873 10965 11055 11056 11079 11102 11171 11265
11463 11464 11499 11526 11600 11626 11746 11799 11853 11932 11936 11962 12075 12474 12494 12638
12694 12811 12849 12944 13027 13034 13156 13449 13547 13621 13772 13833 13976 13989 14125 14144
14166 14272 14404 14673 14679 14680 14689 14760 14904 14989 14992 15237 15339 15534 15660 15733
15960 16097 16183 16192 16193 16372 16401 16603 16619 16711 16725 16810 16891 17024 17127 17153
17173 17175 17219 17235 17236 17343 17472 17637 17639 17682 17700 17818 18006 18024 18298 18348
18595 18701 18845 18986 19195 19499 19503 19600 19622 19691 19865 20190 20276 20287 20460 20568
20576 20643 20732 20754 20820 20821 20828 20832 20838 20840 20842 20895 21070 21071 21072 21110
21112 21148 21253 21737 21747 21832 21894 21925 22014 22029 22074 22101 22104 22192 22196 22289
22425 22447 22482 22488 22494 22495 22502 22790 23099 23113 23118 23127 23128 23208 23209 23234
23384 23450 23577 23581 23656 23671 23672 23720 23829 23855 23864 23865 23944 23999 24084 24156
24300 24443 24452 24462 24516 24573 24604 24614 24621 24674 24692 24760 24853 24860 24890 24918
24958 25008 25057 25067 25179 25191 25192 25193 25203 25260 25261 25423 25494 25512 25544 25554
25578 25579 25603 25604 25688 25756 25784 25845 25931 25957 26009 26023 26037 26067 26191 26263
26280 26303 26352 26557 26559 26674 26850 26860 26869 27016 27066 27299 27311 27376 27391 27392
27421 27483 27522 27556 27624 27659 27690 27719 27726 27843 27920 27929 28083 28170 28222 28245
28307 28386 28476 28551 28564 28592 28786 28787 28853 28880 28933 29018 29183 29197 29210 29229
29288 29295 29323 29390 29412 29432 29465 29634 29715 29927 29931 30013 30061 30062 30063 30079
30235 30353 30362 30402 30549 30667 30765 30776 30819 30861 30956 30987 31110 31131 31236 31318
31369 31414 31514 31522 31592 31606 31658 31725 31771 31802 31948 31951 31984 32038 32092 32175
32176 32227 32250 32252 32374 32404 32446 32525 32634 32718 32765 32863 32869 33035 33134 33284
33339 33340 33402 33547 33587 33592 33651 33659 33665 33677 33784 33813 33814 33857 34000 34027
34051 34074 34083 34091 34095 34121 34142 34431 34489 34530 34614 34666 34723 34828 34958 35008
35037 35081 35140 35264 35408 35466 35484 35536 35584 35634 35721 35730 35973 36046 36424 36433
285: 1140 1170 1287 1442 1452 1475 1508 1517 1672 1705 2149 3092 4559 7762 7790 7905 9379 10263
12097 14112 14189 15306 17875 17976 18806 18989 19214 19257 19574 19575 19576 19577 19578 19579
19580 19581 19582 19899 21422 22137 22201 22773 22799 24916 24978 26121 26531 27832 28571
286: 802 886 1423 1456 1504 1876 3381 3413 3573 3760 3879 4072 5181 5182 5183 7891 9424 10424 15317
15802 18240 18788 18937 18958 20455 20797 20798 21055 21622 22621 24734 24737 26918 31534 35617
36232 36312 36313
287: 704 1458 1502 1828 2014 2092 2103 2153 3191 3470 3475 3875 3904 3968 4252 4306 4521 4784 5220
5241 5350 5438 5500 5510 5606 5992 6467 6595 7012 7091 7113 7238 7663 7742 7873 8221 8271 8297 8567
9215 9421 9825 10019 10180 10186 10207 10370 10436 10625 10701 10794 10795 10796 10918 10977
11149 11440 11566 11637 11871 12047 12480 12580 12699 12844 13160 13501 13558 13685 13712 14472
14577 16122 16700 16824 16987 16991 17191 17252 17355 17521 17564 17656 17759 18249 18307 18880
19044 19943 20371 20714 21306 21544 21683 21742 21983 22098 22252 22356 22392 22514 22694 22914
22933 23101 23362 23482 23572 23698 23734 23742 23968 24016 24729 24953 24984 25022 25116 25402
25617 25780 25847 26742 26934 26992 27033 27211 27276 28022 28090 28338 28396 28462 28739 28993
29146 29352 29393 29415 29571 29651 29714 29911 30017 30124 30180 30216 30262 30483 30625 30848
30873 30916 30961 30992 31033 31351 31363 31406 31516 31724 31759 31859 32046 32188 32221 32230
32271 32380 32393 32898 33142 33182 33246 33252 33256 33365 33534 33678 33780 34058 34291 34350
34398 34514 34726 34889 34907 34993 35024 35110 35138 35221 35511 35570 35626 35902 35991 36181
36386
288: 332 836 837 1191 1392 1579 1964 2016 2138 2178 2182 2836 3226 4061 4080 4120 4238 4256 4301
4521 4784 5220 5241 5350 5438 5500 5510 5606 6039 6595 6836 6962 7012 7091 7113 7238 7419 7494 7663
7742 7900 8221 8271 8297 8544 8567 8726 9215 9254 9696 9825 9873 10019 10180 10207 10370 10436
10625 10734 10794 10795 10796 10918 10977 11149 11237 11440 11566 11596 11637 11871 12047 12178
12509 12580 12699 12751 12844 13160 13501 13558 13841 13899 13966 14429 14577 14842 14962 15040
15368 15678 15919 16700 16824 16987 16991 17191 17252 17355 17564 17656 17720 18249 18307 18711
18917 19081 19320 19632 19953 20440 20605 20714 20818 21306 21683 21742 21983 22168 22252 22356
22392 22514 22710 22935 22936 23101 23362 23482 23572 23698 23734 23742 23968 24016 24576 24729
24953 24984 25116 25139 25196 25536 25617 25744 25780 25847 26710 26742 27033 27150 27211 27276
27306 28022 28090 28158 28239 28338 28396 28739 28993 29146 29352 29393 29415 29571 29651 29714
29911 30017 30124 30180 30216 30483 30669 30734 30735 30848 30873 30895 30916 30961 30992 31033
31351 31363 31516 31759 31818 31859 31891 32046 32188 32221 32230 32271 32380 32393 32472 32710
32733 32898 32974 33142 33182 33246 33252 33256 33365 33450 33678 33780 34058 34291 34350 34398
34514 34726 34889 34907 34993 35024 35110 35138 35221 35511 35570 35626 35746 35902 35916 35991
36133 36181
289: 1347 1363 1773 1775 1976 2146 3391 4464 5870 6856 7894 7906 7907 7914 11196 19345 1935619877
22921 29379
290: 862 1525 1879 1909 2349 2619 3187 3907 3912 4136 4161 4268 4513 4608 4662 4734 4825 4837 5000
5202 5277 5327 5341 5445 5545 5591 5821 5901 6014 6034 6081 6166 6214 6251 6277 6338 6633 6716 6792
6924 7037 7084 7096 7200 7277 7313 7325 7418 7488 7619 7649 7679 7716 8089 8246 8283 8321 8369 8431
8540 8588 8659 8761 8811 8863 8882 8903 9200 9223 9412 9530 9545 9826 9856 9916 10022 10080 10197
10325 10400 10470 10534 10561 10640 10691 10737 10744 10775 10811 10837 10863 10921 10931 10939
10954 10987 11023 11166 11259 11444 11521 11568 11660 11682 11751 11771 11820 11848 11919 12062
12149 12175 12261 12328 12399 12488 12499 12512 12571 12632 12690 12720 12789 12791 12792 12795
12797 12805 12818 12880 12887 12889 12897 12903 12905 12949 12950 12951 12952 12956 12958 12970
13052 13077 13157 13189 13436 13585 13642 13690 13756 13961 13965 14157 14205 14269 14439 14490
14533 14569 14622 14685 14737 14815 14871 14960 15019 15149 15243 15361 15425 15753 15856 15917
15988 16019 16038 16101 16133 16178 16218 16239 16279 16300 16629 16663 16668 16729 16803 16996
16997 16998 16999 17000 17043 17131 17168 17231 17378 17567 17598 17724 17830 17948 18005 18074
18112 18157 18225 18288 18375 18407 18488 18510 18603 18652 18670 18735 18766 18794 18837 18871
19238 19611 19713 19852 20191 20219 20250 20291 20633 20768 20830 20881 20944 20977 20991 21033
21122 21237 21266 21277 21288 21344 21380 21676 21751 21760 21810 22008 22075 22106 22176 22209
22297 22353 22376 22443 22493 22515 22571 22687 23109 23148 23314 23349 23443 23456 23511 23628
23703 23735 23787 23841 23876 23902 23923 23931 23991 24112 24292 24383 24407 24488 24564 24646
24769 24830 24868 24884 24956 24983 25012 25058 25107 25141 25329 25373 25416 25477 25497 25564
25587 25615 25624 25669 25750 25807 25841 25899 25960 26089 26196 26261 26311 26372 26399 26414
26565 26688 26711 26766 26788 26824 26887 27008 27113 27274 27295 27362 27408 27462 27503 27684
27705 27851 27875 27941 27966 28048 28077 28166 28211 28293 28366 28414 28484 28507 28533 28646
28695 28765 28789 28874 28964 28998 29083 29174 29274 29346 29411 29439 29461 29567 29599 29626
29840 29913 29919 29960 29987 30034 30099 30183 30239 30265 30303 30344 30397 30418 30425 30451
30478 30531 30565 30615 30630 30714 30796 30856 30888 30911 30928 30933 30977 31056 31109 31203
31373 31423 31479 31685 31823 31956 32058 32135 32195 32233 32287 32387 32399 32469 32514 32554
32708 32728 32783 32882 32980 33041 33154 33213 33248 33282 33334 33453 33521 33553 33609 33672
33766 33864 34015 34064 34195 34242 34284 34345 34410 34471 34547 34603 34684 34689 34766 34816
34885 34905 34948 34978 35035 35093 35162 35213 35279 35387 35450 35490 35593 35630 35666 35780
35899 35914 36059 36131 36216 36240 36354 36389
291: 2040 2204 2456 3374 3595 4026 4037 4408 6181 6429 7957 9460 11249 11310 13934 14072 17793
19405 19817 20074 21459 21460 21524 21570 21571 22568 22705 27438 27439
292:
293: 639 1116 1436 1537 2691 3175 3465 3902 4131 5710 7349 7788 8385 10538 11216 11218 11370 15749
18640 18648 18933 19583 19979 21477 23918 25817 30514 33507
294: 1013 1521 2093 2191 2216 2217 3754 4332 5778 6059 6925 7972 8563 8819 8952 9014 9089 9152
12680 13210 13990 14403 15530 15687 16344 16516 16517 18861 18995 19879 21617 22020 22728 26017
26958 27034 27242 28134 28197 28238 28361 28372 28461 29015 30545 30697 31362 31438 31896 32131
32916 32964
295: 2370 2478 2521 2711 2868 2998 3104 3241 3873 3903 4010 4089 4113 4497 4629 4655 4801 5052 5358
5401 5457 5555 5607 5784 6061 6147 6394 6403 6415 6544 6573 6623 6684 6809 6905 7010 7085 7160 7262
7452 8004 8005 8071 8192 8280 8515 8590 8666 8748 9248 9552 9566 9747 9829 10021 10086 10295 10378
10488 10627 10828 10866 10972 11542 11595 11641 11696 11760 11778 12014 12544 12722 12841 12976
13136 13447 13562 14004 14248 14428 14445 14808 14927 15028 15262 15324 15882 16063 16076 16142
16333 16570 16821 17049 17178 17239 17353 17394 17442 17494 17846 17847 18314 18473 18655 18891
19648 20345 20614 20763 20909 21027 21064 21137 21257 21565 21566 21605 21697 21731 21870 21932
21981 22263 22329 22342 22394 23123 23158 23265 23379 23474 23525 23774 23943 24011 24073 24372
24693 24786 25147 25215 25289 25508 25782 25851 25943 26210 26376 26542 27026 27380 27410 28013
28093 28189 28248 28332 28480 29264 29324 29449 29555 29588 29649 29723 29880 30015 30076 30150
30347 30703 30867 30996 31229 31388 31491 31960 31997 32101 32127 32199 32266 32379 32454 32699
33096 33205 33343 33377 33462 33555 34055 34163 34213 34272 34323 34430 34475 34546 34632 34707
34965 35041 35073 35167 35185 35389 35495 35580 35890 35929 35936 36097 36213 36415
296: 790 939 1402 1446 1833 2210 2433 2596 2704 2809 2851 3086 3250 3358 3389 3411 3457 3634 3697
3894 4227 5812 7928 7958 8046 8061 13711 13977 18169 20413 21462 21526 21662 22703 22843 22990
23302 23807 24697 26813 32576 34746
297: 917 920 1102 1103 1171 1212 1299 1406 1657 1713 1780 1908 1941 2114 2238 2244 2353 2412 2588
2706 2779 2827 2943 3005 3056 3082 3239 3326 3370 3378 3438 3486 3591 3623 3652 3674 3704 3712 3815
3825 3874 3958 3969 4024 4094 4107 4240 4259 4270 4297 4309 4391 5692 5830 6409 6853 7889 7948 8003
9432 12370 13883 14016 14032 14069 14096 16126 16135 16151 16154 17388 17497 17653 17754 18557
18567 18925 18936 19025 19156 19395 19603 19813 19835 20071 20384 20389 20406 21326 21350 21588
21610 22570 22838 22873 22930 22956 22987 29384 29854 31872
298: 530 606 623 742 1622 2031 2416 2550 2657 2755 3038 3072 3117 3163 3195 3228 3554 3682 3741 3783
3884 3885 4117 4163 4196 4345 4392 4448 4465 4705 4716 4717 4742 4748 4754 5120 5187 5189 5852 6389
6404 6432 6493 6983 8484 8485 8486 8487 8498 9120 9181 9648 9716 10532 10533 10592 11219 11229
11420 12705 12706 12707 12987 12988 13322 13323 13947 14025 14655 15553 15793 15794 16008 16289
17780 18572 18792 18947 19027 19360 19489 20036 20046 20318 20450 21087 21223 21224 21225 21226
21227 21228 21340 21446 21915 21916 21917 22578 22606 23063 23690 24161 24536 24537 24538 24689
24690 24727 24757 24803 24922 24924 24934 25310 25653 25938 25939 26440 26504 26530 26667 26668
26901 26902 26903 26906 26907 26908 26913 26919 27823 28562 29847 30097 31122 31123 31157 31188
31192 31320 31432 31533 31598 31829 31830 31978 32600 32826 34923 35063 35251 36315 36316 36317
36318
299: 530 605 623 660 742 744 845 859 1025 1026 1599 1622 1687 1820 1925 1994 2031 2120 2416 2550
2657 2755 3038 3072 3117 3163 3195 3228 3554 3682 3741 3783 3884 3885 4117 4163 4196 4345 4392 4448
4465 4117 4740 4741 4742 4748 4889 5187 5189 5483 5852 6389 6404 6432 6493 6503 8484 8485 8486 8487
8498 9044 9120 9181 9653 9716 10532 10533 11219 11229 11420 11483 12705 12706 12707 12987 12988
13322 13323 14054 14087 14655 15510 15722 15793 15794 16280 16698 16708 17692 17780 18267 18270
18572 18792 18947 18961 19027 19067 19282 19360 19489 19730 19793 20036 20046 20318 20450 20528
20927 20929 20930 21087 21222 21223 21224 21225 21227 21307 21308 21309 21340 21446 21915 21916
21917 22469 22472 22578 22606 23011 23063 23676 23684 23690 24161 24321 24536 24537 24538 24541
24689 24690 24727 24757 24803 24919 24922 24924 24934 25310 25653 25938 25939 26064 26427 26428
26429 26440 26491 26504 26530 26901 26902 26905 26906 26907 26913 26919 27046 27586 27588 27589
27823 28280 28374 28562 29847 30097 30629 31071 31122 31123 31157 31188 31192 31278 31315 31316
31320 31418 31432 31533 31565 31598 31753 31754 31829 31830 31831 31978 31980 31981 31982 31983
32600 32826 32850 33822 33966 34574 34923 35063 35248 36301 36302 36308 36315 36316 36317 36318
300: 967 1454 2468 2900 3185 3630 4004 5773 6185 9053 10220 10221 10222 11068 13896 14902 17524
17843 21551 21713 23860 26528 26916 26917 26946 31184 31185 36334
301: 933 992 2437 2553 2614 2637 2745 3492 3566 3640 3660 3716 3867 3920 4092 4281 4371 4374 5731
5767 6190 6458 7960 10548 13935 14136 14902 17659 17801 18170 18611 18793 18807 20364 20454 21490
21536 21545 21713 26528 26915 26916 26917 27140 29377 29378 33803 36235 36334
302:
303: 2071
304: 908 938 1053 1243 1370 1472 2228 2992 3424 4099 6869 11234 13208 17834 19458 20078 20375 21312
22816 23030 23667
305: 615 635 662 665 690 863 1008 1021 1087 1185 1296 1369 1380 1399 1545 1597 1635 1664 1678 1681
1692 1771 1872 1898 1929 1945 1974 1991 2021 2051 2088 2089 2090 2110 2116 2122 2132 2224 2248 2298
2335 2455 2460 2506 2561 2564 2610 2683 2689 2694 2695 2765 2784 2811 2839 2840 2847 2849 2867 2894
2944 3013 3224 3377 3388 3446 3450 3484 3531 3537 3546 3547 3560 3577 3593 3659 3707 3843 3983 3984
4022 4054 4057 4133 4141 4232 4248 4293 4402 4403 4976 4977 4978 4980 5171 5655 5750 5862 5871 6498
7606 7908 7915 7916 7931 7946 7969 7970 7983 8030 8049 8817 8942 8943 9347 9361 9362 9380 9434 9646
9942 9943 9944 9945 9946 9947 9948 9949 10125 10126 10127 10137 10266 10279 10283 10881 11067
11069 11199 11200 11262 11330 11838 13222 13223 13555 14561 15281 15628 15629 15800 16068 16148
16152 16349 16613 17021 17395 17397 17533 17699 17757 17768 18264 18442 18543 18564 18598 18667
18668 18949 18965 18968 19029 19071 19072 19165 19167 19170 19203 19225 19243 19245 19288 19292
19341 19373 19431 19764 19849 19858 19881 20012 20016 20049 20369 20388 20430 20642 20644 20652
20984 20985 20986 20987 21093 21245 21332 21402 21412 21415 21416 21487 21516 21600 21615 21616
21801 22651 22792 22841 22889 22913 22949 22968 22974 22976 22980 23687 23913 24075 24940 26053
26442 26603 26605 27042 30139 32158 32925 32926 33230 33798 33945 34365 34369 34370 34371 34372
34373 34374 34375 34376 35477
306: 614 677 693 857 1139 1412 1536 1794 2007 2025 2447 2493 2699 2762 3840 4046 4253 4315 4458 4472
4498 4504 4565 4575 4576 4580 4581 4610 4625 4854 4888 4940 4948 4988 4989 4990 5002 5014 5017 5024
5047 5048 5072 5077 5083 5086 5092 5109 5115 5117 5366 5370 5407 5409 5559 5744 5950 5969 5971 6116
6177 6179 6342 6519 6580 6582 6686 6702 6708 6709 6726 6730 6739 6761 6762 6773 6793 6796 6803 6814
7008 7134 7136 7154 7178 7182 7192 7221 7243 7253 7254 7255 7267 7427 7500 7501 7502 7503 7587 7588
7741 7865 7944 8020 8042 8079 8134 8135 8136 8137 8138 8140 8147 8154 8155 8157 8158 8180 8183 8191
8225 8228 8241 8386 8406 8517 8612 8613 8626 8632 8661 8769 8875 8913 8948 8968 9219 9251 9540 9541
9571 9572 9682 9708 9855 9858 9902 9923 9971 9979 9993 10000 10018 10092 10120 10160 10161 10191
10390 10448 10455 10527 10612 10650 10652 10664 10673 10681 10812 10816 10825 10860 11308 11309
11316 11430 11498 11576 11654 11717 11719 11720 11721 11802 11822 11852 11885 12304 12355 12526
12618 12769 12828 12909 13016 13017 13020 13021 13022 13125 13158 13362 13431 13440 13462 13476
13497 13578 13637 13881 14156 14234 14252 14257 14268 14282 14288 14292 14458 14459 14464 14551
14700 14740 14803 14900 14901 14967 14971 15094 15131 15206 15220 15229 15260 15280 15350 15486
15717 15920 15921 15939 15941 16028 16109 16246 16257 16420 16421 16422 16423 16424 16425 16426
16427 16428 16530 16574 16582 16583 16608 16797 16837 16869 17023 17096 17097 17100 17101 17163
17207 17265 17370 17379 17407 17441 17462 17474 17704 17945 18027 18252 18253 18254 18255 18256
18257 18301 18453 18740 18741 19191 19496 19597 19626 19634 19646 19814 19815 20011 20173 20312
20315 20475 20477 20564 20586 20618 20620 20742 20767 20778 20803 20849 20990 21069 21140 21164
21165 21263 21727 21833 21873 21904 21921 21971 22059 22082 22086 22237 22264 22316 22377 22379
22543 22544 22713 22714 23006 23022 23155 23166 23168 23182 23272 23343 23354 23368 23401 23410
23439 23440 23460 23478 23502 23532 23546 23562 23605 23613 23662 23668 23719 23927 24006 24028
24033 24322 24331 24364 24386 24448 24610 24723 24740 24800 24807 24874 24882 24914 24973 24974
25086 25097 25122 25125 25126 25133 25169 25187 25214 25216 25251 25263 25266 25300 25301 25305
25326 25429 25470 25486 25515 25534 25664 25777 25794 25815 25823 25849 25862 25888 25889 25895
25896 25945 26070 26074 26077 26241 26310 26506 26544 26570 26576 26579 26581 26778 26794 26856
26935 26964 27091 27102 27202 27244 27605 27737 27827 27943 27950 28021 28110 28194 28226 28367
28368 28410 28473 28475 28631 28770 28771 28832 28833 28852 28934 28975 29271 29336 29402 29472
29474 29477 29485 29487 29495 29503 29533 29606 29618 29665 29667 29679 29872 29945 30052 30083
30147 30176 30325 30423 30579 30650 30682 30736 30829 30881 30949 31007 31025 31099 31155 31158
31254 31276 31370 31466 31467 31468 31502 31526 31560 31714 31749 31836 31837 31838 31839 31900
31962 31963 31964 32024 32031 32052 32063 32068 32223 32245 32248 32256 32349 32367 32378 32407
32419 32450 32474 32492 32614 32640 32684 32712 32782 32885 32896 32910 32911 32946 33051 33077
33109 33120 33148 33158 33163 33259 33260 33261 33262 33276 33278 33325 33368 33374 33376 33378
33392 33429 33457 33997 34115 34122 34183 34267 34447 34513 34579 34612 34731 34911 35022 35051
35090 35115 35171 35173 35196 35218 35228 35393 35521 35545 35558 35559 35562 35600 35755 35789
35790 35797 35809 35820 35825 35828 35854 35918 35961 35962 36089 36115 36134 36135 36148 36160
36166 36200 36273 36284 36360 36361 36369

Recombinant DNA constructs are prepared using the DNA encoding each of the identified homologs and the constructs are used to prepare multiple events of transgenic corn, soybean, canola and cotton plants as illustrated in Examples 2-5. Plants are regenerated from the transformed plant cells and used to produce progeny plants and seed that are screened for enhanced water use efficiency, enhanced cold tolerance, increased yield, enhanced nitrogen use efficiency, enhanced seed protein and enhanced seed oil. From each group of multiple events of transgenic plants with a specific recombinant DNA for a homolog the event that produces the greatest enhancement in yield, water use efficiency, nitrogen use efficiency, enhanced cold tolerance, enhanced seed protein and enhanced seed oil is identified and progeny seed is selected for commercial development.

### Example 7

### Consensus Sequence

This example illustrates the identification of consensus amino acid sequence for the proteins and homologs encoded by DNA that is used to prepare the transgenic seed and plants of this invention having enhanced agronomic traits.

ClustalW program was selected for multiple sequence alignments of the amino acid sequence of SEQ ID NO: 311, 314, 316, 333, 344, 346, 365, 373, 383-386, 404-405, 426, 449, 461, 466, 478, 488, 490-491, 512, 541, 543-544, 559, 579, 583, 603, and 610-611 and their homologs. Three major factors affecting the sequence alignments dramatically are (1) protein weight matrices; (2) gap open penalty; (3) gap extension penalty. Protein weight matrices available for ClustalW program include Blosum, Pam and Gonnet series. Those parameters with gap open penalty and gap extension penalty were extensively tested. On the basis of the test results, Blosum weight matrix, gap open penalty of 10 and gap extension penalty of 1 were chosen for multiple sequence alignment.

The consensus amino acid sequence can be used to identify DNA corresponding to the full scope of this invention that is useful in providing transgenic plants, for example corn and soybean plants with enhanced agronomic traits, for example improved nitrogen use efficiency, improved yield, improved water use efficiency and/or improved growth under cold stress, due to the expression in the plants of DNA suppressing a protein with amino acid sequence identical to the consensus amino acid sequence.

The SEQ ID NOs for the identified consensus sequences are reported in table 8 below and the full consensus sequences are provided in the attached sequence listing.

TABLE 8

| PEP SEQ ID NO | Consensus SEQ ID NO |
|---|---|
| 311 | 36447 |
| 314 | 36448 |
| 316 | 36449 |
| 333 | 36450 |
| 344 | 36451 |
| 346 | 36452 |
| 365 | 36453 |
| 373 | 36454 |
| 383 | 36455 |
| 384 | 36456 |
| 385 | 36457 |
| 386 | 36458 |
| 404 | 36459 |
| 405 | 36460 |
| 426 | 36461 |
| 449 | 36462 |
| 461 | 36463 |
| 466 | 36464 |
| 478 | 36465 |
| 488 | 36466 |
| 490 | 36467 |
| 491 | 36468 |
| 512 | 36469 |
| 541 | 36470 |
| 543 | 36471 |
| 544 | 36472 |
| 559 | 36473 |
| 579 | 36474 |
| 583 | 36475 |
| 603 | 36476 |
| 610 | 36477 |
| 611 | 36478 |

### Example 9

### Identification of Amino Acid Domain by Pfam Analysis

This example illustrates the identification of domain and domain module by Pfam analysis.

The amino acid sequence of the expressed proteins that are shown to be associated with an enhanced trait were analyzed for Pfam protein family against the current Pfam collection of multiple sequence alignments and hidden Markov models using the HMMER software. The Pfam protein domains and modules for the proteins of SEQ ID NOs: 308-310, 312-313, 315, 317-332, 334-343, 345, 347-364, 366-372, 374, 382, 387-403, 406-412, 414-425, 427-448, 450-460, 462-465, 467-477, 479-482, 484-487, 493-511, 513-539, 542, 545-558, 560-578, 580-582, 584-598, 600-602, 604-608, and 612-614 are shown in Tables 9, 10 and 11. The Hidden Markov model databases for the identified patent families are also available from the Pfam consortium (ftp.sanger.ac.uk/pub/databases/Pfam/) allowing identification of other homologous proteins and their cognate encoding DNA to enable the full breadth of the invention for a person of ordinary skill in the art. Certain proteins are identified by a single Pfam domain and others by multiple Pfam domains. The function of the identified Pfam domains in proteins providing an enhanced trait in plants was verified by searching identified homologs for the conservation of the identified Pfam domains. The score value for the identified Pfam domains in sequences from table 1 and the minimum score value for the Pfam domain between a protein from table 1 and its identified homologs are reported below in table 9.

TABLE 9
Pfam annotation

| PEP | | | | | | |
|---|---|---|---|---|---|---|
| SEQ | | | | | | |
| ID | Pfam domain | | | | Minumun | |
| NO | name | Begin | Stop | Score | Score | E-value |
| 308 | Aminotran_1_2 | 69 | 470 | 60.3 | −40.6 | 7.20E−15 |
| 309 | Homeobox | 65 | 119 | 67.2 | −2.9 | 6.20E−17 |
| 309 | HALZ | 120 | 164 | 45.5 | 25 | 2.10E−10 |
| 310 | bZIP_1 | 108 | 171 | 40.6 | 27.4 | 6.30E−09 |
| 310 | bZIP_2 | 108 | 162 | 42.2 | 18.1 | 2.10E−09 |
| 312 | Peptidase_S10 | 39 | 504 | 699 | −109.9 | 4.00E−207 |
| 313 | SBP | 103 | 181 | 167.3 | 41.4 | 4.60E−47 |
| 315 | GDPD | 436 | 728 | 154.9 | −14.9 | 2.50E−43 |
| 317 | DUF1292 | 111 | 180 | 19.6 | 16.5 | 4.10E−05 |
| 318 | Aminotran_1_2 | 127 | 473 | 234 | −30.7 | 3.70E−67 |
| 319 | FAD_binding_4 | 50 | 191 | 14.8 | −6.6 | 0.00012 |
| 319 | Lact-deh-memb | 279 | 570 | 657.8 | 75.1 | 9.70E−195 |
| 320 | Fe-ADH | 25 | 365 | 19.5 | −175 | 9.00E−17 |
| 320 | DHQ_synthase | 37 | 346 | −77.9 | −116.5 | 1.00E−06 |
| 321 | AlaDh_PNT_N | 4 | 135 | 277 | −6.3 | 4.20E−80 |
| 321 | AlaDh_PNT_C | 147 | 302 | 315.9 | −13.4 | 8.10E−92 |
| 321 | Shikimate_DH | 152 | 276 | 2 | −4.2 | 0.00013 |
| 322 | Thiolase_N | 14 | 277 | 421.7 | −110.9 | 1.20E−123 |
| 322 | Thiolase_C | 284 | 406 | 219.2 | −5.3 | 1.10E−62 |
| 323 | Thiolase_N | 14 | 277 | 452 | −110.9 | 8.90E−133 |
| 323 | Thiolase_C | 284 | 406 | 218.6 | −5.3 | 1.70E−62 |
| 324 | SOUL | 1 | 150 | 167.1 | 30.2 | 5.20E−47 |
| 325 | AA_kinase | 98 | 373 | 206.1 | −39 | 9.60E−59 |
| 326 | AA_kinase | 10 | 285 | 196.9 | −39 | 5.70E−56 |
| 327 | AA_kinase | 98 | 374 | 191.3 | 78.7 | 2.80E−54 |
| 327 | NAD_binding_3 | 552 | 687 | 164.9 | 4 | 2.40E−46 |
| 327 | Homoserine_dh | 695 | 891 | 378.1 | 59.2 | 1.60E−110 |
| 328 | HLH | 204 | 253 | 56.2 | 35.3 | 1.20E−13 |
| 329 | HLH | 204 | 253 | 56.2 | 35.3 | 1.20E−13 |
| 330 | HLH | 258 | 307 | 70 | 32.4 | 8.90E−18 |
| 331 | HLH | 258 | 307 | 70 | 32.4 | 8.90E−18 |
| 332 | Thioredoxin | 7 | 105 | 42 | −14.2 | 2.40E−09 |
| 332 | Glutaredoxin | 169 | 233 | 94.3 | 24.9 | 4.20E−25 |
| 332 | Glutaredoxin | 298 | 362 | 101.9 | 24.9 | 2.10E−27 |
| 332 | Glutaredoxin | 405 | 469 | 94 | 24.9 | 5.10E−25 |
| 334 | Pkinase | 143 | 405 | 308.2 | 154.7 | 1.80E−89 |
| 334 | Pkinase_Tyr | 143 | 443 | −10.3 | −149.1 | 4.90E−13 |
| 335 | Gln-synt_N | 17 | 97 | 146.5 | 13.5 | 8.20E−41 |
| 335 | Gln-synt_C | 103 | 355 | 291.7 | −123.7 | 1.60E−84 |
| 336 | Pkinase | 143 | 405 | 308.2 | 154.7 | 1.80E−89 |
| 336 | Pkinase_Tyr | 143 | 443 | −10.3 | −149.1 | 4.90E−13 |
| 337 | MethyltransfD12 | 23 | 264 | 358.3 | −38.5 | 1.40E−104 |
| 338 | bZIP_1 | 87 | 150 | 55.9 | 25.5 | 1.60E−13 |
| 338 | bZIP_2 | 87 | 141 | 42 | 29.7 | 2.40E−09 |
| 338 | MethyltransfD12 | 191 | 432 | 360.2 | −38.5 | 4.00E−105 |
| 339 | zf-B_box | 1 | 47 | 29.8 | 22.6 | 1.10E−05 |
| 339 | MethyltransfD12 | 248 | 489 | 358.3 | −38.5 | 1.40E−104 |
| 340 | TPP_enzyme_N | 2 | 177 | 156.7 | −34.7 | 7.20E−44 |
| 340 | TPP_enzyme_M | 193 | 325 | 89 | −7.8 | 1.70E−23 |
| 340 | TPP_enzyme_C | 380 | 517 | 104.2 | 19.9 | 4.40E−28 |
| 341 | RolB_RolC | 11 | 197 | 186.6 | 109.2 | 6.90E−53 |
| 341 | Amino_oxidase | 246 | 729 | 336.5 | 57.4 | 5.10E−98 |
| 342 | Amino_oxidase | 55 | 532 | 117.8 | 45.7 | 3.60E−32 |
| 343 | NIR_SIR_ferr | 131 | 201 | 97.3 | 6.9 | 5.50E−26 |
| 343 | NIR_SIR | 209 | 369 | 171.8 | −18.6 | 2.00E−48 |
| 343 | NIR_SIR_ferr | 390 | 459 | 95 | 6.9 | 2.50E−25 |
| 345 | DEAD | 121 | 290 | 239.3 | 67.8 | 9.80E−69 |
| 345 | Helicase_C | 359 | 435 | 120.7 | 96.2 | 4.90E−33 |
| 347 | LRRNT_2 | 38 | 79 | 46 | 19.6 | 1.40E−10 |
| 347 | LRR_1 | 108 | 130 | 15.4 | 11 | 0.24 |
| 347 | LRR_1 | 132 | 154 | 14.8 | 11 | 0.35 |
| 347 | LRR_1 | 156 | 178 | 12.2 | 11 | 1.4 |
| 347 | LRR_1 | 180 | 201 | 13.7 | 11 | 0.73 |
| 347 | LRR_1 | 228 | 250 | 11.2 | 11 | 2.2 |
| 347 | LRR_1 | 252 | 274 | 14.6 | 11 | 0.42 |
| 347 | LRR_1 | 299 | 321 | 12.7 | 11 | 1.1 |
| 347 | LRR_1 | 347 | 369 | 16.7 | 11 | 0.1 |
| 347 | LRR_1 | 419 | 441 | 12.8 | 11 | 1.1 |
| 347 | LRR_1 | 443 | 465 | 15.3 | 11 | 0.25 |
| 347 | LRR_1 | 467 | 489 | 14.6 | 11 | 0.4 |
| 347 | LRR_1 | 491 | 513 | 16.4 | 11 | 0.12 |
| 347 | LRR_1 | 539 | 560 | 16.7 | 11 | 0.094 |
| 347 | Pkinase | 662 | 927 | 117.1 | 90.1 | 6.00E−32 |
| 347 | Pkinase_Tyr | 662 | 927 | 74.3 | 31.1 | 4.50E−19 |
| 348 | Homeobox | 118 | 172 | 62.6 | 36.3 | 1.50E−15 |
| 348 | HALZ | 173 | 217 | 45.3 | 38.9 | 2.40E−10 |
| 349 | Homeobox | 139 | 193 | 62.6 | 36.3 | 1.50E−15 |
| 349 | HALZ | 194 | 238 | 45.3 | 38.9 | 2.40E−10 |
| 350 | WD40 | 560 | 599 | 34.4 | 22.4 | 4.50E−07 |
| 350 | WD40 | 646 | 683 | 30.5 | 22.4 | 6.80E−06 |
| 351 | C4dic_mal_tran | 199 | 465 | 224.8 | −17.4 | 2.30E−64 |
| 352 | Peroxidase | 85 | 320 | 359.4 | 241.3 | 6.80E−105 |
| 353 | Peroxidase | 85 | 320 | 359.4 | 241.3 | 6.80E−105 |
| 354 | LisH | 7 | 33 | 40.7 | 29.6 | 5.80E−09 |
| 354 | WD40 | 185 | 223 | 46.5 | 23.7 | 1.10E−10 |
| 354 | WD40 | 244 | 282 | 30.2 | 23.7 | 8.60E−06 |
| 354 | WD40 | 285 | 323 | 40.2 | 23.7 | 8.40E−09 |
| 354 | WD40 | 327 | 364 | 23.5 | 23.5 | 0.00089 |
| 354 | WD40 | 368 | 406 | 50.7 | 23.7 | 5.80E−12 |
| 354 | WD40 | 410 | 457 | 46.3 | 23.7 | 1.20E−10 |
| 354 | WD40 | 461 | 501 | 33.6 | 23.7 | 8.20E−07 |
| 355 | bZIP_2 | 325 | 379 | 26.7 | 26.7 | 9.40E−05 |
| 355 | bZIP_1 | 334 | 386 | 28.8 | 28.8 | 2.20E−05 |
| 356 | FAD_binding_4 | 107 | 242 | 132.8 | 15.5 | 1.10E−36 |
| 356 | ALO | 266 | 581 | −44 | −93.7 | 1.40E−07 |
| 357 | NAM | 69 | 200 | 315.8 | 262 | 8.70E−92 |
| 358 | Ribosomal_L21p | 136 | 232 | 164.5 | 45.4 | 3.20E−46 |
| 359 | BTB | 192 | 301 | 85.6 | 19.5 | 1.70E−22 |
| 360 | RWP-RK | 104 | 155 | 106.6 | 89.8 | 8.40E−29 |
| 360 | PB1 | 325 | 408 | 83.2 | 66.1 | 9.20E−22 |
| 361 | Ammonium_transp | 29 | 451 | 263 | −105.1 | 7.00E−76 |
| 362 | BTB | 192 | 301 | 85.6 | 19.5 | 1.70E−22 |
| 363 | OPT | 85 | 744 | 815.2 | −141.6 | 4.10E−242 |
| 364 | DUF506 | 14 | 238 | 260.4 | 45.9 | 4.30E−75 |
| 366 | FTCD_N | 10 | 202 | 184 | −99.6 | 4.10E−52 |
| 367 | FTCD_N | 7 | 199 | 231.8 | −99.6 | 1.70E−66 |
| 368 | SSF | 44 | 461 | −0.7 | −114.1 | 8.50E−09 |
| 369 | Aminotran_5 | 68 | 433 | 435.7 | −139.8 | 7.30E−128 |
| 369 | Beta_elim_lyase | 93 | 373 | −78.8 | −110.3 | 5.00E−05 |
| 370 | ABC_tran | 67 | 262 | 118.5 | 9.8 | 2.10E−32 |
| 371 | Monooxygenase_B | 39 | 418 | 1037 | 175.6 | 0 |
| 372 | PMEI | 34 | 191 | 206.1 | 40.3 | 9.40E−59 |
| 374 | CCT | 141 | 185 | 83.6 | 63 | 7.20E−22 |
| 375 | PHD | 82 | 131 | 38.5 | 27 | 2.70E−08 |
| 375 | SET | 361 | 496 | 154.8 | 52.6 | 2.60E−43 |
| 376 | AP2 | 111 | 163 | 73.7 | 56 | 6.70E−19 |
| 377 | bZIP_2 | 390 | 444 | 33.6 | 21.1 | 8.20E−07 |
| 377 | bZIP_1 | 392 | 453 | 32.9 | 26.8 | 1.30E−06 |
| 378 | AP2 | 24 | 75 | 79.3 | 57 | 1.30E−20 |
| 379 | A_thal_3526 | 17 | 70 | 119.3 | 100.1 | 1.30E−32 |
| 380 | AP2 | 24 | 75 | 78.9 | 57 | 1.80E−20 |
| 381 | Clp_N | 17 | 67 | 39.1 | 1.9 | 1.70E−08 |
| 381 | Clp_N | 96 | 146 | 43.1 | 1.9 | 1.10E−09 |
| 381 | AAA | 202 | 396 | 46 | 14.1 | 1.50E−10 |
| 381 | AAA_2 | 597 | 762 | 361.4 | 28 | 1.70E−105 |
| 381 | AAA_5 | 601 | 767 | 15.6 | 4.1 | 4.80E−05 |
| 381 | ClpB_D2-small | 768 | 857 | 122.4 | 27.9 | 1.50E−33 |
| 382 | eRF1_1 | 1 | 131 | 178 | 0.7 | 2.60E−50 |
| 382 | eRF1_2 | 136 | 268 | 172.7 | 4.1 | 1.00E−48 |
| 382 | eRF1_3 | 271 | 370 | 159.5 | 8.8 | 1.00E−44 |
| 387 | zf-CCCH | 113 | 138 | 31 | 2.1 | 4.80E−06 |
| 388 | zf-B_box | 1 | 47 | 42.6 | 15.5 | 1.50E−09 |
| 388 | zf-B_box | 48 | 90 | 24.8 | 15.5 | 0.00035 |
| 388 | CCT | 350 | 394 | 81.3 | 60.2 | 3.50E−21 |
| 389 | HLH | 128 | 178 | 33.4 | 10.8 | 9.20E−07 |
| 390 | Arginosuc_synth | 100 | 446 | 574.6 | −233.5 | 1.10E−169 |
| 391 | SH3BGR | 204 | 301 | −7.5 | −5.5 | 0.006 |
| 391 | Glutaredoxin | 205 | 279 | 45.1 | 21 | 2.70E−10 |
| 392 | AsnA | 3 | 246 | 612.2 | 27.6 | 5.50E−181 |
| 393 | NAD_binding_1 | 221 | 337 | 150.2 | 19.5 | 6.20E−42 |
| 394 | cNMP_binding | 39 | 131 | 74.2 | 21.2 | 4.70E−19 |
| 394 | Crp | 186 | 217 | 49.9 | 20.8 | 1.00E−11 |
| 395 | PLAC8 | 71 | 166 | 175.3 | 63.2 | 1.80E−49 |
| 396 | HSP20 | 42 | 148 | 109.2 | 34.1 | 1.40E−29 |
| 397 | Myb_DNA-binding | 14 | 61 | 44.2 | 28.5 | 5.30E−10 |
| 397 | Myb_DNA-binding | 67 | 112 | 47.4 | 28.5 | 5.60E−11 |
| 398 | ATP-sulfurylase | 136 | 462 | 529.7 | −150.4 | 3.50E−156 |
| 399 | WRKY | 300 | 360 | 135.4 | 52.5 | 1.80E−37 |
| 400 | Asp | 106 | 439 | −70.2 | −149.4 | 2.10E−09 |
| 401 | Gln-synt_N | 42 | 122 | 26.1 | 14.1 | 4.70E−06 |
| 401 | Gln-synt_C | 141 | 444 | −72.8 | −123.9 | 2.90E−06 |
| 402 | GATase_2 | 27 | 397 | 686.8 | −57.2 | 1.90E−203 |
| 402 | Glu_syn_central | 477 | 773 | 530.8 | 32.1 | 1.70E−156 |
| 402 | Glu_synthase | 830 | 1221 | 802.2 | −154.6 | 3.30E−238 |
| 402 | GXGXG | 1298 | 1493 | 323.1 | 9.5 | 5.60E−94 |
| 403 | Form_Nir_trans | 87 | 343 | 364.7 | −118.6 | 1.70E−106 |
| 406 | NAD_binding_2 | 51 | 213 | 202.4 | 12.2 | 1.20E−57 |
| 407 | NAD_binding_2 | 1 | 162 | 189.3 | 12.2 | 1.00E−53 |
| 408 | Glutaredoxin | 39 | 103 | 76.8 | 30 | 7.70E−20 |
| 409 | Glutaredoxin | 59 | 123 | 111.1 | 55.5 | 3.80E−30 |
| 410 | Homeobox | 139 | 193 | 62.6 | 36.3 | 1.50E−15 |
| 410 | HALZ | 194 | 238 | 45.3 | 38.9 | 2.40E−10 |
| 411 | Homeobox | 137 | 193 | 59.7 | 36.3 | 1.10E−14 |
| 411 | HALZ | 194 | 238 | 45.3 | 38.9 | 2.40E−10 |
| 412 | Homeobox | 139 | 193 | 62.6 | 36.3 | 1.50E−15 |
| 412 | HALZ | 194 | 238 | 45.3 | 38.9 | 2.40E−10 |
| 414 | CCT | 228 | 272 | 90.1 | 74.2 | 7.80E−24 |
| 415 | MBD | 25 | 144 | 62.2 | 36 | 2.00E−15 |
| 416 | MBD | 21 | 94 | 49.3 | 13 | 1.50E−11 |
| 417 | Pkinase | 12 | 263 | 298.2 | 182.4 | 1.70E−86 |
| 417 | Pkinase_Tyr | 12 | 261 | 17.9 | −122.6 | 6.50E−15 |
| 418 | SRF-TF | 3 | 53 | 85.4 | 21.1 | 2.00E−22 |
| 419 | SRF-TF | 11 | 65 | 30.5 | 18.7 | 6.90E−06 |
| 420 | eIF-5a | 85 | 154 | 129 | 10.6 | 1.50E−35 |
| 421 | Homeobox | 33 | 89 | 78.7 | 45.4 | 2.00E−20 |
| 421 | START | 236 | 457 | 196.1 | 6.7 | 9.50E−56 |
| 422 | Homeobox | 33 | 89 | 78.7 | 45.4 | 2.00E−20 |
| 422 | START | 236 | 457 | 196.1 | 6.7 | 9.50E−56 |
| 423 | Homeobox | 33 | 89 | 78.7 | 45.4 | 2.00E−20 |
| 423 | START | 236 | 457 | 196.1 | 6.7 | 9.50E−56 |
| 424 | Homeobox | 33 | 89 | 78.7 | 45.4 | 2.00E−20 |
| 424 | START | 236 | 457 | 196.1 | 6.7 | 9.50E−56 |
| 425 | Arginase | 61 | 336 | 283.6 | 0 | 4.40E−82 |
| 427 | CBFB_NFYA | 147 | 203 | 134.7 | 100.3 | 3.00E−37 |
| 428 | CBFB_NFYA | 148 | 204 | 139.4 | 89.5 | 1.10E−38 |
| 429 | CBFB_NFYA | 93 | 149 | 140.9 | 87.3 | 4.10E−39 |
| 430 | CBFB_NFYA | 70 | 126 | 140.2 | 89.5 | 6.60E−39 |
| 431 | CBFB_NFYA | 73 | 129 | 141.1 | 74.2 | 3.30E−39 |
| 432 | PAS_2 | 103 | 219 | 243 | 82 | 7.50E−70 |
| 432 | GAF | 252 | 433 | 108.7 | 62.8 | 1.90E−29 |
| 432 | Phytochrome | 444 | 623 | 410.7 | 29 | 2.40E−120 |
| 432 | PAS | 654 | 770 | 105.5 | 8.3 | 1.80E−28 |
| 432 | PAS | 785 | 907 | 113.5 | 8.3 | 6.80E−31 |
| 432 | HisKA | 927 | 991 | 52.6 | 25.1 | 1.50E−12 |
| 432 | HATPase_c | 1039 | 1152 | 69.6 | 37.6 | 1.20E−17 |
| 433 | HMG_box | 44 | 113 | 115.8 | 37.7 | 1.40E−31 |
| 434 | SPRY | 77 | 224 | 25.9 | 22.2 | 1.70E−05 |
| 435 | SPRY | 77 | 224 | 25.9 | 22.2 | 1.70E−05 |
| 436 | RRM_1 | 4 | 68 | 69.7 | 29.9 | 1.00E−17 |
| 436 | RRM_1 | 97 | 160 | 52.9 | 29.9 | 1.30E−12 |
| 437 | RRM_1 | 4 | 68 | 69.7 | 29.9 | 1.00E−17 |
| 437 | RRM_1 | 97 | 160 | 52.9 | 29.9 | 1.30E−12 |
| 438 | RRM_1 | 4 | 69 | 56.2 | 51.6 | 1.20E−13 |
| 438 | zf-CCHC | 85 | 102 | 31.4 | 18.3 | 3.10E−06 |
| 439 | RRM_1 | 4 | 69 | 56.2 | 51.6 | 1.20E−13 |
| 439 | zf-CCHC | 85 | 102 | 31.4 | 18.3 | 3.10E−06 |
| 440 | RRM_1 | 4 | 69 | 57.1 | 49.8 | 6.80E−14 |
| 440 | zf-CCHC | 86 | 103 | 33 | 18.3 | 1.20E−06 |
| 441 | RRM_1 | 4 | 69 | 57.1 | 49.8 | 6.80E−14 |
| 441 | zf-CCHC | 86 | 103 | 33 | 18.3 | 1.20E−06 |
| 442 | MMR_HSR1 | 119 | 240 | 131.3 | 66.5 | 3.10E−36 |
| 442 | KH_2 | 345 | 409 | 87.5 | 25.3 | 4.60E−23 |
| 443 | MMR_HSR1 | 119 | 240 | 131.3 | 66.5 | 3.10E−36 |
| 443 | KH_2 | 345 | 409 | 87.5 | 25.3 | 4.60E−23 |
| 444 | MMR_HSR1 | 119 | 240 | 131.3 | 66.5 | 3.10E−36 |
| 444 | KH_2 | 345 | 409 | 87.5 | 25.3 | 4.60E−23 |
| 445 | zf-C3HC4 | 152 | 193 | 40.2 | 29.7 | 8.40E−09 |
| 446 | zf-C3HC4 | 152 | 193 | 40.2 | 29.7 | 8.40E−09 |
| 447 | zf-C3HC4 | 152 | 193 | 40.2 | 29.7 | 8.40E−09 |
| 448 | zf-C3HC4 | 152 | 193 | 40.2 | 29.7 | 8.40E−09 |
| 450 | 2-Hacid_dh | 8 | 324 | 145.9 | 14 | 1.20E−40 |
| 450 | 2-Hacid_dh_C | 113 | 292 | 276.4 | 117.8 | 6.30E−80 |
| 451 | AlaDh_PNT_N | 4 | 136 | 272.7 | −6.3 | 8.50E−79 |
| 451 | AlaDh_PNT_C | 148 | 303 | 309.5 | −13.4 | 7.00E−90 |
| 452 | AlaDh_PNT_N | 4 | 136 | 272.7 | −6.3 | 8.50E−79 |
| 452 | AlaDh_PNT_C | 148 | 303 | 309.5 | −13.4 | 7.00E−90 |
| 453 | tRNA_synt_1c_R1 | 14 | 171 | 220.2 | 40.2 | 5.30E−63 |
| 453 | tRNA_synt_1c_R2 | 172 | 264 | 109.8 | 27.2 | 9.20E−30 |
| 453 | tRNA-synt_1c | 271 | 577 | 565.2 | −112.8 | 7.30E−167 |
| 453 | tRNA-synt_1c_C | 579 | 770 | 242.5 | 25.4 | 1.10E−69 |
| 454 | SBP_bac_3 | 75 | 296 | 114.2 | 56.9 | 4.30E−31 |
| 455 | Gln-synt_N | 17 | 97 | 146.5 | 13.5 | 8.20E−41 |
| 455 | Gln-synt_C | 103 | 355 | 291.7 | −123.7 | 1.60E−84 |
| 456 | bZIP_1 | 76 | 139 | 43.5 | 27.4 | 8.20E−10 |
| 456 | bZIP_2 | 76 | 130 | 42.5 | 27 | 1.70E−09 |
| 457 | bZIP_1 | 76 | 139 | 43.5 | 27.4 | 8.20E−10 |
| 457 | bZIP_2 | 76 | 130 | 42.5 | 27 | 1.70E−09 |
| 458 | BPD_transp_2 | 5 | 354 | 254.5 | 33.4 | 2.60E−73 |
| 459 | BPD_transp_2 | 82 | 431 | 254.5 | 33.4 | 2.60E−73 |
| 460 | Asp_decarbox | 2 | 119 | 198.7 | 156.7 | 1.60E−56 |
| 462 | Glt_symporter | 4 | 370 | 805.4 | −247.2 | 3.70E−239 |
| 463 | Glt_symporter | 4 | 370 | 805.4 | −247.2 | 3.70E−239 |
| 464 | SBP_bac_3 | 75 | 296 | 114.2 | 56.9 | 4.30E−31 |
| 465 | SBP_bac_3 | 75 | 296 | 114.2 | 56.9 | 4.30E−31 |
| 467 | AlaDh_PNT_N | 4 | 136 | 262.3 | −6.3 | 1.20E−75 |
| 467 | AlaDh_PNT_C | 148 | 302 | 275.9 | −13.4 | 9.10E−80 |
| 468 | Nitroreductase | 20 | 181 | 82.5 | 37 | 1.60E−21 |
| 469 | Acid_phosphat_B | 1 | 179 | 89.8 | −18.9 | 9.70E−24 |
| 470 | bZIP_1 | 76 | 139 | 43.5 | 27.4 | 8.20E−10 |
| 470 | bZIP_2 | 76 | 130 | 42.5 | 27 | 1.70E−09 |
| 471 | Ubiquitin | 22 | 92 | 45.4 | 19.6 | 2.20E−10 |
| 472 | LIM | 172 | 228 | 29 | 9.4 | 2.50E−06 |
| 473 | LIM | 147 | 203 | 42 | 2.4 | 2.40E−09 |
| 474 | zf-C3HC4 | 197 | 237 | 43.5 | 21.9 | 8.40E−10 |
| 475 | RCC1 | 95 | 145 | 24.5 | 21.5 | 0.00045 |
| 475 | RCC1 | 266 | 315 | 25.8 | 21.5 | 0.00018 |
| 475 | RCC1 | 350 | 398 | 48.9 | 21.5 | 2.00E−11 |
| 476 | Sulfate_transp | 198 | 510 | 494.9 | −44.7 | 1.10E−145 |
| 476 | STAS | 533 | 652 | 84.7 | 1.5 | 3.30E−22 |
| 477 | RCC1 | 95 | 145 | 24.5 | 21.5 | 0.00045 |
| 477 | RCC1 | 266 | 315 | 25.8 | 21.5 | 0.00018 |
| 477 | RCC1 | 350 | 398 | 48.9 | 21.5 | 2.00E−11 |
| 479 | Glyco_transf_20 | 58 | 544 | 840.5 | −201.3 | 9.90E−250 |
| 479 | Trehalose_PPase | 593 | 829 | 338.3 | −32.2 | 1.50E−98 |
| 480 | RRM_1 | 19 | 89 | 25 | 18 | 0.0003 |
| 481 | Asp | 89 | 438 | −88.1 | −149.4 | 2.40E−08 |
| 482 | Ank | 44 | 81 | 12 | 1.2 | 0.67 |
| 482 | Ank | 83 | 112 | 21.9 | 1.2 | 0.0026 |
| 482 | Ank | 118 | 150 | 16.3 | 1.2 | 0.13 |
| 482 | Ank | 152 | 184 | 17.2 | 1.2 | 0.07 |
| 482 | Ank | 186 | 218 | 31.4 | 1.2 | 3.70E−06 |
| 482 | Ank | 220 | 252 | 24.2 | 1.2 | 0.00055 |
| 482 | Ank | 254 | 287 | 29 | 1.2 | 2.00E−05 |
| 482 | Ank | 288 | 320 | 1.6 | 1.2 | 22 |
| 484 | DSPc | 105 | 240 | 25.3 | −4.2 | 1.90E−06 |
| 485 | Usp | 8 | 162 | 86.9 | 26.5 | 7.20E−23 |
| 486 | U-box | 17 | 94 | 76.1 | 33.2 | 1.30E−19 |
| 487 | U-box | 10 | 84 | 83.9 | 33.2 | 5.70E−22 |
| 493 | MIT | 5 | 73 | 103.3 | 38.4 | 8.20E−28 |
| 493 | AAA | 169 | 353 | 231.7 | 43.1 | 1.80E−66 |
| 493 | Vps4_C | 368 | 433 | 127.3 | −2.2 | 5.10E−35 |
| 494 | AP2 | 80 | 132 | 77.7 | 59.9 | 4.20E−20 |
| 495 | Ssl1 | 37 | 292 | 530.7 | −115.9 | 1.80E−156 |
| 495 | C1_4 | 374 | 422 | 98.3 | 50.7 | 2.60E−26 |
| 496 | POX | 213 | 346 | −18.9 | −23 | 2.80E−05 |
| 496 | Homeobox | 396 | 454 | −2.1 | −3.7 | 0.019 |
| 497 | POX | 214 | 350 | −0.6 | −24.9 | 6.60E−07 |
| 497 | Homeobox | 404 | 462 | −2.5 | −3.7 | 0.021 |
| 498 | Dimerisation | 34 | 82 | 71.7 | 35.1 | 2.70E−18 |
| 498 | Methyltransf_2 | 91 | 342 | 278.1 | −95.8 | 2.00E−80 |
| 499 | DUF640 | 1 | 160 | 251 | 172 | 2.90E−72 |
| 500 | p450 | 2 | 479 | 214.1 | −29.9 | 3.80E−61 |
| 501 | MFS_1 | 47 | 528 | 24.1 | 24.1 | 0.00057 |
| 501 | PTR2 | 109 | 520 | 323.9 | −39.5 | 3.30E−94 |
| 502 | Pkinase | 16 | 269 | 336.6 | 200.3 | 4.70E−98 |
| 502 | Pkinase_Tyr | 16 | 267 | 36.2 | −68.3 | 3.90E−16 |
| 502 | NAF | 311 | 372 | 112.2 | 25.9 | 1.70E−30 |
| 503 | Acyl-ACP_TE | 92 | 367 | 438 | 40.9 | 1.40E−128 |
| 504 | PAS_2 | 69 | 185 | 230.5 | 48 | 4.20E−66 |
| 504 | GAF | 218 | 402 | 111.1 | 45.8 | 3.60E−30 |
| 504 | Phytochrome | 413 | 592 | 421.7 | 29 | 1.20E−123 |
| 504 | PAS | 620 | 735 | 101.9 | 8.3 | 2.20E−27 |
| 504 | PAS_4 | 626 | 740 | 35.5 | 16.4 | 2.10E−07 |
| 504 | PAS | 750 | 875 | 100 | 8.3 | 8.30E−27 |
| 504 | HisKA | 895 | 959 | 44.5 | 22.5 | 4.10E−10 |
| 504 | HATPase_c | 1007 | 1118 | 72.8 | 37.5 | 1.20E−18 |
| 505 | PAS_2 | 69 | 185 | 230.5 | 48 | 4.20E−66 |
| 505 | GAF | 218 | 402 | 108 | 45.8 | 3.20E−29 |
| 505 | Phytochrome | 413 | 592 | 421.7 | 29 | 1.20E−123 |
| 505 | PAS | 620 | 735 | 101.9 | 8.3 | 2.20E−27 |
| 505 | PAS_4 | 626 | 740 | 35.5 | 16.4 | 2.10E−07 |
| 505 | PAS | 750 | 875 | 100 | 8.3 | 8.30E−27 |
| 505 | HisKA | 895 | 959 | 44.5 | 22.5 | 4.10E−10 |
| 505 | HATPase_c | 1007 | 1118 | 72.8 | 37.5 | 1.20E−18 |
| 506 | PAS_2 | 69 | 185 | 230.5 | 48 | 4.20E−66 |
| 506 | GAF | 218 | 402 | 105.4 | 45.8 | 1.90E−28 |
| 506 | Phytochrome | 413 | 592 | 421.7 | 29 | 1.20E−123 |
| 506 | PAS | 620 | 735 | 101.9 | 8.3 | 2.20E−27 |
| 506 | PAS_4 | 626 | 740 | 35.5 | 16.4 | 2.10E−07 |
| 506 | PAS | 750 | 875 | 100 | 8.3 | 8.30E−27 |
| 506 | HisKA | 895 | 959 | 44.5 | 22.5 | 4.10E−10 |
| 506 | HATPase_c | 1007 | 1118 | 72.8 | 37.5 | 1.20E−18 |
| 507 | zf-C3HC4 | 152 | 193 | 40.2 | 29.7 | 8.40E−09 |
| 508 | zf-C3HC4 | 152 | 193 | 40.2 | 29.7 | 8.40E−09 |
| 509 | CSD | 4 | 70 | 156.9 | 6.7 | 6.20E−44 |
| 510 | Pkinase | 17 | 329 | 223.2 | 116.5 | 6.80E−64 |
| 510 | Pkinase_Tyr | 17 | 329 | −72.7 | −149.3 | 6.80E−09 |
| 511 | SIS | 56 | 193 | 64.5 | 12.1 | 4.00E−16 |
| 511 | CBS | 226 | 347 | 78.5 | 25.8 | 2.50E−20 |
| 513 | BCCT | 15 | 504 | 945.1 | −372.2 | 3.30E−281 |
| 514 | Dehydrin | 1 | 93 | 40.4 | 23 | 7.30E−09 |
| 515 | Nitroreductase | 20 | 181 | 82.5 | 37 | 1.60E−21 |
| 516 | Auxin_inducible | 29 | 95 | 139.1 | 43.4 | 1.40E−38 |
| 517 | zf-CCHC | 602 | 619 | 19.6 | 19.6 | 0.00034 |
| 517 | Plus-3 | 699 | 804 | 138.8 | 52.5 | 1.70E−38 |
| 518 | U-box | 7 | 83 | 86.3 | 36.7 | 1.10E−22 |
| 519 | bZIP_1 | 273 | 333 | 45.2 | 30.3 | 2.50E−10 |
| 519 | bZIP_2 | 273 | 327 | 47.2 | 32.8 | 6.40E−11 |
| 520 | FAD_binding_3 | 80 | 438 | −92.9 | −134.3 | 3.00E−06 |
| 520 | FHA | 558 | 634 | 61.7 | 36.3 | 2.80E−15 |
| 521 | LIM | 10 | 66 | 63.1 | 36.4 | 1.00E−15 |
| 521 | LIM | 109 | 165 | 80 | 36.4 | 8.40E−21 |
| 522 | Ndr | 23 | 307 | 507.9 | −78.4 | 1.30E−149 |
| 523 | Pkinase | 81 | 374 | 221.1 | 100.3 | 2.80E−63 |
| 523 | Pkinase_Tyr | 81 | 374 | −78.4 | −126.6 | 1.60E−08 |
| 524 | SRF-TF | 9 | 59 | 121.8 | 12.8 | 2.30E−33 |
| 524 | K-box | 75 | 174 | 156.5 | 74.3 | 7.80E−44 |
| 525 | Sina | 98 | 297 | 406.3 | 16.2 | 5.10E−119 |
| 526 | HLH | 28 | 78 | 10.6 | 9 | 0.013 |
| 527 | Myb_DNA-binding | 10 | 57 | 44.7 | 29.4 | 3.50E−10 |
| 527 | Myb_DNA-binding | 63 | 108 | 52.8 | 29.4 | 1.30E−12 |
| 528 | HLH | 61 | 144 | 22.1 | 22.1 | 0.00078 |
| 529 | DUF828 | 85 | 273 | 300.8 | 11.5 | 2.80E−87 |
| 529 | PH_2 | 290 | 397 | 32.2 | −0.7 | 7.90E−07 |
| 530 | SRF-TF | 9 | 59 | 115.9 | 14.8 | 1.30E−31 |
| 530 | K-box | 74 | 173 | 138.8 | 5.9 | 1.70E−38 |
| 531 | PBP | 20 | 165 | 220.2 | −19.8 | 5.20E−63 |
| 532 | PTR2 | 97 | 499 | 485.3 | 16.2 | 8.10E−143 |
| 533 | ABC_membrane | 71 | 350 | 216 | 33.5 | 1.00E−61 |
| 533 | ABC_tran | 424 | 609 | 222.2 | 71.8 | 1.30E−63 |
| 534 | GATase_2 | 2 | 173 | −17.6 | −89.2 | 1.00E−07 |
| 534 | Asn_synthase | 227 | 460 | 297.7 | −50.9 | 2.60E−86 |
| 535 | Acetate_kinase | 25 | 420 | 605.3 | −200.3 | 6.30E−179 |
| 536 | PTA_PTB | 452 | 770 | 511.1 | −121.8 | 1.50E−150 |
| 537 | OKR_DC_1 | 20 | 491 | 143 | −45.4 | 9.50E−40 |
| 537 | OKR_DC_1_C | 485 | 584 | −4.2 | −51.6 | 1.60E−07 |
| 538 | Mito_carr | 79 | 176 | 131.9 | 23.8 | 2.10E−36 |
| 538 | Mito_carr | 184 | 281 | 107.7 | 23.8 | 4.00E−29 |
| 538 | Mito_carr | 285 | 375 | 88.8 | 23.8 | 1.90E−23 |
| 539 | AP2 | 114 | 165 | 76 | 27 | 1.40E−19 |
| 542 | 60KD_IMP | 128 | 347 | 360.6 | 7.8 | 2.90E−105 |
| 545 | eIF-5a | 86 | 155 | 133.7 | 17.4 | 6.00E−37 |
| 546 | Aa_trans | 32 | 463 | 484.6 | −127.8 | 1.40E−142 |
| 547 | zf-B_box | 1 | 45 | 38.3 | 36.1 | 3.10E−08 |
| 548 | MFS_1 | 47 | 528 | 24.1 | 24.1 | 0.00057 |
| 548 | PTR2 | 109 | 520 | 323.9 | −39.5 | 3.30E−94 |
| 549 | Homeobox | 17 | 71 | 62.8 | −2.9 | 1.30E−15 |
| 549 | HALZ | 72 | 116 | 73.7 | 20 | 6.80E−19 |
| 550 | Homeobox | 68 | 122 | 77 | 12.5 | 6.70E−20 |
| 550 | HALZ | 123 | 167 | 81.9 | 19.5 | 2.30E−21 |
| 551 | Homeobox | 68 | 122 | 77 | 12.5 | 6.70E−20 |
| 551 | HALZ | 123 | 167 | 81.9 | 19.5 | 2.30E−21 |
| 552 | Homeobox | 68 | 122 | 77 | 12.5 | 6.70E−20 |
| 552 | HALZ | 123 | 167 | 81.9 | 19.5 | 2.30E−21 |
| 553 | Homeobox | 71 | 125 | 75.9 | 52.9 | 1.40E−19 |
| 553 | HALZ | 126 | 170 | 38.4 | 19.5 | 2.90E−08 |
| 554 | PP2C | 35 | 319 | 82.4 | 44 | 1.60E−21 |
| 555 | PP2C | 46 | 328 | 119.2 | 44 | 1.40E−32 |
| 556 | PP2C | 40 | 319 | 112.1 | −25.3 | 1.90E−30 |
| 557 | Response_reg | 13 | 125 | 87.6 | 5.1 | 4.60E−23 |
| 557 | Myb_DNA-binding | 202 | 252 | 45.8 | 22.8 | 1.70E−10 |
| 558 | AP2 | 151 | 203 | 84.3 | 58.7 | 4.30E−22 |
| 560 | F-box | 51 | 106 | 29.6 | 15.3 | 1.30E−05 |
| 560 | Tub | 117 | 442 | 535 | 81.2 | 9.00E−158 |
| 561 | AA_permease | 78 | 509 | 33.9 | −118 | 6.60E−07 |
| 562 | AA_permease | 65 | 510 | 41.7 | −118 | 2.90E−09 |
| 563 | Transaldolase | 39 | 258 | 17.3 | −12.6 | 6.50E−10 |
| 564 | Acetate_kinase | 25 | 420 | 605.3 | −200.3 | 6.30E−179 |
| 565 | OKR_DC_1 | 20 | 491 | 143 | −45.4 | 9.50E−40 |
| 565 | OKR_DC_1_C | 485 | 584 | −4.2 | −51.6 | 1.60E−07 |
| 566 | Gal_Lectin | 75 | 152 | 66.5 | 27 | 1.00E−16 |
| 567 | zf-C3HC4 | 107 | 148 | 32.2 | 20.4 | 2.10E−06 |
| 568 | Aa_trans | 32 | 429 | 241.2 | −100.4 | 2.60E−69 |
| 569 | zf-C3HC4 | 85 | 135 | 36.6 | 23.4 | 9.90E−08 |
| 570 | HR_lesion | 1 | 138 | 322 | 103.1 | 1.20E−93 |
| 571 | zf-Dof | 43 | 105 | 140.8 | 27.7 | 4.30E−39 |
| 572 | zf-Dof | 43 | 105 | 140.8 | 27.7 | 4.30E−39 |
| 573 | zf-Dof | 43 | 105 | 140.8 | 27.7 | 4.30E−39 |
| 574 | zf-B_box | 1 | 46 | 30.1 | 22.6 | 9.20E−06 |
| 575 | PAS_2 | 69 | 185 | 230.5 | 48 | 4.20E−66 |
| 575 | GAF | 218 | 402 | 111.1 | 45.8 | 3.60E−30 |
| 575 | Phytochrome | 413 | 592 | 421.7 | 29 | 1.20E−123 |
| 575 | PAS | 620 | 735 | 101.9 | 8.3 | 2.20E−27 |
| 575 | PAS_4 | 626 | 740 | 35.5 | 16.4 | 2.10E−07 |
| 575 | PAS | 750 | 875 | 100 | 8.3 | 8.30E−27 |
| 575 | HisKA | 895 | 959 | 44.5 | 22.5 | 4.10E−10 |
| 575 | HATPase_c | 1007 | 1118 | 72.8 | 35.3 | 1.20E−18 |
| 576 | PAS_2 | 69 | 185 | 230.5 | 48 | 4.20E−66 |
| 576 | GAF | 218 | 402 | 111.1 | 45.8 | 3.60E−30 |
| 576 | Phytochrome | 413 | 592 | 421.7 | 29 | 1.20E−123 |
| 576 | PAS | 620 | 735 | 101.9 | 8.3 | 2.20E−27 |
| 576 | PAS_4 | 626 | 740 | 35.5 | 16.4 | 2.10E−07 |
| 576 | PAS | 750 | 875 | 100 | 8.3 | 8.30E−27 |
| 576 | HisKA | 895 | 959 | 44.5 | 22.5 | 4.10E−10 |
| 576 | HATPase_c | 1007 | 1118 | 72.8 | 35.3 | 1.20E−18 |
| 577 | PAS_2 | 69 | 185 | 230.5 | 48 | 4.20E−66 |
| 577 | GAF | 218 | 402 | 111.1 | 45.8 | 3.60E−30 |
| 577 | Phytochrome | 413 | 592 | 421.7 | 29 | 1.20E−123 |
| 577 | PAS | 620 | 735 | 101.9 | 8.3 | 2.20E−27 |
| 577 | PAS_4 | 626 | 740 | 35.5 | 16.4 | 2.10E−07 |
| 577 | PAS | 750 | 875 | 100 | 8.3 | 8.30E−27 |
| 577 | HisKA | 895 | 959 | 44.5 | 22.5 | 4.10E−10 |
| 577 | HATPase_c | 1007 | 1118 | 72.8 | 35.3 | 1.20E−18 |
| 578 | Ammonium_transp | 47 | 470 | 651.5 | −140.2 | 8.00E−193 |
| 580 | Thg1 | 1 | 255 | 283.3 | 28.8 | 5.50E−82 |
| 580 | Thg1 | 273 | 520 | 352.9 | 28.8 | 6.10E−103 |
| 581 | DUF647 | 177 | 593 | 868.5 | 29.4 | 3.80E−258 |
| 582 | AP2 | 95 | 147 | 79.8 | 52.3 | 1.00E−20 |
| 584 | Peptidase_C2 | 1696 | 1997 | 285.6 | −156.6 | 1.10E−82 |
| 584 | Calpain_III | 1999 | 2149 | −9.8 | −54.1 | 3.00E−06 |
| 585 | HSP70 | 37 | 644 | 1324.4 | 412 | 0 |
| 586 | Pkinase | 4 | 260 | 305.1 | 111.6 | 1.50E−88 |
| 586 | Pkinase_Tyr | 4 | 264 | −38.8 | −125 | 3.80E−11 |
| 587 | WRKY | 165 | 223 | 131.8 | 5.7 | 2.30E−36 |
| 587 | WRKY | 327 | 386 | 148.1 | 5.7 | 2.70E−41 |
| 588 | WRKY | 183 | 241 | 144.9 | 5.7 | 2.60E−40 |
| 588 | WRKY | 361 | 420 | 151.5 | 5.7 | 2.60E−42 |
| 589 | LysM | 103 | 148 | 20.7 | 20.1 | 0.0039 |
| 590 | LRRNT_2 | 357 | 400 | 30.5 | 18.9 | 6.80E−06 |
| 590 | LRR_1 | 431 | 452 | 11.8 | 10.9 | 1.7 |
| 590 | Pkinase_Tyr | 587 | 867 | 120.2 | −144.1 | 6.70E−33 |
| 590 | Pkinase | 595 | 867 | 114.7 | 74.8 | 3.00E−31 |
| 591 | GATase_2 | 2 | 161 | 78.2 | −89.2 | 2.90E−20 |
| 591 | Asn_synthase | 209 | 450 | 337.9 | −46 | 2.00E−98 |
| 592 | GH3 | 31 | 592 | 1167.9 | −249.6 | 0 |
| 593 | MFS_1 | 37 | 502 | 38.5 | 23.9 | 2.70E−08 |
| 593 | PTR2 | 99 | 494 | 362.6 | 91.2 | 7.30E−106 |
| 594 | Glutaredoxin | 89 | 153 | 85.3 | 17.5 | 2.20E−22 |
| 595 | Glutaredoxin | 30 | 94 | 97.7 | 55.5 | 3.90E−26 |
| 595 | Glutaredoxin | 166 | 230 | 90.6 | 55.5 | 5.30E−24 |
| 595 | Glutaredoxin | 273 | 337 | 101.9 | 55.5 | 2.10E−27 |
| 596 | Glutaredoxin | 13 | 75 | 59 | 39.4 | 1.70E−14 |
| 597 | GTP1_OBG | 212 | 377 | 230.5 | −51.4 | 4.30E−66 |
| 597 | MMR_HSR1 | 379 | 502 | 136.2 | 38 | 1.10E−37 |
| 597 | DUF1967 | 585 | 654 | 104.9 | 10.4 | 2.80E−28 |
| 598 | HLH | 70 | 120 | 39.8 | 10.5 | 1.10E−08 |
| 600 | LRR_1 | 116 | 138 | 14.5 | 11.1 | 0.44 |
| 600 | LRR_1 | 140 | 162 | 12.9 | 11.1 | 1 |
| 600 | LRR_1 | 164 | 186 | 15.2 | 11.1 | 0.27 |
| 600 | LRR_1 | 188 | 209 | 13.7 | 11.1 | 0.73 |
| 600 | Pkinase_Tyr | 497 | 764 | 39 | −37.1 | 2.50E−16 |
| 601 | DEAD_2 | 72 | 256 | 297.9 | 29.3 | 2.20E−86 |
| 601 | DUF1227 | 267 | 413 | 296.6 | 165.1 | 5.20E−86 |
| 602 | NUDIX | 32 | 173 | 60.1 | 2.2 | 8.60E−15 |
| 604 | zf-C3HC4 | 27 | 67 | 31.5 | 19.1 | 3.40E−06 |
| 605 | SRF-TF | 9 | 59 | 116 | 14.8 | 1.20E−31 |
| 605 | K-box | 75 | 176 | 99.8 | 5.9 | 9.20E−27 |
| 606 | SRF-TF | 9 | 59 | 109.5 | 14.8 | 1.10E−29 |
| 606 | K-box | 74 | 173 | 96.9 | 5.9 | 7.20E−26 |
| 607 | Aa_trans | 58 | 464 | 166.2 | 133 | 9.40E−47 |
| 608 | Aa_trans | 26 | 463 | 373 | −127.8 | 5.50E−109 |
| 612 | Myb_DNA-binding | 20 | 67 | 46.2 | 26.4 | 1.30E−10 |
| 612 | Myb_DNA-binding | 73 | 118 | 44.6 | 26.4 | 3.80E−10 |
| 613 | Globin | 14 | 123 | 64.6 | −6.9 | 3.80E−16 |
| 613 | FAD_binding_6 | 177 | 276 | 42.5 | −2.2 | 1.60E−09 |
| 613 | NAD_binding_1 | 287 | 396 | 88.4 | −1.7 | 2.60E−23 |
| 614 | Globin | 6 | 103 | 61.7 | 9.9 | 2.80E−15 |
| 614 | FAD_binding_6 | 156 | 262 | 42.7 | −4.5 | 1.40E−09 |
| 614 | NAD_binding_1 | 271 | 379 | 66.1 | −3.4 | 1.30E−16 |

TABLE 10
Pfam module annotation

| PEP SEQ | | |
|---|---|---|
| ID NO | Pfam domain module | Position |
| 308 | Aminotran_1_2 | 69-470 |
| 309 | Homeobox::HALZ | 65-119::120-164 |
| 310 | bZIP_2 | 108-162 |
| 312 | Peptidase_S10 | 39-504 |
| 313 | SBP | 103-181 |
| 315 | GDPD | 436-728 |
| 317 | DUF1292 | 111-180 |
| 318 | Aminotran_1_2 | 127-473 |
| 319 | FAD_binding_4::Lact-deh- | 50-191::279-570 |
| | memb | |
| 320 | Fe-ADH | 25-365 |
| 321 | AlaDh_PNT_N::AlaDh_PNT_C | 4-135::147-302 |
| 322 | Thiolase_N::Thiolase_C | 14-277::284-406 |
| 323 | Thiolase_N::Thiolase_C | 14-277::284-406 |
| 324 | SOUL | 1-150 |
| 325 | AA_kinase | 98-373 |
| 326 | AA_kinase | 10-285 |
| 327 | AA_kinase::NAD_binding_3:: | 98-374::552-687::695-891 |
| | Homoserine_dh | |
| 328 | HLH | 204-253 |
| 329 | HLH | 204-253 |
| 330 | HLH | 258-307 |
| 331 | HLH | 258-307 |
| 332 | Thioredoxin::Glutaredoxin:: | 7-105::169-233::298- |
| | Glutaredoxin::Glutaredoxin | 362::405-469 |
| 334 | Pkinase | 143-405 |
| 335 | Gln-synt_N::Gln-synt_C | 17-97::103-355 |
| 336 | Pkinase | 143-405 |
| 337 | MethyltransfD12 | 23-264 |
| 338 | bZIP_1::MethyltransfD12 | 87-150::191-432 |
| 339 | zf-B_box::MethyltransfD12 | 1-47::248-489 |
| 340 | TPP_enzyme_N::TPP_enzyme_M:: | 2-177::193-325::380-517 |
| | TPP_enzyme_C | |
| 341 | RolB_RolC::Amino_oxidase | 11-197::246-729 |
| 342 | Amino_oxidase | 55-532 |
| 343 | NIR_SIR_ferr::NIR_SIR::NIR_SIR_ferr | 131-201::209-369::390-459 |
| 345 | DEAD::Helicase_C | 121-290::359-435 |
| 347 | LRRNT_2::LRR_1::LRR_1::LRR_1:: | 38-79::108-130::132- |
| | LRR_1::LRR_1::LRR_1:: | 154::156-178::180-201::228- |
| | LRR_1::LRR_1::LRR_1::LRR_1:: | 250::252-274::299-321::347- |
| | LRR_1::LRR_1::LRR_1:: | 369::419-441::443-465::467- |
| | Pkinase | 489::491-513::539-560::662- |
| | | 927 |
| 348 | Homeobox::HALZ | 118-172::173-217 |
| 349 | Homeobox::HALZ | 139-193::194-238 |
| 350 | WD40::WD40 | 560-599::646-683 |
| 351 | C4dic_mal_tran | 199-465 |
| 352 | Peroxidase | 85-320 |
| 353 | Peroxidase | 85-320 |
| 354 | LisH::WD40::WD40::WD40:: | 7-33::185-223::244-282::285- |
| | WD40::WD40::WD40::WD40 | 323::327-364::368-406::410- |
| | | 457::461-501 |
| 355 | bZIP_2::bZIP_1 | 325-379::334-386 |
| 356 | FAD_binding_4::ALO | 107-242::266-581 |
| 357 | NAM | 69-200 |
| 358 | Ribosomal_L21p | 136-232 |
| 359 | BTB | 192-301 |
| 360 | RWP-RK::PB1 | 104-155::325-408 |
| 361 | Ammonium_transp | 29-451 |
| 362 | BTB | 192-301 |
| 363 | OPT | 85-744 |
| 364 | DUF506 | 14-238 |
| 366 | FTCD_N | 10-202 |
| 367 | FTCD_N | 7-199 |
| 368 | SSF | 44-461 |
| 369 | Aniinotran_5 | 68-433 |
| 370 | ABC_tran | 67-262 |
| 371 | Monooxygenase_B | 39-418 |
| 372 | PMEI | 34-191 |
| 374 | CCT | 141-185 |
| 375 | PHD::SET | 82-131::361-496 |
| 376 | AP2 | 111-163 |
| 377 | bZIP_2 | 390-444 |
| 378 | AP2 | 24-75 |
| 379 | A_thal_3526 | 17-70 |
| 380 | AP2 | 24-75 |
| 381 | Clp_N::Clp_N::AAA::AAA_2:: | 17-67::96-146::202-396::597- |
| | ClpB_D2-small | 762::768-857 |
| 382 | eRF1_1::eRF1_2::eRF1_3 | 1-131::136-268::271-370 |
| 387 | zf-CCCH | 113-138 |
| 388 | zf-B_box::zf-B_box::CCT | 1-47::48-90::350-394 |
| 389 | HLH | 128-178 |
| 390 | Arginosuc_synth | 100-446 |
| 391 | Glutaredoxin | 205-279 |
| 392 | AsnA | 3-246 |
| 392 | NAD_binding_1 | 221-337 |
| 394 | cNMP_binding::Crp | 39-131::186-217 |
| 395 | PLAC8 | 71-166 |
| 396 | HSP20 | 42-148 |
| 397 | Myb_DNA- | 14-61::67-112 |
| | binding::Myb_DNA-binding | |
| 398 | ATP-sulfurylase | 136-462 |
| 399 | WRKY | 300-360 |
| 400 | Asp | 106-439 |
| 401 | Gln-synt_N::Gln-synt_C | 42-122::141-444 |
| 402 | GATase_2::Glu_syn_central:: | 27-397::477-773::830- |
| | Glu_synthase::GXGXG | 1221::1298-1493 |
| 403 | Form_Nir_trans | 87-343 |
| 406 | NAD_binding_2 | 51-213 |
| 407 | NAD_binding_2 | 1-162 |
| 408 | Glutaredoxin | 39-103 |
| 409 | Glutaredoxin | 59-123 |
| 410 | Homeobox::HALZ | 139-193::194-238 |
| 411 | Homeobox::HALZ | 137-193::194-238 |
| 412 | Homeobox::HALZ | 139-193::194-238 |
| 414 | CCT | 228-272 |
| 415 | MBD | 25-144 |
| 416 | MBD | 21-94 |
| 417 | Pkinase | 12-263 |
| 418 | SRF-TF | Mar-53 |
| 419 | SRF-TF | Nov-65 |
| 420 | eIF-5a | 85-154 |
| 421 | Homeobox::START | 33-89-236-457 |
| 422 | Homeobox::START | 33-89::236-457 |
| 423 | Homeobox::START | 33-89::236-457 |
| 424 | Homeobox::START | 33-89::236-457 |
| 425 | Arginase | 61-336 |
| 427 | CBFB_NFYA | 147-203 |
| 428 | CBFB_NFYA | 148-204 |
| 429 | CBFB_NFYA | 93-149 |
| 430 | CBFB_NFYA | 70-126 |
| 431 | CBFB_NFYA | 73-129 |
| 432 | PAS_2::GAF::Phytochrome:: | 103-219::252-433::444- |
| | PAS::PAS::HisKA::HATPase_c | 623::654-770::785-907::927- |
| | | 991::1039-1152 |
| 433 | HMG_box | 44-113 |
| 434 | SPRY | 77-224 |
| 435 | SPRY | 77-224 |
| 436 | RRM_1::RRM_1 | 4-68::97-160 |
| 437 | RRM_1::RRM_1 | 4-68::97-160 |
| 438 | RRM_1::zf-CCHC | 4-69::85-102 |
| 439 | RRM_1::zf-CCHC | 4-69::85-102 |
| 440 | RRM_1::zf-CCHC | 4-69::86-103 |
| 441 | RRM_1::zf-CCHC | 4-69::86-103 |
| 442 | MMR_HSR1::KH_2 | 119-240::345-409 |
| 443 | MMR_HSR1::KH_2 | 119-240::345-409 |
| 444 | MMR_HSR1::KH_2 | 119-240::345-409 |
| 445 | zf-C3HC4 | 152-193 |
| 446 | zf-C3HC4 | 152-193 |
| 447 | zf-C3HC4 | 152-193 |
| 448 | zf-C3HC4 | 152-193 |
| 450 | 2-Hacid_dh_C | 113-292 |
| 451 | AlaDh_PNT_N::AlaDh_PNT_C | 4-136::148-303 |
| 452 | AlaDh_PNT_N::AlaDh_PNT_C | 4-136::148-303 |
| 453 | tRNA_synt_1c_R1::tRNA_synt_1c_R2:: | 14-171::172-264::271- |
| | tRNA-synt_1c::tRNA-synt_1c_C | 577::579-770 |
| 454 | SBP_bac_3 | 75-296 |
| 455 | Gln-synt_N::Gln-synt_C | 17-97::103-355 |
| 456 | bZIP_1 | 76-139 |
| 457 | bZIP_1 | 76-139 |
| 458 | BPD_transp_2 | 5-354 |
| 459 | BPD_transp_2 | 82-431 |
| 460 | Asp_decarbox | 2-119 |
| 462 | Glt_symporter | 4-370 |
| 463 | Glt_symporter | 4-370 |
| 464 | SBP_bac_3 | 75-296 |
| 465 | SBP_bac_3 | 75-296 |
| 467 | AlaDh_PNT_N::AlaDh_PNT_C | 4-136::148-302 |
| 468 | Nitroreductase | 20-181 |
| 469 | Acid_phosphat_B | 1-179 |
| 470 | bZIP_1 | 76-139 |
| 471 | Ubiquitin | 22-92 |
| 472 | LIM | 172-228 |
| 473 | LIM | 147-203 |
| 474 | zf-C3HC4 | 197-237 |
| 475 | RCC1::RCC1::RCC1 | 95-145::266-315::350-398 |
| 476 | Sulfate_transp::STAS | 198-510::533-652 |
| 477 | RCC1::RCC1::RCC1 | 95-145::266-315::350-398 |
| 479 | Glyco_transf_20::Trehalose_PPase | 58-544::593-829 |
| 480 | RRM_1 | 19-89 |
| 481 | Asp | 89-438 |
| 482 | Ank::Ank::Ank::Ank::Ank::Ank:: | 44-81::83-112::118-150::152- |
| | Ank::Ank | 184::186-218::220-252::254- |
| | | 287::288-320 |
| 484 | DSPc | 105-240 |
| 485 | Usp | 8-162 |
| 486 | U-box | 17-94 |
| 487 | U-box | Oct-84 |
| 493 | MIT::AAA::Vps4_C | 5-73::169-353::368-433 |
| 494 | AP2 | 80-132 |
| 495 | Ssl1::C1_4 | 37-292::374-422 |
| 496 | POX::Homeobox | 213-346::396-454 |
| 497 | POX::Homeobox | 214-350::404-462 |
| 498 | Dimerisation::Methyltransf_2 | 34-82::91-342 |
| 499 | DUF640 | 1-160 |
| 500 | p450 | 2-479 |
| 501 | PTR2 | 109-520 |
| 502 | Pkinase::NAF | 16-269::311-372 |
| 503 | Acyl-ACP_TE | 92-367 |
| 504 | PAS_2::GAF::Phytochrome:: | 69-185::218-402::413- |
| | PAS::PAS::HisKA::HATPase_c | 592::620-735::750-875::895- |
| | | 959::1007-1118 |
| 505 | PAS_2::GAF::Phytochrome:: | 69-185::218-402::413- |
| | PAS::PAS::HisKA::HATPase_c | 592::620-735::750-875::895- |
| | | 959::1007-1118 |
| 506 | PAS_2::GAF::Phytochrome:: | 69-185::218-402::413- |
| | PAS::PAS::HisKA::HATPase_c | 592::620-735::750-875::895- |
| | | 959::1007-1118 |
| 507 | zf-C3HC4 | 152-193 |
| 508 | zf-C3HC4 | 152-193 |
| 509 | CSD | Apr-70 |
| 510 | Pkinase | 17-329 |
| 511 | SIS::CBS | 56-193::226-347 |
| 513 | BCCT | 15-504 |
| 514 | Dehydrin | Jan-93 |
| 515 | Nitroreductase | 20-181 |
| 516 | Auxin_inducible | 29-95 |
| 517 | zf-CCHC::Plus-3 | 602-619::699-804 |
| 518 | U-box | Jul-83 |
| 519 | bZIP_2 | 273-327 |
| 520 | FAD_binding_3::FHA | 80-438::558-634 |
| 521 | LIM::LIM | 10-66::109-165 |
| 522 | Ndr | 23-307 |
| 523 | Pkinase | 81-374 |
| 524 | SRF-TF::K-box | 9-59::75-174 |
| 525 | Sina | 98-297 |
| 526 | HLH | 28-78 |
| 527 | Myb_DNA- | 10-57::63-108 |
| | binding::Myb_DNA-binding | |
| 528 | HLH | 61-144 |
| 529 | DUF828::PH_2 | 85-273::290-397 |
| 530 | SRF-TF::K-box | 9-59::74-173 |
| 531 | PBP | 20-165 |
| 532 | PTR2 | 97-499 |
| 533 | ABC_membrane::ABC_tran | 71-350::424-609 |
| 534 | GATase_2::Asn_synthase | 2-173::227-460 |
| 535 | Acetate_kinase | 25-420 |
| 536 | PTA_PTB | 452-770 |
| 537 | OKR_DC_1::OKR_DC_1_C | 20-491::485-584 |
| 538 | Mito_carr::Mito_carr::Mito_carr | 79-176::184-281::285-375 |
| 539 | AP2 | 114-165 |
| 542 | 60KD_IMP | 128-347 |
| 545 | eIF-5a | 86-155 |
| 546 | Aa_trans | 32-463 |
| 547 | zf-B_box | Jan-45 |
| 548 | PTR2 | 109-520 |
| 549 | Homeobox::HALZ | 17-71::72-116 |
| 550 | Homeobox::HALZ | 68-122::123-167 |
| 551 | Homeobox::HALZ | 68-122::123-167 |
| 552 | Homeobox::HALZ | 68-122::123-167 |
| 553 | Homeobox::HALZ | 71-125::126-170 |
| 554 | PP2C | 35-319 |
| 555 | PP2C | 46-328 |
| 556 | PP2C | 40-319 |
| 557 | Response_reg::Myb_DNA- | 13-125::202-252 |
| | binding | |
| 558 | AP2 | 151-203 |
| 560 | F-box::Tub | 51-106::117-442 |
| 561 | AA_permease | 78-509 |
| 562 | AA_permease | 65-510 |
| 563 | Transaldolase | 39-258 |
| 564 | Acetate_kinase | 25-420 |
| 565 | OKR_DC_1::OKR_DC_1_C | 20-491::485-584 |
| 566 | Gal_Lectin | 75-152 |
| 567 | zf-C3HC4 | 107-148 |
| 568 | Aa_trans | 32-429 |
| 569 | zf-C3HC4 | 85-135 |
| 570 | HR_lesion | 1-138 |
| 571 | zf-Dof | 43-105 |
| 572 | zf-Dof | 43-105 |
| 573 | zf-Dof | 43-105 |
| 574 | zf-B_box | Jan-46 |
| 575 | PAS_2::GAF::Phytochrome:: | 69-185::218-402::413- |
| | PAS::PAS::HisKA::HATPase_c | 592::620-735::750-875::895- |
| | | 959::1007-1118 |
| 576 | PAS_2::GAF::Phytochrome:: | 69-185::218-402::413- |
| | PAS::PAS::HisKA::HATPase_c | 592::620-735::750-875::895- |
| | | 959::1007-1118 |
| 577 | PAS_2::GAP::Phytochrome:: | 69-185::218-402::413- |
| | PAS::PAS::HisKA::HATPase_c | 592::620-735::750-875::895- |
| | | 959::1007-1118 |
| 578 | Ammonium_transp | 47-470 |
| 580 | Thg1::Thg1 | 1-255::273-520 |
| 581 | DUF647 | 177-593 |
| 582 | AP2 | 95-147 |
| 584 | Peptidase_C2::Calpain_III | 1696-1997::1999-2149 |
| 585 | HSP70 | 37-644 |
| 586 | Pkinase | 4-260 |
| 587 | WRKY::WRKY | 165-223::327-386 |
| 588 | WRKY::WRKY | 183-241::361-420 |
| 589 | LysM | 103-148 |
| 590 | LRRNT_2::LRR_1::Pkinase_Tyr | 357-400::431-452::587-867 |
| 591 | GATase_2::Asn_synthase | 2-161::209-450 |
| 592 | GH3 | 31-592 |
| 593 | PTR2 | 99-494 |
| 594 | Glutaredoxin | 89-153 |
| 595 | Glutaredoxin::Glutaredoxin:: | 30-94::166-230::273-337 |
| | Glutaredoxin | |
| 596 | Glutaredoxin | 13-75 |
| 597 | GTP1_OBG::MMR_HSR1::DUF1967 | 212-377::379-502::585-654 |
| 598 | HLH | 70-120 |
| 600 | LRR_1::LRR_1::LRR_1::LRR_1:: | 116-138::140-162::164- |
| | Pkinase_Tyr | 186::188-209::497-764 |
| 601 | DEAD_2::DUF1227 | 72-256::267-413 |
| 602 | NUDIX | 32-173 |
| 604 | zf-C3HC4 | 27-67 |
| 605 | SRF-TF::K-box | 9-59::75-176 |
| 606 | SRF-TF::K-box | 9-59::74-173 |
| 607 | Aa_trans | 58-464 |
| 608 | Aa_trans | 26-463 |
| 612 | Myb_DNA- | 20-67::73-118 |
| | binding::Myb_DNA-binding | |
| 613 | Globin::FAD_binding_6::NAD_binding_1 | 14-123::177-276::287-396 |
| 614 | Globin::FAD_binding_6::NAD_binding_1 | 6-103::156-262::271-379 |

TABLE 11
Description of Pfam domains

| | Accession | Gathering | |
|---|---|---|---|
| Pfam domain name | number | cutoff | Domain description |
| 14-3-3 | PF00244.12 | 25.0000; | 14-3-3 protein |
| 2-Hacid_dh | PF00389.22 | 11.2000; | D-isomer specific 2-hydroxyacid |
| | | | dehydrogenase, catalytic domain |
| 2-Hacid_dh_C | PF02826.11 | −78.6000; | D-isomer specific 2-hydroxyacid |
| | | | dehydrogenase, NAD binding domain |
| 3-alpha | PF03475.6 | 25.0000; | 3-alpha domain |
| 3HCDH_N | PF02737.10 | −78.7000; | 3-hydroxyacyl-CoA dehydrogenase, NAD |
| | | | binding domain |
| 60KD_IMP | PF02096.12 | −95.0000; | 60 Kd inner membrane protein |
| AAA | PF00004.21 | 12.3000; | ATPase family associated with various cellular |
| | | | activities (AAA) |
| AAA_2 | PF07724.6 | −5.0000; | ATPase family associated with various cellular |
| | | | activities (AAA) |
| AAA_5 | PF07728.6 | 4.0000; | ATPase family associated with various cellular |
| | | | activities (AAA) |
| AA_kinase | PF00696.20 | −40.0000; | Amino acid kinase family |
| AA_permease | PF00324.13 | −120.8000; | Amino acid permease |
| ABC_membrane | PF00664.15 | 19.4000; | ABC transporter transmembrane region |
| ABC_membrane_2 | PF06472.7 | −94.9000; | ABC transporter transmembrane region 2 |
| ABC_tran | PF00005.19 | 9.5000; | ABC transporter |
| ACP_syn_III_C | PF08541.2 | −20.5000; | 3-Oxoacyl-[acyl-carrier-protein (ACP)] |
| | | | synthase III C terminal |
| ACT | PF01842.17 | 0.7000; | ACT domain |
| AFG1_ATPase | PF03969.8 | −177.5000; | AFG1-like ATPase |
| ALO | PF04030.6 | −99.7000; | D-arabinono-1,4-lactone oxidase |
| AMP-binding | PF00501.20 | 0.0000; | AMP-binding enzyme |
| AP2 | PF00847.12 | 21.7000; | AP2 domain |
| APH | PF01636.15 | −1.3000; | Phosphotransferase enzyme family |
| APS_kinase | PF01583.12 | 25.0000; | Adenylylsulphate kinase |
| ATP-sulfurylase | PF01747.9 | −163.3000; | ATP-sulfurylase |
| A_thal_3526 | PF09713.2 | 25.0000; | Plant protein 1589 of unknown function |
| | | | (A_thal_3526) |
| Aa_trans | PF01490.10 | −128.4000; | Transmembrane amino acid transporter protein |
| Abhydrolase_1 | PF00561.12 | 10.3000; | alpha/beta hydrolase fold |
| Abi | PF02517.8 | −5.0000; | CAAX amino terminal protease family |
| Acetate_kinase | PF00871.9 | −206.2000; | Acetokinase family |
| Acetyltransf_1 | PF00583.16 | 17.8000; | Acetyltransferase (GNAT) family |
| Acid_phosphat_B | PF03767.6 | −36.0000; | HAD superfamily, subfamily IIIB (Acid |
| | | | phosphatase) |
| Actin | PF00022.11 | −144.0000; | Actin |
| Acyl-ACP_TE | PF01643.9 | −106.0000; | Acyl-ACP thioesterase |
| AdoHcyase_NAD | PF00670.13 | −23.6000; | S-adenosyl-L-homocysteine hydrolase, NAD |
| | | | binding domain |
| AlaDh_PNT_C | PF01262.13 | −43.4000; | Alanine dehydrogenase/PNT, C-terminal |
| | | | domain |
| AlaDh_PNT_N | PF05222.7 | −11.7000; | Alanine dehydrogenase/PNT, N-terminal |
| | | | domain |
| Ala_racemase_N | PF01168.12 | −34.3000; | Alanine racemase, N-terminal domain |
| Alliinase_C | PF04864.5 | −233.5000; | Allinase |
| Amidase | PF01425.13 | −133.0000; | Amidase |
| Amidohydro_1 | PF01979.12 | −37.4000; | Amidohydrolase family |
| Amino_oxidase | PF01593.16 | −11.4000; | Flavin containing amine oxidoreductase |
| Aminotran_1_2 | PF00155.13 | −57.5000; | Aminotransferase class I and II |
| Aminotran_3 | PF00202.13 | −206.1000; | Aminotransferase class-III |
| Aminotran_5 | PF00266.11 | −164.4000; | Aminotransferase class-V |
| Ammonium_transp | PF00909.13 | −144.0000; | Ammonium Transporter Family |
| Ank | PF00023.22 | 0.0000; | Ankyrin repeat |
| Anth_synt_I_N | PF04715.5 | 25.0000; | Anthranilate synthase component I, N terminal |
| | | | region |
| ApbA | PF02558.8 | −16.2000; | Ketopantoate reductase PanE/ApbA |
| Arginase | PF00491.13 | −48.2000; | Arginase family |
| Arginosuc_synth | PF00764.11 | −236.3000; | Arginosuccinate synthase |
| Arm | PF00514.15 | 17.0000; | Armadillo/beta-catenin-like repeat |
| AsnA | PF03590.7 | 25.0000; | Aspartate-ammonia ligase |
| Asn_synthase | PF00733.13 | −52.8000; | Asparagine synthase |
| Asp | PF00026.15 | −153.8000; | Eukaryotic aspartyl protease |
| Asp_decarbox | PF02261.8 | −44.4000; | Aspartate decarboxylase |
| Auxin_inducible | PF02519.6 | 25.0000; | Auxin responsive protein |
| BAG | PF02179.8 | 25.0000; | BAG domain |
| BAH | PF01426.10 | 7.0000; | BAH domain |
| BCCT | PF02028.9 | −386.1000; | BCCT family transporter |
| BNR | PF02012.12 | 18.0000; | BNR/Asp-box repeat |
| BPD_transp_1 | PF00528.14 | −6.6000; | Binding-protein-dependent transport system |
| | | | inner membrane component |
| BPD_transp_2 | PF02653.8 | −66.1000; | Branched-chain amino acid transport system/ |
| | | | permease component |
| BSD | PF03909.9 | 26.4000; | BSD domain |
| BTB | PF00651.23 | 6.2000; | BTB/POZ domain |
| Bac_DnaA | PF00308.10 | −108.0000; | Bacterial dnaA protein |
| BenE | PF03594.5 | −283.2000; | Benzoate membrane transport protein |
| Beta_elim_lyase | PF01212.13 | −110.3000; | Beta-eliminating lyase |
| Big_2 | PF02368.10 | 0.0000; | Bacterial Ig-like domain (group 2) |
| C1_1 | PF00130.14 | 11.4000; | Phorbol esters/diacylglycerol binding domain |
| | | | (C1 domain) |
| C1_4 | PF07975.4 | 25.0000; | TFIIH C1-like domain |
| C2 | PF00168.22 | 3.7000; | C2 domain |
| C4dic_mal_tran | PF03595.9 | −43.6000; | C4-dicarboxylate transporter/malic acid |
| | | | transport protein |
| CBFB_NFYA | PF02045.7 | 25.0000; | CCAAT-binding transcription factor (CBF- |
| | | | B/NF-YA) subunit B |
| CBS | PF00571.20 | 19.5000; | CBS domain pair |
| CCT | PF06203.6 | 25.0000; | CCT motif |
| CDC48_2 | PF02933.9 | 25.0000; | Cell division protein 48 (CDC48), domain 2 |
| CDC48_N | PF02359.10 | −2.0000; | Cell division protein 48 (CDC48), N-terminal |
| | | | domain |
| CDP-OH_P_transf | PF01066.13 | 0.0000; | CDP-alcohol phosphatidyltransferase |
| CS | PF04969.8 | 8.6000; | CS domain |
| CSD | PF00313.14 | −0.3000; | ‘Cold-shock’ DNA-binding domain |
| CW_binding_2 | PF04122.4 | 0.0000; | Putative cell wall binding repeat 2 |
| Calpain_III | PF01067.14 | −57.0000; | Calpain large subunit, domain III |
| Carbpep_Y_N | PF05388.3 | 2.5000; | Carboxypeptidase Y pro-peptide |
| CbiC | PF02570.7 | 25.0000; | Precorrin-8X methylmutase |
| Chal_sti_synt_C | PF02797.7 | −6.1000; | Chalcone and stilbene synthases, C-terminal |
| | | | domain |
| Chal_sti_synt_N | PF00195.11 | −71.3000; | Chalcone and stilbene synthases, N-terminal |
| | | | domain |
| CheW | PF01584.11 | 25.0000; | CheW-like domain |
| Chorismate_bind | PF00425.10 | −153.0000; | chorismate binding enzyme |
| ClpB_D2-small | PF10431.1 | 21.5000; | C-terminal, D2-small domain, of ClpB protein |
| Clp_N | PF02861.12 | 0.0000; | Clp amino terminal domain |
| Cna_B | PF05738.5 | 7.0000; | Cna protein B-type domain |
| CpcD | PF01383.13 | 3.5000; | CpcD/allophycocyanin linker domain |
| Crp | PF00325.12 | 20.7000; | Bacterial regulatory proteins, crp family |
| Cupin_2 | PF07883.3 | 19.4000; | Cupin domain |
| Cupin_4 | PF08007.4 | −112.1000; | Cupin superfamily protein |
| Cys_Met_Meta_PP | PF01053.12 | −278.4000; | Cys/Met metabolism PLP-dependent enzyme |
| Cytidylate_kin | PF02224.10 | −51.6000; | Cytidylate kinase |
| Cytochrom_C | PF00034.13 | 11.7000; | Cytochrome c |
| Cytokin-bind | PF09265.2 | −143.4000; | Cytokinin dehydrogenase 1, FAD and cytokinin |
| | | | binding |
| DAO | PF01266.16 | −34.9000; | FAD dependent oxidoreductase |
| DEAD | PF00270.21 | 7.2000; | DEAD/DEAH box helicase |
| DEAD_2 | PF06733.7 | 5.2000; | DEAD_2 |
| DHC_N1 | PF08385.4 | −162.0000; | Dynein heavy chain, N-terminal region 1 |
| DHC_N2 | PF08393.5 | −204.1000; | Dynein heavy chain, N-terminal region 2 |
| DHQ_synthase | PF01761.12 | −119.7000; | 3-dehydroquinate synthase |
| DIL | PF01843.11 | 25.0000; | DIL domain |
| DNA_pol_lambd_f | PF10391.1 | 25.0000; | Fingers domain of DNA polymerase lambda |
| DPPIV_N | PF00930.13 | −124.2000; | Dipeptidyl peptidase IV (DPP IV) N-terminal |
| | | | region |
| DRTGG | PF07085.4 | −5.3000; | DRTGG domain |
| DSPc | PF00782.12 | −21.8000; | Dual specificity phosphatase, catalytic domain |
| DUF1227 | PF06777.3 | −22.0000; | Protein of unknown function (DUF1227) |
| DUF1289 | PF06945.5 | 25.0000; | Protein of unknown function (DUF1289) |
| DUF1292 | PF06949.3 | 13.2000; | Protein of unknown function (DUF1292) |
| DUF1348 | PF07080.3 | 25.0000; | Protein of unknown function (DUF1348) |
| DUF1513 | PF07433.3 | −172.5000; | Protein of unknown function (DUF1513) |
| DUF1644 | PF07800.4 | −15.6000; | Protein of unknown function (DUF1644) |
| DUF1685 | PF07939.3 | 25.0000; | Protein of unknown function (DUF1685) |
| DUF1715 | PF08215.4 | 25.0000; | Eukaryotic domain of unknown function |
| | | | (DUF1715) |
| DUF1933 | PF09147.2 | 25.0000; | Domain of unknown function (DUF1933) |
| DUF1967 | PF09269.3 | 9.0000; | Domain of unknown function (DUF1967) |
| DUF1985 | PF09331.3 | 11.9000; | Domain of unknown function (DUF1985) |
| DUF442 | PF04273.5 | −20.4000; | Putative phosphatase (DUF442) |
| DUF506 | PF04720.4 | 25.0000; | Protein of unknown function (DUF506) |
| DUF537 | PF04396.6 | −4.6000; | Protein of unknown function, DUF537 |
| DUF538 | PF04398.4 | −24.3000; | Protein of unknown function, DUF538 |
| DUF547 | PF04784.6 | −24.7000; | Protein of unknown function, DUF547 |
| DUF568 | PF04526.5 | 25.0000; | Protein of unknown function (DUF568) |
| DUF588 | PF04535.4 | 25.0000; | Domain of unknown function (DUF588) |
| DUF6 | PF00892.12 | 21.7000; | Integral membrane protein DUF6 |
| DUF640 | PF04852.4 | −18.1000; | Protein of unknown function (DUF640) |
| DUF647 | PF04884.6 | 25.0000; | Protein of unknown function, DUF647 |
| DUF828 | PF05703.3 | −53.1000; | Plant protein of unknown function (DUF828) |
| DUF836 | PF05768.6 | 2.0000; | Glutaredoxin-like domain (DUF836) |
| DZC | PF08381.3 | 15.3000; | Disease resistance/zinc finger/chromosome |
| | | | condensation-like region |
| DapB_N | PF01113.12 | −20.7000; | Dihydrodipicolinate reductase, N-terminus |
| DbpA | PF03880.7 | 16.6000; | DbpA RNA binding domain |
| DegT_DnrJ_EryC1 | PF01041.9 | −161.3000; | DegT/DnrJ/EryC1/StrS aminotransferase family |
| Dehydrin | PF00257.11 | −4.4000; | Dehydrin |
| Dimerisation | PF08100.3 | 25.0000; | Dimerisation domain |
| DnaJ | PF00226.23 | −8.0000; | DnaJ domain |
| Dockerin_1 | PF00404.10 | 24.1000; | Dockerin type I repeat |
| DoxX | PF07681.4 | 0.3000; | DoxX |
| DpmII | PF04556.4 | 25.0000; | DpmII restriction endonuclease |
| Dynein_heavy | PF03028.7 | −455.0000; | Dynein heavy chain |
| EAL | PF00563.12 | −16.4000; | EAL domain |
| EFG_C | PF00679.16 | 6.0000; | Elongation factor G C-terminus |
| EFG_IV | PF03764.10 | 25.0000; | Elongation factor G, domain IV |
| EIN3 | PF04873.5 | −192.4000; | Ethylene insensitive 3 |
| ENT | PF03735.6 | 14.7000; | ENT domain |
| Exo_endo_phos | PF03372.15 | 11.0000; | Endonuclease/Exonuclease/phosphatase family |
| Exostosin | PF03016.7 | −53.2000; | Exostosin family |
| ExsB | PF06508.5 | −91.9000; | ExsB |
| F-box | PF00646.25 | 14.7000; | F-box domain |
| F420_oxidored | PF03807.9 | −34.5000; | NADP oxidoreductase coenzyme F420- |
| | | | dependent |
| FAD-oxidase_C | PF02913.11 | −36.0000; | FAD linked oxidases, C-terminal domain |
| FAD_binding_1 | PF00667.12 | −79.0000; | FAD binding domain |
| FAD_binding_3 | PF01494.11 | −136.6000; | FAD binding domain |
| FAD_binding_4 | PF01565.15 | −8.1000; | FAD binding domain |
| FAD_binding_5 | PF00941.13 | −57.4000; | FAD binding domain in molybdopterin |
| | | | dehydrogenase |
| FAD_binding_6 | PF00970.16 | −4.9000; | Oxidoreductase FAD-binding domain |
| FAD_binding_8 | PF08022.4 | −10.4000; | FAD-binding domain |
| FAD_binding_9 | PF08021.3 | −22.9000; | Siderophore-interacting FAD-binding domain |
| FA_desaturase | PF00487.16 | 0.2000; | Fatty acid desaturase |
| FCD | PF07729.4 | 29.0000; | FCD domain |
| FHA | PF00498.18 | 25.0000; | FHA domain |
| FLYWCH | PF04500.8 | 12.8000; | FLYWCH zinc finger domain |
| FMN_dh | PF01070.10 | −203.2000; | FMN-dependent dehydrogenase |
| FTCD | PF02971.6 | 25.0000; | Formiminotransferase domain |
| FTCD_C | PF04961.4 | −69.6000; | Formiminotransferase-cyclodeaminase |
| FTCD_N | PF07837.4 | −110.1000; | Formiminotransferase domain, N-terminal |
| | | | subdomain |
| FYRC | PF05965.6 | 17.1000; | F/Y rich C-terminus |
| FYRN | PF05964.6 | 16.3000; | F/Y-rich N-terminus |
| FYVE | PF01363.13 | 3.9000; | FYVE zinc finger |
| F_bP_aldolase | PF01116.12 | −157.6000; | Fructose-bisphosphate aldolase class-II |
| Fe-ADH | PF00465.11 | −189.8000; | Iron-containing alcohol dehydrogenase |
| Fe—S_biosyn | PF01521.12 | −0.4000; | Iron-sulphur cluster biosynthesis |
| FeoB_C | PF07664.4 | 16.6000; | Ferrous iron transport protein B C terminus |
| FeoB_N | PF02421.10 | −0.4000; | Ferrous iron transport protein B |
| Fer2 | PF00111.19 | 11.2000; | 2Fe—2S iron-sulfur cluster binding domain |
| Fer2_BFD | PF04324.7 | 16.0000; | BFD-like [2Fe—2S] binding domain |
| Fer4 | PF00037.19 | 15.5000; | 4Fe—4S binding domain |
| Ferric_reduct | PF01794.11 | −7.0000; | Ferric reductase like transmembrane component |
| Flavodoxin_1 | PF00258.17 | 6.3000; | Flavodoxin |
| FleQ | PF06490.3 | −14.4000; | Flagellar regulatory protein FleQ |
| Form_Nir_trans | PF01226.9 | −131.5000; | Formate/nitrite transporter |
| FrhB_FdhB_C | PF04432.5 | −42.4000; | Coenzyme F420 hydrogenase/dehydrogenase, |
| | | | beta subunit C terminus |
| FrhB_FdhB_N | PF04422.5 | 4.6000; | Coenzyme F420 hydrogenase/dehydrogenase, |
| | | | beta subunit N terminus |
| FtsH_ext | PF06480.7 | 6.8000; | FtsH Extracellular |
| Fumble | PF03630.6 | 25.0000; | Fumble |
| GAF | PF01590.18 | 23.0000; | GAF domain |
| GATA | PF00320.19 | 28.5000; | GATA zinc finger |
| GATase_2 | PF00310.13 | −91.6000; | Glutamine amidotransferases class-II |
| GCC2_GCC3 | PF07699.5 | 30.0000; | GCC2 and GCC3 |
| GDPD | PF03009.9 | −18.0000; | Glycerophosphoryl diester phosphodiesterase |
| | | | family |
| GFO_IDH_MocA | PF01408.14 | 1.0000; | Oxidoreductase family, NAD-binding |
| | | | Rossmann fold |
| GGDEF | PF00990.13 | −45.0000; | GGDEF domain |
| GH3 | PF03321.5 | −336.0000; | GH3 auxin-responsive promoter |
| GRAS | PF03514.6 | −181.6000; | GRAS family transcription factor |
| GSPII_E | PF00437.12 | −98.1000; | Type II/IV secretion system protein |
| GSPII_E_N | PF05157.7 | 5.7000; | GSPII_E N-terminal domain |
| GTP1_OBG | PF01018.14 | −56.2000; | GTP1/OBG |
| GTP_EFTU | PF00009.19 | 8.0000; | Elongation factor Tu GTP binding domain |
| GTP_EFTU_D2 | PF03144.17 | 25.0000; | Elongation factor Tu domain 2 |
| GTP_EFTU_D3 | PF03143.9 | 14.3000; | Elongation factor Tu C-terminal domain |
| GUCT | PF08152.4 | 25.0000; | GUCT (NUC152) domain |
| GXGXG | PF01493.11 | 5.0000; | GXGXG motif |
| Gal_Lectin | PF02140.10 | 25.0000; | Galactose binding lectin domain |
| GatB_Yqey | PF02637.10 | −28.5000; | GatB domain |
| Gate | PF07670.6 | 29.5000; | Nucleoside recognition |
| GcpE | PF04551.6 | −312.3000; | GcpE protein |
| GerE | PF00196.11 | 6.1000; | Bacterial regulatory proteins, luxR family |
| Gln-synt_C | PF00120.16 | −124.0000; | Glutamine synthetase, catalytic domain |
| Gln-synt_N | PF03951.11 | 9.0000; | Glutamine synthetase, beta-Grasp domain |
| Globin | PF00042.14 | −7.3000; | Globin |
| Glt_symporter | PF03616.6 | −272.0000; | Sodium/glutamate symporter |
| Glu_syn_central | PF04898.6 | 25.0000; | Glutamate synthase central domain |
| Glu_synthase | PF01645.9 | −198.4000; | Conserved region in glutamate synthase |
| Glutaredoxin | PF00462.16 | 17.2000; | Glutaredoxin |
| Glyco_hydro_1 | PF00232.10 | −301.8000; | Glycosyl hydrolase family 1 |
| Glyco_hydro_28 | PF00295.9 | −97.0000; | Glycosyl hydrolases family 28 |
| Glyco_hydro_35 | PF01301.11 | −184.5000; | Glycosyl hydrolases family 35 |
| Glyco_hydro_42 | PF02449.7 | −230.8000; | Beta-galactosidase |
| Glyco_transf_20 | PF00982.13 | −243.6000; | Glycosyltransferase family 20 |
| Glyco_transf_9 | PF01075.9 | −62.2000; | Glycosyltransferase family 9 |
| | | | (heptosyltransferase) |
| Glycos_transf_1 | PF00534.12 | −7.3000; | Glycosyl transferases group 1 |
| Glycos_transf_2 | PF00535.18 | 17.6000; | Glycosyl transferase family 2 |
| Got1 | PF04178.4 | 25.0000; | Got1-like family |
| Gp_dh_C | PF02800.12 | −64.1000; | Glyceraldehyde 3-phosphate dehydrogenase, C- |
| | | | terminal domain |
| Guanylate_cyc | PF00211.12 | −23.7000; | Adenylate and Guanylate cyclase catalytic |
| | | | domain |
| HALZ | PF02183.10 | 18.1000; | Homeobox associated leucine zipper |
| HAMP | PF00672.17 | 19.8000; | HAMP domain |
| HATPase_c | PF02518.18 | 22.4000; | Histidine kinase-, DNA gyrase B-, and HSP90- |
| | | | like ATPase |
| HD | PF01966.14 | 17.8000; | HD domain |
| HD-ZIP_N | PF04618.4 | 25.0000; | HD-ZIP protein N terminus |
| HDAC_interact | PF08295.4 | −17.1000; | Histone deacetylase (HDAC) interacting |
| HEPN | PF05168.6 | 0.1000; | IIEPN domain |
| HIG_1_N | PF04588.5 | −5.2000; | Hypoxia induced protein conserved region |
| HLH | PF00010.18 | 8.3000; | Helix-loop-helix DNA-binding domain |
| HMG_box | PF00505.11 | 4.1000; | HMG (high mobility group) box |
| HPPK | PF01288.12 | 25.0000; | 7,8-dihydro-6-hydroxymethylpterin- |
| | | | pyrophosphokinase (HPPK) |
| HR1 | PF02185.8 | 0.0000; | Hr1 repeat |
| HR_lesion | PF05514.3 | 25.0000; | HR-like lesion-inducing |
| HSP20 | PF00011.13 | 13.0000; | Hsp20/alpha crystallin family |
| HSP70 | PF00012.12 | −286.0000; | Hsp70 protein |
| HTH_3 | PF01381.14 | 27.7000; | Helix-turn-helix |
| HTH_6 | PF01418.9 | 0.0000; | Helix-turn-helix domain, rpiR family |
| HTH_8 | PF02954.11 | 17.3000; | Bacterial regulatory protein, Fis family |
| HTH_AraC | PF00165.15 | 22.0000; | Bacterial regulatory helix-turn-helix proteins, |
| | | | AraC family |
| Helicase_C | PF00271.23 | 6.9000; | Helicase conserved C-terminal domain |
| HemolysinCabind | PF00353.11 | 5.0000; | Hemolysin-type calcium-binding repeat (2 |
| | | | copies) |
| HisKA | PF00512.17 | 22.1000; | His Kinase A (phosphoacceptor) domain |
| HisKA_3 | PF07730.5 | 30.0000; | Histidine kinase |
| His_kinase | PF06580.5 | 25.0000; | Histidine kinase |
| Homeobox | PF00046.21 | −4.1000; | Homeobox domain |
| Homoserine_dh | PF00742.11 | −5.0000; | Homoserine dehydrogenase |
| Hpt | PF01627.15 | 25.0000; | Hpt domain |
| Hydrolase | PF00702.18 | 13.6000; | haloacid dehalogenase-like hydrolase |
| Hydrolase_3 | PF08282.4 | −64.8000; | haloacid dehalogenase-like hydrolase |
| IGPD | PF00475.10 | 25.0000; | Imidazoleglycerol-phosphate dehydratase |
| IQ | PF00612.19 | 18.3000; | IQ calmodulin-binding motif |
| IlvN | PF07991.4 | −75.8000; | Acetohydroxy acid isomeroreductase, catalytic |
| | | | domain |
| K-box | PF01486.9 | 0.0000; | K-box region |
| KA1 | PF02149.11 | 25.0000; | Kinase associated domain 1 |
| KH_2 | PF07650.9 | 5.0000; | KH domain |
| KOW | PF00467.21 | 29.1000; | KOW motif |
| KR | PF08659.2 | −74.3000; | KR domain |
| Kelch_1 | PF01344.17 | 11.7000; | Kelch motif |
| Kelch_2 | PF07646.7 | 14.0000; | Kelch motif |
| Kinesin | PF00225.15 | −135.0000; | Kinesin motor domain |
| LIM | PF00412.14 | 0.0000; | LIM domain |
| LRRNT_2 | PF08263.4 | 18.6000; | Leucine rich repeat N-terminal domain |
| LRR_1 | PF00560.25 | 10.9000; | Leucine Rich Repeat |
| LRR_2 | PF07723.5 | 14.5000; | Leucine Rich Repeat |
| LRR_3 | PF07725.4 | 20.0000; | Leucine Rich Repeat |
| Lact-deh-memb | PF09330.3 | 25.0000; | D-lactate dehydrogenase, membrane binding |
| Lactamase_B | PF00753.19 | 24.6000; | Metallo-beta-lactamase superfamily |
| Lectin_legB | PF00139.11 | −110.1000; | Legume lectin domain |
| Lipase_GDSL | PF00657.14 | 13.9000; | GDSL-like Lipase/Acylhydrolase |
| Lipoprotein_9 | PF03180.6 | −121.0000; | NLPA lipoprotein |
| LisH | PF08513.3 | 20.7000; | LisH |
| LysM | PF01476.12 | 20.0000; | LysM domain |
| MBD | PF01429.11 | 12.9000; | Methyl-CpG binding domain |
| MCD | PF05292.3 | 25.0000; | Malonyl-CoA decarboxylase (MCD) |
| MCPsignal | PF00015.13 | −88.3000; | Methyl-accepting chemotaxis protein (MCP) |
| | | | signaling domain |
| MEKHLA | PF08670.3 | −59.7000; | MEKHLA domain |
| MFS_1 | PF07690.8 | 23.5000; | Major Facilitator Superfamily |
| MIT | PF04212.10 | 18.3000; | MIT (microtubule interacting and transport) |
| | | | domain |
| MMR_HSR1 | PF01926.15 | 31.2000; | GTPase of unknown function |
| MOFRL | PF05161.5 | 25.0000; | MOFRL family |
| MORN | PF02493.12 | 0.0000; | MORN repeat |
| MOSC | PF03473.9 | 25.0000; | MOSC domain |
| MOSC_N | PF03476.8 | 25.0000; | MOSC N-terminal beta barrel domain |
| Malic_M | PF03949.7 | −143.9000; | Malic enzyme, NAD binding domain |
| MaoC_dehydratas | PF01575.11 | −10.9000; | MaoC like domain |
| Metal_hydrol | PF10118.1 | −74.9000; | Predicted metal-dependent hydrolase |
| Metallophos | PF00149.20 | 22.0000; | Calcineurin-like phosphoesterase |
| MethyltransfD12 | PF02086.7 | −49.4000; | D12 class N6 adenine-specific DNA |
| | | | methyltransferase |
| Methyltransf_11 | PF08241.4 | 24.4000; | Methyltransferase domain |
| Methyltransf_12 | PF08242.4 | 23.0000; | Methyltransferase domain |
| Methyltransf_16 | PF10294.1 | −43.0000; | Putative methyltransferase |
| Methyltransf_2 | PF00891.10 | −103.8000; | O-methyltransferase |
| Miro | PF08477.5 | 10.8000; | Miro-like protein |
| Mito_carr | PF00153.19 | 0.0000; | Mitochondrial carrier protein |
| Molybdop_Fe4S4 | PF04879.8 | 13.6000; | Molybdopterin oxidoreductase Fe4S4 domain |
| Molybdopterin | PF00384.14 | −50.0000; | Molybdopterin oxidoreductase |
| Molydop_binding | PF01568.13 | 1.1000; | Molydopterin dinucleotide binding domain |
| Monooxygenase_B | PF04744.4 | 25.0000; | Monooxygenase subunit B protein |
| Mur_ligase | PF01225.17 | 4.9000; | Mur ligase family, catalytic domain |
| Mur_ligase_C | PF02875.13 | 8.0000; | Mur ligase family, glutamate ligase domain |
| Mur_ligase_M | PF08245.4 | −44.7000; | Mur ligase middle domain |
| Myb_DNA-binding | PF00249.23 | 21.7000; | Myb-like DNA-binding domain |
| Myosin_N | PF02736.11 | 9.0000; | Myosin N-terminal SH3-like domain |
| Myosin_head | PF00063.13 | −372.4000; | Myosin head (motor domain) |
| N6_N4_Mtase | PF01555.10 | −36.4000; | DNA methylase |
| NAD_Gly3P_dh_N | PF01210.15 | −44.0000; | NAD-dependent glycerol-3-phosphate |
| | | | dehydrogenase N-terminus |
| NAD_binding_1 | PF00175.13 | −3.9000; | Oxidoreductase NAD-binding domain |
| NAD_binding_2 | PF03446.7 | −63.5000; | NAD binding domain of 6-phosphogluconate |
| | | | dehydrogenase |
| NAD_binding_3 | PF03447.8 | −1.7000; | Homoserine dehydrogenase, NAD binding |
| | | | domain |
| NAD_binding_6 | PF08030.4 | −23.6000; | Ferric reductase NAD binding domain |
| NAF | PF03822.6 | 4.5000; | NAF domain |
| NAGLU | PF05089.4 | 25.0000; | Alpha-N-acetylglucosaminidase (NAGLU) |
| NAM | PF02365.7 | −19.0000; | No apical meristem (NAM) protein |
| NB-ARC | PF00931.14 | −79.5000; | NB-ARC domain |
| NIR_SIR | PF01077.14 | −18.7000; | Nitrite and sulphite reductase 4Fe—4S domain |
| NIR_SIR_ferr | PF03460.9 | 2.4000; | Nitrite/Sulfite reductase ferredoxin-like half |
| | | | domain |
| NLE | PF08154.4 | 14.0000; | NLE (NUC135) domain |
| NMT1 | PF09084.3 | 20.0000; | NMT1/THI5 like |
| NOG1 | PF06858.6 | 50.0000; | Nucleolar GTP-binding protein 1 (NOG1) |
| NTP_transf_2 | PF01909.15 | 18.8000; | Nucleotidyltransferase domain |
| NTP_transferase | PF00483.15 | −39.9000; | Nucleotidyl transferase |
| NUDIX | PF00293.20 | 0.2000; | NUDIX domain |
| Ndr | PF03096.6 | −82.0000; | Ndr family |
| NifU | PF01106.9 | −5.6000; | NifU-like domain |
| Nitroreductase | PF00881.16 | 14.2000; | Nitroreductase family |
| Nramp | PF01566.10 | −171.4000; | Natural resistance-associated macrophage |
| | | | protein |
| OB_RNB | PF08206.3 | 4.1000; | Ribonuclease B OB domain |
| OKR_DC_1 | PF01276.12 | −206.6000; | Orn/Lys/Arg decarboxylase, major domain |
| OKR_DC_1_C | PF03711.7 | −52.0000; | Orn/Lys/Arg decarboxylase, C-terminal domain |
| OKR_DC_1_N | PF03709.7 | 7.0000; | Orn/Lys/Arg decarboxylase, N-terminal domain |
| OMPdecase | PF00215.16 | −45.6000; | Orotidine 5′-phosphate decarboxylase/HUMPS |
| | | | family |
| OPT | PF03169.7 | −233.0000; | OPT oligopeptide transporter protein |
| OpuAC | PF04069.4 | −45.4000; | Substrate binding domain of ABC-type glycine |
| | | | betaine transport system |
| Orn_Arg_deC_N | PF02784.8 | −76.0000; | Pyridoxal-dependent decarboxylase, pyridoxal |
| | | | binding domain |
| Orn_DAP_Arg_deC | PF00278.14 | 6.7000; | Pyridoxal-dependent decarboxylase, C-terminal |
| | | | sheet domain |
| P-II | PF00543.14 | −29.0000; | Nitrogen regulatory protein P-II |
| PAH | PF02671.13 | 25.0000; | Paired amphipathic helix repeat |
| PAPS_reduct | PF01507.11 | −53.4000; | Phosphoadenosine phosphosulfate reductase |
| | | | family |
| PAS | PF00989.16 | 0.0000; | PAS fold |
| PAS_2 | PF08446.3 | −2.1000; | PAS fold |
| PAS_3 | PF08447.3 | 17.2000; | PAS fold |
| PAS_4 | PF08448.2 | 16.4000; | PAS fold |
| PB1 | PF00564.16 | 12.3000; | PB1 domain |
| PBP | PF01161.12 | −20.6000; | Phosphatidylethanolamine-binding protein |
| PC_rep | PF01851.14 | 0.0000; | Proteasome/cyclosome repeat |
| PGI | PF00342.11 | −146.0000; | Phosphoglucose isomerase |
| PGM_PMM_I | PF02878.8 | −37.5000; | Phosphoglucomutase/phosphomannomutase, |
| | | | alpha/beta/alpha domain I |
| PGM_PMM_II | PF02879.8 | −20.0000; | Phosphoglucomutase/phosphomannomutase, |
| | | | alpha/beta/alpha domain II |
| PHD | PF00628.21 | 26.2000; | PHD-finger |
| PH_2 | PF08458.2 | −6.6000; | Plant pleckstrin homology-like region |
| PLAC8 | PF04749.9 | −1.1000; | PLAC8 family |
| PMEI | PF04043.7 | 25.0000; | Plant invertase/pectin methylesterase inhibitor |
| PMSR | PF01625.13 | −32.7000; | Peptide methionine sulfoxide reductase |
| PNK3P | PF08645.3 | −67.6000; | Polynucleotide kinase 3 phosphatase |
| PNTB | PF02233.8 | −341.0000; | NAD(P) transhydrogenase beta subunit |
| POX | PF07526.3 | −39.4000; | Associated with HOX |
| PP-binding | PF00550.17 | 25.3000; | Phosphopantetheine attachment site |
| PP2C | PF00481.13 | −36.3000; | Protein phosphatase 2C |
| PPR | PF01535.12 | 1.6000; | PPR repeat |
| PRP4 | PF08799.3 | 25.0000; | pre-mRNA processing factor 4 (PRP4) like |
| PSCyt1 | PF07635.3 | 50.0000; | Planctomycete cytochrome C |
| PTA_PTB | PF01515.11 | −165.5000; | Phosphate acetyl/butaryl transferase |
| PTR2 | PF00854.13 | −50.0000; | POT family |
| PWWP | PF00855.9 | 0.3000; | PWWP domain |
| Pantoate_ligase | PF02569.7 | −106.0000; | Pantoate-beta-alanine ligase |
| Pantoate_transf | PF02548.7 | −93.0000; | Ketopantoate hydroxymethyltransferase |
| PduL | PF06130.4 | 25.0000; | Propanediol utilisation protein PduL |
| Pectinesterase | PF01095.11 | −222.9000; | Pectinesterase |
| PepSY_TM | PF03929.8 | 11.9000; | PepSY-associated TM helix |
| Peptidase_C14 | PF00656.14 | −22.5000; | Caspase domain |
| Peptidase_C2 | PF00648.13 | −183.5000; | Calpain family cysteine protease |
| Peptidase_M3 | PF01432.12 | −146.0000; | Peptidase family M3 |
| Peptidase_M41 | PF01434.10 | −139.8000; | Peptidase family M41 |
| Peptidase_S10 | PF00450.14 | −198.0000; | Serine carboxypeptidase |
| Peptidase_S24 | PF00717.15 | 10.0000; | Peptidase S24-like |
| Pericardin_rpt | PF07054.3 | 0.0000; | Pericardin like repeat |
| Perip1a_BP_1 | PF00532.13 | −44.9000; | Periplasmic binding proteins and sugar binding |
| | | | domain of the LacI family |
| Phage_fiber | PF03335.5 | 0.0000; | Phage tail fibre repeat |
| Phytochrome | PF00360.12 | 13.0000; | Phytochrome region |
| PilZ | PF07238.6 | 19.6000; | PilZ domain |
| Pkinase | PF00069.17 | 70.3000; | Protein kinase domain |
| Pkinase_C | PF00433.16 | 15.3000; | Protein kinase C terminal domain |
| Pkinase_Tyr | PF07714.9 | −155.0000; | Protein tyrosine kinase |
| Plant_zn_clust | PF10533.1 | 25.0000; | Plant zinc cluster domain |
| Plus-3 | PF03126.10 | 25.0000; | Plus-3 domain |
| Pollen_allerg_1 | PF01357.13 | 17.2000; | Pollen allergen |
| Pribosyltran | PF00156.19 | 20.0000; | Phosphoribosyl transferase domain |
| Proteasome | PF00227.18 | −36.7000; | Proteasome A-type and B-type |
| PseudoU_synth_2 | PF00849.14 | −18.0000; | RNA pseudouridylate synthase |
| Pterin_bind | PF00809.14 | −53.7000; | Pterin binding enzyme |
| Pyr_redox | PF00070.19 | 5.0000; | Pyridine nucleotide-disulphide oxidoreductase |
| Pyr_redox_2 | PF07992.6 | −20.0000; | Pyridine nucleotide-disulphide oxidoreductase |
| Pyridox_oxidase | PF01243.12 | 17.1000; | Pyridoxamine 5′-phosphate oxidase |
| Pyrophosphatase | PF00719.11 | −41.0000; | Inorganic pyrophosphatase |
| RCC1 | PF00415.10 | 21.2000; | Regulator of chromosome condensation (RCC1) |
| RIO1 | PF01163.14 | −89.1000; | RIO1 family |
| RNA_Me_trans | PF04252.5 | 25.0000; | Predicted SAM-dependent RNA |
| | | | methyltransferase |
| RNA_pol_L | PF01193.16 | 16.9000; | RNA polymerase Rpb3/Rpb11 dimerisation |
| | | | domain |
| RRM_1 | PF00076.14 | 17.9000; | RNA recognition motif. (a.k.a. RRM, RBD, or |
| | | | RNP domain) |
| RVT_1 | PF00078.19 | 32.0000; | Reverse transcriptase (RNA-dependent DNA |
| | | | polymerase) |
| RVT_2 | PF07727.6 | −180.0000; | Reverse transcriptase (RNA-dependent DNA |
| | | | polymerase) |
| RWP-RK | PF02042.7 | 25.0000; | RWP-RK domain |
| RecA | PF00154.13 | −100.6000; | recA bacterial DNA recombination protein |
| Reg_prop | PF07494.3 | 5.4000; | Two component regulator propeller |
| ResIII | PF04851.7 | 0.8000; | Type III restriction enzyme, res subunit |
| Response_reg | PF00072.16 | 4.1000; | Response regulator receiver domain |
| Rho_N | PF07498.4 | 31.0000; | Rho termination factor, N-terminal domain |
| Rhodanese | PF00581.12 | 25.0000; | Rhodanese-like domain |
| Rhomboid | PF01694.14 | −1.2000; | Rhomboid family |
| Ribosomal_L21p | PF00829.13 | −23.0000; | Ribosomal prokaryotic L21 protein |
| Ribosomal_S19 | PF00203.13 | 11.0000; | Ribosomal protein S19 |
| Ribosomal_S9 | PF00380.11 | −24.0000; | Ribosomal protein S9/S16 |
| Rieske | PF00355.18 | −7.0000; | Rieske [2Fe—2S] domain |
| RolB_RolC | PF02027.9 | 25.0000; | RolB/RolC glucosidase family |
| Rrf2 | PF02082.12 | −17.5000; | Transcriptional regulator |
| RrnaAD | PF00398.12 | −73.3000; | Ribosomal RNA adenine dimethylase |
| Rubredoxin | PF00301.12 | 14.0000; | Rubredoxin |
| S-AdoMet_synt_M | PF02772.8 | 25.0000; | S-adenosylmethionine synthetase, central |
| | | | domain |
| S4 | PF01479.17 | 15.0000; | S4 domain |
| S6PP | PF05116.5 | −113.7000; | Sucrose-6F-phosphate phosphohydrolase |
| SAM_1 | PF00536.22 | 11.3000; | SAM domain (Sterile alpha motif) |
| SAM_2 | PF07647.9 | 0.0000; | SAM domain (Sterile alpha motif) |
| SBP | PF03110.6 | 25.0000; | SBP domain |
| SBP_bac_3 | PF00497.12 | 16.6000; | Bacterial extracellular solute-binding proteins, |
| | | | family 3 |
| SCP | PF00188.18 | 10.0000; | SCP-like extracellular protein |
| SET | PF00856.20 | 23.5000; | SET domain |
| SH3BGR | PF04908.7 | −12.0000; | SH3-binding, glutamic acid-rich protein |
| SIS | PF01380.14 | 0.0000; | SIS domain |
| SLA_LP_auto_ag | PF05889.5 | −170.7000; | Soluble liver antigen/liver pancreas antigen |
| | | | (SLA/LP autoantigen) |
| SMC_N | PF02463.11 | −95.8000; | RecF/RecN/SMC N terminal domain |
| SOUL | PF04832.4 | 25.0000; | SOUL heme-binding protein |
| SPRY | PF00622.20 | 10.0000; | SPRY domain |
| SRF-TF | PF00319.10 | 11.0000; | SRF-type transcription factor (DNA-binding |
| | | | and dimerisation domain) |
| SSF | PF00474.9 | −162.3000; | Sodium: solute symporter family |
| START | PF01852.11 | −20.7000; | START domain |
| STAS | PF01740.13 | 0.0000; | STAS domain |
| SbmA_BacA | PF05992.4 | −145.6000; | SbmA/BacA-like family |
| SelR | PF01641.10 | −66.5000; | SelR domain |
| Shikimate_DH | PF01488.12 | −4.4000; | Shikimate/quinate 5-dehydrogenase |
| Shugoshin_C | PF07557.3 | 25.0000; | Shugoshin C terminus |
| Sigma54_activat | PF00158.18 | −95.0000; | Sigma-54 interaction domain |
| Sina | PF03145.8 | −48.4000; | Seven in absentia protein family |
| SirA | PF01206.9 | 7.0000; | SirA-like protein |
| SpoIIE | PF07228.4 | −13.7000; | Stage II sporulation protein E (SpoIIE) |
| Spore_permease | PF03845.5 | −134.0000; | Spore germination protein |
| Ssl1 | PF04056.6 | −151.8000; | Ssl1-like |
| Suc_Fer-like | PF06999.4 | −42.4000; | Sucrase/ferredoxin-like |
| Sulfate_transp | PF00916.12 | −131.5000; | Sulfate transporter family |
| Synaptobrevin | PF00957.13 | 25.0000; | Synaptobrevin |
| TAT_signal | PF10518.1 | 22.9000; | TAT (twin-arginine translocation) pathway |
| | | | signal sequence |
| TFIID-31 kDa | PF02291.7 | 25.0000; | Transcription initiation factor IID, 31 kD subunit |
| TFIID_90 kDa | PF04494.7 | 25.0000; | WD40 associated region in TFIID subunit |
| TGS | PF02824.13 | 5.6000; | TGS domain |
| TIR | PF01582.12 | 43.5000; | TIR domain |
| TPP_enzyme_C | PF02775.13 | 19.7000; | Thiamine pyrophosphate enzyme, C-terminal |
| | | | TPP binding domain |
| TPP_enzyme_M | PF00205.14 | −8.1000; | Thiamine pyrophosphate enzyme, central |
| | | | domain |
| TPP_enzyme_N | PF02776.10 | −70.0000; | Thiamine pyrophosphate enzyme, N-terminal |
| | | | TPP binding domain |
| TPR_1 | PF00515.20 | 16.1000; | Tetratricopeptide repeat |
| TPR_2 | PF07719.9 | 23.9000; | Tetratricopeptide repeat |
| Thg1 | PF04446.4 | 25.0000; | tRNAHis guanylyltransferase |
| ThiS | PF02597.12 | 19.3000; | ThiS family |
| Thioesterase | PF00975.12 | −10.5000; | Thioesterase domain |
| Thiolase_C | PF02803.10 | −30.7000; | Thiolase, C-terminal domain |
| Thiolase_N | PF00108.15 | −129.5000; | Thiolase, N-terminal domain |
| Thioredoxin | PF00085.12 | −16.6000; | Thioredoxin |
| Thymidylate_kin | PF02223.9 | 2.3000; | Thymidylate kinase |
| Torsin | PF06309.3 | −158.6000; | Torsin |
| TraB | PF01963.9 | −62.0000; | TraB family |
| Trans_reg_C | PF00486.20 | 25.0000; | Transcriptional regulatory protein, C terminal |
| Transaldolase | PF00923.11 | −49.0000; | Transaldolase |
| Trehalose_PPase | PF02358.8 | −49.4000; | Trehalose-phosphatase |
| TrkA_C | PF02080.13 | 25.0000; | TrkA-C domain |
| TrkA_N | PF02254.10 | 4.7000; | TrkA-N domain |
| Trp_Tyr_perm | PF03222.5 | −232.6000; | Tryptophan/tyrosine permease family |
| Trp_halogenase | PF04820.6 | −283.2000; | Tryptophan halogenase |
| Tub | PF01167.10 | −98.0000; | Tub family |
| Tubulin | PF00091.17 | −48.7000; | Tubulin/FtsZ family, GTPase domain |
| Tup_N | PF08581.2 | 25.0000; | Tup N-terminal |
| U-box | PF04564.7 | −7.6000; | U-box domain |
| U3_snoRNA_C | PF09384.2 | 25.0000; | U3 small nucleolar RNA C terminal |
| UBA | PF00627.23 | 22.4000; | UBA/TS-N domain |
| UBA_2 | PF08587.3 | 25.0000; | Ubiquitin associated domain (UBA) |
| UDPG_MGDP_dh_N | PF03721.6 | −67.4000; | UDP-glucose/GDP-mannose dehydrogenase |
| | | | family, NAD binding domain |
| UIM | PF02809.12 | 16.6000; | Ubiquitin interaction motif |
| UVR | PF02151.11 | 16.0000; | UvrB/uvrC motif |
| Usp | PF00582.18 | 22.3000; | Universal stress protein family |
| Utp21 | PF04192.4 | 25.0000; | Utp21 specific WD40 associated putative |
| | | | domain |
| Vps4_C | PF09336.2 | −4.6000; | Vps4 C terminal oligomerisation domain |
| WD40 | PF00400.24 | 21.5000; | WD domain, G-beta repeat |
| WRKY | PF03106.7 | −6.7000; | WRKY DNA -binding domain |
| WW | PF00397.18 | 0.0000; | WW domain |
| XH | PF03469.6 | 25.0000; | XH domain |
| XS | PF03468.6 | 25.0000; | XS domain |
| XYPPX | PF02162.9 | 0.7000; | XYPPX repeat |
| Xan_ur_permease | PF00860.12 | −146.7000; | Permease family |
| YLP | PF02757.9 | 2.7000; | YLP motif |
| Y_Y_Y | PF07495.5 | 5.0000; | Y_Y_Y domain |
| Zn_clus | PF00172.10 | 12.0000; | Fungal Zn(2)-Cys(6) binuclear cluster domain |
| adh_short | PF00106.17 | −40.2000; | short chain dehydrogenase |
| bZIP_1 | PF00170.13 | 24.8000; | bZIP transcription factor |
| bZIP_2 | PF07716.7 | 15.0000; | Basic region leucine zipper |
| cNMP_binding | PF00027.21 | 20.6000; | Cyclic nucleotide-binding domain |
| eIF-5a | PF01287.12 | 9.6000; | Eukaryotic initiation factor 5A hypusine, DNA- |
| | | | binding OB fold |
| eIF2A | PF08662.3 | −71.9000; | Eukaryotic translation initiation factor eIF2A |
| eRF1_1 | PF03463.7 | −12.9000; | eRF1 domain 1 |
| eRF1_2 | PF03464.7 | 3.6000; | eRF1 domain 2 |
| eRF1_3 | PF03465.7 | 1.4000; | eRF1 domain 3 |
| Efhand | PF00036.24 | 21.7000; | EF hand |
| fn3 | PF00041.13 | 7.2000; | Fibronectin type III domain |
| mTERF | PF02536.6 | −60.0000; | mTERF |
| Malic | PF00390.11 | 25.0000; | Malic enzyme, N-terminal domain |
| oligo_HPY | PF08352.4 | 2.6000; | Oligopeptide/dipeptide transporter, C-terminal |
| | | | region |
| p450 | PF00067.14 | −105.0000; | Cytochrome P450 |
| Peroxidase | PF00141.15 | −10.0000; | Peroxidase |
| Rve | PF00665.18 | 32.0000; | Integrase core domain |
| tRNA-synt_1c | PF00749.13 | −130.2000; | tRNA synthetases class I (E and Q), catalytic |
| | | | domain |
| tRNA-synt_1c_C | PF03950.10 | 25.0000; | tRNA synthetases class I (E and Q), anti-codon |
| | | | binding domain |
| tRNA-synt_1g | PF09334.3 | −272.5000; | tRNA synthetases class I (M) |
| tRNA_synt_1c_R1 | PF04558.7 | 25.0000; | Glutaminyl-tRNA synthetase, non-specific |
| | | | RNA binding region part 1 |
| tRNA_synt_1c_R2 | PF04557.7 | 25.0000; | Glutaminyl-tRNA synthetase, non-specific |
| | | | RNA binding region part 2 |
| Ubiquitin | PF00240.15 | 19.4000; | Ubiquitin family |
| zf-B_box | PF00643.16 | 15.3000; | B-box zinc finger |
| zf-C3HC4 | PF00097.17 | 16.0000; | Zinc finger, C3HC4 type (RING finger) |
| zf-CCCH | PF00642.16 | 0.0000; | Zinc finger C-x8-C-x5-C-x3-H type (and |
| | | | similar) |
| zf-CCHC | PF00098.15 | 17.9000; | Zinc knuckle |
| zf-CDGSH | PF09360.2 | 10.4000; | Iron-binding zinc finger CDGSH type |
| zf-CW | PF07496.7 | 25.0000; | CW-type Zinc Finger |
| zf-Dof | PF02701.7 | 25.0000; | Dof domain, zinc finger |
| zf-GRF | PF06839.4 | 22.7000; | GRF zinc finger |
| zf-RanBP | PF00641.10 | 23.0000; | Zn-finger in Ran binding protein and others |
| zf-TRAF | PF02176.10 | 18.6000; | TRAF-type zinc finger |
| zf-dskA_traR | PF01258.9 | 21.0000; | Prokaryotic dksA/traR C4-type zinc finger |

1. (canceled)

2. A plant cell having a recombinant DNA construct comprising a promoter that is functional in said plant cell and is operably linked to a polynucleotide that
a) encodes a polypeptide having an amino acid sequence sharing at least 90% identity over at least 90% of the length of a reference sequence having the amino acid sequence selected from the group consisting of SEQ ID NOs:308-36442 and 36447-36478 when said amino acid sequence is aligned with said reference sequence; or
b) is transcribed into an RNA molecule that suppresses the level of said polypeptide.

3. The plant cell according to claim 2, wherein said recombinant DNA construct is stably integrated into a chromosome in the nucleus of said plant cell.

4. The plant cell according to claim 2, wherein said recombinant DNA construct provides for an enhanced trait as compared to control plants which do not contain said recombinant DNA; and wherein said enhanced trait is enhanced water use efficiency, enhanced cold tolerance, increased yield, enhanced nitrogen use efficiency, enhanced seed protein or enhanced seed oil.

5. The plant cell according to claim 2 further comprising DNA expressing a protein that provides tolerance from exposure to an herbicide comprising an agent applied at levels that are lethal to a wild type of said plant cell.

6. The plant cell according to claim 5 wherein the agent of said herbicide is a glyphosate, dicamba, or glufosinate compound.

7. A transgenic plant or seed comprising a plurality of plant cells of claim 2.

8. The transgenic plant or seed of claim 7, which is homozygous for said recombinant DNA.

9. The transgenic plant or seed according to claim 7, wherein said RNA molecule is a double-stranded RNA or antisense RNA.

10. The transgenic plant or seed according to claim 7, wherein said plant or seed is corn, soybean, cotton, canola, alfalfa, wheat, rice, sugarcane, or sugar beet.

11.-17. (canceled)

18. The plant cell according to claim 2, wherein said polypeptide has an amino acid sequence having at least 95% sequence identity to a reference sequence selected from the group consisting of SEQ ID NOs: 308-36442 and 36447-36478.

19. The plant cell according to claim 18, wherein said polypeptide has an amino acid sequence having at least 97% sequence identity to a reference sequence selected from the group consisting of SEQ ID NOs: 308-36442 and 36447-36478.

20. The plant cell according to claim 19, wherein said polypeptide has an amino acid sequence having at least 99% sequence identity to a reference sequence selected from the group consisting of SEQ ID NOs: 308-36442 and 36447-36478.

21. The plant cell according to claim 20, wherein said polypeptide has an amino acid sequence selected from the group consisting of SEQ ID NOs: 308-36442 and 36447-36478.

22. A method of growing a transgenic plant comprising:
a. planting a transgenic seed comprising a plurality of plant cells having a recombinant DNA construct comprising a promoter that is functional in said plant cell and is operably linked to a polynucleotide that:
i. encodes a polypeptide having an amino acid sequence sharing at least 90% sequence identity to over at least 90% of the length of a reference sequence having the amino acid sequence selected from the group consisting of SEQ ID NOs:308-36442 and 36447-36478; or
ii. is transcribed into an RNA molecule that suppresses the level of said polypeptide; and
b. growing a plant from said transgenic seed.

23. The method according to claim 22, wherein said polypeptide has an amino acid sequence sharing at least 95% sequence identity to a reference sequence selected from the group consisting of SEQ ID NOs:308-36442 and 36447-36478.

24. The method according to claim 23, wherein said polypeptide has an amino acid sequence having at least 97% sequence identity to a reference sequence selected from the group consisting of SEQ ID NOs: 308-36442 and 36447-36478.

25. The method according to claim 22, wherein said seed is from a corn, soybean, cotton, canola, alfalfa, wheat, rice, sugarcane, or sugar beet plant.

26. A transgenic plant or seed comprising a recombinant DNA construct, wherein said recombinant DNA construct comprises:
a. an exogenous promoter region which functions in a plant cell to cause the production of an RNA molecule; which is linked to
b. a structural nucleic acid molecule, wherein said structural nucleic acid molecule has a sequence selected from the group consisting of SEQ ID NOs:1-307.

27. The transgenic plant or seed according to claim 26, wherein said plant or seed is corn, soybean, cotton, canola, alfalfa, wheat, rice, sugarcane, or sugar beet.

28. The transgenic plant or seed according to claim 26, wherein said RNA molecule is a double-stranded RNA or antisense RNA.
